(19) United States
(12) Patent Application Publication
(10) Pub. No.: US 2023/0113563 A1
(43) Pub. Date: Apr. 13, 2023

(54) GUIDANCE AND NAVIGATION CONTROL (GNC) ANTIBODY-LIKE PROTEINSAND METHODS OF MAKING AND USING THEREOF

(71) Applicants: SYSTIMMUNE, INC., Redmond, WA (US); BAILI-BIO (CHENGDU) PHARMACEUTICAL CO., LTD., Chengdu, Sichuan (CN)

(72) Inventors: Yi Zhu, Redmond, WA (US); Dennis R. Goulet, Redmond, WA (US); Tsung-I Tsai, Redmond, WA (US); Soumili Chatterjee, Bothell, WA (US); Blair Renshaw, Renton, WA (US); Christopher G. Vincent, Lynnwood, WA (US); Andrew Waight, Kenmore, WA (US); Nga Sze Amanda Mak, Mukilteo, WA (US)

(21) Appl. No.: 17/909,358

(22) PCT Filed: Mar. 17, 2021
(86) PCT No.: PCT/US21/22849

§ 371 Date: Sep. 2, 2022

Related U.S. Application Data
(60) Provisional application No. 62/991,042, filed on Mar. 17, 2020.

(51) Int. Cl. C07K 16/28; C07K 16/30; A61P 35/00; A61K 47/68

(52) U.S. Cl. CPC C07K 16/2851; C07K 16/2827; C07K 16/2878; C07K 16/2809; C07K 16/30; A61P 35/00; A61K 47/6803; A61K 47/6879; C07K 2317/31; C07K 2317/24; C07K 2317/55; C07K 2317/92

(57) ABSTRACT

The application provides multi-specific antibody-like proteins may comprising one or more binding domains including a first binding domain (D1), a second binding domain (D2), a third binding domain (D3), a fourth binding domain (D4), a fifth binding domain (D5), or a sixth binding domain (D6). The multi-specific antibody-like protein disclosed herein may be mono-specific, bi-specific, tri-specific, tetra-specific, penta-specific or hexa-specific. The binding domains such as D1, D2, D3, D4, D5, and D6 may each independently have a binding affinity to specificity against a T cell activating receptor, an immune cell receptor, an immune checkpoint molecule, a co-stimulation factor, a receptor of a leukocyte, a tumor antigen, a tumor associated antigen (TAA), a receptor of a tissue cell, a receptor of a cancer cell, or a combination thereof.

### CROSS REFERENCE TO RELATED APPLICATIONS

This application claims the benefit of the filing date of U.S. Provisional Application Ser. No. 62/991,042 filed Mar. 17, 2020 under 35 U.S.C. 119(e), the entire disclosures of which are incorporated by reference herein.

### TECHNICAL FIELD

The present application generally relates to the technical field of multi-specific antibody for cancer immunotherapy and more particularly relates to making and using Guidance and Navigation Control (GNC) antibodies with multiple binding activities against surface molecules of both immune cells and tumor cells.

### BACKGROUND

Cancer cells develop various strategies to evade the immune system. There is an urgent need to improve biological therapeutics functionality, specificity, potency, and efficacy. The success of targeted therapies in cancer treatment has also been impeded by various mechanisms of resistance. Tumor plasticity has emerged as a mode of targeted therapy evasion in various cancers, ranging from prostate and lung adenocarcinoma to melanoma and basal cell carcinoma. It is thought that the mechanisms interfering in shaping robust antitumor immune responses include at least some of the following categories: 1) defective tumor antigen processing or presentation; 2) lack of activating mechanisms; 3) inhibitory mechanisms and immunosuppressive state; and 4) resistant tumor cells (4). To overcome these mechanisms of escaping and resisting, new therapeutic strategies are designed to facilitate multiple immune effectors, including but not limited to, T-cell engager, checkpoint inhibitors, and innate immunity into the combination immunotherapy strategy. However, such strategies of combination therapy often mean two or more independent biologics products, which require manufacture of independent biologics as well as approval of the clinical safety and efficacy of each product. Combination therapies may target either immune cells or tumor cells or both. For instance, there are antibody therapies using bispecific antibodies targeting both CD3 and CD19 or CAR-T cell therapy comprising engineered T cells expressing anti-CD19 chimeric antibody. One common side effect from these immunotherapies is cytokine release syndrome, which is indicative of insufficient immune regulation. In this context, new strategies are needed to overcome tumor plasticity, i.e. the heterogeneous and dynamic expression of tumor antigens and/or resistant tumor cells, while acquiring additional immune regulation.

Towards this end, a platform of multi-specific antibodies, also known as Guidance and Navigation Control (GNC), has been established to facilitate multiple targeting of T-cell engagers, co-stimulation factors, checkpoint inhibitors, and tumor antigens (see Applicant's applications, WO/2019/005641, WO2019191120, and PCT/US20/59230, incorporated herein in its entirety). In addition, tetra-specific GNC (tetraGNC) antibodies may be used to manufacture GNC-T cell therapy for treating both liquid and solid tumors. In spite of multifunctional GNC molecules, epitope-negative tumor cells can remain un-targeted and consequently evade immunotherapies. For instance, the expression of NKG2D ligands are tightly regulated to prevent autoimmune tissue damage and therefore normal tissues generally do not express NKG2D ligands. Using the NKG2D receptor may therefore be an effective targeting mechanism for cancer immunotherapy via an innate immune recognition process. In this context, there is a clear need to further develop multi-specific antibodies related cell therapy. While a multi-specificity single drug remains highly desirable and cost-effective, it is technically challenging to design, express, and manufacture a potent and stable multi-specific antibody beyond tetra-GNC antibodies.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

The application provides proteins with binding specificities such as multi-specific antibody-like protein may include multi-specific antibodies, the fragments of these binding proteins may include without limitation scFv domain, Fab region, Fc domain, VH, VL, light chains, heavy chains, variable regions, and complementary determining region (CDR), methods of making and method using the multi-specific antibody-like proteins and fragments thereof.

In one embodiment, the multi-specific antibody-like protein may be multi-specific antibodies, monoclonal antibodies, isolated monoclonal antibodies, or humanized antibodies.

In one embodiment, the proteins may comprise various domains and regions such as binding domains. In one embodiment, the multi-specific antibody-like protein may include one or more binding domains including a first binding domain (D1), a second binding domain (D2), a third binding domain (D3), a fourth binding domain (D4), a fifth binding domain (D5), or a sixth binding domain (D6). The multi-specific antibody-like protein disclosed herein may be mono-specific, bi-specific, tri-specific, tetra-specific, penta-specific or hexa-specific.

In one embodiment, the binding domains such as D1, D2, D3, D4, D5, and D6 may each independently have a binding affinity to specificity against a T cell activating receptor, an immune cell receptor, an immune checkpoint molecule, a co-stimulation factor, a receptor of a leukocyte, a tumor antigen, a tumor associated antigen (TAA), a receptor of a tissue cell, a receptor of a cancer cell, or a combination thereof.

In one embodiment, the T cell activating receptor may comprise CD3. In one embodiment, the immune checkpoint receptor may comprise PD-L1, PD-1, TIGIT, TIM-3, LAG-3, CTLA4, BTLA, VISTA, PD-L2, CD160, LOX-1, siglec-15, CD47, HVEM SIRPα, CSF1R, CD73, Siglec-15, CD47, or a combination thereof. In one embodiment, the co-stimulating receptor may comprise 4-1BB, CD28, OX40, GITR, CD40L, CD40, ICOS, LIGHT, CD27, CD30, or a combination thereof. In one embodiment, the tumor associated antigen may comprise EGFR, HER2, HER3, EGRFVIII, CD19, BCMA, CD20, CD33, CD123, CD22, CD30, ROR1, CEA, LMP1, LMP2A, Mesothelin, PSMA, EpCAM, glypican-3, gpA33, GD2, TROP2, NKG2D ligand, CD39, CLDN18.2, DLL3, HLA-G, FcRH5, GPRC5D, LIV-1, MUC1, CD138, CD70, uPAR, CD38, or a combination thereof.

In one embodiment, the binding domain for a T cell activating receptor is adjacent to the binding domain for a tumor associated antigen (TAA).

In one embodiment, the D1, D3, D4, D5, and D6 may be independently a scFv domain, a receptor, or a ligand. In one embodiment, at least one, two, three, four, or five of D1, D3, D4, D5, and D6 in the hexa-specific antibody-like protein include an scFv domain. In one embodiment, all of D1, D3, D4, D5, and D6 are all scFv domains.

In one embodiment, at least one, two, three, four, or five of D1, D3, D4, D5, and D6 in the hexa-specific antibody-like protein include a receptor. In one embodiment, all of D1, D3, D4, D5, and D6 are all receptors.

In one embodiment, at least one, two, three, four, or five of D1, D3, D4, D5, and D6 in the hexa-specific antibody-like protein include a ligand. In one embodiment, all of D1, D3, D4, D5, and D6 are all ligands.

In one embodiment, the scFv domain may comprise a VH linked to a VL in the orientation of VH-VL or VL-VH. In one embodiment, the scFv domain may comprise a disulphide bond between the VL and the VH. In one embodiment, the disulfide bond is between VL100 and VH44 of the scFv domain. In one embodiment, the scFv domain may comprise a substitution R19S (Kabat) in the VH.

In one embodiment, the multi-specific antibody-like protein may include a Fc region. In one embodiment, the Fc region is engineered to eliminate effector cell functions including without limitation ADCC, ADCP, or CDC. In one embodiment, the Fc region comprise at least one mutation at L234A, L235A, G237A, or K322A (Eu numbering). In one embodiment, the Fc region comprise mutations at L234A/L235A/G237A/K322A. In one embodiment, the Fc region comprise mutations at L234A/L235A/K322A (Eu numbering).

The domains and regions may be linked through linkers. In one embodiment, the linker may comprise a (GₓS_{y})ₙ linker, wherein n, x and y each independently is an integer from 1 to 10. In one embodiment, n is 1, 2, 3, 4, 5, 6, 7, 8, 9, or 10. In one embodiment, x is 1, 2, 3, 4, 5, 6, 7, 8, 9, or 10. In one embodiment, y is 1, 2, 3, 4, 5, 6, 7, 8, 9, or 10.

In one aspect, the application provides hex-specific antibody-like proteins. In one embodiment, the hexa-specific antibody-like protein having a N-terminal and a C-terminal, may include in tandem from the N-terminal to the C-terminal, a first binding domain (D1) at the N-terminal, a Fab region as a second binding domain (D2) may include a light chain, a Fc region, a third binding domain (D3) having a binding affinity to PD-L1, and a fourth binding domain (D4) having a binding affinity to 4-1BB at the C-terminal, wherein the light chain may comprise a fifth binding domain (D5) covalently attached to the C-terminal and a sixth binding domain (D6) covalently attached to the N-terminal, and wherein D1, D2, D5, and D6 each independently may have a binding affinity to a tumor associated antigen (TAA) or CD3.

In one embodiment, the hexa-specific antibody-like protein may have D1 or D2 having binding affinity to CD3. In one embodiment, the hexa-specific antibody-like protein may have D1 having a binding affinity to CD3. In one embodiment, the hexa-specific antibody-like protein may have D2 having a binding affinity to CD3.

In one embodiment, the hexa-specific antibody-like protein may include D1 having a binding specificity against CD3, D2 having a binding specificity against EGFR, EGFRvIII, CD20, mesothelin, Claudin18.2, HER2, CD33or a combination thereof, D3 having a binding specificity against PD-L1, D4 having a binding specificity against 4-1BB, and D5 and D6 each independently having a binding specificity against a tumor associated antigen.

In one embodiment, the hexa-specific antibody-like protein may include D1 having a binding specificity against CD3, D2 having a binding specificity against a tumor associated antigen, D3 having a binding specificity against PD-L1, D4 having a binding specificity against 4-1BB, and D5 and D6 each independently having a binding specificity against NKG2D ligands, HER3, CD19 or a combination thereof.

In one embodiment, the hexa-specific antibody-like protein may include D1 having a binding specificity against EGFR, D2 having a binding specificity against CD3, D3 may have a binding specificity against PD-L1, D4 having a binding specificity against 4-1BB, and D5 having a binding specificity against CD19, and D6 having a binding specificity against HER3.

In one embodiment, the hexa-specific antibody-like protein having D1 may have a binding specificity against EGFR, D2 having a binding specificity against CD3, D3 having a binding specificity against PD-L1, D4 having a binding specificity against 4-1BB, and D5 having a binding specificity against HER3, and D6 having a binding specificity against CD19.

In one embodiment, the hexa-specific antibody-like protein may include D1 having a binding specificity against CD3, D2 having a binding specificity against EGFR, D3 having a binding specificity against PD-L1, D4 having a binding specificity against 4-1BB, and D5 having a binding specificity against HER3, and D6 having a binding specificity against CD19.

In one embodiment, the hexa-specific antibody-like protein may include an amino acid sequence having at least 50%, 60%, 70%, 80%, 85%, 90%, 95%, 98%, 99%, or 100% of sequence identify to SEQ ID NO. 176, 178, 106, 108, 332, 334, 324, 326, 328, or 330.

In one aspect, the application provides tetra-specific or penta-specific antibody-like proteins. In one embodiment, the antibody-like protein having a N-terminal and a C-terminal, may include in tandem from the N-terminal to the C-terminal, a first binding domain (D1) at the N-terminal, a Fab region as a second binding domain (D2) may include a light chain, wherein the light chain optionally may comprise a fifth binding domain (D5) covalently attached to the C-terminal or a sixth binding domain (D6) covalently attached to the N-terminal, a Fc region, a third binding domain (D3), and a fourth binding domain (D4) at the C-terminal. In one embodiment, the multi-specific antibody-like protein comprise an amino acid sequence having at least 50%, 60%, 70%, 80%, 85%, 90%, 95%, 98%, 99%, or 100% of sequence identify to SEQ ID NO. 110, 112, 116, 118, 122, 124, 128, 130, 134, 136, 140, 142, 146, 148, 152, 154, 158, 160, 164, 166, 170, 172, 112,114, 118, 120, 124, 126, 130, 132, 136, 138, 142, 144, 148, 150, 154, 156, 160, 162, 166, 168, 172, 174, 34, 36, 38, 40, 42, 44, 46, 48, 50, 52, 54, 56, 302, 304, 306, or 308.

In one embodiment, the multi-specific antibody-like protein may tetra-specific. In one embodiment, the multi-specific antibody-like protein may penta-specific. In one embodiment, D2, D5, and D6 each independently may have a binding affinity to a tumor associate antigen (TAA).

In one embodiment, the tetra-specific antibody-like protein may have D1 having a binding specificity against CD3, D2 having a binding specificity against a tumor-associated antigen, D3 having a binding specificity against PD-L1, and D4 having a binding specificity against 4-1BB.

In one embodiment, the tetra-specific antibody-like protein may have D1 having a binding specificity against CD3, D2 having a binding specificity against an antigen selected from a group consisting of EGFR, HER2, CD19, CD20, CD22, CD30, CD22, mesothelin, GD2, and Claudin 18.2, D3 having a binding specificity against PD-L1, and D4 having a binding specificity against 4-1BB.

In one embodiment, the penta-specific antibody-like protein may have D1 having a binding specificity against CD3, D2 and D5 independently each having a binding specificity against a tumor-associated antigen, D3 having a binding specificity against PD-L1, and D4 having a binding specificity against 4-1BB.

In one embodiment, the penta-specific antibody-like protein may have D1 having a binding specificity against CD3, D2 having a binding specificity against a tumor-associated antigen, D3 having a binding specificity against PD-L1, D4 having a binding specificity against 4-1BB, and D5 having a binding specificity against HER3.

In one embodiment, the penta-specific antibody-like protein may have D1 having a binding specificity against CD3, D2 having a binding specificity against EGFR or EGFRvIII, D3 having a binding specificity against PD-L1, D4 having a binding specificity against 4-1BB, and D5 having a binding specificity against HER3.

In one embodiment, the penta-specific antibody-like protein may have D1 having a binding specificity against CD3, D2 having a binding specificity against CD20, D3 having a binding specificity against PD-L1, D4 having a binding specificity against 4-1BB, and D5 having a binding specificity against CD19.

In one embodiment, the penta-specific antibody-like protein may have D1 and D6 independently having a binding specificity against a tumor-associated antigen, D2 having a binding specificity against CD3, D3 having a binding specificity against PD-L1, and D4 having a binding specificity against 4-1BB.

In one embodiment, the penta-specific antibody-like protein may have D1 having a binding specificity against EGFR, D2 having a binding specificity against CD3, D3 having a binding specificity against PD-L1, D4 having a binding specificity against 4-1BB, and D6 having a binding specificity against CD19.

In one aspect, the application provides multi-specific antibody-like protein with at least one binding domain as a receptor. In one embodiment, the receptor is NKG2D.

In one embodiment, the multi-specific antibody-like protein having a N-terminal and a C-terminal, may include in tandem from the N-terminal to the C-terminal, optionally a first binding domain (D1) at the N-terminal, a second binding domain (D2) may include a light chain, wherein the light chain optionally may comprise a fifth binding domain (D5) covalently attached to the C-terminal, a sixth binding domain (D6) covalently attached to the N-terminal, or both, a Fc region, optionally a third binding domain (D3), and optionally a fourth binding domain (D4) at the C-terminal, wherein at least one of D1, D2, D3, D4, D5, and D6 is a NKG2D, and wherein D1, D2, D3, D4, D5, and D6 each independently may have a binding affinity to specificity against a T cell activating receptor, an immune cell receptor, an immune checkpoint molecule, a co-stimulation factor, a receptor of a leukocyte, a tumor antigen, a tumor associated antigen (TAA), a receptor of a tissue cell, a receptor of a cancer cell, or a combination thereof.

In one embodiment, the NKG2D containing multi-specific antibody-like protein may mono-specific, bi-specific, tri-specific, tetra-specific or penta-specific.

In one embodiment, the NKG2D containing multi-specific antibody-like protein may have D2 comprising a dimer connected to CL and CH1, wherein the dimer is NKG2D. In one embodiment, the NKG2D containing mono-specific antibody-like protein may include an amino acid sequence having at least 50%, 60%, 70%, 80%, 85%, 90%, 95%, 98%, 99%, or 100% sequence identify to SEQ ID NO. 196 or 198.

In one embodiment, the NKG2D containing multi-specific antibody-like protein may have D1, D2, D3, D4, D5 and D6 each independently having a binding specificity against an antigen selected from EGFR, HER2, HER3, EGFRvIII, ROR1, CD3, CD28, CEA, LMP1, LMP2A, Mesothelin, PSMA, EpCAM, glypican-3, gpA33, GD2, TROP2, NKG2D, NKG2D ligand, BCMA, CD19, CD20, CD33, CD123, CD22, CD30, PD-L1, PD1, OX40, 4-1BB, GITR, TIGIT, TIM-3, LAG-3, CTLA4, CD40, CD40L, VISTA, ICOS, BTLA, LIGHT, HVEM, CSF1R, CD73, CD39, CLDN18.2, DLL3, HLA-G, FcRH5, GPRC5D, LIV-1, MUC1, CD138, CD70, CD16, uPAR, Siglec-15, CD47, CD38, NKp46, PD-L2, CD160, LOX-1, SIRPα, CD27, and wherein the Fc domain may comprise a human IgG Fc domain.

In one embodiment, the NKG2D containing multi-specific antibody-like protein may have D2, D5, and D6 each independently having a binding specificity against a tumor associated antigen. In one embodiment, the NKG2D containing multi-specific antibody-like protein may have D2 having a binding specificity against a tumor associated antigen. In one embodiment, the NKG2D containing multi-specific antibody-like protein may have D1, D2, D3, and D4 each independently having a binding specificity against NKG2D ligands, CD3, PD-L1, 4-1BB or a combination thereof.

In one embodiment, the NKG2D containing multi-specific antibody-like protein an amino acid sequence having at least 50%, 60%, 70%, 80%, 85%, 90%, 95%, 98%, 99%, or 100% of sequence identify to SEQ ID NO. 2, 4, 6, 8, 10, 12, 14, 16, 18, 20, 22, 24, 26, 28, 58, 60, 62, 64, 66, 68, 70, 72, 74, 76, 184, 186, 188, 190, 192, 194, 196, 198, 200, 202, 204, 206, 208, 210, 78, 80, 82, 84, 86, 88, 30 or 32.

In one embodiment, the NKG2D containing multi-specific antibody-like protein D1 having a binding specificity against NKG2D ligands, D2 having a binding specificity against CD3, D3 having a binding specificity against PD-L1, and D4 having a binding specificity against 4-1BB.

In one embodiment, the NKG2D containing multi-specific antibody-like protein D1 having a binding specificity against NKG2D ligands, D2 having a binding specificity against CD3, D3 having a binding specificity against 4-1BB, and D4 having a binding specificity against PD-L1.

In one embodiment, the NKG2D containing multi-specific antibody-like protein D1 having a binding specificity against 4-1BB, D2 having a binding specificity against PD-L1, D3 having a binding specificity against CD3, and D4 having a binding specificity against NKG2D ligands.

In one embodiment, the NKG2D containing multi-specific antibody-like protein D1 having a binding specificity against PD-L1, D2 having a binding specificity against 4-1BB, D3 having a binding specificity against CD3, and D4 having a binding specificity against NKG2D ligands.

In one embodiment, the NKG2D containing multi-specific antibody-like protein D1 having a binding specificity against CD3, D2 having a binding specificity against a tumor-associated antigen, D3 having a binding specificity against PD-L1, D4 having a binding specificity against 4-1BB, and D5 having a specificity against NKG2D ligands.

In one embodiment, the NKG2D containing multi-specific antibody-like protein D1 having a binding specificity against CD3, D2 having a binding specificity against an antigen selected from a group consisting of mesothelin, claudin 18.2, HER2, EGFRvIII, and CD33, D3 having a binding specificity against PD-L1, D4 having a binding specificity against 4-1BB, and D5 having a specificity against NKG2D ligands.

In one embodiment, the NKG2D containing multi-specific antibody-like protein D1 having a binding specificity against CD3 and D2 having a binding specificity against NKG2D ligands.

In one embodiment, the NKG2D containing multi-specific antibody-like protein D1 having a binding specificity against CD3, D2 having a binding specificity against NKG2D ligands, and D6 having a binding specificity against a tumor-associated antigen.

In one embodiment, the NKG2D containing multi-specific antibody-like protein D1 having a binding specificity against CD3, D2 having a binding specificity against NKG2D ligands, and D6 having a binding specificity against CD19.

In one embodiment, the NKG2D containing multi-specific antibody-like protein D1 having a binding specificity against CD3, D2 having a binding specificity against NKG2D ligands, D3 having a binding specificity against PD-L1, and D4 having a binding specificity against 4-1BB.

In one embodiment, the NKG2D containing multi-specific antibody-like protein D1 having a binding specificity against CD3, D2 having a binding specificity against NKG2D ligands, D3 having a binding specificity against PD-L1, D4 having a binding specificity against 4-1BB, and D6 having a binding specificity against a tumor-associated antigen.

In one embodiment, the NKG2D containing multi-specific antibody-like protein D1 having a binding specificity against CD3, D2 having a binding specificity against NKG2D ligands, D3 having a binding specificity against PD-L1, D4 having a binding specificity against 4-1BB, and D6 having a binding specificity against CD19.

In a second aspect, the application provides isolated nucleic acid sequences encoding an amino acid sequence of the multi-specific antibody-like protein as disclosed thereof.

In a third aspect, the application provides expression vector. In one embodiment, the expression vector includes isolated nucleic acid sequence as disclosed herein.

In a further aspect, the application provides host cell including the isolated nucleic acid sequences disclosed herein. In one embodiment, the host cell is a prokaryotic cell. In one embodiment, the host cell is a eukaryotic cell.

In a further aspect, the application provides method for producing a multi-specific antibody-like proteins of their fragments thereof. In one embodiment, the method may include the steps of culturing a host cell may include an isolated nucleic acid sequence such that the DNA sequence encoding the multi-specific antibody or monomer is expressed, and purifying said multi-specific antibody, wherein the isolated nucleic acid sequence encodes an amino acid of the multi-specific antibody-like proteins or their fragments thereof.

In a further aspect, the application provides immunoconjugates. In one embodiment, the immuno-conjugate may include a cytotoxic agent or an imaging agent linked to the multi-specific antibody of claim 30 through a linker, wherein the linker may comprise an ester bond, an ether bond, an amid bond, a disulphide bond, an imide bond, a sulfone bond, a phosphate bond, a phosphorus ester bond, a peptide bond, a hydrophobic poly(ethylene glycol) linker, or a combination thereof. In one embodiment, the cytotoxic agent or the imaging agent may comprise a chemotherapeutic agent, a growth inhibitory agent, a cytotoxic agent from class of calicheamicin, an antimitotic agent, a toxin, a radioactive isotope, a toxin, a therapeutic agent, or a combination thereof.

In a further aspect, the application provides pharmaceutical compositions. In one embodiment, the pharmaceutical composition may include a pharmaceutically acceptable carrier and the multi-specific antibody-protein or their fragments thereof, the immuno-conjugate disclosed herein, or both. In one embodiment, the pharmaceutical composition further may include a therapeutic agent selected from a radioisotope, radionuclide, a toxin, a chemotherapeutic agent or a combination thereof.

In a further aspect, the application provides method for treating or preventing a cancer, an autoimmune disease, or an infectious disease in a subject. In one embodiment, the method includes the steps of administering a pharmaceutical composition that may include a purified multi-specific antibody-like protein of their fragments thereof. In one embodiment, the method may include administering to the subject an effective amount of the purified multi-specific antibody-like protein, immunoconjugates, or pharmaceutical composition disclosed herein.

In one embodiment, the method may further include co-administering an effective amount of a therapeutic agent, wherein the therapeutic agent may comprise an antibody, a chemotherapy agent, an enzyme, an anti-estrogen agent, a receptor tyrosine kinase inhibitor, a kinase inhibitor, a cell cycle inhibitor, a check point inhibitor, a DNA, RNA or protein synthesis inhibitor, a RAS inhibitor, an inhibitor of PD1, PD-L1, CTLA4, 4-1BB, OX40, GITR, ICOS, LIGHT, TIM3, LAG3, TIGIT, CD40, CD27, HVEM, BTLA, VISTA, B7H4, CSF1R, NKG2D ligand, CD73, or a combination thereof.

In one embodiment, the subject is a human. In one embodiment, the subject is a mammal. In one embodiment, the subject is chimpanzee. In one embodiment, the subject is a pet animal.

In a further aspect, the application provides solutions including an effective concentration of the purified multi-specific antibody-like protein or their fragments thereof, immunoconjugates, or pharmaceutical composition as disclosed herein. In one embodiment, the solution is blood plasma in a human subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 depicts the configuration of hexaGNC antibodies having either a Fab region or a dimer receptor as the D2 binding domain, and 5 antigen binding domains added to the heavy (D1, D3, and D4) and light chain (D5 and D6) with diverse structures selected from variable sequence based scFv, and non-variable sequence coded receptor and ligand;

FIG. 2 shows the results of analytical SEC demonstrating the stability and high quality of purified tetraGNC antibodies comprising NKG2D receptor and 41BBL (A-C), and purified NKG2D pentaGNC (D);

FIG. 3 shows TDCC assay measuring the comparative potency of 4 tetraGNC antibodies (SI-49E1, SI-49E2, SI-49E3, SI-49E4) and 2 bispecific control antibodies lacking both αPD-L1 and α41BB domains (SI-49X1 and SI-49X2) when targeting MICA expressing MDA-MB-231 cell line;

FIG. 4 shows TDCC assay measuring the comparative potency of one NKG2D-αMSLN penta GNC (SI-49P1), two αMSLN tetraGNC (SI-51E4 and SI-51E1), and one NKG2D-αMSLN triGNC (SI-51X1, control) when targeting MICA- and mesothelin-expressing MDA-MB-231 cells;

FIG. 5 shows TDCC assay measuring the comparative potency of a group of tetraGNC antibodies having a trio of moiety 1 binding domains and a single moiety 2 binding specificity to variety of tumor antigens when targeting cervical cancer cells (Hela);

FIG. 6 shows TDCC assay measuring the comparative potency of multi-specific GNC antibodies having the same binding specificity to EGFR for all molecules and also HER3 for SI-1 and SI-1P2, in the presence (SI-1P2, SI-55E1 and SI-55E2) and the absence of moiety 1 binding specificity (SI-1) when targeting EGFR-expressing MDA-MB-231 breast cancer cells;

FIG. 7 shows TDCC assay measuring the comparative potency of tetraGNC with (SI-50E6, stapled) and without (SI-50E1) addition disulfide bonds in all of its scFv domains, as compared to a biGNC antibody (SI-50X1) when targeting EGFR-expressing MDA-MB-231 breast cancer cells; and

FIG. 8 depicts the generation of a class of multi-specific GNC antibodies having a trio of moiety 1 binding specificities to CD3, PD-L1, and 4-1BB from D1, D3, and D4 and any combination of moiety 2 binding specificities, i.e. to three tumor antigens, from D2, D5, and D6 by using a modular cloning system.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The disclosure provides, among others, isolated antibodies, methods of making such antibodies, bispecific or multi-specific molecules, antibody-drug conjugates and/or immuno-conjugates composed from such antibodies or antigen binding fragments, pharmaceutical compositions containing the antibodies, bispecific or multi-specific molecules, antibody-drug conjugates and/or immuno-conjugates, the methods for making the molecules and compositions, and the methods for treating cancer using the molecules and compositions disclosed herein.

The present application relates to methods of making and using multi-specific GNC antibodies, in particular, tetra-, penta-, and hexa-specific GNC (tetraGNC, pentaGNC, hexaGNC) antibodies. In general, GNC proteins, such as GNC antibodies, are characterized by comprising two moieties: moiety 1 for engaging immune cells, such as activating T cells, while moiety 2 targeting tumor cells. GNC antibodies retain multiple antigen binding domains for engaging immune cells, such as anti-CD3 for T cell activation, anti-4-1BB for co-stimulation, and anti-PD-L1 for inhibiting immune checkpoint. To improve the efficacy of antibody therapy for treating cancer, GNC antibodies are designed to be structurally stable and compact while retaining the characteristic feature of two moieties in GNC antibodies. This improvement allows an additional binding specificity to a second tumor associated antigen on the same or different tumor cell. GNC antibodies contain an Fc domain that allows for FcRn-mediated recycling and half-life extension, as well as facile protein A-based purification. The Fc receptor-mediated immunity may be incorporated if desired. GNC antibodies are usually larger than an IgG antibody due to increased number of antigen binding domains (AgBD), which provides spatial flexibility for binding to both a T cell and a tumor cell. GNC antibodies may be an efficacious antibody therapeutics for treating cancer by targeting one or more tumor antigens, including but not limited to, BCMA, CD19, CD20, CD33, CD123, CD22, CD30, ROR1, CEA, HER2, HER3, EGFR, EGFRvIII, LMP1, LMP2A, Mesothelin, PSMA, EpCAM, glypican-3, gpA33, GD2, TROP2. Multi-specific T-cell engaging antibodies, such as tetra-GNC and penta-GNC antibodies, have distinctive advantages over conventional immunotherapies. They demonstrate cross-linking functionality of CD3 on T cells with tumor associated antigens (TAAs), which re-directs and guides them to kill tumor cells without the need of removing T cells from the patient and/or genetically modifying them to be specific for tumor cells before re-introducing them back into the patient (also known as chimeric antigen receptor T cells, or CAR-T therapy). A GNC protein-mediated antibody therapy or T cell therapy does not involve genetic modification of T cells, the latter of which may carry the risk of transforming modified T cells to clonal expansion, i.e. T cell leukemia.

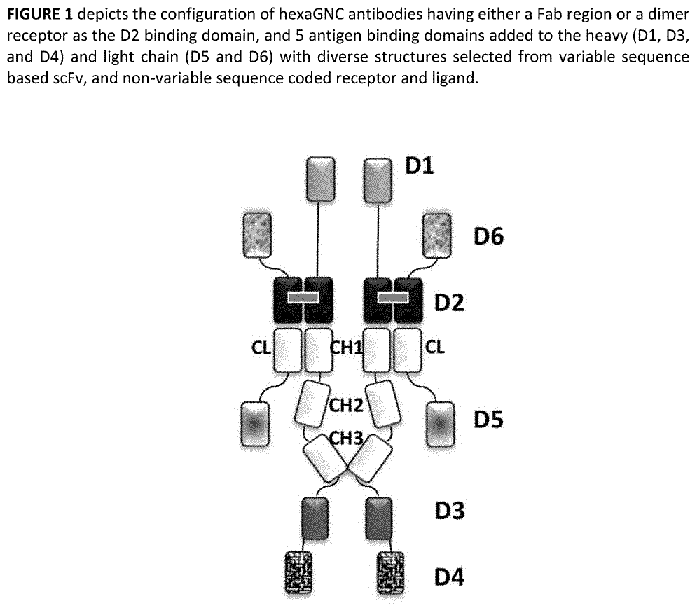

The present application discloses tetra-, penta-, and hexa-specific GNC (tetraGNC, pentaGNC, hexaGNC) antibodies comprising a heavy chain (HC) and a light chain (LC) as elucidated in FIG. 1. The hexaGNC antibodies may be configured to have either a Fab region or a dimer receptor as the D2 binding domain, and 5 antigen binding domains added to the heavy (D1, D3, and D4) and light chain (D5 and D6) with diverse structures selected from variable sequence-based antibody fragments, such as scFv, and non-variable sequence coded receptor and ligand. The VH and VL of the Fab region may be replaced by a non-Fab dimer with or without binding specificity. In one embodiment, the Fc domains of the two chains are engineered to contain complementary mutations, also known as “knobs-into-holes”, to enhance the formation of the heterodimer. A hexaGNC antibody comprises 6 independent binding specificities to at least 6 antigens expressed by immune effector cells or target cancer cells. In comparison to conventional combination therapies, the classes of hexaGNC antibodies are designed for treating cancer as a single drug in order to improve efficacy and reduce manufacturing cost. In this way, the treatment simplifies clinical administration SOP, eases logistical concerns surrounding multi variate dosing, and becomes more affordable to patients.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)₂, and Fv), so long as they exhibit the desired biological activity. In some embodiments, the antibody may be monoclonal, polyclonal, chimeric, scFv, bispecific or bi-effective, human and humanized antibodies as well as active fragments thereof. Examples of active fragments of molecules that bind to known antigens include Fab, F(ab′)₂, scFv and Fv fragments, including the products of an Fab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that immunospecifically bind an antigen. The immunoglobulin can be of any type (IgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to heterotetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The VH and VL regions can be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567).

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

Monoclonal antibodies can be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)). In the present disclosure some antibodies were created by the immunization of rabbits with both human PD-L1 protein and cells transiently expressing human PD-L1 on the cell surface. Rabbits are known to create antibodies of high affinity, diversity and specificity (Weber et al. Exp. Mol. Med. 49:e305). B cells from immunized animals were cultured in vitro and screened for the production of anti-PD-L1 antibodies. Besides immunization of rabbits followed by B cell culture, other common strategies for antibody generation and discovery include immunization of other animals (e.g., mice) followed by hybridoma and/or display on phage, yeast, or mammalian cells; or display using synthetic variable gene libraries. The antibody variable genes were isolated using recombinant DNA techniques and the resulting antibodies were expressed recombinantly and further screened for desired features such as ability to inhibit the binding of PD-L1 to PD-1, the ability to bind to non-human primate PD-L1 and the ability to enhance human T-cell activation. This general method of antibody discovery is similar to that described in Seeber et al. PLOS One. 9:e86184 (2014).

The term “antigen- or epitope-binding portion or fragment” refers to fragments of an antibody that are capable of binding to an antigen. These fragments may be capable of the antigen-binding function and additional functions of the intact antibody. Examples of binding fragments include, but are not limited to a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domains. Antibody fragments are produced using conventional methods known to those skilled in the art. The antibody fragments are can be screened for utility using the same techniques employed with intact antibodies.

The “antigen- or epitope-binding fragments” can be derived from an antibody of the present disclosure by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen.

The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody.

The “light chains” of antibodies (immunoglobulins) from any vertebrate species can be assigned to one of two clearly distinct types, called kappa and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called α, delta, epsilon, γ, and μ, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic a binding specificity.

“Recombinant” means the antibodies are generated using recombinant nucleic acid techniques in exogeneous host cells.

The term “antigen” refers to an entity or fragment thereof which can induce an immune response in an organism, particularly an animal, more particularly a mammal including a human. The term includes immunogens and regions thereof responsible for antigenicity or antigenic determinants.

Also, as used herein, the term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “affinity” refers to a measure of the attraction between two polypeptides, such as antibody/antigen, receptor/ligand, etc. The intrinsic attraction between two polypeptides can be expressed as the binding affinity equilibrium dissociation constant (KD) of a particular interaction. A KD binding affinity constant can be measured, e.g., by Bio-Layer Interferometry, where KD is the ratio of kdis (the dissociation rate constant) to kon (the association rate constant), as KD=kdis/kon.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to the equilibrium dissociation constant of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs. The deviations appearing in the comparison between a given sequence and the above-described sequences of the disclosure may be caused for instance by addition, deletion, substitution, insertion or recombination.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1. Methods and Assays

### Expression and Purification

DNA sequence encoding heavy and light chains was cloned into pTT5 vector from custom gene fragments using restriction cloning and/or Gibson assembly. Plasmid DNA was transfected transiently in ExpiCHO cells (Thermo A29133) according to manufacturer's instructions to generate multispecific GNC protein. Titers were measured after 7-9 days using ForteBio Octet instrument with protein A sensors.

GNC proteins were purified via protein A chromatography (Cytiva, 17549853), washing with phosphate-buffered saline (PBS), and eluting in 50 mM sodium acetate, pH 3.6, followed by immediate neutralization with 1/5 addition of 1M sodium acetate pH 7.0. Analytical size-exclusion chromatography (aSEC) was performed to assess protein quality following affinity purification. aSEC was performed using an Acquity Arc Waters with a XBridge BEH SEC 300 Å, 7.8×300 mm, 3.5 μm column. Proteins were further polished with a preparative SEC step using Superdex 200 Increase 10/300 GL column. All subsequent assays were performed with protein that was at least 90% protein of interest by aSEC.

### T-Cell Dependent Cytotoxicity Assay

T-cell dependent cytotoxicity (TDCC) assay is based on the methods outlined in Nazarian et al. (2014). Target cells from established cancer cell lines (ADCC) are first transduced with a luciferase expressing gene to generate luciferase positive target cells. These target cells are next grown in cell culture flasks and when an appropriate number have been expanded they are lifted, counted and replated into 384 well (Corning 3570) at an appropriate density depending on previous growth characteristics, using a BioTek EL406 liquid dispenser. For adherent cell lines the cells are allowed to adhere to the plates overnight in a CO2 controlled jacketed tissue culture incubator. Next, either PBMC or previously expanded T-cells (dynabeads) are plated at an appropriate effector to target ratio, frequently 5:1, and the plate is dosed with a dilution series of the test T-cell targeting agent. Test article experiments are conducted in quadruplicate as the 96 well dilution block is stamped into 384 well quadrants robotically (Opentrons OT-2 liquid handling robot). The TDCC assay plate is incubated for 72-96 hours. Readout of the cell viability curve is accomplished by the use of the Promega Bright-glo luciferase assay kit. Briefly, 20 uL are added to the TDCC assay plate and incubated for ^{˜}15 min before measuring the resultant luminescence on a BMG Clariostar plate reader. Killing curves and EC50 values are analyzed and plotted in GraphPad Software.

### SEC-MALS

Analytical size exclusion chromatography (SEC) in combination with multi-angle light scattering (MALS) and absorbance (UV) and/or refractive index (RI) concentration detectors. SEC-MALS is typically employed during analytical characterization and early phase clinical trials of antibody to support FDA IND submission. Our size exclusion is performed using an Acquity Arc Waters with a XBridge BEH SEC 300 Å, 7.8×300 mm, 3.5 μm column. The MALS component uses Wyatt miniDAWN TREOS/Optilab T-rEX systems. The molecular weight may be determined from a measurement of the change, Δn, of the solution's refractive index n with the molecular concentration change, Δc, by measuring the dn/dc (=Δn/Δc) value using an Optilab T-rEX differential refractometer. The intensity of light scattered by a molecule, measured by means of a miniDAWN TREOS multi-angle light scattering (MALS) detector, is directly proportional to the molar mass.

### OCTET

ForteBio Octet platform applies Bio-Layer Interferometry (BLI) as a label-free technology for measuring protein-protein interactions. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time. In this method, the binding between an antibody immobilized on the Anti-human IgG Fc Capture (AHC) Biosensors tip surface and an antigen in solution produces an increase in optical thickness at the biosensor tip, which results in a wavelength shift, Δλ, directly reflecting the change in thickness of the biological layer. The interaction of these two molecules are measured in real time, providing the ability to monitor binding specificity, rates of association and dissociation, or concentration, with precision and accuracy. Unbound molecules, changes in the refractive index of the surrounding medium, or changes in flow rate do not affect the interference pattern. Facile analysis of the binding of GNC proteins to targeted antigens can be performed on the Octet system, using AHC tips to immobilize the GNC protein, and using purified antigen as analyte either as a single concentration (e.g., 100 nM) or a series of concentrations (e.g., seven 1:2 serial dilutions starting at 200 nM).

### Tm by Dynamic Light Scattering (DLS)

The hydrodynamic radius (Rh) of antibody samples (1 mg/ml) was measured by using a DynaPro Plate Reader (Wyatt Technology, Santa Barbara, Calif.) from 25° C. to 75° C. in 1° C. increments with ramp rate of 0.26° C./min. A total of 3 acquisitions of 5 s were collected at each temperature. DYNAMICS 7.8.1.3 software (Wyatt Technologies) was used to calculate the radius and the onset temperature at which Rh begins to change significantly and the midpoint of the transition curve (Tm).

Quantitative Flow Cytometry (qFACS)

CountBright absolute counting beads (Thermo C36950) were used for calibration, and primary antibodies panitumumab (EGFR), anti-HER3 from MM111 (HER3), PL221G5 (PD-L1), TF 3H8-1 (CEA), and trastuzumab (HER2) were used to quantify the corresponding receptor number on various tumor cell lines, as described previously (Wang L et al. Curr Protoc. Cytom. 2016).

### Example 2. GNC Antibodies Having NKG2D as One of Binding Domains

NKG2D is a major recognition receptor for the detection and elimination of transformed and infected cells as its ligands are induced during cellular stress, either as a result of infection or genomic stress such as in cancer. In humans, NKG2D is encoded by KLRK1 gene which is located in the NK-gene complex (NKC), it is expressed by NK cells, γδ T cells, and CD8⁺ αβ T cells. Human NKG2D receptor complex assembles into a hexameric structure, while NKG2D itself forms a homodimer whose ectodomains serve for ligand binding. In NK cells, NKG2D serves as an activating receptor, which itself is able to trigger cytotoxicity. The function of NKG2D on CD8⁺ T cells is to send co-stimulatory signals to activate them. The major histocompatibility complex class I polypeptide-related sequence A gene (MICA) encodes a membrane-bound protein acting as a ligand to stimulate an activating receptor, NKG2D, expressed on the surface of essentially all human natural killer (NK), γδ T and CD8⁺ αβ T cells. MICA protein is absent from most cells but can be induced by infections and oncogenic transformation and is frequently expressed in epithelial tumors. Upon binding to MICA, NKG2D activates cytolytic responses of NK and γδ T cells against infected and tumor cells expressing MICA. Therefore, membrane-bound MICA acts as a signal during the early immune response against infection or spontaneously arising tumors. On the other hand, human tumor cells spontaneously release a soluble form of MICA, causing the downregulation of NKG2D and in turn severe impairment of the antitumor immune response of NK and CD8⁺ T cells. This is considered to promote tumor immune evasion and also to compromise host resistance to infections, which may be neutralized by free NKG2D. In this context and by the definition of GNC proteins, NKG2D is one of cytotoxic cell binding moieties (from Applicant's application No. PCT/US2018/039160, incorporated herein in its entirety).

The GNC antibodies having NKG2D ligand binding specificity comprise a common core antibody domain, the Fc region of which can either have or be devoid of effector function. The tetraGNC antibodies having NKG2D dimer as one of binding domain on the heavy chain (HC) have been generated (Table 1). These antibodies had either 2 additional scFv domains, including the binding domain to 4-1BB, a TNF superfamily receptor normally expressed on activated T cells (SI-49E1, SI-49E2, SI-49E3, SI-49E4, Table 1), or 1 additional scFv plus 41BBL (trimer form), which is normally found on antigen-presenting cells (APCs) and binds to 4-1BB (SI-49E11, SI-49E12, SI-49E13, Table 1). The four binding domains (D1 through D4) were fused through G/S linker regions and expressed as a single heavy chain (HC or Chain A or Chain 1). By definition as shown in FIG. 1, these types of tetraGNC antibodies are characterized by having one of the scFvs (D1) located at the N-terminus of the VH domain (D2 or Fab) and two scFvs attached in succession to the Fc region (D3 and D4), whereas the light chain (LC) comprises only the VL domain (D2 or Fab) in natural configuration. To produce pentaGNC antibodies having NKG2D binding specificity, one strategy is to engineer an additional NKG2D tandem repeat homodimer at either N (D6) or C (D5) terminus of LC (FIG. 1). Five D5-pentaGNC antibodies with NKG2D dimer as a binding domain from LC were generated (SI-49P1, SI-49P2, SI-49P3, SI-49P4, and SI-49P5, Table 2). The characteristic feature of these D5-pentaGNC antibodies is a single cancer-targeting moiety (D2) in combination with 4 cytotoxic binding moieties, namely, anti-CD3, anti-PD-L1, anti-4-1BB, and NKG2D. Table 3 listed the information of both exemplary cancer-targeting moieties and cytotoxic binding moieties as independent binding domains. By the definition, PD-L1 is not classified as a cancer-targeting moiety even though it is expressed on cancer cells as an immune checkpoint marker. While these D5-pentaGNC antibodies consist of a core antibody binding domain with additional 3 scFv binding regions on HC and one NKG2D tandem repeat homodimer on LC for a total of 5 different specificities, the configuration may be diversified. For example, the pentaGNC antibodies may have a core antibody binding domain with an additional 2 scFv binding regions plus one TNF superfamily (trimer form) domain, and one NKG2D tandem repeat homodimer for a total of 5 different specificities. Because NKG2D and 41BBL are not typical antigen binding domain structure, i.e. either Fab or scFv, such GNC antibodies with a Fab at the core may be called GNC molecules or GNC proteins elsewhere with the same meaning. In addition, the domain structures may be engineered for stabilizing those multi-specific GNC proteins. For example, each scFv domain may have both, a stapled by a disulphide bond at vL100 and vH44 and a not stapled, variants to be selected for stabilizing the overall structure.

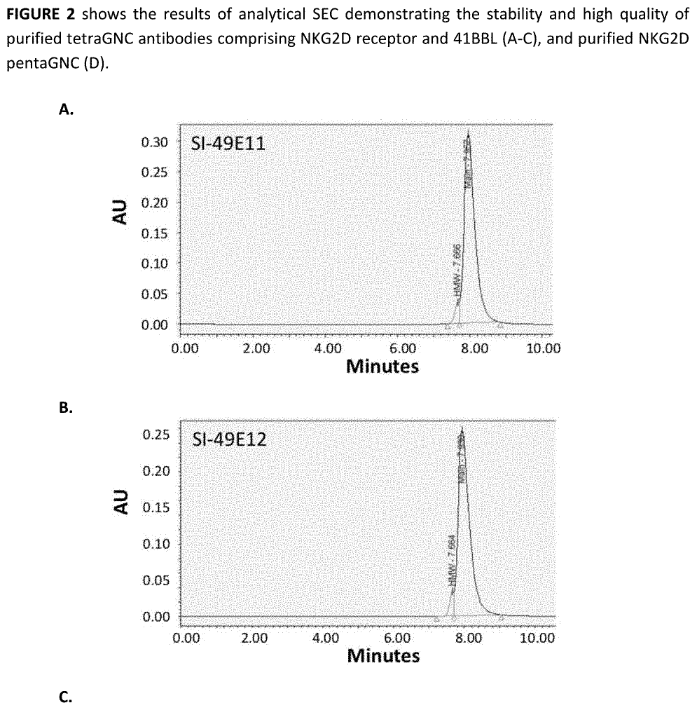

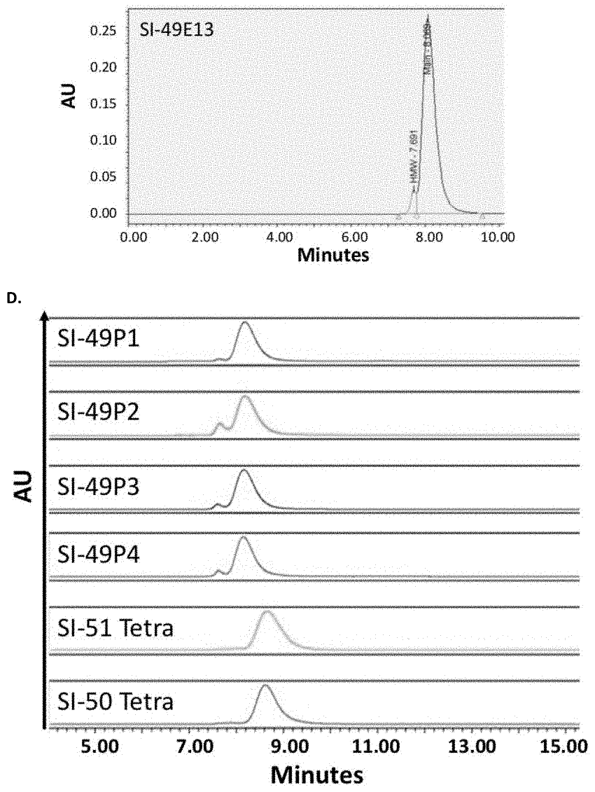

Analytical SEC shows the stability and high quality of purified tetraGNC antibodies comprising NKG2D receptor and 41BBL (FIG. 2A-2C), and several D5-pentaGNC having NKG2D binding specificity from LC (2D), including SI-49E1, SI-49E2, SI-49E3, SI-49E4, SI-49E11, SI-49E12, SI-49E13, SI-49P1, SI-49P2, SI-49P3, and SI-49P4. The results demonstrated that these atypical GNC antibodies can be readily purified and remain stable.

### Example 3. Comparative Potency of GNC Antibodies Having NKG2D as One of Binding Domains

In NK cells, NKG2D serves as an activating receptor, which itself can trigger cytotoxicity, whereas on CD8⁺ T cells the function of NKG2D is to send co-stimulatory signals to activate them. NKG2D forms a homodimer whose ectodomains serve for ligand binding. This feature qualifies NKG2D as a non-variable-sequence-based binding domain in a GNC format and other binding domains can be added to create a class of multispecific NKG2D-GNC protein. In one GNC format, individual NKG2D monomer was incorporated in the D2 position on heavy chain and light chain which formed a dimeric NKG2D receptor on HC/LC dimerization. Thus, NKG2D can act as a receptor for the multi-specific antibody-like protein GNC molecule to bind its ligand. In other GNC format, a NKG2D tandem repeat was designed by adding a (GxSy)n linker between individual NKG2D monomers which homodimerizes and forms a functional dimeric receptor. This NKG2D tandem dimeric structure can be positioned in D1, D3, D4, D5, or D6.

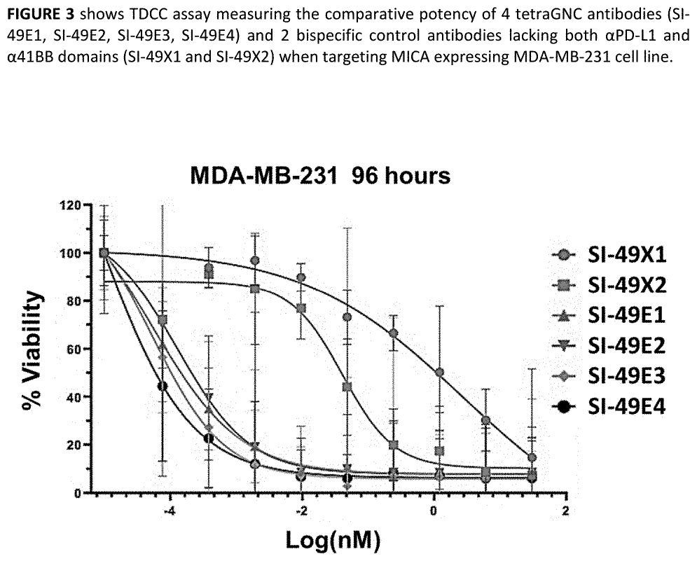

To evaluate the effect of NKG2D position in tetraGNC antibodies, SI-49E1, SI-49E2, SI-49E3, SI-49E4 were used for TDCC assay (FIG. 3). Using MDA-MB-231 cell line expressing MICA all 4 NKG2D tetraGNC antibodies showed higher potency than the control antibodies of SI-49X1, a bispecific NKG2D-αCD3-Fc antibody, and SI-49X2, a bispecific Fc-αCD3-NKG2D antibody, both of which lack anti-PD-L1 and anti-4-1BB scFv. The result indicates that the binding specificity to PD-L1 and/or 4-1BB contributes to the toxicity. The minor differences among the 4 NKG2D tetraGNC antibodies could be significant reflecting the differences in their configuration and accessibility to immune cells.

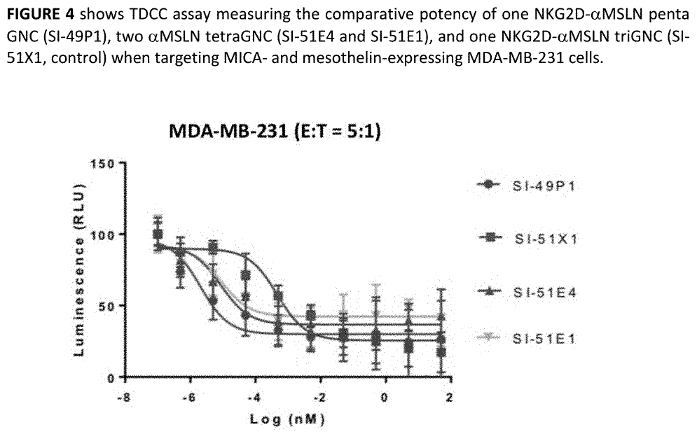

To evaluate the effect of NKG2D position in pentaGNC antibodies, SI-49P1, SI-49P2, SI-49P3, SI-49P4, and SI-49P5 (Table 2) were used for TDCC assay (FIG. 4). SI-49P1, a NKG2D-αMSLN pentaGNC antibody shows cell killing with higher potency than both tetraGNC control antibodies, SI-51E4 and SI-51E1, and NKG2D-αMesothelin (αMSLN) control antibody, SI-51X1. This observation supports the inclination that the addition and position of NKG2D to tetraGNC antibodies improves potency. The fact that a control antibody, SI-51X1, which is a tri-specific αNKG2D-LC/αCD3-αMSLN-Fc without αPD-L1 and α4-1BB, is less potent indicates that both αPD-L1 and α4-1BB domains may enhance the toxicity. The potency of cell killing is powered by pentaGNC>tetraGNC>pentaGNC control antibodies.

To quantify the ability of NKG2D receptor domain dimer to redirect T cells to kill MICA-bearing tumor cells in the context of tetraGNC or pentaGNC antibodies, TDCC assay with MDA-MB-231 target cells was performed. Test articles included a control tetraGNC (SI-38E72, αCD3×αFITC×αPD-L1×α4-1BB) with negative control anti-FITC domain at D2, tetraGNC antibodies (SI-49E1, SI-49E2, SI-49E3, SI-49E4, NKG2D×αCD3×αPD-L1×α4-1BB) with binding domains at different GNC positions, and pentaGNC (SI-49P1, αCD3×αMSLN×αPD-L1×α4-1BB×NKG2D) with additional αMSLN binding domain. The effector:target cell ratio (E:T) was 5:1, and the purified T cells, target cells, and drug dilutions were incubated for 96 hours before reading luminescence, representative of remaining tumor cells. Note that some experiments were performed on different days, and the absolute EC50 value may vary. Nevertheless, the results in Table 4 show that all tetraGNC antibodies containing the NKG2D domain had significantly higher potency of TDCC than the corresponding tetraGNC without NKG2D (SI-38E72), ranged from about 10-fold (SI-49E2) to more than 130-fold (SI-49E4). While the differences among these tetraGNC antibodies may be attributed to configuration of the same group of 4 bind domains, the addition of anti-TAA moiety significantly increased the potency of SI-49P1, a pentaGNC antibody, by more than 600-fold. Thus, adding one cytotoxic binding moiety, such as NKG2D, may improve the potency, and adding an anti-TAA binding moiety can specifically increase potency of T cell-mediated tumor cell killing.

### Example 4. Mutation to Remove Light Chain Contaminants

Antibody-based proteins are most often purified via protein A affinity chromatography, where the protein A resin captures the antibody at a binding site at the C_{H2}-C_{H3} interface in the Fc domain. However, protein A also binds to the V_{H} domain domain of V_{H3} family Fvs. For most antibody-based platforms this is not a problem, since V_{H} domains are generally on the heavy chain. However, when scFvs containing V_{H3} are attached to the light chain, the V_{H} domain can bind to protein A resin during purification, causing light chain monomers and dimers to contaminate the desired heavy-light chain heterotetramer. Thus, a potential hurdle when producing multi-specific antibodies containing any V_{H3} domain on the light chain is the presence of additional contaminants in the protein A elution. This is especially problematic when the light chain expresses more efficiently than the heavy chain, causing an abundance of light chain contaminants to be purified along with the desired protein assembly.

In order to rationally disrupt protein A binding of V_{H3} family members, a structural approach was taken to interrupt the binding interface. Crystal structure 1DEE (Graille M. et al. Proc. Nat. Acad. Sci. 2000.) showed that residue R19 in V_{H3} (Kabat numbering) is in direct contact with two side chains of protein A domain D. In particular, contact with Q32 and D36 could be eliminated to significantly weaken the interaction. Thus, R19 was mutated to serine, which does not form these interactions due to its shorter side-chain. Additionally, S19 exists naturally in other V_{H} family members, suggesting that it may be less immunogenic than other substitutions. The mutation R19S (Kabat numbering) was incorporated into the FR1 region of the V_{H} domain for V_{H3}-containing scFvs on the GNC light chain. Specifically, the hexa-GNC antibody, SI-55-H11, harbored R19S mutation in its light chain sequences encoding the anti-HER3 scFv domain at D5 and the anti-CD19 scFv at domain 6. The residue of interest is at the protein A binding interface, and therefore mutation of R to S disrupts the interaction with protein A. Elimination of protein A binding in light chain scFvs prevents light chain monomers and dimers from binding to protein A during purification. As a result, a more homogeneous product without light chain contaminants can be obtained. For hexa-GNC, which may contain up to two V_{H3} scFvs per light chain, this mutation is especially important in allowing efficient purification of the desired product.

### Example 5. Comparative Potency of Multi-Specific GNC Antibodies with Diverse Structures of Binding Domains

In a general scheme of multi-specific GNC proteins (FIG. 1), each binding domain of multi-specific GNC antibodies may be variable sequence-based Fab, scFv, or non-variable sequence-based ligand, receptor, or other binding structure. To evaluate how the structural diversity of each binding domain may affect the overall functionality of multi-specific GNC antibodies, TDCC assay was performed to elicit T cell-mediated killing of pancreatic cancer cells (BxPC3). As listed in Table 5, 3 pentaGNC antibodies (SI-1P1, SI-55P9, and SI-55P10) and 1 hexaGNC antibodies (SI-55H11) were used to assess the variation of binding specificity to EGFR, HER3, CD19, CD3, and 4-1BB. All test articles included αPD-L1 scFv at positions D3 without variation in either its position or structure. For the binding specificity to EGFR, the variation included positions (D1 vs D2). For the binding, the variation included both structures (stapled vs not stapled) and positions (D1 vs D2). For the binding specificity to 4-1BB, the variation included both structures and binding mechanisms (variable sequence-based scFv vs non-variable sequence-based ligand-receptor interaction via 41BBL, i.e. 4-1BB ligand trimer to 4-1BB receptor). For the binding specificity to CD19, the variation was related humanized variable sequences. And for the binding specificity to HER3, the difference aided to this group as either tetraGNC, pentaGNC, or hexaGNC antibodies, since HER3 was not detectable on the surface of BXPC3 cells see Table 9).

The TDCC assay was set under the same conditions, such as the Effector:Target cell ratio (E:T) was 5:1, and the purified T cells, target cells, and drug dilutions were incubated for 72 hours before reading luminescence, representative of remaining tumor cells. Note that some experiments were performed on different days, the EC50 values may vary from one experiment to the other within an error margin. Nevertheless, the potency of these pentaGNC and hexaGNC antibodies were under 1 pM and within a ten-fold range, indicating the structural variations may improve manufacturing cost and feasibility more significantly than the efficacy of killing BXPC3 cells. In this context, the composition of binding specificity remains to be the determining factor for creating multi-specific GNC antibodies targeting specific forms of cancer.

### Example 6. Composition of Moiety 1 and Moiety 2 for Multi-Specific GNC Antibodies

With the capacity of up to six binding specificities, multi-specific GNC antibodies may become antibody therapy with the highest potency of killing cancer cells, for example, the value of EC50 may be down to the range of pM or even fM from nM. A successful and highly efficacious multi-specific GNC antibody depends on the composition of both moiety 1 and moiety 2 antigens. Table 4 established the comparative potency of four moiety 1 binding specificities (i.e. αCD3, αPD-L1, α4-1BB, and NKG2D) in tetraGNC antibodies in TDCC assay using MDA-MB-231 cells as target breast cancer cells. As compared to the control antibody (SI-38E72) comprising a trio of moiety 1 binding domains (i.e. αCD3, αPD-L1, and α4-1BB as D1, D3, and D4 of HC, respectively), the addition of fourth moiety 1 binding domain, a non-variable sequence-based NKG2D dimer receptor improved the potency by about 10- to 130-fold depending the configurations. However, the addition of an anti-TAA moiety significantly increased the potency of the pentaGNC antibody (SI-55H11) up to 600-fold.

To evaluate the moiety 1 binding specificity in combination with the trio moiety 1 binding domains, TDCC assay was performed using the breast cancer cell line MDA-MB-231 as target cells. All test articles included αCD3, αPD-L1, and α-4-1BB scFvs at positions D1, D3 and D4, respectively. The tetraGNC antibody (SI-38E72) was used as a control for the absence of moiety 1 binding domain, with α-FITC domain at D2 which is not specific for any tumor antigen. Other tetraGNC test articles contained various binding domains at D2 (SI-55E: αEGFR Cetuximab; SI-55E2: αEGFR Panitumumab; SI-50E1: αHER2 Trastuzumab; and SI-51E1: αMesothelin Amatuximab), and the pentaGNC antibody (SI-1P1) contained αEGFR Cetuximab at D2 and αHER3 MM111 scFv at D5. The effector:target cell ratio (E:T) was 5:1 or 10:1, and the purified T cells, target cells, and drug dilutions were incubated for 96 hours before reading luminescence, representative of remaining tumor cells. Note that some experiments were performed on different days, the EC50 values might vary but were within an error margin. Nevertheless, the results demonstrate that all GNC antibodies containing an αTAA domain at D2 elicited significantly (20- to 100-fold) more potent TDCC than the control with αFITC at D2 (Table 6). The EC50 for the pentaGNC antibody (SI-1P1) was similar to that for the tetraGNCs, indicating that domains can be added to increase TAA selectivity while still retaining potent TDCC.

### Example 7. Screening TAAs Using tetraGNC Configuration with a Trio of Moiety 1 Binding

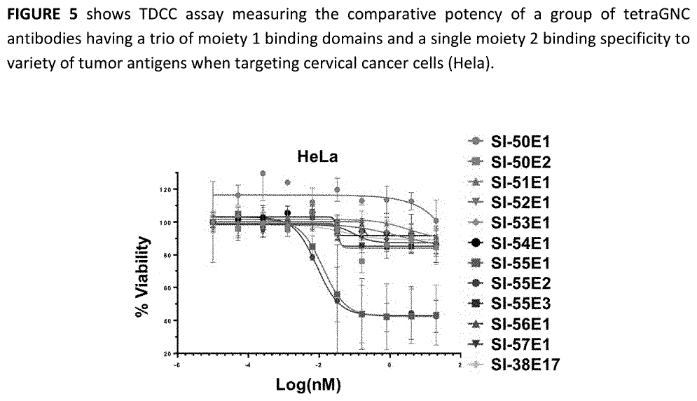

The configuration of the trio of moiety 1 binding domains being fixed at D1, D3, and D4 (Table 6) may be used as a backbone HC to accurately identify new and/or effective moiety 2 binding domains of TAAs. To assess the ability of multi-specific GNC antibodies targeting different tumor antigens to elicit T cell-mediated killing, TDCC assay was performed using the cervical cancer cell line HeLa as target cells. All test articles included αCD3, αPD-L1, and α4-1BB scFvs at positions D1, D3 and D4, respectively, and tetraGNC antibody (SI-38E72) was used as a control for having αFITC domain at D2 which is not specific for any tumor antigen. Other tetraGNC test articles contained various binding domains at D2 as listed in Table 7. The effector:target cell ratio (E:T) was 10:1, and the purified T cells, target cells, and drug dilutions were incubated for 96 hours before reading luminescence, representative of remaining tumor cells. Note that some experiments were performed on different days, so that the EC50 values may vary but were in an error margin. Nevertheless, the result from the same day experiment show that the two anti-EGFR tetraGNC antibodies, SI-55E1 (Cet) and SI-55E2 (Pan), killed more than 50% of cancer cells with a potency at 13 and 9 pM, respectively (FIG. 5). Other test articles (SI-51E1, SI-52E1, SI-50E2, SI-54E1, SI-55E3, SI-56E1, SI-57E1, and SI-38E17) exerted weak killing of less than 20% of seeded cells. Therefore, their EC50 values were not comparable to that of SI-55E1 and SI-55E2. SI-55E3 carried an anti-EGFR binding domain at D2 or Fab adopted from Nimotuzumab, whose binding affinity to EGFR is lower than that of Panitumumab for SI-55E1 and Cetuximab for SI-55E2 (from Applicant's application No. PCT/US2020/059230, incorporated herein in its entirety). Surprisingly, SI-55E3 was among those exhibiting weak killing. This finding indicates that both binding specificity and affinity play roles in multi-specific GNC antibody-mediated TDCC.

### Example 8. Comparative Potency of the Same Multi-Specific GNC Antibody Killing Different Cancer Cells

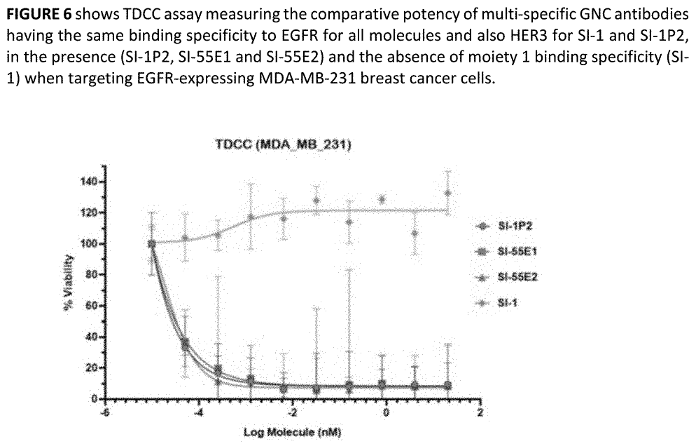

To further characterize the comparative potency of SI-55E1 and SI-55E2 having the same configuration of moiety 1 binding domains and targeting EGFR, MDA-MB-231 cell line was used in TDCC assay. In the same day experiment, two additional antibodies targeting both EGFR and HER3 were used: SI-1P2 is a pentaGNC antibody having the same configuration of moiety 1 binding domains as that of SI-55E1 and SI-55E2 plus an additional moiety 2 domain binding to HER3, and SI-1 is a bispecific antibody against both EGFR and HER3 in the absence of any moiety 1 binding domain. The materials and methods of this TDCC assay were the same as described in Example 1. As shown in FIG. 6, SI-55E1, SI-55E2, and SI-1P2 exerted comparable potency demonstrated by the overlapping dose-viability curves, and their EC50 values were calculated in the range of 17-29 fM (Table 6). In contrast, SI-1 did not reveal any response below the dose in nM in this same day experiment. This result supported the notion that a trio of moiety 1 binding domains (CD3, PD-L1, and 4-1BB) contributes significantly to the potency of multi-specific GNC antibodies.

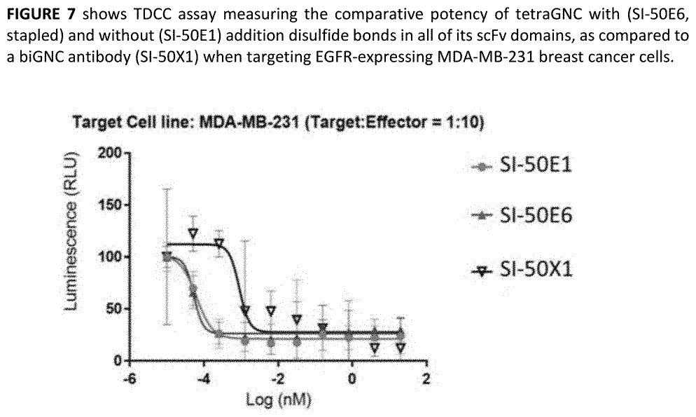

To measure the added effect of moiety 1 binding domain, such as anti-CD3, two tetraGNC antibodies (SI-50E1 and SI-50E6) and one biGNC antibody (SI-50X1) were analyzed in TDCC assay using MDA-MB-231 cell line as target cells. All three antibodies have the same binding specificity to HER2 derived from Trastuzumab (Table 6). SI-50E1 and SI-50E6 have the same configuration of moiety 1 and moiety 2 binding domains, however, the scFv domains of SI-50E6 were engineered with additional disulfide bonds for added stability (i.e. stapled) whereas the scFv domains of SI-50E1 was not stapled. SI-50X1 is a bispecific antibody targeting CD3 and HER2. The TDCC dose-response curves clearly showed that all three GNC antibodies are potent and their EC 50 values were in the range of fM (FIG. 7). The difference in the curves and EC values was due to the absence or the presence of the moiety 2 binding domains to PD-L1 and 4-1BB among the three antibodies.

### Example 9. Selecting TAAs and Assembling Moiety 2 Binding Domains for Multi-Specific GNC Antibodies

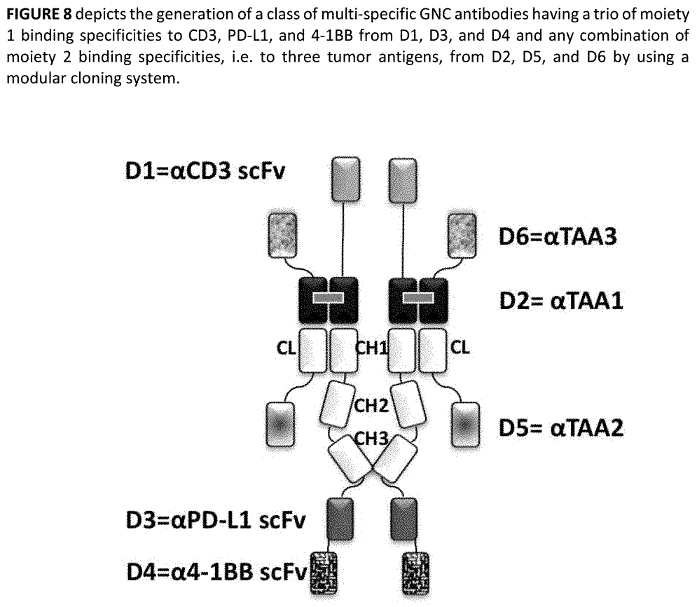

Antibody therapy employs a wide variety of strategy to kill cancer cells either directly or indirectly, and both mechanisms of action depend on binding to surface antigen(s). On the other hand, the cancer cells are evolved to gain their ability of escaping such recognitions, either from the antibody, immune cells, or both. With the capacity of up to six binding specificities, multi-specific GNC antibodies displayed the highest potency as measured by the in vitro EC50 in the range of pM and fM. A highly efficacious multi-specific GNC antibody depends on the composition of both moiety 1 and moiety 2 antigens. Herein, the trio of moiety 1 binding domains (CD3, PD-L1, and 4-1BB) in a configuration (D1, D3, and D4 of HC, respectively) provides the backbone of multi-specific GNC antibodies. Such formatted GNC antibodies allow selecting, screening, and optimizing TAAs of target cancer cells (FIG. 8).

To demonstrate the importance of TAA surface expression profile, quantitative flow cytometry (qFACS) was performed to quantify the approximate number of receptors per cell for various tumor targets. EGFR, HER2, and HER3 are members of the EGFR family whose expression is often upregulated by solid tumors, and PD-L1 is the target for inhibiting the immune checkpoint signaling utilized by a portion of human cancer. Nonetheless, the surface expression of HER3 and PD-L1 was not detectable in MDA-MB-232 cells and Hela cells, respectively (Table 8). The observation may provide an explanation for lacking a synthetic lethal effect when targeting both EGFR and HER3 by SI-1P1 as compared to SI-55E1, SI-55E2, and SI-50E1 (Table 6), each of which targets either EGFR or HER2. It may also explain that the failure of inducing TDCC by control antibody (SI-38E72) is likely due to the absence of PD-L1 on Hela cells (Table 7). In light of evolving and dynamic expression profile of cancer cells, any candidate antibody may be tested for TDCC using different types of cancer cells, such as those shown in Table 9.

To screen for TAAs, a modular cloning platform may be used to efficiently identify TAAs or epitopes of TAAs for assembling moiety 2 binding domains for multi-specific GNC antibodies. For example, TAA-Fc tetraGNC-1 and TAA-Fc tetraGNC-2 are two groups of tetraGNC antibodies with the pair-wise identical binding specificities. The only difference is that all TAA-Fc tetraGNC-2 antibodies have stapled scFv domain D1, D3, and D4 (mutations of VH 44->C, and VL 100->C) of HC. In this case, HC was swapped to produce two groups of tetraGNC antibodies. In another examples, LC may be swapped to produce multi-specific GNC antibodies with an added binding domain to TAA, such as SI-55P10 to SI-55H11, and SI-55E1 to SI-1P1. This modular cloning platform allows efficient assembly of multi-specific GNC antibodies with up to 3 TAAs starting from a single anti-TAA monoclonal antibody.

### Example 10. Functionality of Multi-Specific GNC Proteins with Receptor in the D2 Position

To further validate the flexibility of the GNC platform to accommodate diverse binding domains in each molecular position, a panel of proteins was generated with the NKG2D receptor at the D2 position (Table 10). When monomeric NKG2D is incorporated into the D2 position of both GNC chains, it was predicted that the NKG2D monomers would dimerize upon chain association. SI-49R21 is a monospecific GNC (antibody-like protein) with NKG2D receptor in place of the antibody VH/VL domains. SI-49R22 contains the same format, except that its Fc domain also contains knobs-into-holes mutations (chain A: T366S/L368A/Y407V; and chain B: T366W) for heterodimerization. SI-49R23 is a monospecific protein with NKG2D directly fused to the antibody Fc domain, and SI-49R24 contains this same structure but additionally has knobs-into-holes mutations in the Fc. SI-49R19 is a bispecific GNC with anti-CD3 scFv in D1 and NKG2D in D2, and SI-49R18 is a tri-specific GNC which additionally contains anti-CD19 at D6. SI-49E15 contains anti-CD3 scFv at D1, NKG2D at D2, anti-PD-L1 scFv at D3, and anti-4-1BB scFv at D4; SI-49P6 contains the same domains as SI-49E15, and additionally contains anti-CD19 scFv at D6. SI-49P7 has the same structure as SI-49P6, except it contains 4-1BB ligand trimer at D4 instead of anti 1BB scFv.

All the proteins with NKG2D in D2 were successfully expressed and purified. To validate functionality of the NKG2D receptor in the D2 position, Octet binding was performed. GNC protein was loaded onto AHC sensors at 5 ug/ml, and subsequently bound to 1:2 serial dilutions (top concentration 100 nM) of human MICA (Acro, MIA-H5221). The binding affinities (KD values) resulting from global fit to a 1:1 binding model are shown in Table 11. Mono-, bi-, tri-, tetra-, and penta-GNC proteins with NKG2D at D2 were shown to retain strong binding to the NKG2D ligand MICA, as demonstrated by KD values under 20 nM.

### Example 11. Antigen Binding Independent of GNC Position, Humanization, or Domain Format

To further demonstrate the adaptability of the GNC platform, a panel hexa-specific GNC proteins targeting the same antigens (CD3×EGFR×PD-L1×4-1BB×CD19×HER3) was generated. The entire panel contained anti-PD-L1 scFv at D3, anti-4-1BB scFv at D4, anti-HER3 scFv at D5, and anti-CD19 scFv at D6. Two molecules (SI-77H4 and SI-77H5) contained an anti-CD3 scFv at D1 and anti-EGFR Fab at D2, the difference being that the anti-EGFR domain for SI-77H4 was humanized while that of SI-77H5 retained mouse sequences. Two molecules (SI-55H11 and SI-55H12) contained anti-EGFR scFv at D1 and anti-CD3 Fab at D2, the difference being that the D2 VH/VL contained a disulfide staple in SI-55H11 (VH-44C, VL-100C) but not in SI-55H12. Thus, the panel allowed for clarification of whether D1/D2 positioning affected either protein expression properties or binding affinity to the targeted tumor-associated antigen.

Proteins were expressed transiently in ExpiCHO cells as described in Example 1. After approximately 8 days, GNC titers were measured using protein A sensors on the Octet platform (Table 12). Results demonstrate that the hexa-specific GNC proteins expressed well (≥30 μg/ml) regardless of the positioning and format of the anti-EGFR and anti-CD3 domains. After the first protein A purification step, all proteins had similarly low levels of aggregation with the percent protein of interest ranging from 72 to 85% (Table 12). Next, the affinity of the anti-EGFR domains for human EGFR was assessed by loading GNC proteins onto AHC sensors and using a single concentration (100 nM) of His-tagged human EGFR (expressed in-house) as the analyte. As shown in Table 12, the positioning and format of anti-EGFR and anti-CD3 domains did not significantly affect EGFR binding affinity (KD values within approximately 2-fold). Thus, GNC proteins retain full functionality regardless of the positioning of anti-TAA and anti-CD3 domains between D1 and D2.

### TABLES

TABLE 1
The generation and characterization of a class of tetra-specific GNC (tetraGNC)
antibodies with added binding domains and Fab domain (D1 to D4) on the heavy chain (HC).
NKG2D in D1, D3, D4, D5, and D6 is used as a dimerizing tandem repeat, while NKG2D in D2 is a
monomer that dimerizes upon chain association.

| | | | | |
|---|---|---|---|---|
| SEQ. ID | D1 | D2 | D3 | D4 |

tetraGNC having NKG2D dimer as one of binding domain on HC

| | | | | | |
|---|---|---|---|---|---|
| SI-49E1 | 1-4 | NKG2D-dimer | αCD3 | αPD-L1 | α4-1BB |
| SI-49E2 | 5-8 | NKG2D-dimer | αCD3 | α4-1BB | αPD-L1 |
| SI-49E3 | 9-12 | α4-1BB | αPD-L1 | αCD3 | NKG2D-dimer |
| SI-49E4 | 13-16 | αPD-L1 | α4-1BB | αCD3 | NKG2D-dimer |
| SI-49E11 | 17-20 | NKG2D-dimer | αCD3 | αPD-L1 | 41BBL |
| SI-49E12 | 21-24 | NKG2D-dimer | αCD3 | 41BBL | αPD-L1 |
| SI-49E13 | 25-28 | 41BBL | αPD-L1 | αCD3 | NKG2D-dimer |

TAA-Fab tetraGNC-1

| | | | | | |
|---|---|---|---|---|---|
| SI-50E1 | 109-112 | αCD3 | Tras | αPD-L1 | α4-1BB |
| SI-50E2 | 115-118 | αCD3 | PERT | αPD-L1 | α4-1BB |
| SI-51E1 | 121-124 | αCD3 | αMSLN | αPD-L1 | α4-1BB |
| SI-52E1 | 127-130 | αCD3 | αGD2 | αPD-L1 | α4-1BB |
| SI-53E1 | 133-136 | αCD3 | αClaudin | αPD-L1 | α4-1BB |
| SI-54E1 | 139-142 | αCD3 | Ritu | αPD-L1 | α4-1BB |
| SI-55E1 | 145-148 | αCD3 | Cet | αPD-L1 | α4-1BB |
| SI-55E2 | 151-154 | αCD3 | Pan | αPD-L1 | α4-1BB |
| SI-55E3 | 157-160 | αCD3 | Nimo | αPD-L1 | α4-1BB |
| SI-56E1 | 163-166 | αCD3 | αCD22 | αPD-L1 | α4-1BB |
| SI-57E1 | 169-172 | αCD3 | αCD30 | αPD-L1 | α4-1BB |

TAA-Fab tetraGNC-2 D1, D3, D4 are stapled)

| | | | | | |
|---|---|---|---|---|---|
| SI-50E6 | 111-114 | αCD3 | Tras | αPD-L1 | α4-1BB |
| SI-50E7 | 117-120 | αCD3 | PERT | αPD-L1 | α4-1BB |
| SI-51E4 | 123-126 | αCD3 | αMSLN | αPD-L1 | α4-1BB |
| SI-52E4 | 129-132 | αCD3 | αGD2 | αPD-L1 | α4-1BB |
| SI-53E3 | 135-138 | αCD3 | αClaudin | αPD-L1 | α4-1BB |
| SI-54E3 | 141-144 | αCD3 | Ritu | αPD-L1 | α4-1BB |
| SI-55E8 | 147-150 | αCD3 | Cet | αPD-L1 | α4-1BB |
| SI-55E9 | 153-156 | αCD3 | Pan | αPD-L1 | α4-1BB |
| SI-55E10 | 159-162 | αCD3 | Nimo | αPD-L1 | α4-1BB |
| SI-56E3 | 165-168 | αCD3 | αCD22 | αPD-L1 | α4-1BB |
| SI-57E3 | 171-174 | αCD3 | αCD30 | αPD-L1 | α4-1BB |

TABLE 2
The generation and characterization of penta- and hexa-specific GNC (pentaGNC
and hexaGNC) antibodies, including their sequence identification numbers
(SEQ ID), the binding specificity of each domain, from D1 to D6.

| | | | | | | |
|---|---|---|---|---|---|---|
| SEQ. ID | D1 | D2 | D3 | D4 | D5 | D6 |

D5-pentaGNC

| | | | | | | | |
|---|---|---|---|---|---|---|---|
| SI-1P1 | 33-36 | αCD3 | Cet | αPD-L1 | α4-1BB | MM-111 | n/a |
| SI-1P2 | 37-40 | αCD3 | Cet | αPD-L1 | α4-1BB | MM-111 | n/a |
| SI-1P4 | 41-44 | αCD3 | Pan | αPD-L1 | α4-1BB | MM-111 | n/a |
| SI-39P1 | 45-48 | αCD3 | αVIII | αPD-L1 | α4-1BB | MM-111 | n/a |

D5-pentaGNC

| | | | | | | | |
|---|---|---|---|---|---|---|---|
| SI-38P5 | 49-52 | αCD3 | Ritu | αPD-L1 | α4-1BB | αCD19 | n/a |
| SI-38P6 | 53-56 | αCD3 | Ritu | αPD-L1 | α4-1BB | αCD19 | n/a |

D5-pentaGNC with NKG2D dimer as a binding domain on LC

| | | | | | | | |
|---|---|---|---|---|---|---|---|
| SI-49P1 | 57-60 | αCD3-alt | αMESO | αPD-L1 | α4-1BB | NKG2D- | n/a |
| | | | | | | dimer | |
| SI-49P2 | 61-64 | αCD3-alt | αClaudin | αPD-L1 | α4-1BB | NKG2D- | n/a |
| | | | | | | dimer | |
| SI-49P3 | 65-68 | αCD3-alt | Tras | αPD-L1 | α4-1BB | NKG2D- | n/a |
| | | | | | | dimer | |
| SI-49P4 | 69-72 | αCD3-alt | αVIII | αPD-L1 | α4-1BB | NKG2D- | n/a |
| | | | | | | dimer | |
| SI-49P5 | 73-76 | αCD3-alt | αCD33 | αPD-L1 | α4-1BB | NKG2D- | n/a |
| | | | | | | dimer | |

HexaGNC

| | | | | | | | |
|---|---|---|---|---|---|---|---|
| SI-1H1 | 175-178 | Pan | αCD3 | αPD-L1 | α4-1BB | SI-BU12 | MM-111 |
| SI-77H4 | 323-326 | αCD3-alt | αEGFR | αPD-L1 | α4-1BB | MM-111 | SI-BU12 |
| | | | H7 | | | | |
| SI-77H5 | 327-330 | αCD3-alt | Cet | αPD-L1 | α4-1BB | MM-111 | SI-BU12 |
| SI-55H11 | 105-108 | αEGFR | αCD3 | αPD-L1 | α4-1BB | MM-111 | SI-BU12 |
| | | H7 | | | | | |
| SI-55H12 | 331-334 | αEGFR | αCD3 | αPD-L1 | α4-1BB | MM-111 | SI-BU12 |
| | | H7 | | | | | |

TABLE 3
Annotation of binding domains for their binding
specificity, domain structure, origin,
and sequence identification number (SEQ ID).

| | Binding | | | |
|---|---|---|---|---|
| Target | Domain | Structure | Origin | SEQ ID |
| Claudin18.2 | αClaudin | scFv | Zolbetuximab | 217-220 |
| HER2 | Tras | scFv | Trastuzumab | 221-224 |
| HER2 | PERT | scFv | Pertuzumab | 225-228 |
| MSLN | αMSLN | scFv | Amatuximab | 229-232 |
| GD2 | αGD2 | scFv | Dinutuximab | 233-236 |
| CD20 | Ritu | scFv | Rituximab | 237-240 |
| EGFR | Cet | scFv | Cetuximab | 241-244 |
| EGFR | αEGFR H7 | scFv | Cetuximab | 319-322 |
| | | | (humanized) | |
| EGFR | Pan | scFv | Panitumumab | 245-248 |
| EGFR | Nimo | scFv | Nimotuzumab | 249-252 |
| CD22 | αCD22 | scFv | Inotuzumab | 253-256 |
| CD30 | αCD30 | scFv | Brentuximab | 257-260 |
| HER3 | MM-111 | scFv | MM-111 | 261-264 |
| mutant | αVIII | scFv | ABT-806 | 265-268 |
| EGFRvIII | | | | |
| CD19 | αCD19 | scFv | 21D4 | 269-272 |
| CD33 | αCD33 | scFv | Gemtuzumab | 273-276 |
| CD3 | αCD3 | scFv | 284A10 | 277-280 |
| CD3 | αCD3-alt | scFv | 284A10 FR 1 | 315-318 |
| PD-L1 | αPD-L1 | scFv | PL221G5 | 281-284 |
| 4-1BB | α4-18BB | scFv | 466F6 | 285-288 |
| CD19 | SI-BU12 | scFv | SI-BU12 | 289-292 |
| NKG2D Ligand | NKG2D | Receptor | NKG2D receptor | 293-294 |
| NKG2D Ligand | NKG2D- | Receptor | NKG2D receptor | 309-310 |
| | dimer | | | |
| 4-1BB | 41BBL | Ligand | 4-1BB ligand | 295-296 |

TABLE 4
The comparative potency of multi-specific GNC molecules with NKG2D
receptor dimer as an additional binding moiety to immune cells
for killing breast cancer cells (MDA-MB-231) in TDCC assay.

| | | | | | | | | |
|---|---|---|---|---|---|---|---|---|
| Sample | | | | | | | EC50 | |
| ID | D1 | D2 | D3 | D4 | D5 | D6 | (pM) | E:T |

| | | | | | | | |
|---|---|---|---|---|---|---|---|
| SI-38E72 | αCD3 | αFITC | αPD-L1 | α4-1BB | | 1.230 | 5 |
| SI-49E1 | NKG2D- | αCD3 | αPD-L1 | α4-1BB | | 0.054 | 5 |
| | dimer | | | | | | |
| SI-49E2 | NKG2D- | αCD3 | α4-1BB | αPD-L1 | | 0.129 | 5 |
| | dimer | | | | | | |
| SI-49E3 | α4-1BB | αPD-L1 | αCD3 | NKG2D- | | 0.055 | 5 |
| | | | | dimer | | | |
| SI-49E4 | αPD-L1 | α4-1BB | αCD3 | NKG2D- | | 0.009 | 5 |
| | | | | dimer | | | |
| SI-49P1 | αCD3 | MSLN | αPD-L1 | α4-1BB | NKG2D- | 0.002 | 5 |
| | | | | | dimer | | |
| SI-49X1 | NKG2D- | αCD3 | | | | 2660 | 5 |
| | dimer | | | | | | |
| SI-49X2 | | | αCD3 | NKG2D- | | 40.7 | 5 |
| | | | | dimer | | | |

TABLE 5
The comparative potency of multi-specific GNC antibodies having the same
binding specificity but different domain structures, e.g. scFv or ligand,
for killing pancreatic cancer cells (BXPC-3) in TDCC assay.

| Sample | | | | D4/scFv | | | EC50 | |
|---|---|---|---|---|---|---|---|---|
| ID | D1/scFv | D2/Fab | D3/scFv | or ligand | D5/scFv | D6/scFv | (pM) | E:T |
| SI-1P1 | αCD3 | Cet | αPD-L1 | α4-1BB | MM-111 | | 0.281 | 5 |
| SI-55P9 | αEGFR | αCD3^{a} | αPD-L1 | α4-1BB | | αCD19^{b} | 0.487 | 5 |
| | H7 | | | | | | | |
| SI-55P10 | αEGFR | αCD3^{a} | αPD-L1 | 41BBL | | αCD19^{b} | 0.736 | 5 |
| | H7 | | | | | | | |
| SI-55H11 | αEGFR | αCD3^{a} | αPD-L1 | α41BB | MM-111 | αCD19^{b} | 0.094 | 5 |
| | H7 | | | | | | | |

^{a}284A10, see Applicant's application No. PCT/US2018/039143;
^{b}SI-huBU12, see Applicant's application No. PCT/US2020/059230.

TABLE 6
The comparative potency of multi-specific GNC antibodies targeting
at least one of these tumor antigens, EGFR, HER2, HER3, or MSLN
for killing breast cancer cells (MDA-MB-231) in TDCC assay.

| | | | | | | | | |
|---|---|---|---|---|---|---|---|---|
| Sample | | | | | | | EC50 | |
| ID | D1 | D2 | D3 | D4 | D5 | D6 | (pM) | E:T |

| | | | | | | | |
|---|---|---|---|---|---|---|---|
| SI-38E72 | αCD3 | αFITC | αPD-L1 | α4-1BB | | 1.230 | 5 |
| SI-55E1 | αCD3 | Cet | αPD-L1 | α4-1BB | | 0.017 | 5 |
| SI-55E2 | αCD3 | Pan | αPD-L1 | α4-1BB | | 0.018 | 5 |
| SI-50E1 | αCD3 | Tras | αPD-L1 | α4-1BB | | 0.063 | 10 |
| SI-50E6 | αCD3 | Tras | αPD-L1 | α4-1BB | | 0.053 | 10 |
| SI-50X1 | αCD3 | Tras | | | | 0.902 | 10 |
| SI-51E1 | αCD3 | αMSLN | αPD-L1 | α4-1BB | | 0.009 | 5 |
| SI-51E4 | αCD3 | αMSLN | αPD-L1 | α4-1BB | | 0.009 | 5 |
| SI-51X1 | αCD3 | αMSLN | | | NKG2D | 0.465 | 5 |
| SI-49P1 | αCD3- | αMSLN | αPD-L1 | α4-1BB | NKG2D | 0.002 | 5 |
| | alt | | | | | | |
| SI-1P1 | αCD3 | Cet | αPD-L1 | α4-1BB | MM-111 | 0.029 | 5 |

TABLE 7
The comparative potency of tetraGNC antibodies targeting one additional
tumor antigen for killing cervical cancer cells (Hela) in TDCC assay.

| | | | | | | | | |
|---|---|---|---|---|---|---|---|---|
| Sample | | | | | | | EC50 | |
| ID | D1 | D2 | D3 | D4 | D5 | D6 | (pM) | E:T |

| | | | | | | |
|---|---|---|---|---|---|---|
| SI-38E72 | αCD3 | αFITC | αPD-L1 | α4-1BB | 48000 | 10 |
| SI-50E1 | αCD3 | Tras | αPD-L1 | α4-1BB | nd | 10 |
| SI-50E2 | αCD3 | PERT | αPD-L1 | α4-1BB | 35 | 10 |
| SI-51E1 | αCD3 | αMSLN | αPD-L1 | α4-1BB | 3140 | 10 |
| SI-52E1 | αCD3 | αGD2 | αPD-L1 | α4-1BB | 1740 | 10 |
| SI-53E1 | αCD3 | αClaudin | αPD-L1 | α4-1BB | nd | 10 |
| SI-54E1 | αCD3 | Ritu | αPD-L1 | α4-1BB | 30 | 10 |
| SI-55E1 | αCD3 | Cet | αPD-L1 | α4-1BB | 13 | 10 |
| SI-55E2 | αCD3 | Pan | αPD-Ll | α4-1BB | 9 | 10 |
| SI-55E3 | αCD3 | Nimo | αPD-L1 | α4-1BB | 33 | 10 |
| SI-56E1 | αCD3 | αCD22 | αPD-L1 | α4-1BB | 186 | 10 |
| SI-57E1 | αCD3 | αCD30 | αPD-L1 | α4-1BB | 96 | 10 |
| SI-38E17 | αCD3 | αCD19 | αPD-L1 | α4-1BB | 46 | 10 |

TABLE 8
qFACS analysis of the expression of tumor antigens and PD-L1
on the surface of normal T lymphocytes and cancer cells.

| \Antibody | Panitumumab | Trastuzumab | MM111 | PL221G5 |
|---|---|---|---|---|
| Cell\ | αEGFR | αHER2 | αHER3 | αPD-L1 |
| T Lymphocyte | 0.00 | 0.00 | 0.00 | 2000.00 |
| MDA-MB-231 | 63858.26 | 15403.43 | 0.00 | 25456.74 |
| BxPC3 | 101584.25 | 1575.51 | 0.00 | 11608.69 |
| HeLa | 24908.49 | 10459.99 | 45441.51 | 0.00 |

TABLE 9
The potency of multi-specific GNC antibodies as determined by
the surface expression of tumor antigen(s) on the cancer cells.

| | | | | | | | | |
|---|---|---|---|---|---|---|---|---|
| Sample | | | | | | | EC50 (pM) | EC50 (pM) |
| ID | D1 | D2 | D3 | D4 | D5 | D6 | Hela | MDA-MB-231 |

| | | | | | | |
|---|---|---|---|---|---|---|
| SI-38E72 | αCD3 | αFITC | αPD-L1 | α4-1BB | 48000 | 1.230 |
| SI-55E1 | αCD3 | Cet | αPD-L1 | α4-1BB | 13 | 0.017 |
| SI-55E2 | αCD3 | Pan | αPD-L1 | α4-1BB | 9 | 0.018 |
| SI-51E1 | αCD3 | αMSLN | αPD-L1 | α4-1BB | 3140 | 0.009 |
| SI-50E1 | αCD3 | Tras | αPD-L1 | α4-1BB | n.d. | 0.063 |

TABLE 10
The generation and characterization of mono-, bi-, tri-, tetra-, and penta-
specific GNC antibodies, including their sequence identification numbers
(SEQ ID), the binding specificity of each domain, from D1 to D6.

| | | | | | | | |
|---|---|---|---|---|---|---|---|
| Sample ID | SEQ ID | D1 | D2 | D3 | D4 | D5 | D6 |

| | | | | | | |
|---|---|---|---|---|---|---|
| SI-49R21 | 195-198 | | NKG2D | | | |
| SI-49R22* | 199-204 | | NKG2D | | | |
| SI-49R23** | 205-206 | | NKG2D | | | |
| SI-49R24*** | 207-210 | | NKG2D | | | |
| SI-49R19 | 183-186 | αCD3-alt | NKG2D | | | |
| SI-49R18 | 187-190 | αCD3-alt | NKG2D | | | SI-BU12 |
| SI-49E15 | 191-194 | αCD3-alt | NKG2D | αPD-L1 | α4-1BB | |
| SI-49P6 | 77-80 | αCD3-alt | NKG2D | αPD-L1 | α4-1BB | SI-BU12 |
| SI-49P7 | 81-84 | αCD3-alt | NKG2D | αPD-L1 | 41BBL | SI-BU12 |

*SI-49R22 contains heterodimerizing Fc with knobs-into-holes mutations (chain A: T366S/L368A/Y407V; and chain B: T366W)
**SI-49R23 lacks the CH1/CL domains
***SI-49R24 contains heterodimerizing Fc with knobs-into-holes mutations (chain A: T366S/L368A/Y407V; and chain B: T366W), and lacks CH1/CL domains

TABLE 11
The comparative binding affinity of selected multispecific
GNCs with NKG2D receptor in the D2 position
for NKG2D ligand, MICA.

| Sample ID | MICA K_{D} (nM) |
|---|---|
| SI-49R21 | 8.95 |
| SI-49R22 | 1.93 |
| SI-49R23 | 14.9 |
| SI-49R24 | 17.7 |
| SI-49R19 | 3.93 |
| SI-49R18 | 5.11 |
| SI-49E15 | 3.34 |
| SI-49P6 | 8.7 |
| SI-49P7 | 11.2 |

TABLE 12
Characterization of representative hexa-GNC antibodies comprising
anti-EGFR and anti-CD3 domains at either D1 (scFv) or D2 (Fab).

| Sample | | | | | | | EGFR KD | Titer | aSEC | EC50 |
|---|---|---|---|---|---|---|---|---|---|---|
| ID | D1 | D2 | D3 | D4 | D5 | D6 | (nM) | (μg/ml) | % POI | (pM) |
| SI- | αCD3- | αEGFR | αPD- | α4- | MM- | SI- | 3.29 | 61.1 | 77.25 | 0.147 |
| 77H4 | alt | H7 | L1 | 1BB | 111 | BU12 | | | | |
| SI- | αCD3- | Cet | αPD- | α4- | MM- | SI- | 3.39 | 35 | 72.02 | 0.192 |
| 77H5 | alt | | L1 | 1BB | 111 | BU12 | | | | |
| SI- | αEGFR | αCD3 | αPD- | α4- | MM- | SI- | 4.65 | 30 | 84.42 | 0.082 |
| 55H11* | H7 | | L1 | 1BB | 111 | BU12 | | | | |
| SI- | αEGFR | αCD3 | αPD- | α4- | MM- | SI- | 7.87 | 76 | 80.00 | nd |
| 55H12 | H7 | | L1 | 1BB | 111 | BU12 | | | | |

*SI-55H11 D2 comprises a disulfide staple between VH and VL (VH-44C, VL-100C)

SEQUENCE LISTING

| | | Heavy | | Light | | | |
|---|---|---|---|---|---|---|---|
| Protein | SEQ. | Chain (HC) | | Chain (LC) | | HC2 | HC2 |
| ID | ID | DNA | aa | DNA | aa | DNA | aa |
| SI-49E1 | 1-4 | 1 | 2 | 3 | 4 | | |
| SI-49E2 | 5-8 | 5 | 6 | 7 | 8 | | |
| SI-49E3 | 9-12 | 9 | 10 | 11 | 12 | | |
| SI-49E4 | 13-16 | 13 | 14 | 15 | 16 | | |
| SI-49E11 | 17-20 | 17 | 18 | 19 | 20 | | |
| SI-49E12 | 21-24 | 21 | 22 | 23 | 24 | | |
| SI-49E13 | 25-28 | 25 | 26 | 27 | 28 | | |
| SI-49E14 | 29-32 | 29 | 30 | 31 | 32 | | |
| SI-1P1 | 33-36 | 33 | 34 | 35 | 36 | | |
| SI-1P2 | 37-40 | 37 | 38 | 39 | 40 | | |
| SI-1P4 | 41-44 | 41 | 42 | 43 | 44 | | |
| SI-39P1 | 45-48 | 45 | 46 | 47 | 48 | | |
| SI-38P5 | 49-52 | 49 | 50 | 51 | 52 | | |
| SI-38P6 | 53-56 | 53 | 54 | 55 | 56 | | |
| SI-49P1 | 57-60 | 57 | 58 | 59 | 60 | | |
| SI-49P2 | 61-64 | 61 | 62 | 63 | 64 | | |
| SI-49P3 | 65-68 | 65 | 66 | 67 | 68 | | |
| SI-49P4 | 69-72 | 69 | 70 | 71 | 72 | | |
| SI-49P5 | 73-76 | 73 | 74 | 75 | 76 | | |
| SI-49P6 | 77-80 | 77 | 78 | 79 | 80 | | |
| SI-49P7 | 81-84 | 81 | 82 | 83 | 84 | | |
| SI-49P10 | 85-88 | 85 | 86 | 87 | 88 | | |
| SI-38E17 | 97-100 | 97 | 98 | 99 | 100 | | |
| SI-55H11 | 105-108 | 105 | 106 | 107 | 108 | | |
| SI-50E1 | 109-112 | 109 | 110 | 111 | 112 | | |
| SI-50E2 | 115-118 | 115 | 116 | 117 | 118 | | |
| SI-51E1 | 121-124 | 121 | 122 | 123 | 124 | | |
| SI-52E1 | 127-130 | 127 | 128 | 129 | 130 | | |
| SI-53E1 | 133-136 | 133 | 134 | 135 | 136 | | |
| SI-54E1 | 139-142 | 139 | 140 | 141 | 142 | | |
| SI-55E1 | 145-148 | 145 | 146 | 147 | 148 | | |
| SI-55E2 | 151-154 | 151 | 152 | 153 | 154 | | |
| SI-55E3 | 157-160 | 157 | 158 | 159 | 160 | | |
| SI-56E1 | 163-166 | 163 | 164 | 165 | 166 | | |
| SI-57E1 | 169-172 | 169 | 170 | 171 | 172 | | |
| SI-50E6 | 111-114 | 113 | 114 | 111 | 112 | | |
| SI-50E7 | 117-120 | 119 | 120 | 117 | 118 | | |
| SI-51E4 | 123-126 | 125 | 126 | 123 | 124 | | |
| SI-52E4 | 129-132 | 131 | 132 | 129 | 130 | | |
| SI-53E3 | 135-138 | 137 | 138 | 135 | 136 | | |
| SI-54E3 | 141-144 | 143 | 144 | 141 | 142 | | |
| SI-55E8 | 147-150 | 149 | 150 | 147 | 148 | | |
| SI-55E9 | 153-156 | 155 | 156 | 153 | 154 | | |
| SI-55E10 | 159-162 | 161 | 162 | 159 | 160 | | |
| SI-56E3 | 165-168 | 167 | 168 | 165 | 166 | | |
| SI-57E3 | 171-174 | 173 | 174 | 171 | 172 | | |
| SI-1H1 | 175-178 | 175 | 176 | 177 | 178 | | |
| SI-49R19 | 183-186 | 183 | 184 | 185 | 186 | | |
| SI-49R18 | 187-190 | 187 | 188 | 189 | 190 | | |
| SI-49E15 | 191-194 | 191 | 192 | 193 | 194 | | |
| SI-49R21 | 195-198 | 195 | 196 | 197 | 198 | | |
| SI-49R22 | 199-204 | 199 | 200 | 201 | 202 | 203 | 204 |
| SI-49R23 | 205-206 | 205 | 206 | | | | |
| SI-49R24 | 207-210 | 207 | 208 | 209 | 210 | | |
| SI-55P9 | 301-304 | 301 | 302 | 303 | 304 | | |
| SI-55P10 | 305-308 | 305 | 306 | 307 | 308 | | |
| SI-77H4 | 323-326 | 323 | 324 | 325 | 326 | | |
| SI-77H5 | 327-330 | 327 | 328 | 329 | 330 | | |
| SI-55H12 | 331-334 | 331 | 332 | 333 | 334 | | |

| Binding | | VH/1/A | VH/1/A | VL/2/B | VL/2/B |
|---|---|---|---|---|---|
| Domain | SEQ ID | DNA | AA | DNA | AA |
| αClaudin | 217-220 | 217 | 218 | 219 | 220 |
| Tras | 221-224 | 221 | 222 | 223 | 224 |
| PERT | 225-228 | 225 | 226 | 227 | 228 |
| αMSLN | 229-232 | 229 | 230 | 231 | 232 |
| αGD2 | 233-236 | 233 | 234 | 235 | 236 |
| Ritu | 237-240 | 237 | 238 | 239 | 240 |
| Get | 241-244 | 241 | 242 | 243 | 244 |
| αEGFR H7 | 319-322 | 319 | 320 | 321 | 322 |
| Pan | 245-248 | 245 | 246 | 247 | 248 |
| Nimo | 249-252 | 249 | 250 | 251 | 252 |
| αCD22 | 253-256 | 253 | 254 | 255 | 256 |
| αCD30 | 257-260 | 257 | 258 | 259 | 260 |
| MM-111 | 261-264 | 261 | 262 | 263 | 264 |
| αVIII | 265-268 | 265 | 266 | 267 | 268 |
| αCD19 | 269-272 | 269 | 270 | 271 | 272 |
| αCD33 | 273-276 | 273 | 274 | 275 | 276 |
| αCD3 | 277-280 | 277 | 278 | 279 | 280 |
| αCD3-alt | 315-318 | 315 | 316 | 317 | 318 |
| αPD-L1 | 281-284 | 281 | 282 | 283 | 284 |
| α4-1BB | 285-288 | 285 | 286 | 287 | 288 |
| SI-BU12 | 289-292 | 289 | 290 | 291 | 292 |
| αFITC | 311-314 | 311 | 312 | 313 | 314 |
| NKG2D | 293-294 | 293 | 294 | | |
| NKG2D- | 309-310 | 309 | 310 | | |
| dimer | | | | | |
| 41BBL | 295-296 | 295 | 296 | | |
| αEGFR | 297-298 | 297 | 298 | | |
| αHER3 | 299-300 | 299 | 300 | | |

CDR's underlined in amino acid sequences
Chain A: HC or Chain 1
Chain B: LC or Chain 2

>SEQ ID 1 SI-49E1 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGGAGGCGGTGGCTCAGGCGGAGGCGGCTCAGGAGGTGGCGGTTCA
GGAGGCGGCGGATCTTTCCTAAACTCATTATTCAACCAAGAAGTTCAAATTCCCTTGACCGAAAGTTACTGTGGCCC
ATGTCCTAAAAACTGGATATGTTACAAAAATAACTGCTACCAATTTTTTGATGAGAGTAAAAACTGGTATGAGAGCC
AGGCTTCTTGTATGTCTCAAAATGCCAGCCTTCTGAAAGTATACAGCAAAGAGGACCAGGATTTACTTAAACTGGTG
AAGTCATATCATTGGATGGGACTAGTACACATTCCAACAAATGGATCTTGGCAGTGGGAAGATGGCTCCATTCTCTC
ACCCAACCTACTAACAATAATTGAAATGCAGAAGGGAGACTGTGCACTCTATGCCTCGAGCTTTAAAGGCTATATAG
AAAACTGTTCAACTCCAAATACGTACATCTGCATGCAAAGGACTGTGGTCGACGGCGGTGGAGGGTCCGGCGGTGGT
GGATCAGAGGTGCAGCTGGTGGAGTCTGGGGGAGGCTTGGTCCAGCCTGGGGGGTCCCTGAGACTCTCCTGTGCAGC
CTCTGGATTCACCATCAGTACCAATGCAATGAGCTGGGTCCGCCAGGCTCCAGGGAAGGGGCTGGAGTGGATCGGAG
TCATTACTGGTCGTGATATCACATACTACGCGAGCTGGGCGAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGACGGTGGTTC
TTCTGCTATTACTAGTAACAACATTTGGGGCCAGGGAACCCTGGTCACCGTGTCCTCAGCTAGCACCAAGGGCCCAT
CGGTCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAAAACTCACACATGC
CCACCGTGCCCAGCACCTGAAGCCGCGGGGGCACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTACACCCTGCCCCCATCCC
GGGATGAGCTGACCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTGGCGGTGGAGGGTCCGGCGGTGGTGGATCCGAG
GTGCAGCTGTTGGAGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGGGGCTGGAGTGGATCGCATGCATTG
CTGCTGGTAGTGCTGGTATCACTTACGACGCGAACTGGGCGAAAGGCCGGTTCACCATCTCCAGAGACAATTCCAAG
AACACGCTGTATCTGCAAATGAACAGCCTGAGAGCCGAGGACACGGCCGTATATTACTGTGCGAGATCGGCGTTTTC
GTTCGACTACGCCATGGACCTCTGGGGCCAGGGAACCCTGGTCACCGTCTCGAGCGGTGGAGGCGGATCTGGCGGAG
GTGGTTCCGGCGGTGGCGGCTCCGGTGGAGGCGGCTCTGACATCCAGATGACCCAGTCTCCTTCCACCCTGTCTGCA
TCTGTAGGAGACAGAGTCACCATCACTTGCCAGGCCAGTCAGAGCATTAGTTCCCACTTAAACTGGTATCAGCAGAA
ACCAGGGAAAGCCCCTAAGCTCCTGATCTATAAGGCATCCACTCTGGCATCTGGGGTCCCATCAAGGTTCAGCGGCA
GTGGATCTGGGACAGAATTTACTCTCACCATCAGCAGCCTGCAGCCTGATGATTTTGCAACTTATTACTGCCAACAG
GGTTATAGTTGGGGTAATGTTGATAATGTTTTCGGCGGAGGGACCAAGGTGGAGATCAAAGGCGGTGGAGGGTCCGG
CGGTGGTGGCTCCGGACGGTCGCTGGTGGAGTCTGGGGGAGGCTTGGTCCAGCCTGGGGGGTCCCTGAGACTCTCCT
GTACTGCCTCTGGATTCACCATCAGTAGCTACCACATGCAGTGGGTCCGCCAGGCTCCAGGGAAGGGGCTGGAGTAC
ATCGGAACCATTAGTAGTGGTGGTAATGTATACTACGCAAGCTCCGCTAGAGGCAGATTCACCATCTCCAGACCCTC
GTCCAAGAACACGGTGGATCTTCAAATGAACAGCCTGAGAGCCGAGGACACGGCTGTGTATTACTGTGCGAGAGACT
CTGGTTATAGTGATCCTATGTGGGGCCAGGGAACCCTGGTCACCGTCTCTTCAGGCGGTGGCGGTAGTGGGGGAGGC
GGTTCTGGCGGCGGAGGGTCCGGCGGTGGAGGATCAGACGTTGTGATGACCCAGTCTCCATCTTCCGTGTCTGCATC
TGTAGGAGACAGAGTCACCATCACCTGTCAGGCCAGTCAGAACATTAGGACTTACTTATCCTGGTATCAGCAGAAAC
CAGGGAAAGCCCCTAAGCTCCTGATCTATGCTGCAGCCAATCTGGCATCTGGGGTCCCATCAAGGTTCAGCGGCAGT
GGATCTGGGACAGATTTCACTCTCACCATCAGCGACCTGGAGCCTGGCGATGCTGCAACTTACTATTGTCAGTCTAC
CTATCTTGGTACTGATTATGTTGGCGGTGCTTTCGGCGGAGGGACCAAGGTGGAGATCAAA
>SEQ ID 2 SI-49E1 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVGGGGSGGGGSGGGGS
GGGGSFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTVVDGGGGSGGG
GSEVQLVESGGGLVQPGGSLRLSCAASGFTISTNAMSWVRQAPGKGLEWIGVITGRDITYYASWAKGRFTISRDNSK
NTLYLQMNSLRAEDTAVYYCARDGGSSAITSNNIWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKTHTC
PPCPAPEAAGAPSVFLFPPKPKDTLMISRTPEVTCVVVDVSHEDPEVKFNWYVDGVEVHNAKTKPREEQYNSTYRVV
SVLTVLHQDWLNGKEYKCAVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGGGGGSGGGGSE
VQLLESGGGLVQPGGSLRLSCAASGFSFSSGYDMCWVRQAPGKGLEWIACIAAGSAGITYDANWAKGRFTISRDNSK
NTLYLQMNSLRAEDTAVYYCARSAFSFDYAMDLWGQGTLVTVSSGGGGSGGGGSGGGGSGGGGSDIQMTQSPSTLSA
SVGDRVTITCQASQSISSHLNWYQQKPGKAPKLLIYKASTLASGVPSRFSGSGSGTEFTLTISSLQPDDFATYYCQQ
GYSWGNVDNVFGGGTKVEIKGGGGSGGGGSGRSLVESGGGLVQPGGSLRLSCTASGFTISSYHMQWVRQAPGKGLEY
IGTISSGGNVYYASSARGRFTISRPSSKNTVDLQMNSLRAEDTAVYYCARDSGYSDPMWGQGTLVTVSSGGGGSGGG
GSGGGGSGGGGSDVVMTQSPSSVSASVGDRVTITCQASQNIRTYLSWYQQKPGKAPKLLIYAAANLASGVPSRFSGS
GSGTDFTLTISDLEPGDAATYYCQSTYLGTDYVGGAFGGGTKVEIK
>SEQ ID 3 SI-49E1 chain B nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGT
>SEQ ID 4 SI-49E1 chain B aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>SEQ ID 5 SI-49E2 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGGAGGCGGTGGCTCAGGCGGAGGCGGCTCAGGAGGTGGCGGTTCA
GGAGGCGGCGGATCTTTCCTAAACTCATTATTCAACCAAGAAGTTCAAATTCCCTTGACCGAAAGTTACTGTGGCCC
ATGTCCTAAAAACTGGATATGTTACAAAAATAACTGCTACCAATTTTTTGATGAGAGTAAAAACTGGTATGAGAGCC
AGGCTTCTTGTATGTCTCAAAATGCCAGCCTTCTGAAAGTATACAGCAAAGAGGACCAGGATTTACTTAAACTGGTG
AAGTCATATCATTGGATGGGACTAGTACACATTCCAACAAATGGATCTTGGCAGTGGGAAGATGGCTCCATTCTCTC
ACCCAACCTACTAACAATAATTGAAATGCAGAAGGGAGACTGTGCACTCTATGCCTCGAGCTTTAAAGGCTATATAG
AAAACTGTTCAACTCCAAATACGTACATCTGCATGCAAAGGACTGTGGTCGACGGCGGTGGAGGGTCCGGCGGTGGT
GGATCAGAGGTGCAGCTGGTGGAGTCTGGGGGAGGCTTGGTCCAGCCTGGGGGGTCCCTGAGACTCTCCTGTGCAGC
CTCTGGATTCACCATCAGTACCAATGCAATGAGCTGGGTCCGCCAGGCTCCAGGGAAGGGGCTGGAGTGGATCGGAG
TCATTACTGGTCGTGATATCACATACTACGCGAGCTGGGCGAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGACGGTGGTTC
TTCTGCTATTACTAGTAACAACATTTGGGGCCAGGGAACCCTGGTCACCGTGTCCTCAGCTAGCACCAAGGGCCCAT
CGGTCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAAAACTCACACATGC
CCACCGTGCCCAGCACCTGAAGCCGCGGGGGCACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTACACCCTGCCCCCATCCC
GGGATGAGCTGACCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTGGCGGTGGAGGGTCCGGCGGTGGTGGATCCCGG
TCGCTGGTGGAGTCTGGGGGAGGCTTGGTCCAGCCTGGGGGGTCCCTGAGACTCTCCTGTACTGCCTCTGGATTCAC
CATCAGTAGCTACCACATGCAGTGGGTCCGCCAGGCTCCAGGGAAGGGGCTGGAGTACATCGGAACCATTAGTAGTG
GTGGTAATGTATACTACGCGAGCTCCGCGAGAGGCAGATTCACCATCTCCAGACCCTCGTCCAAGAACACGGTGGAT
CTTCAAATGAACAGCCTGAGAGCCGAGGACACGGCTGTGTATTACTGTGCGAGAGACTCTGGTTATAGTGATCCTAT
GTGGGGCCAGGGAACCCTGGTCACCGTCTCTTCAGGCGGTGGCGGTAGTGGGGGAGGCGGTTCTGGCGGCGGAGGGT
CCGGCGGTGGAGGATCAGACGTTGTGATGACCCAGTCTCCATCTTCCGTGTCTGCATCTGTAGGAGACAGAGTCACC
ATCACCTGTCAGGCCAGTCAGAACATTAGGACTTACTTATCCTGGTATCAGCAGAAACCAGGGAAAGCCCCTAAGCT
CCTGATCTATGCTGCAGCCAATCTGGCATCTGGGGTCCCATCAAGGTTCAGCGGCAGTGGATCTGGGACAGATTTCA
CTCTCACCATCAGCGACCTGGAGCCTGGCGATGCTGCAACTTACTATTGTCAGTCTACCTATCTTGGTACTGATTAT
GTTGGCGGTGCTTTCGGCGGAGGGACCAAGGTGGAGATCAAAGGCGGTGGAGGGTCCGGCGGTGGTGGCTCCGGAGA
GGTGCAGCTGTTGGAGTCTGGGGGAGGCTTGGTACAGCCTGGGGGGTCCCTGAGACTCTCCTGTGCAGCCTCTGGAT
TCTCCTTCAGTAGCGGGTACGACATGTGCTGGGTCCGCCAGGCTCCAGGGAAGGGGCTGGAGTGGATCGCATGCATT
GCTGCTGGTAGTGCTGGTATCACTTACGACGCGAACTGGGCGAAAGGCCGGTTCACCATCTCCAGAGACAATTCCAA
GAACACGCTGTATCTGCAAATGAACAGCCTGAGAGCCGAGGACACGGCCGTATATTACTGTGCGAGATCGGCGTTTT
CGTTCGACTACGCCATGGACCTCTGGGGCCAGGGAACCCTGGTCACCGTCTCGAGCGGCGGTGGCGGTAGTGGGGGA
GGCGGTTCTGGCGGCGGAGGGTCCGGCGGTGGAGGATCAGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAACTGGTATCAGCAGA
AACCAGGGAAAGCCCCTAAGCTCCTGATCTATAAGGCATCCACTCTGGCATCTGGGGTCCCATCAAGGTTCAGCGGC
AGTGGATCTGGGACAGAATTTACTCTCACCATCAGCAGCCTGCAGCCTGATGATTTTGCAACTTATTACTGCCAACA
GGGTTATAGTTGGGGTAATGTTGATAATGTTTTCGGCGGAGGGACCAAGGTGGAGATCAAA
>SEQ ID 6 SI-49E2 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVGGGGSGGGGSGGGGS
GGGGSFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTVVDGGGGSGGG
GSEVQLVESGGGLVQPGGSLRLSCAASGFTISTNAMSWVRQAPGKGLEWIGVITGRDITYYASWAKGRFTISRDNSK
NTLYLQMNSLRAEDTAVYYCARDGGSSAITSNNIWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKTHTC
PPCPAPEAAGAPSVFLFPPKPKDTLMISRTPEVTCVVVDVSHEDPEVKFNWYVDGVEVHNAKTKPREEQYNSTYRVV
SVLTVLHQDWLNGKEYKCAVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGGGGGSGGGGSR
SLVESGGGLVQPGGSLRLSCTASGFTISSYHMQWVRQAPGKGLEYIGTISSGGNVYYASSARGRFTISRPSSKNTVD
LQMNSLRAEDTAVYYCARDSGYSDPMWGQGTLVTVSSGGGGSGGGGSGGGGSGGGGSDVVMTQSPSSVSASVGDRVT
ITCQASQNIRTYLSWYQQKPGKAPKLLIYAAANLASGVPSRFSGSGSGTDFTLTISDLEPGDAATYYCQSTYLGTDY
VGGAFGGGTKVEIKGGGGSGGGGSGEVQLLESGGGLVQPGGSLRLSCAASGFSFSSGYDMCWVRQAPGKGLEWIACI
AAGSAGITYDANWAKGRFTISRDNSKNTLYLQMNSLRAEDTAVYYCARSAFSFDYAMDLWGQGTLVTVSSGGGGSGG
GGSGGGGSGGGGSDIQMTQSPSTLSASVGDRVTITCQASQSISSHLNWYQQKPGKAPKLLIYKASTLASGVPSRFSG
SGSGTEFTLTISSLQPDDFATYYCQQGYSWGNVDNVFGGGTKVEIK
>SEQ ID 7 SI-49E2 chain B nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGT
>SEQ ID 8 SI-49E2 chain B aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>SEQ ID 9 SI-49E3 chain A nt
GACGTTGTGATGACCCAGTCTCCATCTTCCGTGTCTGCATCTGTAGGAGACAGAGTCACCATCACCTGTCAGGCCAG
TCAGAACATTAGGACTTACTTATCCTGGTATCAGCAGAAACCAGGGAAAGCCCCTAAGCTCCTGATCTATGCTGCAG
CCAATCTGGCATCTGGGGTCCCATCAAGGTTCAGCGGCAGTGGATCTGGGACAGATTTCACTCTCACCATCAGCGAC
CTGGAGCCTGGCGATGCTGCAACTTACTATTGTCAGTCTACCTATCTTGGTACTGATTATGTTGGCGGTGCTTTCGG
CGGAGGGACCAAGGTGGAGATCAAAGGCGGTGGCGGTAGTGGGGGAGGCGGTTCTGGCGGCGGAGGGTCCGGCGGTG
GAGGATCACGGTCGCTGGTGGAGTCTGGGGGAGGCTTGGTCCAGCCTGGGGGGTCCCTGAGACTCTCCTGTACAGCC
TCTGGATTCACCATCAGTAGCTACCACATGCAGTGGGTCCGCCAGGCTCCAGGGAAGGGGCTGGAGTACATCGGAAC
CATTAGTAGTGGTGGTAATGTATACTACGCAAGCTCCGCTAGAGGCAGATTCACCATCTCCAGACCCTCGTCCAAGA
ACACGGTGGATCTTCAAATGAACAGCCTGAGAGCCGAGGACACGGCTGTGTATTACTGTGCGAGAGACTCTGGTTAT
AGTGATCCTATGTGGGGCCAGGGAACCCTGGTCACCGTGTCGACAGGCGGTGGAGGGTCCGGCGGTGGTGGATCAGA
GGTGCAGCTGTTGGAGTCTGGGGGAGGCTTGGTACAGCCTGGGGGGTCCCTGAGACTCTCCTGTGCAGCCTCTGGAT
TCTCCTTCAGTAGCGGGTACGACATGTGCTGGGTCCGCCAGGCTCCAGGGAAGGGGCTGGAGTGGATCGCATGTATT
GCTGCTGGTAGTGCTGGTATCACTTACGACGCGAACTGGGCGAAAGGCCGGTTCACCATCTCCAGAGACAATTCCAA
GAACACGCTGTATCTGCAAATGAACAGCCTGAGAGCCGAGGACACGGCCGTATATTACTGTGCGAGATCGGCGTTTT
CGTTCGACTACGCCATGGACCTCTGGGGCCAGGGAACCCTGGTCACCGTCTCGAGCGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAACTCACACATGCCC
ACCGTGCCCAGCACCTGAAGCCGCGGGGGCACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCGCGGTCTCCAACAAAGCCCTCCCAG
CCCCCATCGAGAAAACCATCTCCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTGGCGGTGGAGGGTCCGGCGGTGGTGGATCCGAGGT
GCAGCTGGTGGAGTCTGGGGGAGGCTTGGTCCAGCCTGGGGGGTCCCTGAGACTCTCCTGTGCAGCCTCTGGATTCA
CCATCAGTACCAATGCAATGAGCTGGGTCCGCCAGGCTCCAGGGAAGGGGCTGGAGTGGATCGGAGTCATTACTGGT
CGTGATATCACATACTACGCGAGCTGGGCGAAAGGCAGATTCACCATCTCCAGAGACAATTCCAAGAACACGCTGTA
TCTTCAAATGAACAGCCTGAGAGCCGAGGACACGGCTGTGTATTACTGTGCGAGAGACGGTGGTTCTTCTGCTATTA
CTAGTAACAACATTTGGGGCCAGGGAACCCTGGTCACCGTCTCTTCAGGCGGTGGCGGTAGTGGGGGAGGCGGTTCT
GGCGGCGGAGGGTCCGGCGGTGGAGGATCAGACGTCGTGATGACCCAGTCTCCTTCCACCCTGTCTGCATCTGTAGG
AGACAGAGTCACCATCAATTGCCAAGCCAGTGAGAGCATTAGCAGTTGGTTAGCCTGGTATCAGCAGAAACCAGGGA
AAGCCCCTAAGCTCCTGATCTATGAAGCATCCAAACTGGCATCTGGGGTCCCATCAAGGTTCAGCGGCAGTGGATCT
GGGACAGAATTTACTCTCACCATCAGCAGCCTGCAGCCTGATGATTTTGCAACTTATTACTGCCAAGGCTATTTTTA
TTTTATTAGTCGTACTTATGTAAATTCTTTCGGCGGAGGGACCAAGGTGGAGATCAAAGGCGGTGGAGGGTCCGGCG
GTGGTGGCTCCGGATTTCTTAATTCCCTTTTCAACCAAGAGGTTCAGATCCCCTTGACTGAAAGCTATTGCGGCCCT
TGTCCGAAAAACTGGATATGTTACAAGAATAATTGTTACCAATTCTTCGACGAAAGCAAGAACTGGTATGAGAGTCA
GGCGTCTTGTATGAGTCAGAATGCCAGCCTGCTTAAGGTTTATTCAAAAGAAGACCAGGATCTGCTTAAGTTGGTAA
AGAGCTACCACTGGATGGGGCTGGTACATATCCCAACGAATGGGTCATGGCAGTGGGAGGACGGTTCTATTCTGAGT
CCAAATCTCCTGACGATCATCGAAATGCAGAAAGGGGACTGTGCCCTGTATGCATCATCCTTCAAGGGGTACATCGA
GAACTGCAGTACCCCAAATACCTACATTTGTATGCAAAGAACGGTTGGAGGCGGTGGCTCAGGCGGAGGCGGCTCAG
GAGGTGGCGGTTCAGGAGGCGGCGGATCTTTCCTAAACTCATTATTCAACCAAGAAGTTCAAATTCCCTTGACCGAA
AGTTACTGTGGCCCATGTCCTAAAAACTGGATATGTTACAAAAATAACTGCTACCAATTTTTTGATGAGAGTAAAAA
CTGGTATGAGAGCCAGGCTTCTTGTATGTCTCAAAATGCCAGCCTTCTGAAAGTATACAGCAAAGAGGACCAGGATT
TACTTAAACTGGTGAAGTCATATCATTGGATGGGACTAGTACACATTCCAACAAATGGATCTTGGCAGTGGGAAGAT
GGCTCCATTCTCTCACCCAACCTACTAACAATAATTGAAATGCAGAAGGGAGACTGTGCACTCTATGCCTCGAGCTT
TAAAGGCTATATAGAAAACTGTTCAACTCCAAATACGTACATCTGCATGCAAAGGACTGTGTAA
>SEQ ID 10 SI-49E3 chain A aa
DVVMTQSPSSVSASVGDRVTITCQASQNIRTYLSWYQQKPGKAPKLLIYAAANLASGVPSRFSGSGSGTDFTLTISD
LEPGDAATYYCQSTYLGTDYVGGAFGGGTKVEIKGGGGSGGGGSGGGGSGGGGSRSLVESGGGLVQPGGSLRLSCTA
SGFTISSYHMQWVRQAPGKGLEYIGTISSGGNVYYASSARGRFTISRPSSKNTVDLQMNSLRAEDTAVYYCARDSGY
SDPMWGQGTLVTVSTGGGGSGGGGSEVQLLESGGGLVQPGGSLRLSCAASGFSFSSGYDMCWVRQAPGKGLEWIACI
AAGSAGITYDANWAKGRFTISRDNSKNTLYLQMNSLRAEDTAVYYCARSAFSFDYAMDLWGQGTLVTVSSASTKGPS
VFPLAPSSKSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPEAAGAPSVFLFPPKPKDTLMISRTPEVTCVVVDVSHEDPEVKFNWY
VDGVEVHNAKTKPREEQYNSTYRVVSVLTVLHQDWLNGKEYKCAVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVMHEAL
HNHYTQKSLSLSPGGGGGSGGGGSEVQLVESGGGLVQPGGSLRLSCAASGFTISTNAMSWVRQAPGKGLEWIGVITG
RDITYYASWAKGRFTISRDNSKNTLYLQMNSLRAEDTAVYYCARDGGSSAITSNNIWGQGTLVTVSSGGGGSGGGGS
GGGGSGGGGSDVVMTQSPSTLSASVGDRVTINCQASESISSWLAWYQQKPGKAPKLLIYEASKLASGVPSRFSGSGS
GTEFTLTISSLQPDDFATYYCQGYFYFISRTYVNSFGGGTKVEIKGGGGSGGGGSGFLNSLFNQEVQIPLTESYCGP
CPKNWICYKNNCYQFFDESKNWYESQASCMSQNASLLKVYSKEDQDLLKLVKSYHWMGLVHIPTNGSWQWEDGSILS
PNLLTIIEMQKGDCALYASSFKGYIENCSTPNTYICMQRTVGGGGSGGGGSGGGGSGGGGSFLNSLFNQEVQIPLTE
SYCGPCPKNWICYKNNCYQFFDESKNWYESQASCMSQNASLLKVYSKEDQDLLKLVKSYHWMGLVHIPTNGSWQWED
GSILSPNLLTIIEMQKGDCALYASSFKGYIENCSTPNTYICMQRTV
>SEQ ID 11 SI-49E3 chain B nt
GACATCCAGATGACCCAGTCTCCTTCCACCCTGTCTGCATCTGTAGGAGACAGAGTCACCATCACTTGCCAGGCCAG
TCAGAGCATTAGTTCCCACTTAAACTGGTATCAGCAGAAACCAGGGAAAGCCCCTAAGCTCCTGATCTATAAGGCAT
CCACTCTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAACAGGGTTATAGTTGGGGTAATGTTGATAATGTTTTCGGCGG
AGGGACCAAGGTGGAGATCAAACGTACGGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGCGAAGTCACCCATCAGGGCCTGAGCT
CGCCCGTCACAAAGAGCTTCAACAGGGGAGAGTGT
>SEQ ID 12 SI-49E3 chain B aa
DIQMTQSPSTLSASVGDRVTITCQASQSISSHLNWYQQKPGKAPKLLIYKASTLASGVPSRFSGSGSGTEFTLTISS
LQPDDFATYYCQQGYSWGNVDNVFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGEC
>SEQ ID 13 SI-49E4 chain A nt
GACATCCAGATGACCCAGTCTCCTTCCACCCTGTCTGCATCTGTAGGAGACAGAGTCACCATCACTTGCCAGGCCAG
TCAGAGCATTAGTTCCCACTTAAACTGGTATCAGCAGAAACCAGGGAAAGCCCCTAAGCTCCTGATCTATAAGGCAT
CCACTCTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAACAGGGTTATAGTTGGGGTAATGTTGATAATGTTTTCGGCGG
AGGGACCAAGGTGGAGATCAAAGGCGGTGGCGGTAGTGGGGGAGGCGGTTCTGGCGGCGGAGGGTCCGGCGGTGGAG
GATCAGAGGTGCAGCTGTTGGAGTCTGGGGGAGGCTTGGTACAGCCTGGGGGGTCCCTGAGACTCTCCTGTGCAGCC
TCTGGATTCTCCTTCAGTAGCGGGTACGACATGTGCTGGGTCCGCCAGGCTCCAGGGAAGGGGCTGGAGTGGATCGC
ATGTATTGCTGCTGGTAGTGCTGGTATCACTTACGACGCGAACTGGGCGAAAGGCCGGTTCACCATCTCCAGAGACA
ATTCCAAGAACACGCTGTATCTGCAAATGAACAGCCTGAGAGCCGAGGACACGGCCGTATATTACTGTGCGAGATCG
GCGTTTTCGTTCGACTACGCCATGGACCTCTGGGGCCAGGGAACCCTGGTCACCGTGTCGACAGGCGGTGGAGGGTC
CGGCGGTGGTGGATCACGGTCGCTGGTGGAGTCTGGGGGAGGCTTGGTCCAGCCTGGGGGGTCCCTGAGACTCTCCT
GTACTGCCTCTGGATTCACCATCAGTAGCTACCACATGCAGTGGGTCCGCCAGGCTCCAGGGAAGGGGCTGGAGTAC
ATCGGAACCATTAGTAGTGGTGGTAATGTATACTACGCAAGCTCCGCTAGAGGCAGATTCACCATCTCCAGACCCTC
GTCCAAGAACACGGTGGATCTTCAAATGAACAGCCTGAGAGCCGAGGACACGGCTGTGTATTACTGTGCGAGAGACT
CTGGTTATAGTGATCCTATGTGGGGCCAGGGAACCCTGGTCACCGTCTCTTCAGCTAGCACCAAGGGCCCATCGGTC
TTCCCCCTGGCACCCTCCTCCAAGAGCACCTCTGGGGGCACAGCGGCCCTGGGCTGCCTGGTCAAGGACTACTTCCC
CGAACCGGTGACGGTGTCGTGGAACTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGCACCCAGACCTACATCTGCAACGTG
AATCACAAGCCCAGCAACACCAAGGTGGACAAGAGAGTTGAGCCCAAATCTTGTGACAAAACTCACACATGCCCACC
GTGCCCAGCACCTGAAGCCGCGGGGGCACCGTCAGTCTTCCTCTTCCCCCCAAAACCCAAGGACACCCTCATGATCT
CCCGGACCCCTGAGGTCACATGCGTGGTGGTGGACGTGAGCCACGAAGACCCTGAGGTCAAGTTCAACTGGTACGTG
GACGGCGTGGAGGTGCATAATGCCAAGACAAAGCCGCGGGAGGAGCAGTACAACAGCACGTACCGTGTGGTCAGCGT
CCTCACCGTCCTGCACCAGGACTGGCTGAATGGCAAGGAGTACAAGTGCGCGGTCTCCAACAAAGCCCTCCCAGCCC
CCATCGAGAAAACCATCTCCAAAGCCAAAGGGCAGCCCCGAGAACCACAGGTGTACACCCTGCCCCCATCCCGGGAT
GAGCTGACCAAGAACCAGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCTTCTTCCTCTATA
GCAAGCTCACCGTGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTGGCGGTGGAGGGTCCGGCGGTGGTGGATCCGAGGTGCA
GCTGGTGGAGTCTGGGGGAGGCTTGGTCCAGCCTGGGGGGTCCCTGAGACTCTCCTGTGCAGCCTCTGGATTCACCA
TCAGTACCAATGCAATGAGCTGGGTCCGCCAGGCTCCAGGGAAGGGGCTGGAGTGGATCGGAGTCATTACTGGTCGT
GATATCACATACTACGCGAGCTGGGCGAAAGGCAGATTCACCATCTCCAGAGACAATTCCAAGAACACGCTGTATCT
TCAAATGAACAGCCTGAGAGCCGAGGACACGGCTGTGTATTACTGTGCGAGAGACGGTGGTTCTTCTGCTATTACTA
GTAACAACATTTGGGGCCAGGGAACCCTGGTCACCGTCTCTTCAGGCGGTGGCGGTAGTGGGGGAGGCGGTTCTGGC
GGCGGAGGGTCCGGCGGTGGAGGATCAGACGTCGTGATGACCCAGTCTCCTTCCACCCTGTCTGCATCTGTAGGAGA
CAGAGTCACCATCAATTGCCAAGCCAGTGAGAGCATTAGCAGTTGGTTAGCCTGGTATCAGCAGAAACCAGGGAAAG
CCCCTAAGCTCCTGATCTATGAAGCATCCAAACTGGCATCTGGGGTCCCATCAAGGTTCAGCGGCAGTGGATCTGGG
ACAGAATTTACTCTCACCATCAGCAGCCTGCAGCCTGATGATTTTGCAACTTATTACTGCCAAGGCTATTTTTATTT
TATTAGTCGTACTTATGTAAATTCTTTCGGCGGAGGGACCAAGGTGGAGATCAAAGGCGGTGGAGGGTCCGGCGGTG
GTGGCTCCGGATTTCTTAATTCCCTTTTCAACCAAGAGGTTCAGATCCCCTTGACTGAAAGCTATTGCGGCCCTTGT
CCGAAAAACTGGATATGTTACAAGAATAATTGTTACCAATTCTTCGACGAAAGCAAGAACTGGTATGAGAGTCAGGC
GTCTTGTATGAGTCAGAATGCCAGCCTGCTTAAGGTTTATTCAAAAGAAGACCAGGATCTGCTTAAGTTGGTAAAGA
GCTACCACTGGATGGGGCTGGTACATATCCCAACGAATGGGTCATGGCAGTGGGAGGACGGTTCTATTCTGAGTCCA
AATCTCCTGACGATCATCGAAATGCAGAAAGGGGACTGTGCCCTGTATGCATCATCCTTCAAGGGGTACATCGAGAA
CTGCAGTACCCCAAATACCTACATTTGTATGCAAAGAACGGTTGGAGGCGGTGGCTCAGGCGGAGGCGGCTCAGGAG
GTGGCGGTTCAGGAGGCGGCGGATCTTTCCTAAACTCATTATTCAACCAAGAAGTTCAAATTCCCTTGACCGAAAGT
TACTGTGGCCCATGTCCTAAAAACTGGATATGTTACAAAAATAACTGCTACCAATTTTTTGATGAGAGTAAAAACTG
GTATGAGAGCCAGGCTTCTTGTATGTCTCAAAATGCCAGCCTTCTGAAAGTATACAGCAAAGAGGACCAGGATTTAC
TTAAACTGGTGAAGTCATATCATTGGATGGGACTAGTACACATTCCAACAAATGGATCTTGGCAGTGGGAAGATGGC
TCCATTCTCTCACCCAACCTACTAACAATAATTGAAATGCAGAAGGGAGACTGTGCACTCTATGCCTCGAGCTTTAA
AGGCTATATAGAAAACTGTTCAACTCCAAATACGTACATCTGCATGCAAAGGACTGTG
>SEQ ID 14 SI-49E4 chain A aa
DIQMTQSPSTLSASVGDRVTITCQASQSISSHLNWYQQKPGKAPKLLIYKASTLASGVPSRFSGSGSGTEFTLTISS
LQPDDFATYYCQQGYSWGNVDNVFGGGTKVEIKGGGGSGGGGSGGGGSGGGGSEVQLLESGGGLVQPGGSLRLSCAA
SGFSFSSGYDMCWVRQAPGKGLEWIACIAAGSAGITYDANWAKGRFTISRDNSKNTLYLQMNSLRAEDTAVYYCARS
AFSFDYAMDLWGQGTLVTVSTGGGGSGGGGSRSLVESGGGLVQPGGSLRLSCTASGFTISSYHMQWVRQAPGKGLEY
IGTISSGGNVYYASSARGRFTISRPSSKNTVDLQMNSLRAEDTAVYYCARDSGYSDPMWGQGTLVTVSSASTKGPSV
FPLAPSSKSTSGGTAALGCLVKDYFPEPVTVSWNSGALTSGVHTFPAVLQSSGLYSLSSVVTVPSSSLGTQTYICNV
NHKPSNTKVDKRVEPKSCDKTHTCPPCPAPEAAGAPSVFLFPPKPKDTLMISRTPEVTCVVVDVSHEDPEVKFNWYV
DGVEVHNAKTKPREEQYNSTYRVVSVLTVLHQDWLNGKEYKCAVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVFSCSVMHEALH
NHYTQKSLSLSPGGGGGSGGGGSEVQLVESGGGLVQPGGSLRLSCAASGFTISTNAMSWVRQAPGKGLEWIGVITGR
DITYYASWAKGRFTISRDNSKNTLYLQMNSLRAEDTAVYYCARDGGSSAITSNNIWGQGTLVTVSSGGGGSGGGGSG
GGGSGGGGSDVVMTQSPSTLSASVGDRVTINCQASESISSWLAWYQQKPGKAPKLLIYEASKLASGVPSRFSGSGSG
TEFTLTISSLQPDDFATYYCQGYFYFISRTYVNSFGGGTKVEIKGGGGSGGGGSGFLNSLFNQEVQIPLTESYCGPC
PKNWICYKNNCYQFFDESKNWYESQASCMSQNASLLKVYSKEDQDLLKLVKSYHWMGLVHIPTNGSWQWEDGSILSP
NLLTIIEMQKGDCALYASSFKGYIENCSTPNTYICMQRTVGGGGSGGGGSGGGGSGGGGSFLNSLFNQEVQIPLTES
YCGPCPKNWICYKNNCYQFFDESKNWYESQASCMSQNASLLKVYSKEDQDLLKLVKSYHWMGLVHIPTNGSWQWEDG
SILSPNLLTIIEMQKGDCALYASSFKGYIENCSTPNTYICMQRTV
>SEQ ID 15 SI-49E4 chain B nt
GACGTTGTGATGACCCAGTCTCCATCTTCCGTGTCTGCATCTGTAGGAGACAGAGTCACCATCACCTGTCAGGCCAG
TCAGAACATTAGGACTTACTTATCCTGGTATCAGCAGAAACCAGGGAAAGCCCCTAAGCTCCTGATCTATGCTGCAG
CCAATCTGGCATCTGGGGTCCCATCAAGGTTCAGCGGCAGTGGATCTGGGACAGATTTCACTCTCACCATCAGCGAC
CTGGAGCCTGGCGATGCTGCAACTTACTATTGTCAGTCTACCTATCTTGGTACTGATTATGTTGGCGGTGCTTTCGG
CGGAGGGACCAAGGTGGAGATCAAACGTACGGTGGCTGCACCATCTGTCTTCATCTTCCCGCCATCTGATGAGCAGT
TGAAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTCAG
CAGCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGT
>SEQ ID 16 SI-49E4 chain B aa
DVVMTQSPSSVSASVGDRVTITCQASQNIRTYLSWYQQKPGKAPKLLIYAAANLASGVPSRFSGSGSGTDFTLTISD
LEPGDAATYYCQSTYLGTDYVGGAFGGGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
>SEQ ID 17 SI-49E11 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGGAGGCGGTGGCTCAGGCGGAGGCGGCTCAGGAGGTGGCGGTTCA
GGAGGCGGCGGATCTTTCCTAAACTCATTATTCAACCAAGAAGTTCAAATTCCCTTGACCGAAAGTTACTGTGGCCC
ATGTCCTAAAAACTGGATATGTTACAAAAATAACTGCTACCAATTTTTTGATGAGAGTAAAAACTGGTATGAGAGCC
AGGCTTCTTGTATGTCTCAAAATGCCAGCCTTCTGAAAGTATACAGCAAAGAGGACCAGGATTTACTTAAACTGGTG
AAGTCATATCATTGGATGGGACTAGTACACATTCCAACAAATGGATCTTGGCAGTGGGAAGATGGCTCCATTCTCTC
ACCCAACCTACTAACAATAATTGAAATGCAGAAGGGAGACTGTGCACTCTATGCCTCGAGCTTTAAAGGCTATATAG
AAAACTGTTCAACTCCAAATACGTACATCTGCATGCAAAGGACTGTGGTCGACGGCGGTGGAGGGTCCGGCGGTGGT
GGATCAGAGGTGCAGCTGGTGGAGTCTGGGGGAGGCTTGGTCCAGCCTGGGGGGTCCCTGAGACTCTCCTGTGCAGC
CTCTGGATTCACCATCAGTACCAATGCAATGAGCTGGGTCCGCCAGGCTCCAGGGAAGGGGCTGGAGTGGATCGGAG
TCATTACTGGTCGTGATATCACATACTACGCGAGCTGGGCGAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGACGGTGGTTC
TTCTGCTATTACTAGTAACAACATTTGGGGCCAGGGAACCCTGGTCACCGTGTCCTCAGCTAGCACCAAGGGCCCAT
CGGTCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAAAACTCACACATGC
CCACCGTGCCCAGCACCTGAAGCCGCGGGGGCACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTACACCCTGCCCCCATCCC
GGGATGAGCTGACCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTGGCGGTGGAGGGTCCGGCGGTGGTGGATCCGAG
GTGCAGCTGTTGGAGTCTGGGGGAGGCTTGGTACAGCCTGGGGGGTCCCTGAGACTCTCCTGTGCAGCCTCTGGATT
CTCCTTCAGTAGCGGGTACGACATGTGCTGGGTCCGCCAGGCTCCAGGGAAGGGGCTGGAGTGGATCGCATGCATTG
CTGCTGGTAGTGCTGGTATCACTTACGACGCGAACTGGGCGAAAGGCCGGTTCACCATCTCCAGAGACAATTCCAAG
AACACGCTGTATCTGCAAATGAACAGCCTGAGAGCCGAGGACACGGCCGTATATTACTGTGCGAGATCGGCGTTTTC
GTTCGACTACGCCATGGACCTCTGGGGCCAGGGAACCCTGGTCACCGTCTCGAGCGGTGGAGGCGGATCTGGCGGAG
GTGGTTCCGGCGGTGGCGGCTCCGGTGGAGGCGGCTCTGACATCCAGATGACCCAGTCTCCTTCCACCCTGTCTGCA
TCTGTAGGAGACAGAGTCACCATCACTTGCCAGGCCAGTCAGAGCATTAGTTCCCACTTAAACTGGTATCAGCAGAA
ACCAGGGAAAGCCCCTAAGCTCCTGATCTATAAGGCATCCACTCTGGCATCTGGGGTCCCATCAAGGTTCAGCGGCA
GTGGATCTGGGACAGAATTTACTCTCACCATCAGCAGCCTGCAGCCTGATGATTTTGCAACTTATTACTGCCAACAG
GGTTATAGTTGGGGTAATGTTGATAATGTTTTCGGCGGAGGGACCAAGGTGGAGATCAAAGGCGGTGGAGGGTCCGG
CGGTGGTGGCTCCGGACGAGAGGGCCCCGAGCTGTCTCCTGATGACCCAGCAGGCCTCTTGGACTTGCGGCAGGGTA
TGTTCGCTCAACTTGTGGCTCAGAATGTTCTGCTCATTGATGGACCACTCTCTTGGTATAGTGACCCCGGTCTGGCC
GGGGTGAGTCTGACCGGCGGGCTCTCTTATAAAGAGGATACTAAGGAACTGGTCGTAGCAAAAGCGGGCGTTTATTA
CGTTTTTTTTCAGCTGGAGCTCAGGCGCGTGGTGGCCGGCGAGGGCAGTGGCTCTGTGTCCCTGGCCCTGCACTTAC
AGCCCTTGAGAAGCGCTGCAGGTGCTGCCGCACTGGCTTTAACTGTTGACCTCCCTCCGGCCTCTTCTGAAGCTAGA
AACAGCGCTTTCGGCTTCCAAGGGCGCCTGCTGCACCTGAGCGCAGGCCAGCGCTTAGGTGTGCACCTTCATACAGA
GGCCAGGGCCCGACACGCTTGGCAGCTCACACAGGGTGCCACGGTTCTCGGACTTTTCCGCGTTACTCCCGAGATCC
CCGCTGGCCTCGGAAGTACTGGTTCTGGGTCTAAACCCGGTTCCGGCGAAGGTAGTACTAAAGGACGAGAAGGGCCA
GAGTTAAGTCCAGATGACCCTGCTGGGCTTTTGGACCTGCGGCAGGGCATGTTCGCTCAACTGGTGGCTCAGAACGT
GCTGCTGATCGATGGCCCCCTGAGTTGGTACAGCGATCCCGGGCTGGCAGGCGTGTCACTTACAGGGGGCCTCTCTT
ACAAGGAAGACACCAAGGAGTTAGTGGTCGCTAAGGCTGGCGTGTATTACGTGTTCTTCCAACTGGAGCTGAGAAGG
GTTGTGGCAGGAGAGGGTAGCGGCAGCGTGTCTTTAGCCCTTCACTTGCAGCCCCTGAGGTCTGCTGCAGGTGCAGC
CGCTCTCGCGCTCACCGTGGATCTCCCCCCAGCCTCATCTGAAGCTAGGAACAGTGCATTTGGCTTTCAGGGACGCT
TGCTGCACCTCTCCGCTGGACAGAGGCTGGGCGTGCACCTTCACACAGAGGCCCGTGCCAGGCATGCATGGCAGCTC
ACTCAGGGGGCAACAGTGCTGGGTCTCTTCCGCGTGACTCCTGAAATACCAGCTGGACTTGGCGGTGGAGGCAGCGG
CGGAGGAGGATCTCGTGAGGGGCCAGAACTGTCCCCCGATGACCCAGCCGGACTGCTCGATCTCAGACAGGGCATGT
TCGCTCAGCTTGTAGCCCAAAATGTCCTCCTGATTGACGGCCCTTTGAGCTGGTATAGTGATCCCGGCTTGGCCGGG
GTATCTCTGACCGGAGGCCTCTCCTACAAGGAAGACACCAAAGAGCTGGTGGTGGCAAAAGCGGGGGTGTATTATGT
GTTCTTTCAGCTCGAGCTGCGGAGAGTTGTGGCCGGGGAAGGGTCTGGGAGCGTATCTCTTGCACTTCACCTGCAGC
CCCTGCGCAGCGCCGCTGGAGCCGCCGCCCTTGCTCTTACTGTGGATCTGCCTCCTGCTTCCTCAGAAGCACGCAAC
AGCGCCTTCGGCTTTCAAGGACGTCTCCTGCACTTGTCCGCAGGACAGAGGTTGGGCGTCCATTTACACACTGAGGC
ACGGGCACGGCACGCTTGGCAGCTTACCCAGGGAGCCACCGTGCTGGGACTCTTTAGAGTGACACCCGAGATCCCCG
CTGGCTTG
>SEQ ID 18 SI-49E11 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVGGGGSGGGGSGGGGS
GGGGSFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTVVDGGGGSGGG
GSEVQLVESGGGLVQPGGSLRLSCAASGFTISTNAMSWVRQAPGKGLEWIGVITGRDITYYASWAKGRFTISRDNSK
NTLYLQMNSLRAEDTAVYYCARDGGSSAITSNNIWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKTHTC
PPCPAPEAAGAPSVFLFPPKPKDTLMISRTPEVTCVVVDVSHEDPEVKFNWYVDGVEVHNAKTKPREEQYNSTYRVV
SVLTVLHQDWLNGKEYKCAVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGGGGGSGGGGSE
VQLLESGGGLVQPGGSLRLSCAASGFSFSSGYDMCWVRQAPGKGLEWIACIAAGSAGITYDANWAKGRETISRDNSK
NTLYLQMNSLRAEDTAVYYCARSAFSFDYAMDLWGQGTLVTVSSGGGGSGGGGSGGGGSGGGGSDIQMTQSPSTLSA
SVGDRVTITCQASQSISSHLNWYQQKPGKAPKLLIYKASTLASGVPSRFSGSGSGTEFTLTISSLQPDDFATYYCQQ
GYSWGNVDNVFGGGTKVEIKGGGGSGGGGSGREGPELSPDDPAGLLDLRQGMFAQLVAQNVLLIDGPLSWYSDPGLA
GVSLTGGLSYKEDTKELVVAKAGVYYVFFQLELRRVVAGEGSGSVSLALHLQPLRSAAGAAALALTVDLPPASSEAR
NSAFGFQGRLLHLSAGQRLGVHLHTEARARHAWQLTQGATVLGLFRVTPEIPAGLGSTGSGSKPGSGEGSTKGREGP
ELSPDDPAGLLDLRQGMFAQLVAQNVLLIDGPLSWYSDPGLAGVSLTGGLSYKEDTKELVVAKAGVYYVFFQLELRR
VVAGEGSGSVSLALHLQPLRSAAGAAALALTVDLPPASSEARNSAFGFQGRLLHLSAGQRLGVHLHTEARARHAWQL
TQGATVLGLFRVTPEIPAGLGGGGSGGGGSREGPELSPDDPAGLLDLRQGMFAQLVAQNVLLIDGPLSWYSDPGLAG
VSLTGGLSYKEDTKELVVAKAGVYYVFFQLELRRVVAGEGSGSVSLALHLQPLRSAAGAAALALTVDLPPASSEARN
SAFGFQGRLLHLSAGQRLGVHLHTEARARHAWQLTQGATVLGLFRVTPEIPAGL
>SEQ ID 19 SI-49E11 chain B nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGT
>SEQ ID 20 SI-49E11 chain B aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>SEQ ID 21 SI-49E12 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGGAGGCGGTGGCTCAGGCGGAGGCGGCTCAGGAGGTGGCGGTTCA
GGAGGCGGCGGATCTTTCCTAAACTCATTATTCAACCAAGAAGTTCAAATTCCCTTGACCGAAAGTTACTGTGGCCC
ATGTCCTAAAAACTGGATATGTTACAAAAATAACTGCTACCAATTTTTTGATGAGAGTAAAAACTGGTATGAGAGCC
AGGCTTCTTGTATGTCTCAAAATGCCAGCCTTCTGAAAGTATACAGCAAAGAGGACCAGGATTTACTTAAACTGGTG
AAGTCATATCATTGGATGGGACTAGTACACATTCCAACAAATGGATCTTGGCAGTGGGAAGATGGCTCCATTCTCTC
ACCCAACCTACTAACAATAATTGAAATGCAGAAGGGAGACTGTGCACTCTATGCCTCGAGCTTTAAAGGCTATATAG
AAAACTGTTCAACTCCAAATACGTACATCTGCATGCAAAGGACTGTGGTCGACGGCGGTGGAGGGTCCGGCGGTGGT
GGATCAGAGGTGCAGCTGGTGGAGTCTGGGGGAGGCTTGGTCCAGCCTGGGGGGTCCCTGAGACTCTCCTGTGCAGC
CTCTGGATTCACCATCAGTACCAATGCAATGAGCTGGGTCCGCCAGGCTCCAGGGAAGGGGCTGGAGTGGATCGGAG
TCATTACTGGTCGTGATATCACATACTACGCGAGCTGGGCGAAAGGCAGATTCACCATCTCCAGAGACAATTCCAAG
AACACGCTGTATCTTCAAATGAACAGCCTGAGAGCCGAGGACACGGCTGTGTATTACTGTGCGAGAGACGGTGGTTC
TTCTGCTATTACTAGTAACAACATTTGGGGCCAGGGAACCCTGGTCACCGTGTCCTCAGCTAGCACCAAGGGCCCAT
CGGTCTTCCCCCTGGCACCCTCCTCCAAGAGCACCTCTGGGGGCACAGCGGCCCTGGGCTGCCTGGTCAAGGACTAC
TTCCCCGAACCGGTGACGGTGTCGTGGAACTCAGGCGCCCTGACCAGCGGCGTGCACACCTTCCCGGCTGTCCTACA
GTCCTCAGGACTCTACTCCCTCAGCAGCGTGGTGACCGTGCCCTCCAGCAGCTTGGGCACCCAGACCTACATCTGCA
ACGTGAATCACAAGCCCAGCAACACCAAGGTGGACAAGAGAGTTGAGCCCAAATCTTGTGACAAAACTCACACATGC
CCACCGTGCCCAGCACCTGAAGCCGCGGGGGCACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCGCGGTCTCCAACAAAGCCCTCCC
AGCCCCCATCGAGAAAACCATCTCCAAAGCCAAAGGGCAGCCCCGAGAACCACAGGTGTACACCCTGCCCCCATCCC
GGGATGAGCTGACCAAGAACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCCGACGGCTCCTTCTTCCT
CTATAGCAAGCTCACCGTGGACAAGAGCAGGTGGCAGCAGGGGAACGTCTTCTCATGCTCCGTGATGCATGAGGCTC
TGCACAACCACTACACGCAGAAGAGCCTCTCCCTGTCTCCGGGTGGCGGTGGAGGGTCCGGCGGTGGTGGATCCCGA
GAGGGCCCCGAGCTGTCTCCTGATGACCCAGCAGGCCTCTTGGACTTGCGGCAGGGTATGTTCGCTCAACTTGTGGC
TCAGAATGTTCTGCTCATTGATGGACCACTCTCTTGGTATAGTGACCCCGGTCTGGCCGGGGTGAGTCTGACCGGCG
GGCTCTCTTATAAAGAGGATACTAAGGAACTGGTCGTAGCAAAAGCGGGCGTTTATTACGTTTTTTTTCAGCTGGAG
CTCAGGCGCGTGGTGGCCGGCGAGGGCAGTGGCTCTGTGTCCCTGGCCCTGCACTTACAGCCCTTGAGAAGCGCTGC
AGGTGCTGCCGCACTGGCTTTAACTGTTGACCTCCCTCCGGCCTCTTCTGAAGCTAGAAACAGCGCTTTCGGCTTCC
AAGGGCGCCTGCTGCACCTGAGCGCAGGCCAGCGCTTAGGTGTGCACCTTCATACAGAGGCCAGGGCCCGACACGCT
TGGCAGCTCACACAGGGTGCCACGGTTCTCGGACTTTTCCGCGTTACTCCCGAGATCCCCGCTGGCCTCGGAAGTAC
TGGTTCTGGGTCTAAACCCGGTTCCGGCGAAGGTAGTACTAAAGGACGAGAAGGGCCAGAGTTAAGTCCAGATGACC
CTGCTGGGCTTTTGGACCTGCGGCAGGGCATGTTCGCTCAACTGGTGGCTCAGAACGTGCTGCTGATCGATGGCCCC
CTGAGTTGGTACAGCGATCCCGGGCTGGCAGGCGTGTCACTTACAGGGGGCCTCTCTTACAAGGAAGACACCAAGGA
GTTAGTGGTCGCTAAGGCTGGCGTGTATTACGTGTTCTTCCAACTGGAGCTGAGAAGGGTTGTGGCAGGAGAGGGTA
GCGGCAGCGTGTCTTTAGCCCTTCACTTGCAGCCCCTGAGGTCTGCTGCAGGTGCAGCCGCTCTCGCGCTCACCGTG
GATCTCCCCCCAGCCTCATCTGAAGCTAGGAACAGTGCATTTGGCTTTCAGGGACGCTTGCTGCACCTCTCCGCTGG
ACAGAGGCTGGGCGTGCACCTTCACACAGAGGCCCGTGCCAGGCATGCATGGCAGCTCACTCAGGGGGCAACAGTGC
TGGGTCTCTTCCGCGTGACTCCTGAAATACCAGCTGGACTTGGCGGTGGAGGCAGCGGCGGAGGAGGATCTCGTGAG
GGGCCAGAACTGTCCCCCGATGACCCAGCCGGACTGCTCGATCTCAGACAGGGCATGTTCGCTCAGCTTGTAGCCCA
AAATGTCCTCCTGATTGACGGCCCTTTGAGCTGGTATAGTGATCCCGGCTTGGCCGGGGTATCTCTGACCGGAGGCC
TCTCCTACAAGGAAGACACCAAAGAGCTGGTGGTGGCAAAAGCGGGGGTGTATTATGTGTTCTTTCAGCTCGAGCTG
CGGAGAGTTGTGGCCGGGGAAGGGTCTGGGAGCGTATCTCTTGCACTTCACCTGCAGCCCCTGCGCAGCGCCGCTGG
AGCCGCCGCCCTTGCTCTTACTGTGGATCTGCCTCCTGCTTCCTCAGAAGCACGCAACAGCGCCTTCGGCTTTCAAG
GACGTCTCCTGCACTTGTCCGCAGGACAGAGGTTGGGCGTCCATTTACACACTGAGGCACGGGCACGGCACGCTTGG
CAGCTTACCCAGGGAGCCACCGTGCTGGGACTCTTTAGAGTGACACCCGAGATCCCCGCTGGCTTGGGCGGTGGAGG
GTCCGGCGGTGGTGGCTCCGGAGAGGTGCAGCTGTTGGAGTCTGGGGGAGGCTTGGTACAGCCTGGGGGGTCCCTGA
GACTCTCCTGTGCAGCCTCTGGATTCTCCTTCAGTAGCGGGTACGACATGTGCTGGGTCCGCCAGGCTCCAGGGAAG
GGGCTGGAGTGGATCGCATGCATTGCTGCTGGTAGTGCTGGTATCACTTACGACGCGAACTGGGCGAAAGGCCGGTT
CACCATCTCCAGAGACAATTCCAAGAACACGCTGTATCTGCAAATGAACAGCCTGAGAGCCGAGGACACGGCCGTAT
ATTACTGTGCGAGATCGGCGTTTTCGTTCGACTACGCCATGGACCTCTGGGGCCAGGGAACCCTGGTCACCGTCTCG
AGCGGCGGTGGCGGTAGTGGGGGAGGCGGTTCTGGCGGCGGAGGGTCCGGCGGTGGAGGATCAGACATCCAGATGAC
CCAGTCTCCTTCCACCCTGTCTGCATCTGTAGGAGACAGAGTCACCATCACTTGCCAGGCCAGTCAGAGCATTAGTT
CCCACTTAAACTGGTATCAGCAGAAACCAGGGAAAGCCCCTAAGCTCCTGATCTATAAGGCATCCACTCTGGCATCT
GGGGTCCCATCAAGGTTCAGCGGCAGTGGATCTGGGACAGAATTTACTCTCACCATCAGCAGCCTGCAGCCTGATGA
TTTTGCAACTTATTACTGCCAACAGGGTTATAGTTGGGGTAATGTTGATAATGTTTTCGGCGGAGGGACCAAGGTGG
AGATCAAA
>SEQ ID 22 SI-49E12 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVGGGGSGGGGSGGGGS
GGGGSFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTVVDGGGGSGGG
GSEVQLVESGGGLVQPGGSLRLSCAASGFTISTNAMSWVRQAPGKGLEWIGVITGRDITYYASWAKGRFTISRDNSK
NTLYLQMNSLRAEDTAVYYCARDGGSSAITSNNIWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKRVEPKSCDKTHTC
PPCPAPEAAGAPSVFLFPPKPKDTLMISRTPEVTCVVVDVSHEDPEVKFNWYVDGVEVHNAKTKPREEQYNSTYRVV
SVLTVLHQDWLNGKEYKCAVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGGGGGSGGGGSR
EGPELSPDDPAGLLDLRQGMFAQLVAQNVLLIDGPLSWYSDPGLAGVSLTGGLSYKEDTKELVVAKAGVYYVFFQLE
LRRVVAGEGSGSVSLALHLQPLRSAAGAAALALTVDLPPASSEARNSAFGFQGRLLHLSAGQRLGVHLHTEARARHA
WQLTQGATVLGLFRVTPEIPAGLGSTGSGSKPGSGEGSTKGREGPELSPDDPAGLLDLRQGMFAQLVAQNVLLIDGP
LSWYSDPGLAGVSLTGGLSYKEDTKELVVAKAGVYYVFFQLELRRVVAGEGSGSVSLALHLQPLRSAAGAAALALTV
DLPPASSEARNSAFGFQGRLLHLSAGQRLGVHLHTEARARHAWQLTQGATVLGLFRVTPEIPAGLGGGGSGGGGSRE
GPELSPDDPAGLLDLRQGMFAQLVAQNVLLIDGPLSWYSDPGLAGVSLTGGLSYKEDTKELVVAKAGVYYVFFQLEL
RRVVAGEGSGSVSLALHLQPLRSAAGAAALALTVDLPPASSEARNSAFGFQGRLLHLSAGQRLGVHLHTEARARHAW
QLTQGATVLGLFRVTPEIPAGLGGGGSGGGGSGEVQLLESGGGLVQPGGSLRLSCAASGFSFSSGYDMCWVRQAPGK
GLEWIACIAAGSAGITYDANWAKGRFTISRDNSKNTLYLQMNSLRAEDTAVYYCARSAFSFDYAMDLWGQGTLVTVS
SGGGGSGGGGSGGGGSGGGGSDIQMTQSPSTLSASVGDRVTITCQASQSISSHLNWYQQKPGKAPKLLIYKASTLAS
GVPSRFSGSGSGTEFTLTISSLQPDDFATYYCQQGYSWGNVDNVFGGGTKVEIK
>SEQ ID 23 SI-49E12 chain B nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGT
>SEQ ID 24 SI-49E12 chain B aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>SEQ ID 25 SI-49E13 chain A nt
CGAGAGGGCCCCGAGCTGTCTCCTGATGACCCAGCAGGCCTCTTGGACTTGCGGCAGGGTATGTTCGCTCAACTTGT
GGCTCAGAATGTTCTGCTCATTGATGGACCACTCTCTTGGTATAGTGACCCCGGTCTGGCCGGGGTGAGTCTGACCG
GCGGGCTCTCTTATAAAGAGGATACTAAGGAACTGGTCGTAGCAAAAGCGGGCGTTTATTACGTTTTTTTTCAGCTG
GAGCTCAGGCGCGTGGTGGCCGGCGAGGGCAGTGGCTCTGTGTCCCTGGCCCTGCACTTACAGCCCTTGAGAAGCGC
TGCAGGTGCTGCCGCACTGGCTTTAACTGTTGACCTCCCTCCGGCCTCTTCTGAAGCTAGAAACAGCGCTTTCGGCT
TCCAAGGGCGCCTGCTGCACCTGAGCGCAGGCCAGCGCTTAGGTGTGCACCTTCATACAGAGGCCAGGGCCCGACAC
GCTTGGCAGCTCACACAGGGTGCCACGGTTCTCGGACTTTTCCGCGTTACTCCCGAGATCCCCGCTGGCCTCGGAAG
TACTGGTTCTGGGTCTAAACCCGGTTCCGGCGAAGGTAGTACTAAAGGACGAGAAGGGCCAGAGTTAAGTCCAGATG
ACCCTGCTGGGCTTTTGGACCTGCGGCAGGGCATGTTCGCTCAACTGGTGGCTCAGAACGTGCTGCTGATCGATGGC
CCCCTGAGTTGGTACAGCGATCCCGGGCTGGCAGGCGTGTCACTTACAGGGGGCCTCTCTTACAAGGAAGACACCAA
GGAGTTAGTGGTCGCTAAGGCTGGCGTGTATTACGTGTTCTTCCAACTGGAGCTGAGAAGGGTTGTGGCAGGAGAGG
GTAGCGGCAGCGTGTCTTTAGCCCTTCACTTGCAGCCCCTGAGGTCTGCTGCAGGTGCAGCCGCTCTCGCGCTCACC
GTGGATCTCCCCCCAGCCTCATCTGAAGCTAGGAACAGTGCATTTGGCTTTCAGGGACGCTTGCTGCACCTCTCCGC
TGGACAGAGGCTGGGCGTGCACCTTCACACAGAGGCCCGTGCCAGGCATGCATGGCAGCTCACTCAGGGGGCAACAG
TGCTGGGTCTCTTCCGCGTGACTCCTGAAATACCAGCTGGACTTGGCGGTGGAGGCAGCGGCGGAGGAGGATCTCGT
GAGGGGCCAGAACTGTCCCCCGATGACCCAGCCGGACTGCTCGATCTCAGACAGGGCATGTTCGCTCAGCTTGTAGC
CCAAAATGTCCTCCTGATTGACGGCCCTTTGAGCTGGTATAGTGATCCCGGCTTGGCCGGGGTATCTCTGACCGGAG
GCCTCTCCTACAAGGAAGACACCAAAGAGCTGGTGGTGGCAAAAGCGGGGGTGTATTATGTGTTCTTTCAGCTCGAG
CTGCGGAGAGTTGTGGCCGGGGAAGGGTCTGGGAGCGTATCTCTTGCACTTCACCTGCAGCCCCTGCGCAGCGCCGC
TGGAGCCGCCGCCCTTGCTCTTACTGTGGATCTGCCTCCTGCTTCCTCAGAAGCACGCAACAGCGCCTTCGGCTTTC
AAGGACGTCTCCTGCACTTGTCCGCAGGACAGAGGTTGGGCGTCCATTTACACACTGAGGCACGGGCACGGCACGCT
TGGCAGCTTACCCAGGGAGCCACCGTGCTGGGACTCTTTAGAGTGACACCCGAGATCCCCGCTGGCTTGTCGACAGG
CGGTGGAGGGTCCGGCGGTGGTGGATCAGAGGTGCAGCTGTTGGAGTCTGGGGGAGGCTTGGTACAGCCTGGGGGGT
CCCTGAGACTCTCCTGTGCAGCCTCTGGATTCTCCTTCAGTAGCGGGTACGACATGTGCTGGGTCCGCCAGGCTCCA
GGGAAGGGGCTGGAGTGGATCGCATGTATTGCTGCTGGTAGTGCTGGTATCACTTACGACGCGAACTGGGCGAAAGG
CCGGTTCACCATCTCCAGAGACAATTCCAAGAACACGCTGTATCTGCAAATGAACAGCCTGAGAGCCGAGGACACGG
CCGTATATTACTGTGCGAGATCGGCGTTTTCGTTCGACTACGCCATGGACCTCTGGGGCCAGGGAACCCTGGTCACC
GTCTCGAGCGCTAGCACCAAGGGCCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTGACCGTGCCCTCCAGC
AGCTTGGGCACCCAGACCTACATCTGCAACGTGAATCACAAGCCCAGCAACACCAAGGTGGACAAGAGAGTTGAGCC
CAAATCTTGTGACAAAACTCACACATGCCCACCGTGCCCAGCACCTGAAGCCGCGGGGGCACCGTCAGTCTTCCTCT
TCCCCCCAAAACCCAAGGACACCCTCATGATCTCCCGGACCCCTGAGGTCACATGCGTGGTGGTGGACGTGAGCCAC
GAAGACCCTGAGGTCAAGTTCAACTGGTACGTGGACGGCGTGGAGGTGCATAATGCCAAGACAAAGCCGCGGGAGGA
GCAGTACAACAGCACGTACCGTGTGGTCAGCGTCCTCACCGTCCTGCACCAGGACTGGCTGAATGGCAAGGAGTACA
AGTGCGCGGTCTCCAACAAAGCCCTCCCAGCCCCCATCGAGAAAACCATCTCCAAAGCCAAAGGGCAGCCCCGAGAA
CCACAGGTGTACACCCTGCCCCCATCCCGGGATGAGCTGACCAAGAACCAGGTCAGCCTGACCTGCCTGGTCAAAGG
CTTCTATCCCAGCGACATCGCCGTGGAGTGGGAGAGCAATGGGCAGCCGGAGAACAACTACAAGACCACGCCTCCCG
TGCTGGACTCCGACGGCTCCTTCTTCCTCTATAGCAAGCTCACCGTGGACAAGAGCAGGTGGCAGCAGGGGAACGTC
TTCTCATGCTCCGTGATGCATGAGGCTCTGCACAACCACTACACGCAGAAGAGCCTCTCCCTGTCTCCGGGTGGCGG
TGGAGGGTCCGGCGGTGGTGGATCCGAGGTGCAGCTGGTGGAGTCTGGGGGAGGCTTGGTCCAGCCTGGGGGGTCCC
TGAGACTCTCCTGTGCAGCCTCTGGATTCACCATCAGTACCAATGCAATGAGCTGGGTCCGCCAGGCTCCAGGGAAG
GGGCTGGAGTGGATCGGAGTCATTACTGGTCGTGATATCACATACTACGCGAGCTGGGCGAAAGGCAGATTCACCAT
CTCCAGAGACAATTCCAAGAACACGCTGTATCTTCAAATGAACAGCCTGAGAGCCGAGGACACGGCTGTGTATTACT
GTGCGAGAGACGGTGGTTCTTCTGCTATTACTAGTAACAACATTTGGGGCCAGGGAACCCTGGTCACCGTCTCTTCA
GGCGGTGGCGGTAGTGGGGGAGGCGGTTCTGGCGGCGGAGGGTCCGGCGGTGGAGGATCAGACGTCGTGATGACCCA
GTCTCCTTCCACCCTGTCTGCATCTGTAGGAGACAGAGTCACCATCAATTGCCAAGCCAGTGAGAGCATTAGCAGTT
GGTTAGCCTGGTATCAGCAGAAACCAGGGAAAGCCCCTAAGCTCCTGATCTATGAAGCATCCAAACTGGCATCTGGG
GTCCCATCAAGGTTCAGCGGCAGTGGATCTGGGACAGAATTTACTCTCACCATCAGCAGCCTGCAGCCTGATGATTT
TGCAACTTATTACTGCCAAGGCTATTTTTATTTTATTAGTCGTACTTATGTAAATTCTTTCGGCGGAGGGACCAAGG
TGGAGATCAAAGGCGGTGGAGGGTCCGGCGGTGGTGGCTCCGGATTTCTTAATTCCCTTTTCAACCAAGAGGTTCAG
ATCCCCTTGACTGAAAGCTATTGCGGCCCTTGTCCGAAAAACTGGATATGTTACAAGAATAATTGTTACCAATTCTT
CGACGAAAGCAAGAACTGGTATGAGAGTCAGGCGTCTTGTATGAGTCAGAATGCCAGCCTGCTTAAGGTTTATTCAA
AAGAAGACCAGGATCTGCTTAAGTTGGTAAAGAGCTACCACTGGATGGGGCTGGTACATATCCCAACGAATGGGTCA
TGGCAGTGGGAGGACGGTTCTATTCTGAGTCCAAATCTCCTGACGATCATCGAAATGCAGAAAGGGGACTGTGCCCT
GTATGCATCATCCTTCAAGGGGTACATCGAGAACTGCAGTACCCCAAATACCTACATTTGTATGCAAAGAACGGTTG
GAGGCGGTGGCTCAGGCGGAGGCGGCTCAGGAGGTGGCGGTTCAGGAGGCGGCGGATCTTTCCTAAACTCATTATTC
AACCAAGAAGTTCAAATTCCCTTGACCGAAAGTTACTGTGGCCCATGTCCTAAAAACTGGATATGTTACAAAAATAA
CTGCTACCAATTTTTTGATGAGAGTAAAAACTGGTATGAGAGCCAGGCTTCTTGTATGTCTCAAAATGCCAGCCTTC
TGAAAGTATACAGCAAAGAGGACCAGGATTTACTTAAACTGGTGAAGTCATATCATTGGATGGGACTAGTACACATT
CCAACAAATGGATCTTGGCAGTGGGAAGATGGCTCCATTCTCTCACCCAACCTACTAACAATAATTGAAATGCAGAA
GGGAGACTGTGCACTCTATGCCTCGAGCTTTAAAGGCTATATAGAAAACTGTTCAACTCCAAATACGTACATCTGCA
TGCAAAGGACTGTG
>SEQ ID 26 SI-49E13 chain A aa
REGPELSPDDPAGLLDLRQGMFAQLVAQNVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSAFGFQGRLLHLSAGQRLGVHLHTEARARH
AWQLTQGATVLGLFRVTPEIPAGLGSTGSGSKPGSGEGSTKGREGPELSPDDPAGLLDLRQGMFAQLVAQNVLLIDG
PLSWYSDPGLAGVSLTGGLSYKEDTKELVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGATVLGLFRVTPEIPAGLGGGGSGGGGSR
EGPELSPDDPAGLLDLRQGMFAQLVAQNVLLIDGPLSWYSDPGLAGVSLTGGLSYKEDTKELVVAKAGVYYVFFQLE
LRRVVAGEGSGSVSLALHLQPLRSAAGAAALALTVDLPPASSEARNSAFGFQGRLLHLSAGQRLGVHLHTEARARHA
WQLTQGATVLGLFRVTPEIPAGLSTGGGGSGGGGSEVQLLESGGGLVQPGGSLRLSCAASGFSFSSGYDMCWVRQAP
GKGLEWIACIAAGSAGITYDANWAKGRFTISRDNSKNTLYLQMNSLRAEDTAVYYCARSAFSFDYAMDLWGQGTLVT
VSSASTKGPSVFPLAPSSKSTSGGTAALGCLVKDYFPEPVTVSWNSGALTSGVHTFPAVLQSSGLYSLSSVVTVPSS
SLGTQTYICNVNHKPSNTKVDKRVEPKSCDKTHTCPPCPAPEAAGAPSVFLFPPKPKDTLMISRTPEVTCVVVDVSH
EDPEVKFNWYVDGVEVHNAKTKPREEQYNSTYRVVSVLTVLHQDWLNGKEYKCAVSNKALPAPIEKTISKAKGQPRE
PQVYTLPPSRDELTKNQVSLTCLVKGFYPSDIAVEWESNGQPENNYKTTPPVLDSDGSFFLYSKLTVDKSRWQQGNV
FSCSVMHEALHNHYTQKSLSLSPGGGGGSGGGGSEVQLVESGGGLVQPGGSLRLSCAASGFTISTNAMSWVRQAPGK
GLEWIGVITGRDITYYASWAKGRFTISRDNSKNTLYLQMNSLRAEDTAVYYCARDGGSSAITSNNIWGQGTLVTVSS
GGGGSGGGGSGGGGSGGGGSDVVMTQSPSTLSASVGDRVTINCQASESISSWLAWYQQKPGKAPKLLIYEASKLASG
VPSRFSGSGSGTEFTLTISSLQPDDFATYYCQGYFYFISRTYVNSFGGGTKVEIKGGGGSGGGGSGFLNSLFNQEVQ
IPLTESYCGPCPKNWICYKNNCYQFFDESKNWYESQASCMSQNASLLKVYSKEDQDLLKLVKSYHWMGLVHIPTNGS
WQWEDGSILSPNLLTIIEMQKGDCALYASSFKGYIENCSTPNTYICMQRTVGGGGSGGGGSGGGGSGGGGSFLNSLF
NQEVQIPLTESYCGPCPKNWICYKNNCYQFFDESKNWYESQASCMSQNASLLKVYSKEDQDLLKLVKSYHWMGLVHI
PTNGSWQWEDGSILSPNLLTIIEMQKGDCALYASSFKGYIENCSTPNTYICMQRTV
>SEQ ID 27 SI-49E13 chain B nt
GACATCCAGATGACCCAGTCTCCTTCCACCCTGTCTGCATCTGTAGGAGACAGAGTCACCATCACTTGCCAGGCCAG
TCAGAGCATTAGTTCCCACTTAAACTGGTATCAGCAGAAACCAGGGAAAGCCCCTAAGCTCCTGATCTATAAGGCAT
CCACTCTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAACAGGGTTATAGTTGGGGTAATGTTGATAATGTTTTCGGCGG
AGGGACCAAGGTGGAGATCAAACGTACGGTGGCTGCACCATCTGTCTTCATCTTCCCGCCATCTGATGAGCAGTTGA
AATCTGGAACTGCCTCTGTTGTGTGCCTGCTGAATAACTTCTATCCCAGAGAGGCCAAAGTACAGTGGAAGGTGGAT
AACGCCCTCCAATCGGGTAACTCCCAGGAGAGTGTCACAGAGCAGGACAGCAAGGACAGCACCTACAGCCTCAGCAG
CACCCTGACGCTGAGCAAAGCAGACTACGAGAAACACAAAGTCTACGCCTGCGAAGTCACCCATCAGGGCCTGAGCT
CGCCCGTCACAAAGAGCTTCAACAGGGGAGAGTGT
>SEQ ID 28 SI-49E13 chain B aa
DIQMTQSPSTLSASVGDRVTITCQASQSISSHLNWYQQKPGKAPKLLIYKASTLASGVPSRFSGSGSGTEFTLTISS
LQPDDFATYYCQQGYSWGNVDNVFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGEC
>SEQ ID 29 SI-49E14 chain A nt
GACATCCAGATGACCCAGTCTCCTTCCACCCTGTCTGCATCTGTAGGAGACAGAGTCACCATCACTTGCCAGGCCAG
TCAGAGCATTAGTTCCCACTTAAACTGGTATCAGCAGAAACCAGGGAAAGCCCCTAAGCTCCTGATCTATAAGGCAT
CCACTCTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAACAGGGTTATAGTTGGGGTAATGTTGATAATGTTTTCGGCGG
AGGGACCAAGGTGGAGATCAAAGGCGGTGGCGGTAGTGGGGGAGGCGGTTCTGGCGGCGGAGGGTCCGGCGGTGGAG
GATCAGAGGTGCAGCTGTTGGAGTCTGGGGGAGGCTTGGTACAGCCTGGGGGGTCCCTGAGACTCTCCTGTGCAGCC
TCTGGATTCTCCTTCAGTAGCGGGTACGACATGTGCTGGGTCCGCCAGGCTCCAGGGAAGGGGCTGGAGTGGATCGC
ATGTATTGCTGCTGGTAGTGCTGGTATCACTTACGACGCGAACTGGGCGAAAGGCCGGTTCACCATCTCCAGAGACA
ATTCCAAGAACACGCTGTATCTGCAAATGAACAGCCTGAGAGCCGAGGACACGGCCGTATATTACTGTGCGAGATCG
GCGTTTTCGTTCGACTACGCCATGGACCTCTGGGGCCAGGGAACCCTGGTCACCGTGTCGACAGGCGGTGGAGGGTC
CGGCGGTGGTGGATCACGTGAGGGGCCAGAACTGTCCCCCGATGACCCAGCCGGACTGCTCGATCTCAGACAGGGCA
TGTTCGCTCAGCTTGTAGCCCAAAATGTCCTCCTGATTGACGGCCCTTTGAGCTGGTATAGTGATCCCGGCTTGGCC
GGGGTATCTCTGACCGGAGGCCTCTCCTACAAGGAAGACACCAAAGAGCTGGTGGTGGCAAAAGCGGGGGTGTATTA
TGTGTTCTTTCAGCTCGAGCTGCGGAGAGTTGTGGCCGGGGAAGGGTCTGGGAGCGTATCTCTTGCACTTCACCTGC
AGCCCCTGCGCAGCGCCGCTGGAGCCGCCGCCCTTGCTCTTACTGTGGATCTGCCTCCTGCTTCCTCAGAAGCACGC
AACAGCGCCTTCGGCTTTCAAGGACGTCTCCTGCACTTGTCCGCAGGACAGAGGTTGGGCGTCCATTTACACACTGA
GGCACGGGCACGGCACGCTTGGCAGCTTACCCAGGGAGCCACCGTGCTGGGACTCTTTAGAGTGACACCCGAGATCC
CCGCTGGCTTGGCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCGTGGTGACCGTGCCCTCCA
GCAGCTTGGGCACCCAGACCTACATCTGCAACGTGAATCACAAGCCCAGCAACACCAAGGTGGACAAGAGAGTTGAG
CCCAAATCTTGTGACAAAACTCACACATGCCCACCGTGCCCAGCACCTGAAGCCGCGGGGGCACCGTCAGTCTTCCT
CTTCCCCCCAAAACCCAAGGACACCCTCATGATCTCCCGGACCCCTGAGGTCACATGCGTGGTGGTGGACGTGAGCC
ACGAAGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGGAGTA
CAAGTGCGCGGTCTCCAACAAAGCCCTCCCAGCCCCCATCGAGAAAACCATCTCCAAAGCCAAAGGGCAGCCCCGAG
AACCACAGGTGTACACCCTGCCCCCATCCCGGGATGAGCTGACCAAGAACCAGGTCAGCCTGACCTGCCTGGTCAAA
GGCTTCTATCCCAGCGACATCGCCGTGGAGTGGGAGAGCAATGGGCAGCCGGAGAACAACTACAAGACCACGCCTCC
CGTGCTGGACTCCGACGGCTCCTTCTTCCTCTATAGCAAGCTCACCGTGGACAAGAGCAGGTGGCAGCAGGGGAACG
TCTTCTCATGCTCCGTGATGCATGAGGCTCTGCACAACCACTACACGCAGAAGAGCCTCTCCCTGTCTCCGGGTGGC
GGTGGAGGGTCCGGCGGTGGTGGATCCGAGGTGCAGCTGGTGGAGTCTGGGGGAGGCTTGGTCCAGCCTGGGGGGTC
CCTGAGACTCTCCTGTGCAGCCTCTGGATTCACCATCAGTACCAATGCAATGAGCTGGGTCCGCCAGGCTCCAGGGA
AGGGGCTGGAGTGGATCGGAGTCATTACTGGTCGTGATATCACATACTACGCGAGCTGGGCGAAAGGCAGATTCACC
ATCTCCAGAGACAATTCCAAGAACACGCTGTATCTTCAAATGAACAGCCTGAGAGCCGAGGACACGGCTGTGTATTA
CTGTGCGAGAGACGGTGGTTCTTCTGCTATTACTAGTAACAACATTTGGGGCCAGGGAACCCTGGTCACCGTCTCTT
CAGGCGGTGGCGGTAGTGGGGGAGGCGGTTCTGGCGGCGGAGGGTCCGGCGGTGGAGGATCAGACGTCGTGATGACC
CAGTCTCCTTCCACCCTGTCTGCATCTGTAGGAGACAGAGTCACCATCAATTGCCAAGCCAGTGAGAGCATTAGCAG
TTGGTTAGCCTGGTATCAGCAGAAACCAGGGAAAGCCCCTAAGCTCCTGATCTATGAAGCATCCAAACTGGCATCTG
GGGTCCCATCAAGGTTCAGCGGCAGTGGATCTGGGACAGAATTTACTCTCACCATCAGCAGCCTGCAGCCTGATGAT
TTTGCAACTTATTACTGCCAAGGCTATTTTTATTTTATTAGTCGTACTTATGTAAATTCTTTCGGCGGAGGGACCAA
GGTGGAGATCAAAGGCGGTGGAGGGTCCGGCGGTGGTGGCTCCGGATTTCTTAATTCCCTTTTCAACCAAGAGGTTC
AGATCCCCTTGACTGAAAGCTATTGCGGCCCTTGTCCGAAAAACTGGATATGTTACAAGAATAATTGTTACCAATTC
TTCGACGAAAGCAAGAACTGGTATGAGAGTCAGGCGTCTTGTATGAGTCAGAATGCCAGCCTGCTTAAGGTTTATTC
AAAAGAAGACCAGGATCTGCTTAAGTTGGTAAAGAGCTACCACTGGATGGGGCTGGTACATATCCCAACGAATGGGT
CATGGCAGTGGGAGGACGGTTCTATTCTGAGTCCAAATCTCCTGACGATCATCGAAATGCAGAAAGGGGACTGTGCC
CTGTATGCATCATCCTTCAAGGGGTACATCGAGAACTGCAGTACCCCAAATACCTACATTTGTATGCAAAGAACGGT
TGGAGGCGGTGGCTCAGGCGGAGGCGGCTCAGGAGGTGGCGGTTCAGGAGGCGGCGGATCTTTCCTAAACTCATTAT
TCAACCAAGAAGTTCAAATTCCCTTGACCGAAAGTTACTGTGGCCCATGTCCTAAAAACTGGATATGTTACAAAAAT
AACTGCTACCAATTTTTTGATGAGAGTAAAAACTGGTATGAGAGCCAGGCTTCTTGTATGTCTCAAAATGCCAGCCT
TCTGAAAGTATACAGCAAAGAGGACCAGGATTTACTTAAACTGGTGAAGTCATATCATTGGATGGGACTAGTACACA
TTCCAACAAATGGATCTTGGCAGTGGGAAGATGGCTCCATTCTCTCACCCAACCTACTAACAATAATTGAAATGCAG
AAGGGAGACTGTGCACTCTATGCCTCGAGCTTTAAAGGCTATATAGAAAACTGTTCAACTCCAAATACGTACATCTG
CATGCAAAGGACTGTG
>SEQ ID 30 SI-49E14 chain A aa
DIQMTQSPSTLSASVGDRVTITCQASQSISSHLNWYQQKPGKAPKLLIYKASTLASGVPSRFSGSGSGTEFTLTISS
LQPDDFATYYCQQGYSWGNVDNVFGGGTKVEIKGGGGSGGGGSGGGGSGGGGSEVQLLESGGGLVQPGGSLRLSCAA
SGFSFSSGYDMCWVRQAPGKGLEWIACIAAGSAGITYDANWAKGRFTISRDNSKNTLYLQMNSLRAEDTAVYYCARS
AFSFDYAMDLWGQGTLVTVSTGGGGSGGGGSREGPELSPDDPAGLLDLRQGMFAQLVAQNVLLIDGPLSWYSDPGLA
GVSLTGGLSYKEDTKELVVAKAGVYYVFFQLELRRVVAGEGSGSVSLALHLQPLRSAAGAAALALTVDLPPASSEAR
NSAFGFQGRLLHLSAGQRLGVHLHTEARARHAWQLTQGATVLGLFRVTPEIPAGLASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKRVE
PKSCDKTHTCPPCPAPEAAGAPSVFLFPPKPKDTLMISRTPEVTCVVVDVSHEDPEVKFNWYVDGVEVHNAKTKPRE
EQYNSTYRVVSVLTVLHQDWLNGKEYKCAVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEALHNHYTQKSLSLSPGG
GGGSGGGGSEVQLVESGGGLVQPGGSLRLSCAASGFTISTNAMSWVRQAPGKGLEWIGVITGRDITYYASWAKGRFT
ISRDNSKNTLYLQMNSLRAEDTAVYYCARDGGSSAITSNNIWGQGTLVTVSSGGGGSGGGGSGGGGSGGGGSDVVMT
QSPSTLSASVGDRVTINCQASESISSWLAWYQQKPGKAPKLLIYEASKLASGVPSRFSGSGSGTEFTLTISSLQPDD
FATYYCQGYFYFISRTYVNSFGGGTKVEIKGGGGSGGGGSGFLNSLFNQEVQIPLTESYCGPCPKNWICYKNNCYQF
FDESKNWYESQASCMSQNASLLKVYSKEDQDLLKLVKSYHWMGLVHIPTNGSWQWEDGSILSPNLLTIIEMQKGDCA
LYASSFKGYIENCSTPNTYICMQRTVGGGGSGGGGSGGGGSGGGGSFLNSLFNQEVQIPLTESYCGPCPKNWICYKN
NCYQFFDESKNWYESQASCMSQNASLLKVYSKEDQDLLKLVKSYHWMGLVHIPTNGSWQWEDGSILSPNLLTIIEMQ
KGDCALYASSFKGYIENCSTPNTYICMQRTV
>SEQ ID 31 SI-49E14 chain B nt
CGAGAGGGCCCCGAGCTGTCTCCTGATGACCCAGCAGGCCTCTTGGACTTGCGGCAGGGTATGTTCGCTCAACTTGT
GGCTCAGAATGTTCTGCTCATTGATGGACCACTCTCTTGGTATAGTGACCCCGGTCTGGCCGGGGTGAGTCTGACCG
GCGGGCTCTCTTATAAAGAGGATACTAAGGAACTGGTCGTAGCAAAAGCGGGCGTTTATTACGTTTTTTTTCAGCTG
GAGCTCAGGCGCGTGGTGGCCGGCGAGGGCAGTGGCTCTGTGTCCCTGGCCCTGCACTTACAGCCCTTGAGAAGCGC
TGCAGGTGCTGCCGCACTGGCTTTAACTGTTGACCTCCCTCCGGCCTCTTCTGAAGCTAGAAACAGCGCTTTCGGCT
TCCAAGGGCGCCTGCTGCACCTGAGCGCAGGCCAGCGCTTAGGTGTGCACCTTCATACAGAGGCCAGGGCCCGACAC
GCTTGGCAGCTCACACAGGGTGCCACGGTTCTCGGACTTTTCCGCGTTACTCCCGAGATCCCCGCTGGCCTCGGAAG
TACTGGTTCTGGGTCTAAACCCGGTTCCGGCGAAGGTAGTACTAAAGGACGAGAAGGGCCAGAGTTAAGTCCAGATG
ACCCTGCTGGGCTTTTGGACCTGCGGCAGGGCATGTTCGCTCAACTGGTGGCTCAGAACGTGCTGCTGATCGATGGC
CCCCTGAGTTGGTACAGCGATCCCGGGCTGGCAGGCGTGTCACTTACAGGGGGCCTCTCTTACAAGGAAGACACCAA
GGAGTTAGTGGTCGCTAAGGCTGGCGTGTATTACGTGTTCTTCCAACTGGAGCTGAGAAGGGTTGTGGCAGGAGAGG
GTAGCGGCAGCGTGTCTTTAGCCCTTCACTTGCAGCCCCTGAGGTCTGCTGCAGGTGCAGCCGCTCTCGCGCTCACC
GTGGATCTCCCCCCAGCCTCATCTGAAGCTAGGAACAGTGCATTTGGCTTTCAGGGACGCTTGCTGCACCTCTCCGC
TGGACAGAGGCTGGGCGTGCACCTTCACACAGAGGCCCGTGCCAGGCATGCATGGCAGCTCACTCAGGGGGCAACAG
TGCTGGGTCTCTTCCGCGTGACTCCTGAAATACCAGCTGGACTTCGTACGGTGGCTGCACCATCTGTCTTCATCTTC
CCGCCATCTGATGAGCAGTTGAAATCTGGAACTGCCTCTGTTGTGTGCCTGCTGAATAACTTCTATCCCAGAGAGGC
CAAAGTACAGTGGAAGGTGGATAACGCCCTCCAATCGGGTAACTCCCAGGAGAGTGTCACAGAGCAGGACAGCAAGG
ACAGCACCTACAGCCTCAGCAGCACCCTGACGCTGAGCAAAGCAGACTACGAGAAACACAAAGTCTACGCCTGCGAA
GTCACCCATCAGGGCCTGAGCTCGCCCGTCACAAAGAGCTTCAACAGGGGAGAGTGT
>SEQ ID 32 SI-49E14 chain B aa
REGPELSPDDPAGLLDLRQGMFAQLVAQNVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSAFGFQGRLLHLSAGQRLGVHLHTEARARH
AWQLTQGATVLGLFRVTPEIPAGLGSTGSGSKPGSGEGSTKGREGPELSPDDPAGLLDLRQGMFAQLVAQNVLLIDG
PLSWYSDPGLAGVSLTGGLSYKEDTKELVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGATVLGLFRVTPEIPAGLRTVAAPSVFIF
PPSDEQLKSGTASVVCLLNNFYPREAKVQWKVDNALQSGNSQESVTEQDSKDSTYSLSSTLTLSKADYEKHKVYACE
VTHQGLSSPVTKSFNRGEC
>SEQ ID 33 SI-1P1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGAAGCAGTCAGGACCTGGCCTAGTGCAGCCCTCACAGAGCCTGTCCATCA
CCTGCACAGTCTCTGGTTTCTCATTAACTAACTATGGTGTACACTGGGTTCGCCAGTCTCCAGGAAAGGGTCTGGAG
TGGCTGGGAGTGATATGGAGTGGTGGAAACACAGACTATAATACACCTTTCACATCCAGACTGAGCATCAACAAGGA
CAATTCCAAGAGCCAAGTTTTCTTTAAAATGAACAGTCTGCAATCTAATGACACAGCCATATATTACTGTGCCAGAG
CCCTCACCTACTATGATTACGAGTTTGCTTACTGGGGCCAAGGGACTCTGGTCACTGTCTCTAGCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGCACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGGGGCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGATCT
GGCGGAGGTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATCAAAGGCGGTGGAG
GGTCCGGCGGTGGTGGCTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGCCAGGCTCCAGGGAAGGGGCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACCTGGAGCCTGGCGATGCTGCAACTTACTATTGTC
AGTCTACCTATCTTGGTACTGATTATGTTGGCGGTGCTTTCGGCGGAGGGACCAAGGTGGAGATCAAA
>SEQ ID 34 SI-1P1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLKQSGPGLVQPSQSLSITCTVSGFSLTNYGVHWVRQSPGKGLE
WLGVIWSGGNTDYNTPFTSRLSINKDNSKSQVFFKMNSLQSNDTAIYYCARALTYYDYEFAYWGQGTLVTVSSASTK
GPSVFPLAPSSKSTSGGTAALGCLVKDYFPEPVTVSWNSGALTSGVHTFPAVLOSSGLYSLSSVVTVPSSSLGTQTY
ICNVNHKPSNTKVDKRVEPKSCDKTHTCPPCPAPEAAGAPSVFLFPPKPKDTLMISRTPEVTCVVVDVSHEDPEVKE
NWYVDGVEVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVEIKGGGGSGGGGSGRSLVESGGGLVQPGGSLR
LSCTASGFTISSYHMQWVRQAPGKGLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGGGTKVEIK
>SEQ ID 35 SI-1P1 chain B nt
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTGGCGGTGGAGGGTCCGGCGGTGGTGGATCCCAGTCTGCCCTGACTCAGCCT
GCCTCCGTGTCTGGGTCTCCTGGACAGTCGATCACCATCTCCTGCACTGGAACCAGCAGTGACGTTGGTGGTTATAA
CTTTGTCTCCTGGTACCAACAACACCCAGGCAAAGCCCCCAAACTCATGATCTATGATGTCAGTGATCGGCCCTCAG
GGGTGTCTGATCGCTTCTCCGGCTCCAAGTCTGGCAACACGGCCTCCCTGATCATCTCTGGCCTCCAGGCTGACGAC
GAGGCTGATTATTACTGCAGCTCATATGGGAGCAGCAGCACTCATGTGATTTTCGGCGGAGGGACCAAGGTGACCGT
CCTAGGTGGAGGCGGTTCAGGCGGAGGTGGTTCCGGCGGTGGCGGCTCCGGTGGAGGCGGCTCTCAGGTGCAATTGC
AGGAGTCGGGGGGAGGCCTGGTCAAGCCTGGAGGGTCCCTGAGACTCTCCTGTGCAGCCTCTGGATTCACCTTTAGT
AGTTATTGGATGAGCTGGGTCCGCCAGGCTCCAGGGAAGGGGCTGGAGTGGGTGGCCAACATAAACCGCGATGGAAG
TGCGAGTTACTATGTGGACTCTGTGAAGGGCCGATTCACCATCTCCAGAGACGACGCCAAGAACTCACTGTATCTGC
AAATGAACAGCCTGAGAGCTGAGGACACGGCTGTGTATTACTGTGCGAGAGATCGTGGGGTGGGCTACTTCGATCTC
TGGGGCCGTGGCACCCTGGTCACCGTCTCGAGC
>SEQ ID 36 SI-1P1 chain B aa
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSQSALTQP
ASVSGSPGQSITISCTGTSSDVGGYNFVSWYQQHPGKAPKLMIYDVSDRPSGVSDRFSGSKSGNTASLIISGLQADD
EADYYCSSYGSSSTHVIFGGGTKVTVLGGGGSGGGGSGGGGSGGGGSQVQLQESGGGLVKPGGSLRLSCAASGFTFS
SYWMSWVRQAPGKGLEWVANINRDGSASYYVDSVKGRFTISRDDAKNSLYLQMNSLRAEDTAVYYCARDRGVGYFDL
WGRGTLVTVSS
>SEQ ID 37 SI-1P2 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGAAGCAGTCAGGACCTGGCCTAGTGCAGCCCTCACAGAGCCTGTCCATCA
CCTGCACAGTCTCTGGTTTCTCATTAACTAACTATGGTGTACACTGGGTTCGCCAGTCTCCAGGAAAGGGTCTGGAG
TGGCTGGGAGTGATATGGAGTGGTGGAAACACAGACTATAATACACCTTTCACATCCAGACTGAGCATCAACAAGGA
CAATTCCAAGAGCCAAGTTTTCTTTAAAATGAACAGTCTGCAATCTAATGACACAGCCATATATTACTGTGCCAGAG
CCCTCACCTACTATGATTACGAGTTTGCTTACTGGGGCCAAGGGACTCTGGTCACTGTCTCTAGCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACGCCAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGTGCCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGTTCA
GGCGGAGGTGGAAGTGGTGGTGGCGGCTCTGGAGGCGGCGGATCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCTGCGGGACCAAGGTGGAGATCAAAGGTGGTGGCG
GCTCTGGAGGAGGAGGGTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGGCAGGCACCTGGGAAGTGCCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACTTGGAACCTGGCGATGCTGCAACTTACTATTGTC
AGTCTACCTATCTTGGTACTGATTATGTTGGCGGTGCTTTCGGCTGTGGGACCAAGGTGGAGATCAAA
>SEQ ID 38 SI-1P2 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLKQSGPGLVQPSQSLSITCTVSGFSLTNYGVHWVRQSPGKGLE
WLGVIWSGGNTDYNTPFTSRLSINKDNSKSQVFFKMNSLQSNDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKCLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGCGTKVEIKGGGGSGGGGSGRSLVESGGGLVQPGGSLR
LSCTASGFTISSYHMQWVRQAPGKCLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGCGTKVEIK
>SEQ ID 39 SI-1P2 chain B nt
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTGGCGGTGGAGGGTCCGGCGGTGGTGGATCCCAGTCTGCCCTGACTCAGCCT
GCCTCCGTGTCTGGGTCTCCTGGACAGTCGATCACCATCTCCTGCACTGGAACCAGCAGTGACGTTGGTGGTTATAA
CTTTGTCTCCTGGTACCAACAACACCCAGGCAAAGCCCCCAAACTCATGATCTATGATGTCAGTGATCGGCCCTCAG
GGGTGTCTGATCGCTTCTCCGGCTCCAAGTCTGGCAACACGGCCTCCCTGATCATCTCTGGCCTCCAGGCTGACGAC
GAGGCTGATTATTACTGCAGCTCATATGGGAGCAGCAGCACTCATGTGATTTTCGGCTGCGGGACCAAGGTGACCGT
CCTAGGTGGAGGCGGTTCAGGCGGAGGTGGTTCCGGCGGTGGCGGCTCCGGTGGAGGCGGCTCTCAGGTGCAATTGC
AGGAGTCGGGGGGAGGCCTGGTCAAGCCTGGAGGGTCCCTGAGACTCTCCTGTGCAGCCTCTGGATTCACCTTTAGT
AGTTATTGGATGAGCTGGGTCCGCCAGGCTCCAGGGAAGTGCCTGGAGTGGGTGGCCAACATAAACCGCGATGGAAG
TGCGAGTTACTATGTGGACTCTGTGAAGGGCCGATTCACCATCTCCAGAGACGACGCCAAGAACTCACTGTATCTGC
AAATGAACAGCCTGAGAGCTGAGGACACGGCTGTGTATTACTGTGCGAGAGATCGTGGGGTGGGCTACTTCGATCTC
TGGGGCCGTGGCACCCTGGTCACCGTCTCGAGC
>SEQ ID 40 SI-1P2 chain B aa
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSQSALTQP
ASVSGSPGQSITISCTGTSSDVGGYNFVSWYQQHPGKAPKLMIYDVSDRPSGVSDRFSGSKSGNTASLIISGLQADD
EADYYCSSYGSSSTHVIFGCGTKVTVLGGGGSGGGGSGGGGSGGGGSQVQLQESGGGLVKPGGSLRLSCAASGFTFS
SYWMSWVRQAPGKCLEWVANINRDGSASYYVDSVKGRFTISRDDAKNSLYLQMNSLRAEDTAVYYCARDRGVGYFDL
WGRGTLVTVSS
>SEQ ID 41 SI-1P4 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGGAGTCGGGCCCAGGACTGGTGAAGCCTTCGGAGACCCTGTCCCTCA
CCTGCACTGTCTCTGGTGGCTCCGTCAGCAGTGGTGATTACTACTGGACCTGGATCCGGCAGTCCCCAGGGAAGGGA
CTGGAGTGGATTGGACACATCTATTACAGTGGGAACACCAATTATAACCCCTCCCTCAAGAGCCGACTCACCATATC
AATTGACACGTCCAAGACTCAGTTCTCCCTGAAGCTGAGTTCTGTGACCGCTGCGGACACGGCCATTTATTACTGTG
TGCGAGATCGAGTGACTGGTGCTTTTGATATCTGGGGCCAAGGGACAATGGTCACCGTCTCGAGCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACGCCAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGTGCCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGTTCA
GGCGGAGGTGGAAGTGGTGGTGGCGGCTCTGGAGGCGGCGGATCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCTGCGGGACCAAGGTGGAGATCAAAGGTGGTGGCG
GCTCTGGAGGAGGAGGGTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGGCAGGCACCTGGGAAGTGCCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACTTGGAACCTGGCGATGCTGCAACTTACTATTGTC
AGTCTACCTATCTTGGTACTGATTATGTTGGCGGTGCTTTCGGCTGTGGGACCAAGGTGGAGATCAAA
>SEQ ID 42 SI-1P4 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLQESGPGLVKPSETLSLTCTVSGGSVSSGDYYWTWIRQSPGKG
LEWIGHIYYSGNTNYNPSLKSRLTISIDTSKTQFSLKLSSVTAADTAIYYCVRDRVTGAFDIWGQGTMVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKCLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGCGTKVEIKGGGGSGGGGSGRSLVESGGGLVQPGGSLR
LSCTASGFTISSYHMQWVRQAPGKCLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGCGTKVEIK
>SEQ ID 43 SI-1P4 chain B nt
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCGGAGGGACCAA
GGTGGAAATTAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTGGCGGTGGAGGGTCCGGCGGTGGTGGATCCCAGTCTGCCCTGACTCAGCCT
GCCTCCGTGTCTGGGTCTCCTGGACAGTCGATCACCATCTCCTGCACTGGAACCAGCAGTGACGTTGGTGGTTATAA
CTTTGTCTCCTGGTACCAACAACACCCAGGCAAAGCCCCCAAACTCATGATCTATGATGTCAGTGATCGGCCCTCAG
GGGTGTCTGATCGCTTCTCCGGCTCCAAGTCTGGCAACACGGCCTCCCTGATCATCTCTGGCCTCCAGGCTGACGAC
GAGGCTGATTATTACTGCAGCTCATATGGGAGCAGCAGCACTCATGTGATTTTCGGCTGCGGGACCAAGGTGACCGT
CCTAGGTGGAGGCGGTTCAGGCGGAGGTGGTTCCGGCGGTGGCGGCTCCGGTGGAGGCGGCTCTCAGGTGCAATTGC
AGGAGTCGGGGGGAGGCCTGGTCAAGCCTGGAGGGTCCCTGAGACTCTCCTGTGCAGCCTCTGGATTCACCTTTAGT
AGTTATTGGATGAGCTGGGTCCGCCAGGCTCCAGGGAAGTGCCTGGAGTGGGTGGCCAACATAAACCGCGATGGAAG
TGCGAGTTACTATGTGGACTCTGTGAAGGGCCGATTCACCATCTCCAGAGACGACGCCAAGAACTCACTGTATCTGC
AAATGAACAGCCTGAGAGCTGAGGACACGGCTGTGTATTACTGTGCGAGAGATCGTGGGGTGGGCTACTTCGATCTC
TGGGGCCGTGGCACCCTGGTCACCGTCTCGAGC
>SEQ ID 44 SI-1P4 chain B aa
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSQSALTQP
ASVSGSPGQSITISCTGTSSDVGGYNFVSWYQQHPGKAPKLMIYDVSDRPSGVSDRFSGSKSGNTASLIISGLQADD
EADYYCSSYGSSSTHVIFGCGTKVTVLGGGGSGGGGSGGGGSGGGGSQVQLQESGGGLVKPGGSLRLSCAASGFTFS
SYWMSWVRQAPGKCLEWVANINRDGSASYYVDSVKGRFTISRDDAKNSLYLQMNSLRAEDTAVYYCARDRGVGYFDL
WGRGTLVTVSS
>SEQ ID 45 SI-39P1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAGTTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGCGCGACGGT
GGATCATCTGCTATTACTAGTAACAACATTTGGGGCCAAGGAACTCTGGTCACCGTTTCTTCAGGCGGTGGAGGGTC
CGGCGGTGGTGGATCCGATGTGCAGCTTCAGGAGTCGGGACCTAGCCTGGTGAAACCTTCTCAGTCTCTGTCCCTCA
CCTGCACTGTCACTGGCTACTCAATCACCAGTGATTTTGCCTGGAACTGGATTCGGCAGTTTCCAGGAAACAAGCTG
GAGTGGATGGGCTACATAAGTTATAGTGGTAACACTAGGTACAACCCATCTCTCAAAAGTCGAATCTCTATCACTCG
CGACACATCCAAGAACCAATTCTTCCTGCAGTTGAACTCTGTGACTATTGAGGACACAGCCACATATTACTGTGTAA
CGGCGGGACGCGGGTTTCCTTATTGGGGCCAAGGGACTCTGGTCACTGTCTCTGCAGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAACTCACACATGCCC
ACCGTGCCCAGCACCTGAAGCCGCGGGGGCACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACAACAGCACGTACCGTGTGGTCAG
CGTCCTCACCGTCCTGCACCAGGACTGGCTGAATGGCAAGGAGTACAAGTGCGCGGTCTCCAACAAAGCCCTCCCAG
CCCCCATCGAGAAAACCATCTCCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTGGCGGTGGAGGGTCCGGCGGTGGTGGATCCGAGGT
GCAGCTGTTGGAGTCTGGGGGAGGCTTGGTACAGCCTGGGGGGTCCCTGAGACTCTCCTGTGCAGCCTCTGGATTCT
CCTTCAGTAGCGGGTACGACATGTGCTGGGTCCGCCAGGCTCCAGGGAAGGGGCTGGAGTGGATCGCATGCATTGCT
GCTGGTAGTGCTGGTATCACTTACGACGCGAACTGGGCGAAAGGCCGGTTCACCATCTCCAGAGACAATTCCAAGAA
CACGCTGTATCTGCAAATGAACAGCCTGAGAGCCGAGGACACGGCCGTATATTACTGTGCGAGATCGGCGTTTTCGT
TCGACTACGCCATGGACCTCTGGGGCCAGGGAACCCTGGTCACCGTCTCGAGCGGCGGTGGCGGTAGTGGGGGAGGC
GGTTCTGGCGGCGGAGGGTCCGGCGGTGGAGGATCAGACATCCAGATGACCCAGTCTCCTTCCACCCTGTCTGCATC
TGTAGGAGACAGAGTCACCATCACTTGCCAGGCCAGTCAGAGCATTAGTTCCCACTTAAACTGGTATCAGCAGAAAC
CAGGGAAAGCCCCTAAGCTCCTGATCTATAAGGCATCCACTCTGGCATCTGGGGTCCCATCAAGGTTCAGCGGCAGT
GGATCTGGGACAGAATTTACTCTCACCATCAGCAGCCTGCAGCCTGATGATTTTGCAACTTATTACTGCCAACAGGG
TTATAGTTGGGGTAATGTTGATAATGTTTTCGGCGGAGGGACCAAGGTGGAGATCAAAGGCGGTGGAGGGTCCGGCG
GTGGTGGATCCCAGTCGCTGGTGGAGTCTGGGGGAGGCTTGGTACAGCCTGGGGGGTCCCTGAGACTCTCCTGTGCA
GCCTCTGGATTCTCCTTCAGTAGCAACTACTGGATATGCTGGGTCCGCCAGGCTCCAGGGAAGGGGCTGGAGTGGAT
CGCATGTATTTATGTTGGTAGTAGTGGTGACACTTACTACGCGAGCTCCGCGAAAGGCCGGTTCACCATCTCCAGAG
ACAATTCCAAGAACACGCTGTATCTGCAAATGAACAGCCTGAGAGCCGAGGACACGGCCGTATATTACTGTGCGAGA
GATAGTAGTAGTTATTATATGTTTAACTTGTGGGGCCAGGGAACCCTGGTCACCGTCTCTTCAGGCGGTGGCGGTAG
TGGGGGAGGCGGTTCTGGCGGCGGAGGGTCCGGCGGTGGAGGATCAGCCCTTGTGATGACCCAGTCTCCTTCCACCC
TGTCTGCATCTGTAGGAGACAGAGTCACCATCAATTGCCAGGCCAGTGAGGACATTGATACCTATTTAGCCTGGTAT
CAGCAGAAACCAGGGAAAGCCCCTAAGCTCCTGATCTTTTACGCATCCGATCTGGCATCTGGGGTCCCATCAAGGTT
CAGCGGCAGTGGATCTGGGACAGAATTTACTCTCACCATCAGCAGCCTGCAGCCTGATGATTTTGCAACTTATTACT
GCCAAGGCGGTTACTATACTAGTAGTGCTGATACGAGGGGTGCTTTCGGCGGAGGGACCAAGGTGGAGATCAAA
>SEQ ID 46 SI-39P1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSSGGGGSGGGGSDVQLQESGPSLVKPSQSLSLTCTVTGYSITSDFAWNWIRQFPGNKL
EWMGYISYSGNTRYNPSLKSRISITRDTSKNQFFLQLNSVTIEDTATYYCVTAGRGFPYWGQGTLVTVSAASTKGPS
VFPLAPSSKSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPEAAGAPSVFLFPPKPKDTLMISRTPEVTCVVVDVSHEDPEVKFNWY
VDGVEVHNAKTKPREEQYNSTYRVVSVLTVLHQDWLNGKEYKCAVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVMHEAL
HNHYTQKSLSLSPGGGGGSGGGGSEVQLLESGGGLVQPGGSLRLSCAASGFSFSSGYDMCWVRQAPGKGLEWIACIA
AGSAGITYDANWAKGRFTISRDNSKNTLYLQMNSLRAEDTAVYYCARSAFSFDYAMDLWGQGTLVTVSSGGGGSGGG
GSGGGGSGGGGSDIQMTQSPSTLSASVGDRVTITCQASQSISSHLNWYQQKPGKAPKLLIYKASTLASGVPSRFSGS
GSGTEFTLTISSLQPDDFATYYCQQGYSWGNVDNVFGGGTKVEIKGGGGSGGGGSQSLVESGGGLVQPGGSLRLSCA
ASGFSFSSNYWICWVRQAPGKGLEWIACIYVGSSGDTYYASSAKGRFTISRDNSKNTLYLQMNSLRAEDTAVYYCAR
DSSSYYMFNLWGQGTLVTVSSGGGGSGGGGSGGGGSGGGGSALVMTQSPSTLSASVGDRVTINCQASEDIDTYLAWY
QQKPGKAPKLLIFYASDLASGVPSRFSGSGSGTEFTLTISSLQPDDFATYYCQGGYYTSSADTRGAFGGGTKVEIK
>SEQ ID 47 SI-39P1 chain B nt
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTGGCGGTGGAGGGTCCGGCGGTGGTGGATCCCAGTCTGCCCTGACTCAGCCT
GCCTCCGTGTCTGGGTCTCCTGGACAGTCGATCACCATCTCCTGCACTGGAACCAGCAGTGACGTTGGTGGTTATAA
CTTTGTCTCCTGGTACCAACAACACCCAGGCAAAGCCCCCAAACTCATGATCTATGATGTCAGTGATCGGCCCTCAG
GGGTGTCTGATCGCTTCTCCGGCTCCAAGTCTGGCAACACGGCCTCCCTGATCATCTCTGGCCTCCAGGCTGACGAC
GAGGCTGATTATTACTGCAGCTCATATGGGAGCAGCAGCACTCATGTGATTTTCGGCGGAGGGACCAAGGTGACCGT
CCTAGGTGGAGGCGGTTCAGGCGGAGGTGGTTCCGGCGGTGGCGGCTCCGGTGGAGGCGGCTCTCAGGTGCAATTGC
AGGAGTCGGGGGGAGGCCTGGTCAAGCCTGGAGGGTCCCTGAGACTCTCCTGTGCAGCCTCTGGATTCACCTTTAGT
AGTTATTGGATGAGCTGGGTCCGCCAGGCTCCAGGGAAGGGGCTGGAGTGGGTGGCCAACATAAACCGCGATGGAAG
TGCGAGTTACTATGTGGACTCTGTGAAGGGCCGATTCACCATCTCCAGAGACGACGCCAAGAACTCACTGTATCTGC
AAATGAACAGCCTGAGAGCTGAGGACACGGCTGTGTATTACTGTGCGAGAGATCGTGGGGTGGGCTACTTCGATCTC
TGGGGCCGTGGCACCCTGGTCACCGTCTCGAGC
>SEQ ID 48 SI-39P1 chain B aa
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSQSALTQP
ASVSGSPGQSITISCTGTSSDVGGYNFVSWYQQHPGKAPKLMIYDVSDRPSGVSDRFSGSKSGNTASLIISGLQADD
EADYYCSSYGSSSTHVIFGGGTKVTVLGGGGSGGGGSGGGGSGGGGSQVQLQESGGGLVKPGGSLRLSCAASGFTFS
SYWMSWVRQAPGKGLEWVANINRDGSASYYVDSVKGRFTISRDDAKNSLYLQMNSLRAEDTAVYYCARDRGVGYFDL
WGRGTLVTVSS
>SEQ ID 49 SI-38P5 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGCAGCCCGGCGCCGAGCTGGTGAAGCCCGGCGCCAGCGTGAAGATGA
GCTGCAAGGCCAGCGGCTACACCTTCACCAGCTACAACATGCACTGGGTGAAGCAGACCCCCGGCCGGGGCCTGGAG
TGGATCGGCGCCATCTACCCCGGCAACGGCGACACCAGCTACAACCAGAAGTTCAAGGGCAAGGCCACCCTGACCGC
CGACAAGAGCAGCAGCACCGCCTACATGCAGCTGAGCAGCCTGACCAGCGAGGACAGCGCCGTGTACTACTGCGCCC
GGAGCACCTACTACGGCGGCGACTGGTACTTCAACGTGTGGGGCGCCGGCACCACCGTGACCGTGAGCGCCGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGCACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTG
CAGCCTCTGGATTCTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGTTCACCATCTCCAG
AGACAATTCCAAGAACACGCTGTATCTGCAAATGAACAGCCTGAGAGCCGAGGACACGGCCGTATATTACTGTGCGA
GATCGGCGTTTTCGTTCGACTACGCCATGGACCTCTGGGGCCAGGGAACCCTGGTCACCGTCTCGAGCGGTGGAGGC
GGATCTGGCGGAGGTGGTTCCGGCGGTGGCGGCTCCGGTGGAGGCGGCTCTGACATCCAGATGACCCAGTCTCCTTC
CACCCTGTCTGCATCTGTAGGAGACAGAGTCACCATCACTTGCCAGGCCAGTCAGAGCATTAGTTCCCACTTAAACT
GGTATCAGCAGAAACCAGGGAAAGCCCCTAAGCTCCTGATCTATAAGGCATCCACTCTGGCATCTGGGGTCCCATCA
AGGTTCAGCGGCAGTGGATCTGGGACAGAATTTACTCTCACCATCAGCAGCCTGCAGCCTGATGATTTTGCAACTTA
TTACTGCCAACAGGGTTATAGTTGGGGTAATGTTGATAATGTTTTCGGCGGAGGGACCAAGGTGGAGATCAAAGGCG
GTGGAGGGTCCGGCGGTGGTGGCTCCGGACGGTCGCTGGTGGAGTCTGGGGGAGGCTTGGTCCAGCCTGGGGGGTCC
CTGAGACTCTCCTGTACTGCCTCTGGATTCACCATCAGTAGCTACCACATGCAGTGGGTCCGCCAGGCTCCAGGGAA
GGGGCTGGAGTACATCGGAACCATTAGTAGTGGTGGTAATGTATACTACGCAAGCTCCGCTAGAGGCAGATTCACCA
TCTCCAGACCCTCGTCCAAGAACACGGTGGATCTTCAAATGAACAGCCTGAGAGCCGAGGACACGGCTGTGTATTAC
TGTGCGAGAGACTCTGGTTATAGTGATCCTATGTGGGGCCAGGGAACCCTGGTCACCGTCTCTTCAGGCGGTGGCGG
TAGTGGGGGAGGCGGTTCTGGCGGCGGAGGGTCCGGCGGTGGAGGATCAGACGTTGTGATGACCCAGTCTCCATCTT
CCGTGTCTGCATCTGTAGGAGACAGAGTCACCATCACCTGTCAGGCCAGTCAGAACATTAGGACTTACTTATCCTGG
TATCAGCAGAAACCAGGGAAAGCCCCTAAGCTCCTGATCTATGCTGCAGCCAATCTGGCATCTGGGGTCCCATCAAG
GTTCAGCGGCAGTGGATCTGGGACAGATTTCACTCTCACCATCAGCGACCTGGAGCCTGGCGATGCTGCAACTTACT
ATTGTCAGTCTACCTATCTTGGTACTGATTATGTTGGCGGTGCTTTCGGCGGAGGGACCAAGGTGGAGATCAAA
>SEQ ID 50 SI-38P5 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLQQPGAELVKPGASVKMSCKASGYTFTSYNMHWVKQTPGRGLE
WIGAIYPGNGDTSYNQKFKGKATLTADKSSSTAYMQLSSLTSEDSAVYYCARSTYYGGDWYFNVWGAGTTVTVSAAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVTCVVVDVSHEDPEV
KFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSFDYAMDLWGQGTLVTVSSGGG
GSGGGGSGGGGSGGGGSDIQMTQSPSTLSASVGDRVTITCQASQSISSHLNWYQQKPGKAPKLLIYKASTLASGVPS
RFSGSGSGTEFTLTISSLQPDDFATYYCQQGYSWGNVDNVFGGGTKVEIKGGGGSGGGGSGRSLVESGGGLVQPGGS
LRLSCTASGFTISSYHMQWVRQAPGKGLEYIGTISSGGNVYYASSARGRFTISRPSSKNTVDLQMNSLRAEDTAVYY
CARDSGYSDPMWGQGTLVTVSSGGGGSGGGGSGGGGSGGGGSDVVMTQSPSSVSASVGDRVTITCQASQNIRTYLSW
YQQKPGKAPKLLIYAAANLASGVPSRFSGSGSGTDFTLTISDLEPGDAATYYCQSTYLGTDYVGGAFGGGTKVEIK
>SEQ ID 51 SI-38P5 chain B nt
CAGATCGTGCTGAGCCAGAGCCCCGCCATCCTGAGCGCCAGCCCCGGCGAGAAGGTGACCATGACCTGCCGGGCCAG
CAGCAGCGTGAGCTACATCCACTGGTTCCAGCAGAAGCCCGGCAGCAGCCCCAAGCCCTGGATCTACGCCACCAGCA
ACCTGGCCAGCGGCGTGCCCGTGCGGTTCAGCGGCAGCGGCAGCGGCACCAGCTACAGCCTGACCATCAGCCGGGTG
GAGGCCGAGGACGCCGCCACCTACTACTGCCAGCAGTGGACCAGCAACCCCCCCACCTTCGGCGGCGGCACCAAGCT
GGAGATCAAGCGTACGGTGGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTGGCGGTGGAGGGTCCGGCGGTGGTGGATCCGAGGTGCAGCTGGTGCAGTCTGGA
GCAGAGGTGAAGAAACCAGGAGAGTCTCTGAAGATCTCCTGTAAGGGTTCTGGATACAGCTTTAGCAGTTCATGGAT
CGGCTGGGTGCGCCAGGCACCTGGGAAAGGCCTGGAATGGATGGGGATCATCTATCCTGATGACTCTGATACCAGAT
ACAGTCCATCCTTCCAAGGCCAGGTCACCATCTCAGCCGACAAGTCCATCAGGACTGCCTACCTGCAGTGGAGTAGC
CTGAAGGCCTCGGACACCGCTATGTATTACTGTGCGAGACATGTTACTATGATTTGGGGAGTTATTATTGACTTCTG
GGGCCAGGGAACCCTGGTCACCGTCTCCTCAGGCGGTGGCGGTAGTGGGGGAGGCGGTTCTGGCGGCGGAGGGTCCG
GCGGTGGAGGATCAGCCATCCAGTTGACCCAGTCTCCATCCTCCCTGTCTGCATCTGTAGGAGACAGAGTCACCATC
ACTTGCCGGGCAAGTCAGGGCATTAGCAGTGCTTTAGCCTGGTATCAGCAGAAACCAGGGAAAGCTCCTAAGCTCCT
GATCTATGATGCCTCCAGTTTGGAAAGTGGGGTCCCATCAAGGTTCAGCGGCAGTGGATCTGGGACAGATTTCACTC
TCACCATCAGCAGCCTGCAGCCTGAAGATTTTGCAACTTATTACTGTCAACAGTTTAATAGTTACCCATTCACTTTC
GGCCCTGGGACCAAAGTGGATATCAAA
>SEQ ID 52 SI-38P5 chain B aa
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKLEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGECGGGGSGGGGSEVQLVQSG
AEVKKPGESLKISCKGSGYSFSSSWIGWVRQAPGKGLEWMGIIYPDDSDTRYSPSFQGQVTISADKSIRTAYLQWSS
LKASDTAMYYCARHVTMIWGVIIDFWGQGTLVTVSSGGGGSGGGGSGGGGSGGGGSAIQLTQSPSSLSASVGDRVTI
TCRASQGISSALAWYQQKPGKAPKLLIYDASSLESGVPSRFSGSGSGTDFTLTISSLQPEDFATYYCQQFNSYPFTF
GPGTKVDIK
>SEQ ID 53 SI-38P6 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCCCAGGTGCAGCTGCAGCAGCCCGGCGCCGAGCTGGTGAAGCCCGGCGCCAGCGTGAAGATGA
GCTGCAAGGCCAGCGGCTACACCTTCACCAGCTACAACATGCACTGGGTGAAGCAGACCCCCGGCCGGGGCCTGGAG
TGGATCGGCGCCATCTACCCCGGCAACGGCGACACCAGCTACAACCAGAAGTTCAAGGGCAAGGCCACCCTGACCGC
CGACAAGAGCAGCAGCACCGCCTACATGCAGCTGAGCAGCCTGACCAGCGAGGACAGCGCCGTGTACTACTGCGCCC
GGAGCACCTACTACGGCGGCGACTGGTACTTCAACGTGTGGGGCGCCGGCACCACCGTGACCGTGAGCGCCGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACGCCAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTG
CAGCCTCTGGATTCTCCTTCAGTAGCGGGTACGACATGTGCTGGGTCCGCCAGGCTCCAGGGAAGTGCCTGGAGTGG
ATCGCATGCATTGCTGCTGGTAGTGCTGGTATCACTTACGACGCGAACTGGGCGAAAGGCCGGTTCACCATCTCCAG
AGACAATTCCAAGAACACGCTGTATCTGCAAATGAACAGCCTGAGAGCCGAGGACACGGCCGTATATTACTGTGCGA
GATCGGCGTTTTCGTTCGACTACGCCATGGACCTCTGGGGCCAGGGAACCCTGGTCACCGTCTCGAGCGGTGGAGGC
GGTTCAGGCGGAGGTGGAAGTGGTGGTGGCGGCTCTGGAGGCGGCGGATCTGACATCCAGATGACCCAGTCTCCTTC
CACCCTGTCTGCATCTGTAGGAGACAGAGTCACCATCACTTGCCAGGCCAGTCAGAGCATTAGTTCCCACTTAAACT
GGTATCAGCAGAAACCAGGGAAAGCCCCTAAGCTCCTGATCTATAAGGCATCCACTCTGGCATCTGGGGTCCCATCA
AGGTTCAGCGGCAGTGGATCTGGGACAGAATTTACTCTCACCATCAGCAGCCTGCAGCCTGATGATTTTGCAACTTA
TTACTGCCAACAGGGTTATAGTTGGGGTAATGTTGATAATGTTTTCGGCTGCGGGACCAAGGTGGAGATCAAAGGTG
GTGGCGGCTCTGGAGGAGGAGGGTCCGGACGGTCGCTGGTGGAGTCTGGGGGAGGCTTGGTCCAGCCTGGGGGGTCC
CTGAGACTCTCCTGTACTGCCTCTGGATTCACCATCAGTAGCTACCACATGCAGTGGGTCCGGCAGGCACCTGGGAA
GTGCCTGGAGTACATCGGAACCATTAGTAGTGGTGGTAATGTATACTACGCAAGCTCCGCTAGAGGCAGATTCACCA
TCTCCAGACCCTCGTCCAAGAACACGGTGGATCTTCAAATGAACAGCCTGAGAGCCGAGGACACGGCTGTGTATTAC
TGTGCGAGAGACTCTGGTTATAGTGATCCTATGTGGGGCCAGGGAACCCTGGTCACCGTCTCTTCAGGCGGTGGCGG
TAGTGGGGGAGGCGGTTCTGGCGGCGGAGGGTCCGGCGGTGGAGGATCAGACGTTGTGATGACCCAGTCTCCATCTT
CCGTGTCTGCATCTGTAGGAGACAGAGTCACCATCACCTGTCAGGCCAGTCAGAACATTAGGACTTACTTATCCTGG
TATCAGCAGAAACCAGGGAAAGCCCCTAAGCTCCTGATCTATGCTGCAGCCAATCTGGCATCTGGGGTCCCATCAAG
GTTCAGCGGCAGTGGATCTGGGACAGATTTCACTCTCACCATCAGCGACTTGGAACCTGGCGATGCTGCAACTTACT
ATTGTCAGTCTACCTATCTTGGTACTGATTATGTTGGCGGTGCTTTCGGCTGTGGGACCAAGGTGGAGATCAAA
>SEQ ID 54 SI-38P6 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLQQPGAELVKPGASVKMSCKASGYTFTSYNMHWVKQTPGRGLE
WIGAIYPGNGDTSYNQKFKGKATLTADKSSSTAYMQLSSLTSEDSAVYYCARSTYYGGDWYFNVWGAGTTVTVSAAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGGPSVFLFPPKPKDTLMISRTPEVTCVVVDVSHEDPEV
KFNWYVDGVEVHNAKTKPREEQYASTYRvvSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGGGGGSGGGGSEVQLLESGGGLVQPGGSLRLSCAASGFSFSSGYDMCWVRQAPGKCLEW
IACIAAGSAGITYDANWAKGRFTISRDNSKNTLYLQMNSLRAEDTAVYYCARSAFSFDYAMDLWGQGTLVTVSSGGG
GSGGGGSGGGGSGGGGSDIQMTQSPSTLSASVGDRVTITCQASQSISSHLNWYQQKPGKAPKLLIYKASTLASGVPS
RFSGSGSGTEFTLTISSLQPDDFATYYCQQGYSWGNVDNVFGCGTKVEIKGGGGSGGGGSGRSLVESGGGLVQPGGS
LRLSCTASGFTISSYHMQWVRQAPGKCLEYIGTISSGGNVYYASSARGRFTISRPSSKNTVDLQMNSLRAEDTAVYY
CARDSGYSDPMWGQGTLVTVSSGGGGSGGGGSGGGGSGGGGSDVVMTQSPSSVSASVGDRVTITCQASQNIRTYLSW
YQQKPGKAPKLLIYAAANLASGVPSRFSGSGSGTDFTLTISDLEPGDAATYYCQSTYLGTDYVGGAFGCGTKVEIK
>SEQ ID 55 SI-38P6 chain B nt
CAGATCGTGCTGAGCCAGAGCCCCGCCATCCTGAGCGCCAGCCCCGGCGAGAAGGTGACCATGACCTGCCGGGCCAG
CAGCAGCGTGAGCTACATCCACTGGTTCCAGCAGAAGCCCGGCAGCAGCCCCAAGCCCTGGATCTACGCCACCAGCA
ACCTGGCCAGCGGCGTGCCCGTGCGGTTCAGCGGCAGCGGCAGCGGCACCAGCTACAGCCTGACCATCAGCCGGGTG
GAGGCCGAGGACGCCGCCACCTACTACTGCCAGCAGTGGACCAGCAACCCCCCCACCTTCGGCGGCGGCACCAAGCT
GGAGATCAAGCGTACGGTGGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTGGCGGTGGAGGGTCCGGCGGTGGTGGATCCGAGGTGCAGCTGGTGCAGTCTGGA
GCAGAGGTGAAGAAACCAGGAGAGTCTCTGAAGATCTCCTGTAAGGGTTCTGGATACAGCTTTAGCAGTTCATGGAT
CGGCTGGGTGCGCCAGGCACCTGGGAAATGTCTGGAATGGATGGGGATCATCTATCCTGATGACTCTGATACCAGAT
ACAGTCCATCCTTCCAAGGCCAGGTCACCATCTCAGCCGACAAGTCCATCAGGACTGCCTACCTGCAGTGGAGTAGC
CTGAAGGCCTCGGACACCGCTATGTATTACTGTGCGAGACATGTTACTATGATTTGGGGAGTTATTATTGACTTCTG
GGGCCAGGGAACCCTGGTCACCGTCTCCTCAGGCGGTGGCGGTAGTGGGGGAGGCGGTTCTGGCGGCGGAGGGTCCG
GCGGTGGAGGATCAGCCATCCAGTTGACCCAGTCTCCATCCTCCCTGTCTGCATCTGTAGGAGACAGAGTCACCATC
ACTTGCCGGGCAAGTCAGGGCATTAGCAGTGCTTTAGCCTGGTATCAGCAGAAACCAGGGAAAGCTCCTAAGCTCCT
GATCTATGATGCCTCCAGTTTGGAAAGTGGGGTCCCATCAAGGTTCAGCGGCAGTGGATCTGGGACAGATTTCACTC
TCACCATCAGCAGCCTGCAGCCTGAAGATTTTGCAACTTATTACTGTCAACAGTTTAATAGTTACCCATTCACTTTC
GGCTGTGGGACCAAAGTGGATATCAAA
>SEQ ID 56 SI-38P6 chain B aa
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKLEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGECGGGGSGGGGSEVQLVQSG
AEVKKPGESLKISCKGSGYSFSSSWIGWVRQAPGKCLEWMGIIYPDDSDTRYSPSFQGQVTISADKSIRTAYLQWSS
LKASDTAMYYCARHVTMIWGVIIDFWGQGTLVTVSSGGGGSGGGGSGGGGSGGGGSAIQLTQSPSSLSASVGDRVTI
TCRASQGISSALAWYQQKPGKAPKLLIYDASSLESGVPSRFSGSGSGTDFTLTISSLQPEDFATYYCQQFNSYPFTF
GCGTKVDIK
>SEQ ID 57 SI-49P1 chain A nt
GAGATCGTGATGACCCAGAGCCCCAGCACCCTGAGCGCCAGCGTGGGCGACAGGGTGATCATCACCTGCCAGGCCAG
CGAGAGCATCAGCAGCTGGCTGGCCTGGTACCAGCAGAAGCCCGGCAAGGCCCCCAAGCTGCTGATCTACGAGGCCA
GCAAGCTGGCCAGCGGCGTGCCCAGCAGGTTCAGCGGCAGCGGCAGCGGCGCCGAGTTCACCCTGACCATCAGCAGC
CTGCAGCCCGACGACTTCGCCACCTACTACTGCCAGGGCTACTTCTACTTCATCAGCAGGACCTACGTGAACAGCTT
CGGCTGCGGCACCAAGCTGACCGTGCTGGGCGGTGGCGGTAGTGGGGGAGGCGGTTCTGGCGGCGGAGGGTCCGGCG
GTGGAGGATCAGAGGTGCAGCTGGTGGAGAGCGGCGGCGGCCTGGTGCAGCCCGGCGGCAGCCTGAGGCTGAGCTGC
ACCGCCAGCGGCTTCACCATCAGCACCAACGCCATGAGCTGGGTGAGGCAGGCCCCCGGCAAGTGCCTGGAGTGGGT
GGGCGTGATCACCGGCAGGGACATCACCTACTACGCCAGCTGGGCCAAGGGCAGGTTCACCATCAGCAGGGACACCA
GCAAGAACACCGTGTACCTGCAGATGAACAGCCTGAGGGCCGAGGACACCGCCGTGTACTACTGCGCCAGGGACGGC
GGCAGCAGCGCCATCACCAGCAACAACATCTGGGGCCAGGGCACCCTGGTGACCGTGTCGACAGGCGGTGGAGGGTC
CGGCGGTGGTGGATCACAGGTACAACTGCAGCAGTCTGGGCCTGAGCTGGAGAAGCCTGGCGCTTCAGTGAAGATAT
CCTGCAAGGCTTCTGGTTACTCATTCACTGGCTACACCATGAACTGGGTGAAGCAGAGCCATGGAAAGAGCCTTGAG
TGGATTGGACTTATTACTCCTTACAATGGTGCTTCTAGCTACAACCAGAAGTTCAGGGGCAAGGCCACATTAACTGT
AGACAAGTCATCCAGCACAGCCTACATGGACCTCCTCAGTCTGACATCTGAAGACTCTGCAGTCTATTTCTGTGCAA
GGGGGGGTTACGACGGGAGGGGTTTTGACTACTGGGGATCCGGGACCCCGGTCACCGTCTCCTCAGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACGCCAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGTGCCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGTTCA
GGCGGAGGTGGAAGTGGTGGTGGCGGCTCTGGAGGCGGCGGATCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCTGCGGGACCAAGGTGGAGATCAAAGGTGGTGGCG
GCTCTGGAGGAGGAGGGTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGGCAGGCACCTGGGAAGTGCCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACTTGGAACCTGGCGATGCTGCAACTTACTATTGTC
AGTCTACCTATCTTGGTACTGATTATGTTGGCGGTGCTTTCGGCTGTGGGACCAAGGTGGAGATCAAATGA
>SEQ ID 58 SI-49P1 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGGGGSGGGGSGGGGSGGGGSEVQLVESGGGLVQPGGSLRLSC
TASGFTISTNAMSWVRQAPGKCLEWVGVITGRDITYYASWAKGRFTISRDTSKNTVYLQMNSLRAEDTAVYYCARDG
GSSAITSNNIWGQGTLVTVSTGGGGSGGGGSQVQLQQSGPELEKPGASVKISCKASGYSFTGYTMNWVKQSHGKSLE
WIGLITPYNGASSYNQKFRGKATLTVDKSSSTAYMDLLSLTSEDSAVYFCARGGYDGRGFDYWGSGTPVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKCLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGCGTKVEIKGGGGSGGGGSGRSLVESGGGLVQPGGSLR
LSCTASGFTISSYHMQWVRQAPGKCLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGCGTKVEIK
>SEQ ID 59 SI-49P1 chain B nt
GACATCGAGCTCACTCAGTCTCCAGCAATCATGTCTGCATCTCCAGGGGAGAAGGTCACCATGACCTGCAGTGCCAG
CTCAAGTGTAAGTTACATGCACTGGTACCAGCAGAAGTCAGGCACCTCCCCCAAAAGATGGATTTATGACACATCCA
AACTGGCTTCTGGAGTCCCAGGTCGCTTCAGTGGCAGTGGGTCTGGAAACTCTTACTCTCTCACAATCAGCAGCGTG
GAGGCTGAAGATGATGCAACTTATTACTGCCAGCAGTGGAGTAAGCACCCTCTCACGTTCGGATCCGGGACCAAGGT
GGAAATCAAACGTACGGTGGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTGGCGGTGGAGGGTCCGGCGGTGGTGGATCCTTTCTTAATTCCCTTTTCAACCAA
GAGGTTCAGATCCCCTTGACTGAAAGCTATTGCGGCCCTTGTCCGAAAAACTGGATATGTTACAAGAATAATTGTTA
CCAATTCTTCGACGAAAGCAAGAACTGGTATGAGAGTCAGGCGTCTTGTATGAGTCAGAATGCCAGCCTGCTTAAGG
TTTATTCAAAAGAAGACCAGGATCTGCTTAAGTTGGTAAAGAGCTACCACTGGATGGGGCTGGTACATATCCCAACG
AATGGGTCATGGCAGTGGGAGGACGGTTCTATTCTGAGTCCAAATCTCCTGACGATCATCGAAATGCAGAAAGGGGA
CTGTGCCCTGTATGCATCATCCTTCAAGGGGTACATCGAGAACTGCAGTACCCCAAATACCTACATTTGTATGCAAA
GAACGGTTGGAGGCGGTGGCTCAGGCGGAGGCGGCTCAGGAGGTGGCGGTTCAGGAGGCGGCGGATCTTTCCTAAAC
TCATTATTCAACCAAGAAGTTCAAATTCCCTTGACCGAAAGTTACTGTGGCCCATGTCCTAAAAACTGGATATGTTA
CAAAAATAACTGCTACCAATTTTTTGATGAGAGTAAAAACTGGTATGAGAGCCAGGCTTCTTGTATGTCTCAAAATG
CCAGCCTTCTGAAAGTATACAGCAAAGAGGACCAGGATTTACTTAAACTGGTGAAGTCATATCATTGGATGGGACTA
GTACACATTCCAACAAATGGATCTTGGCAGTGGGAAGATGGCTCCATTCTCTCACCCAACCTACTAACAATAATTGA
AATGCAGAAGGGAGACTGTGCACTCTATGCCTCGAGCTTTAAAGGCTATATAGAAAACTGTTCAACTCCAAATACGT
ACATCTGCATGCAAAGGACTGTGTAG
>SEQ ID 60 SI-49P1 chain B aa
DIELTQSPAIMSASPGEKVTMTCSASSSVSYMHWYQQKSGTSPKRWIYDTSKLASGVPGRFSGSGSGNSYSLTISSV
EAEDDATYYCQQWSKHPLTFGSGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGECGGGGSGGGGSFLNSLFNQ
EVQIPLTESYCGPCPKNWICYKNNCYQFFDESKNWYESQASCMSQNASLLKVYSKEDQDLLKLVKSYHWMGLVHIPT
NGSWQWEDGSILSPNLLTIIEMQKGDCALYASSFKGYIENCSTPNTYICMQRTVGGGGSGGGGSGGGGSGGGGSFLN
SLFNQEVQIPLTESYCGPCPKNWICYKNNCYQFFDESKNWYESQASCMSQNASLLKVYSKEDQDLLKLVKSYHWMGL
VHIPTNGSWQWEDGSILSPNLLTIIEMQKGDCALYASSFKGYIENCSTPNTYICMQRTV
>SEQ ID 61 SI-49P2 chain A nt
GAGATCGTGATGACCCAGAGCCCCAGCACCCTGAGCGCCAGCGTGGGCGACAGGGTGATCATCACCTGCCAGGCCAG
CGAGAGCATCAGCAGCTGGCTGGCCTGGTACCAGCAGAAGCCCGGCAAGGCCCCCAAGCTGCTGATCTACGAGGCCA
GCAAGCTGGCCAGCGGCGTGCCCAGCAGGTTCAGCGGCAGCGGCAGCGGCGCCGAGTTCACCCTGACCATCAGCAGC
CTGCAGCCCGACGACTTCGCCACCTACTACTGCCAGGGCTACTTCTACTTCATCAGCAGGACCTACGTGAACAGCTT
CGGCTGCGGCACCAAGCTGACCGTGCTGGGCGGTGGCGGTAGTGGGGGAGGCGGTTCTGGCGGCGGAGGGTCCGGCG
GTGGAGGATCAGAGGTGCAGCTGGTGGAGAGCGGCGGCGGCCTGGTGCAGCCCGGCGGCAGCCTGAGGCTGAGCTGC
ACCGCCAGCGGCTTCACCATCAGCACCAACGCCATGAGCTGGGTGAGGCAGGCCCCCGGCAAGTGCCTGGAGTGGGT
GGGCGTGATCACCGGCAGGGACATCACCTACTACGCCAGCTGGGCCAAGGGCAGGTTCACCATCAGCAGGGACACCA
GCAAGAACACCGTGTACCTGCAGATGAACAGCCTGAGGGCCGAGGACACCGCCGTGTACTACTGCGCCAGGGACGGC
GGCAGCAGCGCCATCACCAGCAACAACATCTGGGGCCAGGGCACCCTGGTGACCGTGTCGACAGGCGGTGGAGGGTC
CGGCGGTGGTGGATCACAGGTCCAACTGCAGCAGCCTGGGGCTGAGCTGGTGAGGCCTGGGGCTTCAGTGAAGCTGT
CCTGCAAGGCTTCTGGCTACACCTTCACCAGCTACTGGATAAACTGGGTGAAGCAGAGGCCTGGACAAGGCCTTGAG
TGGATCGGAAATATTTATCCTTCTGATAGTTATACTAACTACAATCAAAAGTTCAAGGACAAGGCCACATTGACTGT
AGACAAATCCTCCAGCACAGCCTACATGCAGCTCAGCAGCCCGACATCTGAGGACTCTGCGGTCTATTACTGTACAA
GATCGTGGAGGGGTAACTCCTTTGACTACTGGGGCCAAGGCACCACTCTCACAGTCTCCTCAGCTAGCACCAAGGGC
CCATCGGTCTTCCCCCTGGCACCCTCCTCCAAGAGCACCTCTGGGGGCACAGCGGCCCTGGGCTGCCTGGTCAAGGA
CTACTTCCCCGAACCGGTGACGGTGTCGTGGAACTCAGGCGCCCTGACCAGCGGCGTGCACACCTTCCCGGCTGTCC
TACAGTCCTCAGGACTCTACTCCCTCAGCAGCGTGGTGACCGTGCCCTCCAGCAGCTTGGGCACCCAGACCTACATC
TGCAACGTGAATCACAAGCCCAGCAACACCAAGGTGGACAAGAGAGTTGAGCCCAAATCTTGTGACAAAACTCACAC
ATGCCCACCGTGCCCAGCACCTGAAGCCGCGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACGCCAGCACGTACCGTGT
GGTCAGCGTCCTCACCGTCCTGCACCAGGACTGGCTGAATGGCAAGGAGTACAAGTGCGCGGTCTCCAACAAAGCCC
TCCCAGCCCCCATCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCT
TCCTCTATAGCAAGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTGGCGGTGGAGGGTCCGGCGGTGGTGGATC
CGAGGTGCAGCTGTTGGAGTCTGGGGGAGGCTTGGTACAGCCTGGGGGGTCCCTGAGACTCTCCTGTGCAGCCTCTG
GATTCTCCTTCAGTAGCGGGTACGACATGTGCTGGGTCCGCCAGGCTCCAGGGAAGTGCCTGGAGTGGATCGCATGC
ATTGCTGCTGGTAGTGCTGGTATCACTTACGACGCGAACTGGGCGAAAGGCCGGTTCACCATCTCCAGAGACAATTC
CAAGAACACGCTGTATCTGCAAATGAACAGCCTGAGAGCCGAGGACACGGCCGTATATTACTGTGCGAGATCGGCGT
TTTCGTTCGACTACGCCATGGACCTCTGGGGCCAGGGAACCCTGGTCACCGTCTCGAGCGGTGGAGGCGGTTCAGGC
GGAGGTGGAAGTGGTGGTGGCGGCTCTGGAGGCGGCGGATCTGACATCCAGATGACCCAGTCTCCTTCCACCCTGTC
TGCATCTGTAGGAGACAGAGTCACCATCACTTGCCAGGCCAGTCAGAGCATTAGTTCCCACTTAAACTGGTATCAGC
AGAAACCAGGGAAAGCCCCTAAGCTCCTGATCTATAAGGCATCCACTCTGGCATCTGGGGTCCCATCAAGGTTCAGC
GGCAGTGGATCTGGGACAGAATTTACTCTCACCATCAGCAGCCTGCAGCCTGATGATTTTGCAACTTATTACTGCCA
ACAGGGTTATAGTTGGGGTAATGTTGATAATGTTTTCGGCTGCGGGACCAAGGTGGAGATCAAAGGTGGTGGCGGCT
CTGGAGGAGGAGGGTCCGGACGGTCGCTGGTGGAGTCTGGGGGAGGCTTGGTCCAGCCTGGGGGGTCCCTGAGACTC
TCCTGTACTGCCTCTGGATTCACCATCAGTAGCTACCACATGCAGTGGGTCCGGCAGGCACCTGGGAAGTGCCTGGA
GTACATCGGAACCATTAGTAGTGGTGGTAATGTATACTACGCAAGCTCCGCTAGAGGCAGATTCACCATCTCCAGAC
CCTCGTCCAAGAACACGGTGGATCTTCAAATGAACAGCCTGAGAGCCGAGGACACGGCTGTGTATTACTGTGCGAGA
GACTCTGGTTATAGTGATCCTATGTGGGGCCAGGGAACCCTGGTCACCGTCTCTTCAGGCGGTGGCGGTAGTGGGGG
AGGCGGTTCTGGCGGCGGAGGGTCCGGCGGTGGAGGATCAGACGTTGTGATGACCCAGTCTCCATCTTCCGTGTCTG
CATCTGTAGGAGACAGAGTCACCATCACCTGTCAGGCCAGTCAGAACATTAGGACTTACTTATCCTGGTATCAGCAG
AAACCAGGGAAAGCCCCTAAGCTCCTGATCTATGCTGCAGCCAATCTGGCATCTGGGGTCCCATCAAGGTTCAGCGG
CAGTGGATCTGGGACAGATTTCACTCTCACCATCAGCGACTTGGAACCTGGCGATGCTGCAACTTACTATTGTCAGT
CTACCTATCTTGGTACTGATTATGTTGGCGGTGCTTTCGGCTGTGGGACCAAGGTGGAGATCAAATGA
>SEQ ID 62 SI-49P2 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGGGGSGGGGSGGGGSGGGGSEVQLVESGGGLVQPGGSLRLSC
TASGFTISTNAMSWVRQAPGKCLEWVGVITGRDITYYASWAKGRFTISRDTSKNTVYLQMNSLRAEDTAVYYCARDG
GSSAITSNNIWGQGTLVTVSTGGGGSGGGGSQVQLQQPGAELVRPGASVKLSCKASGYTFTSYWINWVKQRPGQGLE
WIGNIYPSDSYTNYNQKFKDKATLTVDKSSSTAYMQLSSPTSEDSAVYYCTRSWRGNSFDYWGQGTTLTVSSASTKG
PSVFPLAPSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPEAAGGPSVFLFPPKPKDTLMISRTPEVTCVVVDVSHEDPEVKFN
WYVDGVEVHNAKTKPREEQYASTYRVVSVLTVLHQDWLNGKEYKCAVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQGNVFSCSVMHE
ALHNHYTQKSLSLSPGGGGGSGGGGSEVQLLESGGGLVQPGGSLRLSCAASGFSFSSGYDMCWVRQAPGKCLEWIAC
IAAGSAGITYDANWAKGRFTISRDNSKNTLYLQMNSLRAEDTAVYYCARSAFSFDYAMDLWGQGTLVTVSSGGGGSG
GGGSGGGGSGGGGSDIQMTQSPSTLSASVGDRVTITCQASQSISSHLNWYQQKPGKAPKLLIYKASTLASGVPSRFS
GSGSGTEFTLTISSLQPDDFATYYCQQGYSWGNVDNVFGCGTKVEIKGGGGSGGGGSGRSLVESGGGLVQPGGSLRL
SCTASGFTISSYHMQWVRQAPGKCLEYIGTISSGGNVYYASSARGRFTISRPSSKNTVDLQMNSLRAEDTAVYYCAR
DSGYSDPMWGQGTLVTVSSGGGGSGGGGSGGGGSGGGGSDVVMTQSPSSVSASVGDRVTITCQASQNIRTYLSWYQQ
KPGKAPKLLIYAAANLASGVPSRFSGSGSGTDFTLTISDLEPGDAATYYCQSTYLGTDYVGGAFGCGTKVEIK
>SEQ ID 63 SI-49P2 chain B nt
GACATTGTGATGACACAGTCTCCATCCTCCCTGACTGTGACAGCAGGAGAGAAGGTCACTATGAGCTGCAAGTCCAG
TCAGAGTCTGTTAAACAGTGGAAATCAAAAGAACTACTTGACCTGGTACCAGCAGAAACCAGGGCAGCCTCCTAAAC
TGTTGATCTACTGGGCATCCACTAGGGAATCTGGGGTCCCTGATCGCTTCACAGGCAGTGGATCTGGAACAGATTTC
ACTCTCACCATCAGCAGTGTGCAGGCTGAAGACCTGGCAGTTTATTACTGTCAGAATGATTATAGTTATCCATTCAC
GTTCGGCTCGGGGACAAAGTTGGAAATAAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTGCGAAGTCACCCATCAGG
GCCTGAGCTCGCCCGTCACAAAGAGCTTCAACAGGGGAGAGTGTGGCGGTGGAGGGTCCGGCGGTGGTGGATCCTTT
CTTAATTCCCTTTTCAACCAAGAGGTTCAGATCCCCTTGACTGAAAGCTATTGCGGCCCTTGTCCGAAAAACTGGAT
ATGTTACAAGAATAATTGTTACCAATTCTTCGACGAAAGCAAGAACTGGTATGAGAGTCAGGCGTCTTGTATGAGTC
AGAATGCCAGCCTGCTTAAGGTTTATTCAAAAGAAGACCAGGATCTGCTTAAGTTGGTAAAGAGCTACCACTGGATG
GGGCTGGTACATATCCCAACGAATGGGTCATGGCAGTGGGAGGACGGTTCTATTCTGAGTCCAAATCTCCTGACGAT
CATCGAAATGCAGAAAGGGGACTGTGCCCTGTATGCATCATCCTTCAAGGGGTACATCGAGAACTGCAGTACCCCAA
ATACCTACATTTGTATGCAAAGAACGGTTGGAGGCGGTGGCTCAGGCGGAGGCGGCTCAGGAGGTGGCGGTTCAGGA
GGCGGCGGATCTTTCCTAAACTCATTATTCAACCAAGAAGTTCAAATTCCCTTGACCGAAAGTTACTGTGGCCCATG
TCCTAAAAACTGGATATGTTACAAAAATAACTGCTACCAATTTTTTGATGAGAGTAAAAACTGGTATGAGAGCCAGG
CTTCTTGTATGTCTCAAAATGCCAGCCTTCTGAAAGTATACAGCAAAGAGGACCAGGATTTACTTAAACTGGTGAAG
TCATATCATTGGATGGGACTAGTACACATTCCAACAAATGGATCTTGGCAGTGGGAAGATGGCTCCATTCTCTCACC
CAACCTACTAACAATAATTGAAATGCAGAAGGGAGACTGTGCACTCTATGCCTCGAGCTTTAAAGGCTATATAGAAA
ACTGTTCAACTCCAAATACGTACATCTGCATGCAAAGGACTGTGTAG
>SEQ ID 64 SI-49P2 chain B aa
DIVMTQSPSSLTVTAGEKVTMSCKSSQSLLNSGNQKNYLTWYQQKPGQPPKLLIYWASTRESGVPDRFTGSGSGTDF
TLTISSVQAEDLAVYYCQNDYSYPFTFGSGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGECGGGGSGGGGSF
LNSLFNQEVQIPLTESYCGPCPKNWICYKNNCYQFFDESKNWYESQASCMSQNASLLKVYSKEDQDLLKLVKSYHWM
GLVHIPTNGSWQWEDGSILSPNLLTIIEMQKGDCALYASSFKGYIENCSTPNTYICMQRTVGGGGSGGGGSGGGGSG
GGGSFLNSLFNQEVQIPLTESYCGPCPKNWICYKNNCYQFFDESKNWYESQASCMSQNASLLKVYSKEDQDLLKLVK
SYHWMGLVHIPTNGSWQWEDGSILSPNLLTIIEMQKGDCALYASSFKGYIENCSTPNTYICMQRT
>SEQ ID 65 SI-49P3 chain A nt
GAGATCGTGATGACCCAGAGCCCCAGCACCCTGAGCGCCAGCGTGGGCGACAGGGTGATCATCACCTGCCAGGCCAG
CGAGAGCATCAGCAGCTGGCTGGCCTGGTACCAGCAGAAGCCCGGCAAGGCCCCCAAGCTGCTGATCTACGAGGCCA
GCAAGCTGGCCAGCGGCGTGCCCAGCAGGTTCAGCGGCAGCGGCAGCGGCGCCGAGTTCACCCTGACCATCAGCAGC
CTGCAGCCCGACGACTTCGCCACCTACTACTGCCAGGGCTACTTCTACTTCATCAGCAGGACCTACGTGAACAGCTT
CGGCTGCGGCACCAAGCTGACCGTGCTGGGCGGTGGCGGTAGTGGGGGAGGCGGTTCTGGCGGCGGAGGGTCCGGCG
GTGGAGGATCAGAGGTGCAGCTGGTGGAGAGCGGCGGCGGCCTGGTGCAGCCCGGCGGCAGCCTGAGGCTGAGCTGC
ACCGCCAGCGGCTTCACCATCAGCACCAACGCCATGAGCTGGGTGAGGCAGGCCCCCGGCAAGTGCCTGGAGTGGGT
GGGCGTGATCACCGGCAGGGACATCACCTACTACGCCAGCTGGGCCAAGGGCAGGTTCACCATCAGCAGGGACACCA
GCAAGAACACCGTGTACCTGCAGATGAACAGCCTGAGGGCCGAGGACACCGCCGTGTACTACTGCGCCAGGGACGGC
GGCAGCAGCGCCATCACCAGCAACAACATCTGGGGCCAGGGCACCCTGGTGACCGTGTCGACAGGCGGTGGAGGGTC
CGGCGGTGGTGGATCAGAGGTTCAGCTGGTGGAGTCTGGCGGTGGCCTGGTGCAGCCAGGGGGCTCACTCCGTTTGT
CCTGTGCAGCTTCTGGCTTCAACATTAAAGACACCTATATACACTGGGTGCGTCAGGCCCCGGGTAAGGGCCTGGAA
TGGGTTGCAAGGATTTATCCTACGAATGGTTATACTAGATATGCCGATAGCGTCAAGGGCCGTTTCACTATAAGCGC
AGACACATCCAAAAACACAGCCTACCTGCAGATGAACAGCCTGCGTGCTGAGGACACTGCCGTCTATTATTGTTCTA
GATGGGGAGGGGACGGCTTCTATGCTATGGACTACTGGGGTCAAGGAACCCTGGTCACCGTCTCCTCGGCTAGCACC
AAGGGCCCATCGGTCTTCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAGAGTTGAGCCCAAATCTTGTGACAAAAC
TCACACATGCCCACCGTGCCCAGCACCTGAAGCCGCGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACGCCAGCACGTA
CCGTGTGGTCAGCGTCCTCACCGTCCTGCACCAGGACTGGCTGAATGGCAAGGAGTACAAGTGCGCGGTCTCCAACA
AAGCCCTCCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTGGCGGTGGAGGGTCCGGCGGTGG
TGGATCCGAGGTGCAGCTGTTGGAGTCTGGGGGAGGCTTGGTACAGCCTGGGGGGTCCCTGAGACTCTCCTGTGCAG
CCTCTGGATTCTCCTTCAGTAGCGGGTACGACATGTGCTGGGTCCGCCAGGCTCCAGGGAAGTGCCTGGAGTGGATC
GCATGCATTGCTGCTGGTAGTGCTGGTATCACTTACGACGCGAACTGGGCGAAAGGCCGGTTCACCATCTCCAGAGA
CAATTCCAAGAACACGCTGTATCTGCAAATGAACAGCCTGAGAGCCGAGGACACGGCCGTATATTACTGTGCGAGAT
CGGCGTTTTCGTTCGACTACGCCATGGACCTCTGGGGCCAGGGAACCCTGGTCACCGTCTCGAGCGGTGGAGGCGGT
TCAGGCGGAGGTGGAAGTGGTGGTGGCGGCTCTGGAGGCGGCGGATCTGACATCCAGATGACCCAGTCTCCTTCCAC
CCTGTCTGCATCTGTAGGAGACAGAGTCACCATCACTTGCCAGGCCAGTCAGAGCATTAGTTCCCACTTAAACTGGT
ATCAGCAGAAACCAGGGAAAGCCCCTAAGCTCCTGATCTATAAGGCATCCACTCTGGCATCTGGGGTCCCATCAAGG
TTCAGCGGCAGTGGATCTGGGACAGAATTTACTCTCACCATCAGCAGCCTGCAGCCTGATGATTTTGCAACTTATTA
CTGCCAACAGGGTTATAGTTGGGGTAATGTTGATAATGTTTTCGGCTGCGGGACCAAGGTGGAGATCAAAGGTGGTG
GCGGCTCTGGAGGAGGAGGGTCCGGACGGTCGCTGGTGGAGTCTGGGGGAGGCTTGGTCCAGCCTGGGGGGTCCCTG
AGACTCTCCTGTACTGCCTCTGGATTCACCATCAGTAGCTACCACATGCAGTGGGTCCGGCAGGCACCTGGGAAGTG
CCTGGAGTACATCGGAACCATTAGTAGTGGTGGTAATGTATACTACGCAAGCTCCGCTAGAGGCAGATTCACCATCT
CCAGACCCTCGTCCAAGAACACGGTGGATCTTCAAATGAACAGCCTGAGAGCCGAGGACACGGCTGTGTATTACTGT
GCGAGAGACTCTGGTTATAGTGATCCTATGTGGGGCCAGGGAACCCTGGTCACCGTCTCTTCAGGCGGTGGCGGTAG
TGGGGGAGGCGGTTCTGGCGGCGGAGGGTCCGGCGGTGGAGGATCAGACGTTGTGATGACCCAGTCTCCATCTTCCG
TGTCTGCATCTGTAGGAGACAGAGTCACCATCACCTGTCAGGCCAGTCAGAACATTAGGACTTACTTATCCTGGTAT
CAGCAGAAACCAGGGAAAGCCCCTAAGCTCCTGATCTATGCTGCAGCCAATCTGGCATCTGGGGTCCCATCAAGGTT
CAGCGGCAGTGGATCTGGGACAGATTTCACTCTCACCATCAGCGACTTGGAACCTGGCGATGCTGCAACTTACTATT
GTCAGTCTACCTATCTTGGTACTGATTATGTTGGCGGTGCTTTCGGCTGTGGGACCAAGGTGGAGATCAAATGA
>SEQ ID 66 SI-49P3 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGGGGSGGGGSGGGGSGGGGSEVQLVESGGGLVQPGGSLRLSC
TASGFTISTNAMSWVRQAPGKCLEWVGVITGRDITYYASWAKGRFTISRDTSKNTVYLQMNSLRAEDTAVYYCARDG
GSSAITSNNIWGQGTLVTVSTGGGGSGGGGSEVQLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFYAMDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPEAAGGPSVFLFPPKPKDTLMISRTPEVTCVVVDVSHEDPEVK
FNWYVDGVEVHNAKTKPREEQYASTYRVVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGGSGGGGSEVQLLESGGGLVQPGGSLRLSCAASGFSFSSGYDMCWVRQAPGKCLEWI
ACIAAGSAGITYDANWAKGRFTISRDNSKNTLYLQMNSLRAEDTAVYYCARSAFSFDYAMDLWGQGTLVTVSSGGGG
SGGGGSGGGGSGGGGSDIQMTQSPSTLSASVGDRVTITCQASQSISSHLNWYQQKPGKAPKLLIYKASTLASGVPSR
FSGSGSGTEFTLTISSLQPDDFATYYCQQGYSWGNVDNVFGCGTKVEIKGGGGSGGGGSGRSLVESGGGLVQPGGSL
RLSCTASGFTISSYHMQWVRQAPGKCLEYIGTISSGGNVYYASSARGRFTISRPSSKNTVDLQMNSLRAEDTAVYYC
ARDSGYSDPMWGQGTLVTVSSGGGGSGGGGSGGGGSGGGGSDVVMTQSPSSVSASVGDRVTITCQASQNIRTYLSWY
QQKPGKAPKLLIYAAANLASGVPSRFSGSGSGTDFTLTISDLEPGDAATYYCQSTYLGTDYVGGAFGCGTKVEIK
>SEQ ID 67 SI-49P3 chain B nt
GATATCCAGATGACCCAGTCCCCGAGCTCCCTGTCCGCCTCTGTGGGCGATAGGGTCACCATCACCTGCCGTGCCAG
TCAGGATGTGAATACTGCTGTAGCCTGGTATCAACAGAAACCAGGAAAAGCTCCGAAACTACTGATTTACTCGGCAT
CCTTCCTCTACTCTGGAGTCCCTTCTCGCTTCTCTGGCTCCAGATCTGGGACGGATTTCACTCTGACCATCAGCAGT
CTGCAGCCGGAAGACTTCGCAACTTATTACTGTCAGCAACATTATACTACTCCTCCCACGTTCGGACAGGGTACCAA
GGTGGAGATCAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTGGCGGTGGAGGGTCCGGCGGTGGTGGATCCTTTCTTAATTCCCTTTTCAAC
CAAGAGGTTCAGATCCCCTTGACTGAAAGCTATTGCGGCCCTTGTCCGAAAAACTGGATATGTTACAAGAATAATTG
TTACCAATTCTTCGACGAAAGCAAGAACTGGTATGAGAGTCAGGCGTCTTGTATGAGTCAGAATGCCAGCCTGCTTA
AGGTTTATTCAAAAGAAGACCAGGATCTGCTTAAGTTGGTAAAGAGCTACCACTGGATGGGGCTGGTACATATCCCA
ACGAATGGGTCATGGCAGTGGGAGGACGGTTCTATTCTGAGTCCAAATCTCCTGACGATCATCGAAATGCAGAAAGG
GGACTGTGCCCTGTATGCATCATCCTTCAAGGGGTACATCGAGAACTGCAGTACCCCAAATACCTACATTTGTATGC
AAAGAACGGTTGGAGGCGGTGGCTCAGGCGGAGGCGGCTCAGGAGGTGGCGGTTCAGGAGGCGGCGGATCTTTCCTA
AACTCATTATTCAACCAAGAAGTTCAAATTCCCTTGACCGAAAGTTACTGTGGCCCATGTCCTAAAAACTGGATATG
TTACAAAAATAACTGCTACCAATTTTTTGATGAGAGTAAAAACTGGTATGAGAGCCAGGCTTCTTGTATGTCTCAAA
ATGCCAGCCTTCTGAAAGTATACAGCAAAGAGGACCAGGATTTACTTAAACTGGTGAAGTCATATCATTGGATGGGA
CTAGTACACATTCCAACAAATGGATCTTGGCAGTGGGAAGATGGCTCCATTCTCTCACCCAACCTACTAACAATAAT
TGAAATGCAGAAGGGAGACTGTGCACTCTATGCCTCGAGCTTTAAAGGCTATATAGAAAACTGTTCAACTCCAAATA
CGTACATCTGCATGCAAAGGACTGTGTAG
>SEQ ID 68 SI-49P3 chain B aa
DIQMTQSPSSLSASVGDRVTITCRASQDVNTAVAWYQQKPGKAPKLLIYSASFLYSGVPSRFSGSRSGTDFTLTISS
LQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSFLNSLFN
QEVQIPLTESYCGPCPKNWICYKNNCYQFFDESKNWYESQASCMSQNASLLKVYSKEDQDLLKLVKSYHWMGLVHIP
TNGSWQWEDGSILSPNLLTIIEMQKGDCALYASSFKGYIENCSTPNTYICMQRTVGGGGSGGGGSGGGGSGGGGSFL
NSLFNQEVQIPLTESYCGPCPKNWICYKNNCYQFFDESKNWYESQASCMSQNASLLKVYSKEDQDLLKLVKSYHWMG
LVHIPTNGSWQWEDGSILSPNLLTIIEMQKGDCALYASSFKGYIENCSTPNTYICMQRTV
>SEQ ID 69 SI-49P4 chain A nt
GAGATCGTGATGACCCAGAGCCCCAGCACCCTGAGCGCCAGCGTGGGCGACAGGGTGATCATCACCTGCCAGGCCAG
CGAGAGCATCAGCAGCTGGCTGGCCTGGTACCAGCAGAAGCCCGGCAAGGCCCCCAAGCTGCTGATCTACGAGGCCA
GCAAGCTGGCCAGCGGCGTGCCCAGCAGGTTCAGCGGCAGCGGCAGCGGCGCCGAGTTCACCCTGACCATCAGCAGC
CTGCAGCCCGACGACTTCGCCACCTACTACTGCCAGGGCTACTTCTACTTCATCAGCAGGACCTACGTGAACAGCTT
CGGCTGCGGCACCAAGCTGACCGTGCTGGGCGGTGGCGGTAGTGGGGGAGGCGGTTCTGGCGGCGGAGGGTCCGGCG
GTGGAGGATCAGAGGTGCAGCTGGTGGAGAGCGGCGGCGGCCTGGTGCAGCCCGGCGGCAGCCTGAGGCTGAGCTGC
ACCGCCAGCGGCTTCACCATCAGCACCAACGCCATGAGCTGGGTGAGGCAGGCCCCCGGCAAGTGCCTGGAGTGGGT
GGGCGTGATCACCGGCAGGGACATCACCTACTACGCCAGCTGGGCCAAGGGCAGGTTCACCATCAGCAGGGACACCA
GCAAGAACACCGTGTACCTGCAGATGAACAGCCTGAGGGCCGAGGACACCGCCGTGTACTACTGCGCCAGGGACGGC
GGCAGCAGCGCCATCACCAGCAACAACATCTGGGGCCAGGGCACCCTGGTGACCGTGTCGACAGGCGGTGGAGGGTC
CGGCGGTGGTGGATCAGATGTGCAGCTTCAGGAGTCGGGACCTAGCCTGGTGAAACCTTCTCAGTCTCTGTCCCTCA
CCTGCACTGTCACTGGCTACTCAATCACCAGTGATTTTGCCTGGAACTGGATTCGGCAGTTTCCAGGAAACAAGCTG
GAGTGGATGGGCTACATAAGTTATAGTGGTAACACTAGGTACAACCCATCTCTCAAAAGTCGAATCTCTATCACTCG
CGACACATCCAAGAACCAATTCTTCCTGCAGTTGAACTCTGTGACTATTGAGGACACAGCCACATATTACTGTGTAA
CGGCGGGACGCGGGTTTCCTTATTGGGGCCAAGGGACTCTGGTCACTGTCTCTGCAGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAACTCACACATGCCC
ACCGTGCCCAGCACCTGAAGCCGCGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACGCCAGCACGTACCGTGTGGTCAG
CGTCCTCACCGTCCTGCACCAGGACTGGCTGAATGGCAAGGAGTACAAGTGCGCGGTCTCCAACAAAGCCCTCCCAG
CCCCCATCGAGAAAACCATCTCCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTGGCGGTGGAGGGTCCGGCGGTGGTGGATCCGAGGT
GCAGCTGTTGGAGTCTGGGGGAGGCTTGGTACAGCCTGGGGGGTCCCTGAGACTCTCCTGTGCAGCCTCTGGATTCT
CCTTCAGTAGCGGGTACGACATGTGCTGGGTCCGCCAGGCTCCAGGGAAGTGCCTGGAGTGGATCGCATGCATTGCT
GCTGGTAGTGCTGGTATCACTTACGACGCGAACTGGGCGAAAGGCCGGTTCACCATCTCCAGAGACAATTCCAAGAA
CACGCTGTATCTGCAAATGAACAGCCTGAGAGCCGAGGACACGGCCGTATATTACTGTGCGAGATCGGCGTTTTCGT
TCGACTACGCCATGGACCTCTGGGGCCAGGGAACCCTGGTCACCGTCTCGAGCGGTGGAGGCGGTTCAGGCGGAGGT
GGAAGTGGTGGTGGCGGCTCTGGAGGCGGCGGATCTGACATCCAGATGACCCAGTCTCCTTCCACCCTGTCTGCATC
TGTAGGAGACAGAGTCACCATCACTTGCCAGGCCAGTCAGAGCATTAGTTCCCACTTAAACTGGTATCAGCAGAAAC
CAGGGAAAGCCCCTAAGCTCCTGATCTATAAGGCATCCACTCTGGCATCTGGGGTCCCATCAAGGTTCAGCGGCAGT
GGATCTGGGACAGAATTTACTCTCACCATCAGCAGCCTGCAGCCTGATGATTTTGCAACTTATTACTGCCAACAGGG
TTATAGTTGGGGTAATGTTGATAATGTTTTCGGCTGCGGGACCAAGGTGGAGATCAAAGGTGGTGGCGGCTCTGGAG
GAGGAGGGTCCGGACGGTCGCTGGTGGAGTCTGGGGGAGGCTTGGTCCAGCCTGGGGGGTCCCTGAGACTCTCCTGT
ACTGCCTCTGGATTCACCATCAGTAGCTACCACATGCAGTGGGTCCGGCAGGCACCTGGGAAGTGCCTGGAGTACAT
CGGAACCATTAGTAGTGGTGGTAATGTATACTACGCAAGCTCCGCTAGAGGCAGATTCACCATCTCCAGACCCTCGT
CCAAGAACACGGTGGATCTTCAAATGAACAGCCTGAGAGCCGAGGACACGGCTGTGTATTACTGTGCGAGAGACTCT
GGTTATAGTGATCCTATGTGGGGCCAGGGAACCCTGGTCACCGTCTCTTCAGGCGGTGGCGGTAGTGGGGGAGGCGG
TTCTGGCGGCGGAGGGTCCGGCGGTGGAGGATCAGACGTTGTGATGACCCAGTCTCCATCTTCCGTGTCTGCATCTG
TAGGAGACAGAGTCACCATCACCTGTCAGGCCAGTCAGAACATTAGGACTTACTTATCCTGGTATCAGCAGAAACCA
GGGAAAGCCCCTAAGCTCCTGATCTATGCTGCAGCCAATCTGGCATCTGGGGTCCCATCAAGGTTCAGCGGCAGTGG
ATCTGGGACAGATTTCACTCTCACCATCAGCGACTTGGAACCTGGCGATGCTGCAACTTACTATTGTCAGTCTACCT
ATCTTGGTACTGATTATGTTGGCGGTGCTTTCGGCTGTGGGACCAAGGTGGAGATCAAATGA
>SEQ ID 70 SI-49P4 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGGGGSGGGGSGGGGSGGGGSEVQLVESGGGLVQPGGSLRLSC
TASGFTISTNAMSWVRQAPGKCLEWVGVITGRDITYYASWAKGRFTISRDTSKNTVYLQMNSLRAEDTAVYYCARDG
GSSAITSNNIWGQGTLVTVSTGGGGSGGGGSDVQLQESGPSLVKPSQSLSLTCTVTGYSITSDFAWNWIRQFPGNKL
EWMGYISYSGNTRYNPSLKSRISITRDTSKNQFFLQLNSVTIEDTATYYCVTAGRGFPYWGQGTLVTVSAASTKGPS
VFPLAPSSKSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPEAAGGPSVFLFPPKPKDTLMISRTPEVTCVVVDVSHEDPEVKFNWY
VDGVEVHNAKTKPREEQYASTYRVVSVLTVLHQDWLNGKEYKCAVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVMHEAL
HNHYTQKSLSLSPGGGGGSGGGGSEVQLLESGGGLVQPGGSLRLSCAASGFSFSSGYDMCWVRQAPGKCLEWIACIA
AGSAGITYDANWAKGRFTISRDNSKNTLYLQMNSLRAEDTAVYYCARSAFSFDYAMDLWGQGTLVTVSSGGGGSGGG
GSGGGGSGGGGSDIQMTQSPSTLSASVGDRVTITCQASQSISSHLNWYQQKPGKAPKLLIYKASTLASGVPSRFSGS
GSGTEFTLTISSLQPDDFATYYCQQGYSWGNVDNVFGCGTKVEIKGGGGSGGGGSGRSLVESGGGLVQPGGSLRLSC
TASGFTISSYHMQWVRQAPGKCLEYIGTISSGGNVYYASSARGRFTISRPSSKNTVDLQMNSLRAEDTAVYYCARDS
GYSDPMWGQGTLVTVSSGGGGSGGGGSGGGGSGGGGSDVVMTQSPSSVSASVGDRVTITCQASQNIRTYLSWYQQKP
GKAPKLLIYAAANLASGVPSRFSGSGSGTDFTLTISDLEPGDAATYYCQSTYLGTDYVGGAFGCGTKVEIK
>SEQ ID 71 SI-49P4 chain B nt
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTGGCGGTGGAGGGTCCGGCGGTGGTGGATCCTTTCTTAATTCCCTTTTCAAC
CAAGAGGTTCAGATCCCCTTGACTGAAAGCTATTGCGGCCCTTGTCCGAAAAACTGGATATGTTACAAGAATAATTG
TTACCAATTCTTCGACGAAAGCAAGAACTGGTATGAGAGTCAGGCGTCTTGTATGAGTCAGAATGCCAGCCTGCTTA
AGGTTTATTCAAAAGAAGACCAGGATCTGCTTAAGTTGGTAAAGAGCTACCACTGGATGGGGCTGGTACATATCCCA
ACGAATGGGTCATGGCAGTGGGAGGACGGTTCTATTCTGAGTCCAAATCTCCTGACGATCATCGAAATGCAGAAAGG
GGACTGTGCCCTGTATGCATCATCCTTCAAGGGGTACATCGAGAACTGCAGTACCCCAAATACCTACATTTGTATGC
AAAGAACGGTTGGAGGCGGTGGCTCAGGCGGAGGCGGCTCAGGAGGTGGCGGTTCAGGAGGCGGCGGATCTTTCCTA
AACTCATTATTCAACCAAGAAGTTCAAATTCCCTTGACCGAAAGTTACTGTGGCCCATGTCCTAAAAACTGGATATG
TTACAAAAATAACTGCTACCAATTTTTTGATGAGAGTAAAAACTGGTATGAGAGCCAGGCTTCTTGTATGTCTCAAA
ATGCCAGCCTTCTGAAAGTATACAGCAAAGAGGACCAGGATTTACTTAAACTGGTGAAGTCATATCATTGGATGGGA
CTAGTACACATTCCAACAAATGGATCTTGGCAGTGGGAAGATGGCTCCATTCTCTCACCCAACCTACTAACAATAAT
TGAAATGCAGAAGGGAGACTGTGCACTCTATGCCTCGAGCTTTAAAGGCTATATAGAAAACTGTTCAACTCCAAATA
CGTACATCTGCATGCAAAGGACTGTGTAG
>SEQ ID 72 SI-49P4 chain B aa
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGGGGSGGGGSFLNSLFN
QEVQIPLTESYCGPCPKNWICYKNNCYQFFDESKNWYESQASCMSQNASLLKVYSKEDQDLLKLVKSYHWMGLVHIP
TNGSWQWEDGSILSPNLLTIIEMQKGDCALYASSFKGYIENCSTPNTYICMQRTVGGGGSGGGGSGGGGSGGGGSFL
NSLFNQEVQIPLTESYCGPCPKNWICYKNNCYQFFDESKNWYESQASCMSQNASLLKVYSKEDQDLLKLVKSYHWMG
LVHIPTNGSWQWEDGSILSPNLLTIIEMQKGDCALYASSFKGYIENCSTPNTYICMQRTV
>SEQ ID 73 SI-49P5 chain A nt
GAGATCGTGATGACCCAGAGCCCCAGCACCCTGAGCGCCAGCGTGGGCGACAGGGTGATCATCACCTGCCAGGCCAG
CGAGAGCATCAGCAGCTGGCTGGCCTGGTACCAGCAGAAGCCCGGCAAGGCCCCCAAGCTGCTGATCTACGAGGCCA
GCAAGCTGGCCAGCGGCGTGCCCAGCAGGTTCAGCGGCAGCGGCAGCGGCGCCGAGTTCACCCTGACCATCAGCAGC
CTGCAGCCCGACGACTTCGCCACCTACTACTGCCAGGGCTACTTCTACTTCATCAGCAGGACCTACGTGAACAGCTT
CGGCTGCGGCACCAAGCTGACCGTGCTGGGCGGTGGCGGTAGTGGGGGAGGCGGTTCTGGCGGCGGAGGGTCCGGCG
GTGGAGGATCAGAGGTGCAGCTGGTGGAGAGCGGCGGCGGCCTGGTGCAGCCCGGCGGCAGCCTGAGGCTGAGCTGC
ACCGCCAGCGGCTTCACCATCAGCACCAACGCCATGAGCTGGGTGAGGCAGGCCCCCGGCAAGTGCCTGGAGTGGGT
GGGCGTGATCACCGGCAGGGACATCACCTACTACGCCAGCTGGGCCAAGGGCAGGTTCACCATCAGCAGGGACACCA
GCAAGAACACCGTGTACCTGCAGATGAACAGCCTGAGGGCCGAGGACACCGCCGTGTACTACTGCGCCAGGGACGGC
GGCAGCAGCGCCATCACCAGCAACAACATCTGGGGCCAGGGCACCCTGGTGACCGTGTCGACAGGCGGTGGAGGGTC
CGGCGGTGGTGGATCAGAGGTCCAATTGGTACAATCCGGCGCTGAGGTTAAGAAACCGGGTTCCTCAGTCAAAGTTA
GTTGTAAGGCATCAGGTTACACGATCACTGATAGTAACATACATTGGGTGAGACAGGCTCCTGGGCAGTCCCTTGAG
TGGATTGGATACATTTATCCATATAATGGCGGCACGGATTACAACCAGAAATTTAAGAATAGAGCGACACTGACCGT
TGACAACCCCACCAATACCGCGTATATGGAATTGTCAAGCCTGAGGTCTGAGGACACGGCCTTCTACTATTGCGTAA
ACGGTAACCCGTGGCTGGCCTACTGGGGGCAGGGGACACTCGTAACCGTCTCTTCTGCTAGCACCAAGGGCCCATCG
GTCTTCCCCCTGGCACCCTCCTCCAAGAGCACCTCTGGGGGCACAGCGGCCCTGGGCTGCCTGGTCAAGGACTACTT
CCCCGAACCGGTGACGGTGTCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCACCCAGACCTACATCTGCAAC
GTGAATCACAAGCCCAGCAACACCAAGGTGGACAAGAGAGTTGAGCCCAAATCTTGTGACAAAACTCACACATGCCC
ACCGTGCCCAGCACCTGAAGCCGCGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGCAGTACGCCAGCACGTACCGTGTGGTCAG
CGTCCTCACCGTCCTGCACCAGGACTGGCTGAATGGCAAGGAGTACAAGTGCGCGGTCTCCAACAAAGCCCTCCCAG
CCCCCATCGAGAAAACCATCTCCAAAGCCAAAGGGCAGCCCCGAGAACCACAGGTGTACACCCTGCCCCCATCCCGG
GATGAGCTGACCAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCTCCTTCTTCCTCT
ATAGCAAGCTCACCGTGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCGGGTGGCGGTGGAGGGTCCGGCGGTGGTGGATCCGAGGT
GCAGCTGTTGGAGTCTGGGGGAGGCTTGGTACAGCCTGGGGGGTCCCTGAGACTCTCCTGTGCAGCCTCTGGATTCT
CCTTCAGTAGCGGGTACGACATGTGCTGGGTCCGCCAGGCTCCAGGGAAGTGCCTGGAGTGGATCGCATGCATTGCT
GCTGGTAGTGCTGGTATCACTTACGACGCGAACTGGGCGAAAGGCCGGTTCACCATCTCCAGAGACAATTCCAAGAA
CACGCTGTATCTGCAAATGAACAGCCTGAGAGCCGAGGACACGGCCGTATATTACTGTGCGAGATCGGCGTTTTCGT
TCGACTACGCCATGGACCTCTGGGGCCAGGGAACCCTGGTCACCGTCTCGAGCGGTGGAGGCGGTTCAGGCGGAGGT
GGAAGTGGTGGTGGCGGCTCTGGAGGCGGCGGATCTGACATCCAGATGACCCAGTCTCCTTCCACCCTGTCTGCATC
TGTAGGAGACAGAGTCACCATCACTTGCCAGGCCAGTCAGAGCATTAGTTCCCACTTAAACTGGTATCAGCAGAAAC
CAGGGAAAGCCCCTAAGCTCCTGATCTATAAGGCATCCACTCTGGCATCTGGGGTCCCATCAAGGTTCAGCGGCAGT
GGATCTGGGACAGAATTTACTCTCACCATCAGCAGCCTGCAGCCTGATGATTTTGCAACTTATTACTGCCAACAGGG
TTATAGTTGGGGTAATGTTGATAATGTTTTCGGCTGCGGGACCAAGGTGGAGATCAAAGGTGGTGGCGGCTCTGGAG
GAGGAGGGTCCGGACGGTCGCTGGTGGAGTCTGGGGGAGGCTTGGTCCAGCCTGGGGGGTCCCTGAGACTCTCCTGT
ACTGCCTCTGGATTCACCATCAGTAGCTACCACATGCAGTGGGTCCGGCAGGCACCTGGGAAGTGCCTGGAGTACAT
CGGAACCATTAGTAGTGGTGGTAATGTATACTACGCAAGCTCCGCTAGAGGCAGATTCACCATCTCCAGACCCTCGT
CCAAGAACACGGTGGATCTTCAAATGAACAGCCTGAGAGCCGAGGACACGGCTGTGTATTACTGTGCGAGAGACTCT
GGTTATAGTGATCCTATGTGGGGCCAGGGAACCCTGGTCACCGTCTCTTCAGGCGGTGGCGGTAGTGGGGGAGGCGG
TTCTGGCGGCGGAGGGTCCGGCGGTGGAGGATCAGACGTTGTGATGACCCAGTCTCCATCTTCCGTGTCTGCATCTG
TAGGAGACAGAGTCACCATCACCTGTCAGGCCAGTCAGAACATTAGGACTTACTTATCCTGGTATCAGCAGAAACCA
GGGAAAGCCCCTAAGCTCCTGATCTATGCTGCAGCCAATCTGGCATCTGGGGTCCCATCAAGGTTCAGCGGCAGTGG
ATCTGGGACAGATTTCACTCTCACCATCAGCGACTTGGAACCTGGCGATGCTGCAACTTACTATTGTCAGTCTACCT
ATCTTGGTACTGATTATGTTGGCGGTGCTTTCGGCTGTGGGACCAAGGTGGAGATCAAATGA
>SEQ ID 74 SI-49P5 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGGGGSGGGGSGGGGSGGGGSEVQLVESGGGLVQPGGSLRLSC
TASGFTISTNAMSWVRQAPGKCLEWVGVITGRDITYYASWAKGRFTISRDTSKNTVYLQMNSLRAEDTAVYYCARDG
GSSAITSNNIWGQGTLVTVSTGGGGSGGGGSEVQLVQSGAEVKKPGSSVKVSCKASGYTITDSNIHWVRQAPGQSLE
WIGYIYPYNGGTDYNQKFKNRATLTVDNPTNTAYMELSSLRSEDTAFYYCVNGNPWLAYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTYICN
VNHKPSNTKVDKRVEPKSCDKTHTCPPCPAPEAAGGPSVFLFPPKPKDTLMISRTPEVTCVVVDVSHEDPEVKFNWY
VDGVEVHNAKTKPREEQYASTYRVVSVLTVLHQDWLNGKEYKCAVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQGNVFSCSVMHEAL
HNHYTQKSLSLSPGGGGGSGGGGSEVQLLESGGGLVQPGGSLRLSCAASGFSFSSGYDMCWVRQAPGKCLEWIACIA
AGSAGITYDANWAKGRFTISRDNSKNTLYLQMNSLRAEDTAVYYCARSAFSFDYAMDLWGQGTLVTVSSGGGGSGGG
GSGGGGSGGGGSDIQMTQSPSTLSASVGDRVTITCQASQSISSHLNWYQQKPGKAPKLLIYKASTLASGVPSRFSGS
GSGTEFTLTISSLQPDDFATYYCQQGYSWGNVDNVFGCGTKVEIKGGGGSGGGGSGRSLVESGGGLVQPGGSLRLSC
TASGFTISSYHMQWVRQAPGKCLEYIGTISSGGNVYYASSARGRFTISRPSSKNTVDLQMNSLRAEDTAVYYCARDS
GYSDPMWGQGTLVTVSSGGGGSGGGGSGGGGSGGGGSDVVMTQSPSSVSASVGDRVTITCQASQNIRTYLSWYQQKP
GKAPKLLIYAAANLASGVPSRFSGSGSGTDFTLTISDLEPGDAATYYCQSTYLGTDYVGGAFGCGTKVEIK
>SEQ ID 75 SI-49P5 chain B nt
GATATACAACTTACGCAGTCTCCCTCCACTCTGTCAGCGTCCGTAGGGGACAGAGTAACGATAACATGCAGGGCCTC
AGAAAGTCTTGACAACTACGGCATAAGGTTCCTCACATGGTTTCAACAGAAGCCGGGTAAAGCACCAAAACTCCTGA
TGTATGCTGCGTCTAACCAGGGGTCCGGTGTTCCTAGTCGGTTTTCAGGCAGCGGTAGCGGTACCGAGTTCACTCTC
ACAATTTCCAGTCTTCAGCCTGACGATTTCGCAACGTATTATTGCCAACAAACTAAGGAAGTGCCTTGGAGCTTTGG
GCAAGGCACCAAGGTCGAGGTGAAGCGTACGGTGGCTGCACCATCTGTCTTCATCTTCCCGCCATCTGATGAGCAGT
TGAAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTCAG
CAGCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGTGGCGGTGGAGGGTCCGGCGGTGGTGGATCCTTTCTTAAT
TCCCTTTTCAACCAAGAGGTTCAGATCCCCTTGACTGAAAGCTATTGCGGCCCTTGTCCGAAAAACTGGATATGTTA
CAAGAATAATTGTTACCAATTCTTCGACGAAAGCAAGAACTGGTATGAGAGTCAGGCGTCTTGTATGAGTCAGAATG
CCAGCCTGCTTAAGGTTTATTCAAAAGAAGACCAGGATCTGCTTAAGTTGGTAAAGAGCTACCACTGGATGGGGCTG
GTACATATCCCAACGAATGGGTCATGGCAGTGGGAGGACGGTTCTATTCTGAGTCCAAATCTCCTGACGATCATCGA
AATGCAGAAAGGGGACTGTGCCCTGTATGCATCATCCTTCAAGGGGTACATCGAGAACTGCAGTACCCCAAATACCT
ACATTTGTATGCAAAGAACGGTTGGAGGCGGTGGCTCAGGCGGAGGCGGCTCAGGAGGTGGCGGTTCAGGAGGCGGC
GGATCTTTCCTAAACTCATTATTCAACCAAGAAGTTCAAATTCCCTTGACCGAAAGTTACTGTGGCCCATGTCCTAA
AAACTGGATATGTTACAAAAATAACTGCTACCAATTTTTTGATGAGAGTAAAAACTGGTATGAGAGCCAGGCTTCTT
GTATGTCTCAAAATGCCAGCCTTCTGAAAGTATACAGCAAAGAGGACCAGGATTTACTTAAACTGGTGAAGTCATAT
CATTGGATGGGACTAGTACACATTCCAACAAATGGATCTTGGCAGTGGGAAGATGGCTCCATTCTCTCACCCAACCT
ACTAACAATAATTGAAATGCAGAAGGGAGACTGTGCACTCTATGCCTCGAGCTTTAAAGGCTATATAGAAAACTGTT
CAACTCCAAATACGTACATCTGCATGCAAAGGACTGTGTAG
>SEQ ID 76 SI-49P5 chain B aa
DIQLTQSPSTLSASVGDRVTITCRASESLDNYGIRFLTWFQQKPGKAPKLLMYAASNQGSGVPSRFSGSGSGTEETL
TISSLQPDDFATYYCQQTKEVPWSFGQGTKVEVKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECGGGGSGGGGSFLN
SLFNQEVQIPLTESYCGPCPKNWICYKNNCYQFFDESKNWYESQASCMSQNASLLKVYSKEDQDLLKLVKSYHWMGL
VHIPTNGSWQWEDGSILSPNLLTIIEMQKGDCALYASSFKGYIENCSTPNTYICMQRTVGGGGSGGGGSGGGGSGGG
GSFLNSLFNQEVQIPLTESYCGPCPKNWICYKNNCYQFFDESKNWYESQASCMSQNASLLKVYSKEDQDLLKLVKSY
HWMGLVHIPTNGSWQWEDGSILSPNLLTIIEMQKGDCALYASSFKGYIENGSTPNTYICMQRTV
>SEQ ID 77 SI-49P6 chain A nt
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGTGGTGGGGGTAGTGGCGG
CGGAGGTAGCTTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCGAGGTGCAGCTGTTGGAGT
CTGGGGGAGGCTTGGTACAGCCTGGGGGGTCCCTGAGACTCTCCTGTGCAGCCTCTGGATTCTCCTTCAGTAGCGGG
TACGACATGTGCTGGGTCCGCCAGGCTCCAGGGAAGTGCCTGGAGTGGATCGCATGCATTGCTGCTGGTAGTGCTGG
TATCACTTACGACGCGAACTGGGCGAAAGGCCGGTTCACCATCTCCAGAGACAATTCCAAGAACACGCTGTATCTGC
AAATGAACAGCCTGAGAGCCGAGGACACGGCCGTATATTACTGTGCGAGATCGGCGTTTTCGTTCGACTACGCCATG
GACCTCTGGGGCCAGGGAACCCTGGTCACCGTCTCGAGCGGTGGAGGCGGTTCAGGCGGAGGTGGAAGTGGTGGTGG
CGGCTCTGGAGGCGGCGGATCTGACATCCAGATGACCCAGTCTCCTTCCACCCTGTCTGCATCTGTAGGAGACAGAG
TCACCATCACTTGCCAGGCCAGTCAGAGCATTAGTTCCCACTTAAACTGGTATCAGCAGAAACCAGGGAAAGCCCCT
AAGCTCCTGATCTATAAGGCATCCACTCTGGCATCTGGGGTCCCATCAAGGTTCAGCGGCAGTGGATCTGGGACAGA
ATTTACTCTCACCATCAGCAGCCTGCAGCCTGATGATTTTGCAACTTATTACTGCCAACAGGGTTATAGTTGGGGTA
ATGTTGATAATGTTTTCGGCTGCGGGACCAAGGTGGAGATCAAAGGTGGTGGCGGCTCTGGAGGAGGAGGGTCCGGA
CGGTCGCTGGTGGAGTCTGGGGGAGGCTTGGTCCAGCCTGGGGGGTCCCTGAGACTCTCCTGTACTGCCTCTGGATT
CACCATCAGTAGCTACCACATGCAGTGGGTCCGGCAGGCACCTGGGAAGTGCCTGGAGTACATCGGAACCATTAGTA
GTGGTGGTAATGTATACTACGCAAGCTCCGCTAGAGGCAGATTCACCATCTCCAGACCCTCGTCCAAGAACACGGTG
GATCTTCAAATGAACAGCCTGAGAGCCGAGGACACGGCTGTGTATTACTGTGCGAGAGACTCTGGTTATAGTGATCC
TATGTGGGGCCAGGGAACCCTGGTCACCGTCTCTTCAGGCGGTGGCGGTAGTGGGGGAGGCGGTTCTGGCGGCGGAG
GGTCCGGCGGTGGAGGATCAGACGTTGTGATGACCCAGTCTCCATCTTCCGTGTCTGCATCTGTAGGAGACAGAGTC
ACCATCACCTGTCAGGCCAGTCAGAACATTAGGACTTACTTATCCTGGTATCAGCAGAAACCAGGGAAAGCCCCTAA
GCTCCTGATCTATGCTGCAGCCAATCTGGCATCTGGGGTCCCATCAAGGTTCAGCGGCAGTGGATCTGGGACAGATT
TCACTCTCACCATCAGCGACTTGGAACCTGGCGATGCTGCAACTTACTATTGTCAGTCTACCTATCTTGGTACTGAT
TATGTTGGCGGTGCTTTCGGCTGTGGGACCAAGGTGGAGATCAAA
>SEQ ID 78 SI-49P6 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSEVQLLESGGGLVQPGGSLRLSCAASGFSFSSG
YDMCWVRQAPGKCLEWIACIAAGSAGITYDANWAKGRFTISRDNSKNTLYLQMNSLRAEDTAVYYCARSAFSFDYAM
DLWGQGTLVTVSSGGGGSGGGGSGGGGSGGGGSDIQMTQSPSTLSASVGDRVTITCQASQSISSHLNWYQQKPGKAP
KLLIYKASTLASGVPSRFSGSGSGTEFTLTISSLQPDDFATYYCQQGYSWGNVDNVFGCGTKVEIKGGGGSGGGGSG
RSLVESGGGLVQPGGSLRLSCTASGFTISSYHMQWVRQAPGKCLEYIGTISSGGNVYYASSARGRFTISRPSSKNTV
DLQMNSLRAEDTAVYYCARDSGYSDPMWGQGTLVTVSSGGGGSGGGGSGGGGSGGGGSDVVMTQSPSSVSASVGDRV
TITCQASQNIRTYLSWYQQKPGKAPKLLIYAAANLASGVPSRFSGSGSGTDFTLTISDLEPGDAATYYCQSTYLGTD
YVGGAFGCGTKVEIK
>SEQ ID 79 SI-49P6 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAATT
GGAGATAAAGGGAAGTACTGGTTCTGGGTCTAAACCCGGTTCCGGCGAAGGTAGTACTAAAGGACAGGTCACATTGA
AGGAATCTGGCCCCGGCCTTGTTCAGCCAGGACAGACCCTTAGGCTCACCTGTGCCTTCAGTGGTTTTTCTCTTAGC
ACTAGCGGTATGGGGGTCGGCTGGATTCGGCAGCCTCCCGGCAAAGGTCTTGAGTGGTTGGCTCACATTTGGTGGGA
CGACGACAAACGGTATAATCCTGCCTTGAAAAGTCGGCTGACCATTAGTAAGGATACCTCAAAAAATCAAGTGTACT
TGCAAATGAATAGCCTTGACGCCGAGGATACGGCTGTATATTATTGCGCGCGGATGGAACTCTGGTCTTACTACTTT
GATTATTGGGGGCAGGGGACTCTCGTCACGGTGTCCTCTGGTGGCGGCGGCTCCGGGGGTGGCGGATCAGGTGGTGG
AGGATCCTTCCTAAACTCATTATTCAACCAAGAAGTTCAAATTCCCTTGACCGAAAGTTACTGTGGCCCATGTCCTA
AAAACTGGATATGTTACAAAAATAACTGCTACCAATTTTTTGATGAGAGTAAAAACTGGTATGAGAGCCAGGCTTCT
TGTATGTCTCAAAATGCCAGCCTTCTGAAAGTATACAGCAAAGAGGACCAGGATTTACTTAAACTGGTGAAGTCATA
TCATTGGATGGGACTAGTACACATTCCAACAAATGGATCTTGGCAGTGGGAAGATGGCTCCATTCTCTCACCCAACC
TACTAACAATAATTGAAATGCAGAAGGGAGACTGTGCACTCTATGCCTCGAGCTTTAAAGGCTATATAGAAAACTGT
TCAACTCCAAATACGTACATCTGCATGCAAAGGACTGTGCGTACGGTGGCTGCACCATCTGTCTTCATCTTCCCGCC
ATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGTTAG
>SEQ ID 80 SI-49P6 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKLEIKGSTGSGSKPGSGEGSTKGQVTLKESGPGLVQPGQTLRLTCAFSGFSLS
TSGMGVGWIRQPPGKGLEWLAHIWWDDDKRYNPALKSRLTISKDTSKNQVYLQMNSLDAEDTAVYYCARMELWSYYF
DYWGQGTLVTVSSGGGGSGGGGSGGGGSFLNSLFNQEVQIPLTESYCGPCPKNWICYKNNCYQFFDESKNWYESQAS
CMSQNASLLKVYSKEDQDLLKLVKSYHWMGLVHIPTNGSWQWEDGSILSPNLLTIIEMQKGDCALYASSFKGYIENC
STPNTYICMQRTVRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
>SEQ ID 81 SI-49P7 chain A nt
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGTGGTGGGGGTAGTGGCGG
CGGAGGTAGCTTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCGAGGTGCAGCTGTTGGAGT
CTGGGGGAGGCTTGGTACAGCCTGGGGGGTCCCTGAGACTCTCCTGTGCAGCCTCTGGATTCTCCTTCAGTAGCGGG
TACGACATGTGCTGGGTCCGCCAGGCTCCAGGGAAGTGCCTGGAGTGGATCGCATGCATTGCTGCTGGTAGTGCTGG
TATCACTTACGACGCGAACTGGGCGAAAGGCCGGTTCACCATCTCCAGAGACAATTCCAAGAACACGCTGTATCTGC
AAATGAACAGCCTGAGAGCCGAGGACACGGCCGTATATTACTGTGCGAGATCGGCGTTTTCGTTCGACTACGCCATG
GACCTCTGGGGCCAGGGAACCCTGGTCACCGTCTCGAGCGGTGGAGGCGGTTCAGGCGGAGGTGGAAGTGGTGGTGG
CGGCTCTGGAGGCGGCGGATCTGACATCCAGATGACCCAGTCTCCTTCCACCCTGTCTGCATCTGTAGGAGACAGAG
TCACCATCACTTGCCAGGCCAGTCAGAGCATTAGTTCCCACTTAAACTGGTATCAGCAGAAACCAGGGAAAGCCCCT
AAGCTCCTGATCTATAAGGCATCCACTCTGGCATCTGGGGTCCCATCAAGGTTCAGCGGCAGTGGATCTGGGACAGA
ATTTACTCTCACCATCAGCAGCCTGCAGCCTGATGATTTTGCAACTTATTACTGCCAACAGGGTTATAGTTGGGGTA
ATGTTGATAATGTTTTCGGCTGCGGGACCAAGGTGGAGATCAAAGGTGGTGGCGGCTCTGGAGGAGGAGGGTCCGGA
CGAGAGGGCCCCGAGCTGTCTCCTGATGACCCAGCAGGCCTCTTGGACTTGCGGCAGGGTATGTTCGCTCAACTTGT
GGCTCAGAATGTTCTGCTCATTGATGGACCACTCTCTTGGTATAGTGACCCCGGTCTGGCCGGGGTGAGTCTGACCG
GCGGGCTCTCTTATAAAGAGGATACTAAGGAACTGGTCGTAGCAAAAGCGGGCGTTTATTACGTTTTTTTTCAGCTG
GAGCTCAGGCGCGTGGTGGCCGGCGAGGGCAGTGGCTCTGTGTCCCTGGCCCTGCACTTACAGCCCTTGAGAAGCGC
TGCAGGTGCTGCCGCACTGGCTTTAACTGTTGACCTCCCTCCGGCCTCTTCTGAAGCTAGAAACAGCGCTTTCGGCT
TCCAAGGGCGCCTGCTGCACCTGAGCGCAGGCCAGCGCTTAGGTGTGCACCTTCATACAGAGGCCAGGGCCCGACAC
GCTTGGCAGCTCACACAGGGTGCCACGGTTCTCGGACTTTTCCGCGTTACTCCCGAGATCCCCGCTGGCCTCGGAAG
TACTGGTTCTGGGTCTAAACCCGGTTCCGGCGAAGGTAGTACTAAAGGACGAGAAGGGCCAGAGTTAAGTCCAGATG
ACCCTGCTGGGCTTTTGGACCTGCGGCAGGGCATGTTCGCTCAACTGGTGGCTCAGAACGTGCTGCTGATCGATGGC
CCCCTGAGTTGGTACAGCGATCCCGGGCTGGCAGGCGTGTCACTTACAGGGGGCCTCTCTTACAAGGAAGACACCAA
GGAGTTAGTGGTCGCTAAGGCTGGCGTGTATTACGTGTTCTTCCAACTGGAGCTGAGAAGGGTTGTGGCAGGAGAGG
GTAGCGGCAGCGTGTCTTTAGCCCTTCACTTGCAGCCCCTGAGGTCTGCTGCAGGTGCAGCCGCTCTCGCGCTCACC
GTGGATCTCCCCCCAGCCTCATCTGAAGCTAGGAACAGTGCATTTGGCTTTCAGGGACGCTTGCTGCACCTCTCCGC
TGGACAGAGGCTGGGCGTGCACCTTCACACAGAGGCCCGTGCCAGGCATGCATGGCAGCTCACTCAGGGGGCAACAG
TGCTGGGTCTCTTCCGCGTGACTCCTGAAATACCAGCTGGACTTGGCGGTGGAGGCAGCGGCGGAGGAGGATCTCGT
GAGGGGCCAGAACTGTCCCCCGATGACCCAGCCGGACTGCTCGATCTCAGACAGGGCATGTTCGCTCAGCTTGTAGC
CCAAAATGTCCTCCTGATTGACGGCCCTTTGAGCTGGTATAGTGATCCCGGCTTGGCCGGGGTATCTCTGACCGGAG
GCCTCTCCTACAAGGAAGACACCAAAGAGCTGGTGGTGGCAAAAGCGGGGGTGTATTATGTGTTCTTTCAGCTCGAG
CTGCGGAGAGTTGTGGCCGGGGAAGGGTCTGGGAGCGTATCTCTTGCACTTCACCTGCAGCCCCTGCGCAGCGCCGC
TGGAGCCGCCGCCCTTGCTCTTACTGTGGATCTGCCTCCTGCTTCCTCAGAAGCACGCAACAGCGCCTTCGGCTTTC
AAGGACGTCTCCTGCACTTGTCCGCAGGACAGAGGTTGGGCGTCCATTTACACACTGAGGCACGGGCACGGCACGCT
TGGCAGCTTACCCAGGGAGCCACCGTGCTGGGACTCTTTAGAGTGACACCCGAGATCCCCGCTGGCTTG
>SEQ ID 82 SI-49P7 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSEVQLLESGGGLVQPGGSLRLSCAASGFSFSSG
YDMCWVRQAPGKCLEWIACIAAGSAGITYDANWAKGRFTISRDNSKNTLYLQMNSLRAEDTAVYYCARSAFSFDYAM
DLWGQGTLVTVSSGGGGSGGGGSGGGGSGGGGSDIQMTQSPSTLSASVGDRVTITCQASQSISSHLNWYQQKPGKAP
KLLIYKASTLASGVPSRFSGSGSGTEFTLTISSLQPDDFATYYCQQGYSWGNVDNVFGCGTKVEIKGGGGSGGGGSG
REGPELSPDDPAGLLDLRQGMFAQLVAQNVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSAFGFQGRLLHLSAGQRLGVHLHTEARARH
AWQLTQGATVLGLFRVTPEIPAGLGSTGSGSKPGSGEGSTKGREGPELSPDDPAGLLDLRQGMFAQLVAQNVLLIDG
PLSWYSDPGLAGVSLTGGLSYKEDTKELVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGATVLGLFRVTPEIPAGLGGGGSGGGGSR
EGPELSPDDPAGLLDLRQGMFAQLVAQNVLLIDGPLSWYSDPGLAGVSLTGGLSYKEDTKELVVAKAGVYYVFFQLE
LRRVVAGEGSGSVSLALHLQPLRSAAGAAALALTVDLPPASSEARNSAFGFQGRLLHLSAGQRLGVHLHTEARARHA
WQLTQGATVLGLFRVTPEIPAGL
>SEQ ID 83 SI-49P7 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAATT
GGAGATAAAGGGAAGTACTGGTTCTGGGTCTAAACCCGGTTCCGGCGAAGGTAGTACTAAAGGACAGGTCACATTGA
AGGAATCTGGCCCCGGCCTTGTTCAGCCAGGACAGACCCTTAGGCTCACCTGTGCCTTCAGTGGTTTTTCTCTTAGC
ACTAGCGGTATGGGGGTCGGCTGGATTCGGCAGCCTCCCGGCAAAGGTCTTGAGTGGTTGGCTCACATTTGGTGGGA
CGACGACAAACGGTATAATCCTGCCTTGAAAAGTCGGCTGACCATTAGTAAGGATACCTCAAAAAATCAAGTGTACT
TGCAAATGAATAGCCTTGACGCCGAGGATACGGCTGTATATTATTGCGCGCGGATGGAACTCTGGTCTTACTACTTT
GATTATTGGGGGCAGGGGACTCTCGTCACGGTGTCCTCTGGTGGCGGCGGCTCCGGGGGTGGCGGATCAGGTGGTGG
AGGATCCTTCCTAAACTCATTATTCAACCAAGAAGTTCAAATTCCCTTGACCGAAAGTTACTGTGGCCCATGTCCTA
AAAACTGGATATGTTACAAAAATAACTGCTACCAATTTTTTGATGAGAGTAAAAACTGGTATGAGAGCCAGGCTTCT
TGTATGTCTCAAAATGCCAGCCTTCTGAAAGTATACAGCAAAGAGGACCAGGATTTACTTAAACTGGTGAAGTCATA
TCATTGGATGGGACTAGTACACATTCCAACAAATGGATCTTGGCAGTGGGAAGATGGCTCCATTCTCTCACCCAACC
TACTAACAATAATTGAAATGCAGAAGGGAGACTGTGCACTCTATGCCTCGAGCTTTAAAGGCTATATAGAAAACTGT
TCAACTCCAAATACGTACATCTGCATGCAAAGGACTGTGCGTACGGTGGCTGCACCATCTGTCTTCATCTTCCCGCC
ATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
>SEQ ID 84 SI-49P7 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKLEIKGSTGSGSKPGSGEGSTKGQVTLKESGPGLVQPGQTLRLTCAFSGFSLS
TSGMGVGWIRQPPGKGLEWLAHIWWDDDKRYNPALKSRLTISKDTSKNQVYLQMNSLDAEDTAVYYCARMELWSYYF
DYWGQGTLVTVSSGGGGSGGGGSGGGGSFLNSLFNQEVQIPLTESYCGPCPKNWICYKNNCYQFFDESKNWYESQAS
CMSQNASLLKVYSKEDQDLLKLVKSYHWMGLVHIPTNGSWQWEDGSILSPNLLTIIEMQKGDCALYASSFKGYIENC
STPNTYICMQRTVRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
>SEQ ID 85 SI-49P10 chain A nt
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGTGGTGGGGGTAGTGGCGG
CGGAGGTAGCTTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCGAGGTGCAGCTGTTGGAGT
CTGGGGGAGGCTTGGTACAGCCTGGGGGGTCCCTGAGACTCTCCTGTGCAGCCTCTGGATTCTCCTTCAGTAGCGGG
TACGACATGTGCTGGGTCCGCCAGGCTCCAGGGAAGTGCCTGGAGTGGATCGCATGCATTGCTGCTGGTAGTGCTGG
TATCACTTACGACGCGAACTGGGCGAAAGGCCGGTTCACCATCTCCAGAGACAATTCCAAGAACACGCTGTATCTGC
AAATGAACAGCCTGAGAGCCGAGGACACGGCCGTATATTACTGTGCGAGATCGGCGTTTTCGTTCGACTACGCCATG
GACCTCTGGGGCCAGGGAACCCTGGTCACCGTCTCGAGCGGTGGAGGCGGTTCAGGCGGAGGTGGAAGTGGTGGTGG
CGGCTCTGGAGGCGGCGGATCTGACATCCAGATGACCCAGTCTCCTTCCACCCTGTCTGCATCTGTAGGAGACAGAG
TCACCATCACTTGCCAGGCCAGTCAGAGCATTAGTTCCCACTTAAACTGGTATCAGCAGAAACCAGGGAAAGCCCCT
AAGCTCCTGATCTATAAGGCATCCACTCTGGCATCTGGGGTCCCATCAAGGTTCAGCGGCAGTGGATCTGGGACAGA
ATTTACTCTCACCATCAGCAGCCTGCAGCCTGATGATTTTGCAACTTATTACTGCCAACAGGGTTATAGTTGGGGTA
ATGTTGATAATGTTTTCGGCTGCGGGACCAAGGTGGAGATCAAAGGTGGTGGCGGCTCTGGAGGAGGAGGGTCCGGA
CGGTCGCTGGTGGAGTCTGGGGGAGGCTTGGTCCAGCCTGGGGGGTCCCTGAGACTCTCCTGTACTGCCTCTGGATT
CACCATCAGTAGCTACCACATGCAGTGGGTCCGGCAGGCACCTGGGAAGTGCCTGGAGTACATCGGAACCATTAGTA
GTGGTGGTAATGTATACTACGCAAGCTCCGCTAGAGGCAGATTCACCATCTCCAGACCCTCGTCCAAGAACACGGTG
GATCTTCAAATGAACAGCCTGAGAGCCGAGGACACGGCTGTGTATTACTGTGCGAGAGACTCTGGTTATAGTGATCC
TATGTGGGGCCAGGGAACCCTGGTCACCGTCTCTTCAGGCGGTGGCGGTAGTGGGGGAGGCGGTTCTGGCGGCGGAG
GGTCCGGCGGTGGAGGATCAGACGTTGTGATGACCCAGTCTCCATCTTCCGTGTCTGCATCTGTAGGAGACAGAGTC
ACCATCACCTGTCAGGCCAGTCAGAACATTAGGACTTACTTATCCTGGTATCAGCAGAAACCAGGGAAAGCCCCTAA
GCTCCTGATCTATGCTGCAGCCAATCTGGCATCTGGGGTCCCATCAAGGTTCAGCGGCAGTGGATCTGGGACAGATT
TCACTCTCACCATCAGCGACTTGGAACCTGGCGATGCTGCAACTTACTATTGTCAGTCTACCTATCTTGGTACTGAT
TATGTTGGCGGTGCTTTCGGCTGTGGGACCAAGGTGGAGATCAAA
>SEQ ID 86 SI-49P10 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSEVQLLESGGGLVQPGGSLRLSCAASGFSFSSG
YDMCWVRQAPGKCLEWIACIAAGSAGITYDANWAKGRFTISRDNSKNTLYLQMNSLRAEDTAVYYCARSAFSFDYAM
DLWGQGTLVTVSSGGGGSGGGGSGGGGSGGGGSDIQMTQSPSTLSASVGDRVTITCQASQSISSHLNWYQQKPGKAP
KLLIYKASTLASGVPSRFSGSGSGTEFTLTISSLQPDDFATYYCQQGYSWGNVDNVFGCGTKVEIKGGGGSGGGGSG
RSLVESGGGLVQPGGSLRLSCTASGFTISSYHMQWVRQAPGKCLEYIGTISSGGNVYYASSARGRFTISRPSSKNTV
DLQMNSLRAEDTAVYYCARDSGYSDPMWGQGTLVTVSSGGGGSGGGGSGGGGSGGGGSDVVMTQSPSSVSASVGDRV
TITCQASQNIRTYLSWYQQKPGKAPKLLIYAAANLASGVPSRFSGSGSGTDFTLTISDLEPGDAATYYCQSTYLGTD
YVGGAFGCGTKVEIK
>SEQ ID 87 SI-49P10 chain B nt
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCGGAGGGACCAA
GGTGGAAATTAAAGGAAGTACTGGTTCTGGGTCTAAACCCGGTTCCGGCGAAGGTAGTACTAAAGGACAGGTGCAGC
TGCAGGAGTCGGGCCCAGGACTGGTGAAGCCTTCGGAGACCCTGTCCCTCACCTGCACTGTCTCTGGTGGCTCCGTC
AGCAGTGGTGATTACTACTGGACCTGGATCCGGCAGTCCCCAGGGAAGGGACTGGAGTGGATTGGACACATCTATTA
CAGTGGGAACACCAATTATAACCCCTCCCTCAAGAGCCGACTCACCATATCAATTGACACGTCCAAGACTCAGTTCT
CCCTGAAGCTGAGTTCTGTGACCGCTGCGGACACGGCCATTTATTACTGTGTGCGAGATCGAGTGACTGGTGCTTTT
GATATCTGGGGCCAAGGGACAATGGTCACCGTCTCGAGCGGTGGCGGCGGCTCCGGGGGTGGCGGATCAGGTGGTGG
AGGCTCTTTCCTAAACTCATTATTCAACCAAGAAGTTCAAATTCCCTTGACCGAAAGTTACTGTGGCCCATGTCCTA
AAAACTGGATATGTTACAAAAATAACTGCTACCAATTTTTTGATGAGAGTAAAAACTGGTATGAGAGCCAGGCTTCT
TGTATGTCTCAAAATGCCAGCCTTCTGAAAGTATACAGCAAAGAGGACCAGGATTTACTTAAACTGGTGAAGTCATA
TCATTGGATGGGACTAGTACACATTCCAACAAATGGATCTTGGCAGTGGGAAGATGGCTCCATTCTCTCACCCAACC
TACTAACAATAATTGAAATGCAGAAGGGAGACTGTGCACTCTATGCCTCGAGCTTTAAAGGCTATATAGAAAACTGT
TCAACTCCAAATACGTACATCTGCATGCAAAGGACTGTGCGTACGGTGGCTGCACCATCTGTCTTCATCTTCCCGCC
ATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
>SEQ ID 88 SI-49P10 chain B aa
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIKGSTGSGSKPGSGEGSTKGQVQLQESGPGLVKPSETLSLTCTVSGGSV
SSGDYYWTWIRQSPGKGLEWIGHIYYSGNTNYNPSLKSRLTISIDTSKTQFSLKLSSVTAADTAIYYCVRDRVTGAF
DIWGQGTMVTVSSGGGGSGGGGSGGGGSFLNSLFNQEVQIPLTESYCGPCPKNWICYKNNCYQFFDESKNWYESQAS
CMSQNASLLKVYSKEDQDLLKLVKSYHWMGLVHIPTNGSWQWEDGSILSPNLLTIIEMQKGDCALYASSFKGYIENG
STPNTYICMQRTVRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
>SEQ ID 97 SI-38E17 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAGTTCACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGCGCGACGGT
GGATCATCTGCTATTACTAGTAACAACATTTGGGGCCAAGGAACTCTGGTCACCGTTTCTTCAGGCGGTGGAGGGTC
CGGCGGTGGTGGATCCGAGGTGCAGCTGGTGCAGTCTGGAGCAGAGGTGAAGAAACCAGGAGAGTCTCTGAAGATCT
CCTGTAAGGGTTCTGGATACAGCTTTAGCAGTTCATGGATCGGCTGGGTGCGCCAGGCACCTGGGAAAGGCCTGGAA
TGGATGGGGATCATCTATCCTGATGACTCTGATACCAGATACAGTCCATCCTTCCAAGGCCAGGTCACCATCTCAGC
CGACAAGTCCATCAGGACTGCCTACCTGCAGTGGAGTAGCCTGAAGGCCTCGGACACCGCTATGTATTACTGTGCGA
GACATGTTACTATGATTTGGGGAGTTATTATTGACTTCTGGGGCCAGGGAACCCTGGTCACCGTCTCCTCAGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGCACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTATACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTG
CAGCCTCTGGATTCTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGTTCACCATCTCCAG
AGACAATTCCAAGAACACGCTGTATCTGCAAATGAACAGCCTGAGAGCCGAGGACACGGCCGTATATTACTGTGCGA
GATCGGCGTTTTCGTTCGACTACGCCATGGACCTCTGGGGCCAGGGAACCCTGGTCACCGTCTCGAGCGGTGGAGGC
GGATCTGGCGGAGGTGGTTCCGGCGGTGGCGGCTCCGGTGGAGGCGGCTCTGACATCCAGATGACCCAGTCTCCTTC
CACCCTGTCTGCATCTGTAGGAGACAGAGTCACCATCACTTGCCAGGCCAGTCAGAGCATTAGTTCCCACTTAAACT
GGTATCAGCAGAAACCAGGGAAAGCCCCTAAGCTCCTGATCTATAAGGCATCCACTCTGGCATCTGGGGTCCCATCA
AGGTTCAGCGGCAGTGGATCTGGGACAGAATTTACTCTCACCATCAGCAGCCTGCAGCCTGATGATTTTGCAACTTA
TTACTGCCAACAGGGTTATAGTTGGGGTAATGTTGATAATGTTTTCGGCGGAGGGACCAAGGTGGAGATCAAAGGCG
GTGGAGGGTCCGGCGGTGGTGGATCCCGGTCGCTGGTGGAGTCTGGGGGAGGCTTGGTCCAGCCTGGGGGGTCCCTG
AGACTCTCCTGTACAGCCTCTGGATTCACCATCAGTAGCTACCACATGCAGTGGGTCCGCCAGGCTCCAGGGAAGGG
GCTGGAGTACATCGGAACCATTAGTAGTGGTGGTAATGTATACTACGCGAGCTCCGCGAGAGGCAGATTCACCATCT
CCAGACCCTCGTCCAAGAACACGGTGGATCTTCAAATGAACAGCCTGAGAGCCGAGGACACGGCTGTGTATTACTGT
GCGAGAGACTCTGGTTATAGTGATCCTATGTGGGGCCAGGGAACCCTGGTCACCGTCTCGAGCGGCGGTGGCGGTAG
TGGGGGAGGCGGTTCTGGCGGCGGAGGGTCCGGCGGTGGAGGATCAGACGTTGTGATGACCCAGTCTCCATCTTCCG
TGTCTGCATCTGTAGGAGACAGAGTCACCATCACCTGTCAGGCCAGTCAGAACATTAGGACTTACTTATCCTGGTAT
CAGCAGAAACCAGGGAAAGCCCCTAAGCTCCTGATCTATGCTGCAGCCAATCTGGCATCTGGGGTCCCATCAAGGTT
CAGCGGCAGTGGATCTGGGACAGATTTCACTCTCACCATCAGCGACCTGGAGCCTGGCGATGCTGCAACTTACTATT
GTCAGTCTACCTATCTTGGTACTGATTATGTTGGCGGTGCTTTCGGCGGAGGGACCAAGGTGGAGATCAAATAA
>SEQ ID 98 SI-38E17 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSSGGGGSGGGGSEVQLVQSGAEVKKPGESLKISCKGSGYSFSSSWIGWVRQAPGKGLE
WMGIIYPDDSDTRYSPSFQGQVTISADKSIRTAYLQWSSLKASDTAMYYCARHVTMIWGVIIDFWGQGTLVTVSSAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVTCVVVDVSHEDPEV
KFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSFDYAMDLWGQGTLVTVSSGGG
GSGGGGSGGGGSGGGGSDIQMTQSPSTLSASVGDRVTITCQASQSISSHLNWYQQKPGKAPKLLIYKASTLASGVPS
RFSGSGSGTEFTLTISSLQPDDFATYYCQQGYSWGNVDNVFGGGTKVEIKGGGGSGGGGSRSLVESGGGLVQPGGSL
RLSCTASGFTISSYHMQWVRQAPGKGLEYIGTISSGGNVYYASSARGRFTISRPSSKNTVDLQMNSLRAEDTAVYYC
ARDSGYSDPMWGQGTLVTVSSGGGGSGGGGSGGGGSGGGGSDVVMTQSPSSVSASVGDRVTITCQASQNIRTYLSWY
QQKPGKAPKLLIYAAANLASGVPSRFSGSGSGTDFTLTISDLEPGDAATYYCQSTYLGTDYVGGAFGGGTKVEIK
>SEQ ID 99 SI-38E17 chain B nt
GCCATCCAGTTGACCCAGTCTCCATCCTCCCTGTCTGCATCTGTAGGAGACAGAGTCACCATCACTTGCCGGGCAAG
TCAGGGCATTAGCAGTGCTTTAGCCTGGTATCAGCAGAAACCAGGGAAAGCTCCTAAGCTCCTGATCTATGATGCCT
CCAGTTTGGAAAGTGGGGTCCCATCAAGGTTCAGCGGCAGTGGATCTGGGACAGATTTCACTCTCACCATCAGCAGC
CTGCAGCCTGAAGATTTTGCAACTTATTACTGTCAACAGTTTAATAGTTACCCATTCACTTTCGGCCCTGGGACCAA
AGTGGATATCAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>SEQ ID 100 SI-38E17 chain B aa
AIQLTQSPSSLSASVGDRVTITCRASQGISSALAWYQQKPGKAPKLLIYDASSLESGVPSRFSGSGSGTDFTLTISS
LQPEDFATYYCQQFNSYPFTFGPGTKVDIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>SEQ ID 105 SI-55H11 chain A nt
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGTGGAGGCGGATCTGGCGGAGGTGGTTCCGG
CGGTGGCGGCTCCGGTGGAGGCGGCTCTGAGGTGCAGCTGGTGGAGTCTGGGGGAGGCTTGGTCCAGCCTGGGGGGT
CCCTGAGACTCTCCTGTGCAGCCTCTGGATTCACCATCAGTACCAATGCAATGAGCTGGGTCCGCCAGGCTCCAGGG
AAGTGTCTGGAGTGGATCGGAGTCATTACTGGTCGTGATATCACATACTACGCGAGCTGGGCGAAAGGCAGATTCAC
CATCTCCAGAGACAATTCCAAGAACACGCTGTATCTTCAAATGAACAGCCTGAGAGCCGAGGACACGGCTGTGTATT
ACTGTGCGAGAGACGGTGGTTCTTCTGCTATTACTAGTAACAACATTTGGGGCCAGGGAACCCTGGTCACCGTGTCC
TCAGCTAGCACCAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAGTTGAGCCCAAATC
TTGTGACAAAACTCACACATGCCCACCGTGCCCAGCACCTGAAGCCGCGGGGGCACCGTCAGTCTTCCTCTTCCCCC
CAAAACCCAAGGACACCCTCATGATCTCCCGGACCCCTGAGGTCACATGCGTGGTGGTGGACGTGAGCCACGAAGAC
CCTGAGGTCAAGTTCAACTGGTACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAG
GTGTACACCCTGCCCCCATCCCGGGATGAGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCGGTGGAGG
GTCCGGCGGTGGTGGATCCGAGGTGCAGCTGTTGGAGTCTGGGGGAGGCTTGGTACAGCCTGGGGGGTCCCTGAGAC
TCTCCTGTGCAGCCTCTGGATTCTCCTTCAGTAGCGGGTACGACATGTGCTGGGTCCGCCAGGCTCCAGGGAAGGGG
CTGGAGTGGATCGCATGCATTGCTGCTGGTAGTGCTGGTATCACTTACGACGCGAACTGGGCGAAAGGCCGGTTCAC
CATCTCCAGAGACAATTCCAAGAACACGCTGTATCTGCAAATGAACAGCCTGAGAGCCGAGGACACGGCCGTATATT
ACTGTGCGAGATCGGCGTTTTCGTTCGACTACGCCATGGACCTCTGGGGCCAGGGAACCCTGGTCACCGTGTCGAGC
GGTGGAGGCGGATCTGGCGGAGGTGGTTCCGGCGGTGGCGGCTCCGGTGGAGGCGGCTCTGACATCCAGATGACCCA
GTCTCCTTCCACCCTGTCTGCATCTGTAGGAGACAGAGTCACCATCACTTGCCAGGCCAGTCAGAGCATTAGTTCCC
ACTTAAACTGGTATCAGCAGAAACCAGGGAAAGCCCCTAAGCTCCTGATCTATAAGGCATCCACTCTGGCATCTGGG
GTCCCATCAAGGTTCAGCGGCAGTGGATCTGGGACAGAATTTACTCTCACCATCAGCAGCCTGCAGCCTGATGATTT
TGCAACTTATTACTGCCAACAGGGTTATAGTTGGGGTAATGTTGATAATGTTTTCGGCGGAGGGACCAAGGTGGAGA
TCAAAGGCGGTGGAGGGTCCGGCGGTGGTGGCTCCGGACGGTCGCTGGTGGAGTCTGGGGGAGGCTTGGTCCAGCCT
GGGGGGTCCCTGAGACTCTCCTGTACTGCCTCTGGATTCACCATCAGTAGCTACCACATGCAGTGGGTCCGCCAGGC
TCCAGGGAAGGGGCTGGAGTACATCGGAACCATTAGTAGTGGTGGTAATGTATACTACGCAAGCTCCGCTAGAGGCA
GATTCACCATCTCCAGACCCTCGTCCAAGAACACGGTGGATCTTCAAATGAACAGCCTGAGAGCCGAGGACACGGCT
GTGTATTACTGTGCGAGAGACTCTGGTTATAGTGATCCTATGTGGGGCCAGGGAACCCTGGTCACCGTCTCTTCAGG
CGGTGGCGGTAGTGGGGGAGGCGGTTCTGGCGGCGGAGGGTCCGGCGGTGGAGGATCAGACGTTGTGATGACCCAGT
CTCCATCTTCCGTGTCTGCATCTGTAGGAGACAGAGTCACCATCACCTGTCAGGCCAGTCAGAACATTAGGACTTAC
TTATCCTGGTATCAGCAGAAACCAGGGAAAGCCCCTAAGCTCCTGATCTATGCTGCAGCCAATCTGGCATCTGGGGT
CCCATCAAGGTTCAGCGGCAGTGGATCTGGGACAGATTTCACTCTCACCATCAGCGACCTGGAGCCTGGCGATGCTG
CAACTTACTATTGTCAGTCTACCTATCTTGGTACTGATTATGTTGGCGGTGCTTTCGGCGGAGGGACCAAGGTGGAG
ATCAAATGA
>SEQ ID 106 SI-55H11 chain A aa
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSGGGGSGGGGSEVQLVESGGGLVQPGGSLRLSCAASGFTISTNAMSWVRQAPG
KCLEWIGVITGRDITYYASWAKGRFTISRDNSKNTLYLQMNSLRAEDTAVYYCARDGGSSAITSNNIWGQGTLVTVS
SASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQQGNVFS
CSVMHEALHNHYTQKSLSLSPGGGGGSGGGGSEVQLLESGGGLVQPGGSLRLSCAASGFSFSSGYDMCWVRQAPGKG
LEWIACIAAGSAGITYDANWAKGRFTISRDNSKNTLYLQMNSLRAEDTAVYYCARSAFSFDYAMDLWGQGTLVTVSS
GGGGSGGGGSGGGGSGGGGSDIQMTQSPSTLSASVGDRVTITCQASQSISSHLNWYQQKPGKAPKLLIYKASTLASG
VPSRFSGSGSGTEFTLTISSLQPDDFATYYCQQGYSWGNVDNVFGGGTKVEIKGGGGSGGGGSGRSLVESGGGLVQP
GGSLRLSCTASGFTISSYHMQWVRQAPGKGLEYIGTISSGGNVYYASSARGRFTISRPSSKNTVDLQMNSLRAEDTA
VYYCARDSGYSDPMWGQGTLVTVSSGGGGSGGGGSGGGGSGGGGSDVVMTQSPSSVSASVGDRVTITCQASQNIRTY
LSWYQQKPGKAPKLLIYAAANLASGVPSRFSGSGSGTDFTLTISDLEPGDAATYYCQSTYLGTDYVGGAFGGGTKVE
IK
>SEQ ID 107 SI-55H11 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCTCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTTACTCTCACCATCAGCAGCCT
GCAGCCTGATGATTTTGCAACTTATTACTGCCAAGGCTATTTTTATTTTATTAGTCGTACTTATGTAAATTCTTTCG
GCTGTGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGCGGTGGCGGTAGCGGTGGCGGCGGAAGTGGTGGCGG
AGGATCCCAGTCTGCCCTGACTCAGCCTGCCTCCGTGTCTGGGTCTCCTGGACAGTCGATCACCATCTCCTGCACTG
GAACCAGCAGTGACGTTGGTGGTTATAACTTTGTCTCCTGGTACCAACAACACCCAGGCAAAGCCCCCAAACTCATG
ATCTATGATGTCAGTGATCGGCCCTCAGGGGTGTCTGATCGCTTCTCCGGCTCCAAGTCTGGCAACACGGCCTCCCT
GATCATCTCTGGCCTCCAGGCTGACGACGAGGCTGATTATTACTGCAGCTCATATGGGAGCAGCAGCACTCATGTGA
TTTTCGGCGGAGGGACCAAGGTGACCGTCCTAGGTGGAGGCGGTTCAGGCGGAGGTGGTTCCGGCGGTGGCGGCTCC
GGTGGAGGCGGCTCTCAGGTGCAATTGCAGGAGTCGGGGGGAGGCCTGGTCAAGCCTGGAGGGTCCCTGAGTCTCTC
CTGTGCAGCCTCTGGATTCACCTTTAGTAGTTATTGGATGAGCTGGGTCCGCCAGGCTCCAGGGAAGGGGCTGGAGT
GGGTGGCCAACATAAACCGCGATGGAAGTGCGAGTTACTATGTGGACTCTGTGAAGGGCCGATTCACCATCTCCAGA
GACGACGCCAAGAACTCACTGTATCTGCAAATGAACAGCCTGAGAGCTGAGGACACGGCTGTGTATTACTGTGCGAG
AGATCGTGGGGTGGGCTACTTCGATCTCTGGGGCCGTGGCACCCTGGTCACCGTCTCTAGCTGA
>SEQ ID 108 SI-55H11 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGCGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGECGGGGSGGGGSGGGGSQSALTQPASVSGSPGQSITISCTGTSSDVGGYNFVSWYQQHPGKAPKLM
IYDVSDRPSGVSDRFSGSKSGNTASLIISGLQADDEADYYCSSYGSSSTHVIFGGGTKVTVLGGGGSGGGGSGGGGS
GGGGSQVQLQESGGGLVKPGGSLSLSCAASGFTFSSYWMSWVRQAPGKGLEWVANINRDGSASYYVDSVKGRFTISR
DDAKNSLYLQMNSLRAEDTAVYYCARDRGVGYFDLWGRGTLVTVSS
>SEQ ID 109 SI-50E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAGGTTCAGCTGGTGGAGTCTGGCGGTGGCCTGGTGCAGCCAGGGGGCTCACTCCGTTTGT
CCTGTGCAGCTTCTGGCTTCAACATTAAAGACACCTATATACACTGGGTGCGTCAGGCCCCGGGTAAGGGCCTGGAA
TGGGTTGCAAGGATTTATCCTACGAATGGTTATACTAGATATGCCGATAGCGTCAAGGGCCGTTTCACTATAAGCGC
AGACACATCCAAAAACACAGCCTACCTGCAGATGAACAGCCTGCGTGCTGAGGACACTGCCGTCTATTATTGTTCTA
GATGGGGAGGGGACGGCTTCTATGCTATGGACTACTGGGGTCAAGGAACCCTGGTCACCGTCTCCTCGGCTAGCACC
AAGGGCCCATCGGTCTTCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAGAGTTGAGCCCAAATCTTGTGACAAAAC
TCACACATGCCCACCGTGCCCAGCACCTGAAGCCGCGGGGGCACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACAACAGCACGTA
CCGTGTGGTCAGCGTCCTCACCGTCCTGCACCAGGACTGGCTGAATGGCAAGGAGTACAAGTGCGCGGTCTCCAACA
AAGCCCTCCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTGGCGGTGGAGGGTCCGGCGGTGG
TGGATCCGAGGTGCAGCTGTTGGAGTCTGGGGGAGGCTTGGTACAGCCTGGGGGGTCCCTGAGACTCTCCTGTGCAG
CCTCTGGATTCTCCTTCAGTAGCGGGTACGACATGTGCTGGGTCCGCCAGGCTCCAGGGAAGGGGCTGGAGTGGATC
GCATGCATTGCTGCTGGTAGTGCTGGTATCACTTACGACGCGAACTGGGCGAAAGGCCGGTTCACCATCTCCAGAGA
CAATTCCAAGAACACGCTGTATCTGCAAATGAACAGCCTGAGAGCCGAGGACACGGCCGTATATTACTGTGCGAGAT
CGGCGTTTTCGTTCGACTACGCCATGGACCTCTGGGGCCAGGGAACCCTGGTCACCGTCTCGAGCGGTGGAGGCGGA
TCTGGCGGAGGTGGTTCCGGCGGTGGCGGCTCCGGTGGAGGCGGCTCTGACATCCAGATGACCCAGTCTCCTTCCAC
CCTGTCTGCATCTGTAGGAGACAGAGTCACCATCACTTGCCAGGCCAGTCAGAGCATTAGTTCCCACTTAAACTGGT
ATCAGCAGAAACCAGGGAAAGCCCCTAAGCTCCTGATCTATAAGGCATCCACTCTGGCATCTGGGGTCCCATCAAGG
TTCAGCGGCAGTGGATCTGGGACAGAATTTACTCTCACCATCAGCAGCCTGCAGCCTGATGATTTTGCAACTTATTA
CTGCCAACAGGGTTATAGTTGGGGTAATGTTGATAATGTTTTCGGCGGAGGGACCAAGGTGGAGATCAAAGGCGGTG
GAGGGTCCGGCGGTGGTGGCTCCGGACGGTCGCTGGTGGAGTCTGGGGGAGGCTTGGTCCAGCCTGGGGGGTCCCTG
AGACTCTCCTGTACTGCCTCTGGATTCACCATCAGTAGCTACCACATGCAGTGGGTCCGCCAGGCTCCAGGGAAGGG
GCTGGAGTACATCGGAACCATTAGTAGTGGTGGTAATGTATACTACGCAAGCTCCGCTAGAGGCAGATTCACCATCT
CCAGACCCTCGTCCAAGAACACGGTGGATCTTCAAATGAACAGCCTGAGAGCCGAGGACACGGCTGTGTATTACTGT
GCGAGAGACTCTGGTTATAGTGATCCTATGTGGGGCCAGGGAACCCTGGTCACCGTCTCTTCAGGCGGTGGCGGTAG
TGGGGGAGGCGGTTCTGGCGGCGGAGGGTCCGGCGGTGGAGGATCAGACGTTGTGATGACCCAGTCTCCATCTTCCG
TGTCTGCATCTGTAGGAGACAGAGTCACCATCACCTGTCAGGCCAGTCAGAACATTAGGACTTACTTATCCTGGTAT
CAGCAGAAACCAGGGAAAGCCCCTAAGCTCCTGATCTATGCTGCAGCCAATCTGGCATCTGGGGTCCCATCAAGGTT
CAGCGGCAGTGGATCTGGGACAGATTTCACTCTCACCATCAGCGACCTGGAGCCTGGCGATGCTGCAACTTACTATT
GTCAGTCTACCTATCTTGGTACTGATTATGTTGGCGGTGCTTTCGGCGGAGGGACCAAGGTGGAGATCAAATGA
>SEQ ID 110 SI-50E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSEVQLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFYAMDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPEAAGAPSVFLFPPKPKDTLMISRTPEVTCVVVDVSHEDPEVK
FNWYVDGVEVHNAKTKPREEQYNSTYRVVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGGSGGGGSEVQLLESGGGLVQPGGSLRLSCAASGFSFSSGYDMCWVRQAPGKGLEWI
ACIAAGSAGITYDANWAKGRFTISRDNSKNTLYLQMNSLRAEDTAVYYCARSAFSFDYAMDLWGQGTLVTVSSGGGG
SGGGGSGGGGSGGGGSDIQMTQSPSTLSASVGDRVTITCQASQSISSHLNWYQQKPGKAPKLLIYKASTLASGVPSR
FSGSGSGTEFTLTISSLQPDDFATYYCQQGYSWGNVDNVFGGGTKVEIKGGGGSGGGGSGRSLVESGGGLVQPGGSL
RLSCTASGFTISSYHMQWVRQAPGKGLEYIGTISSGGNVYYASSARGRFTISRPSSKNTVDLQMNSLRAEDTAVYYC
ARDSGYSDPMWGQGTLVTVSSGGGGSGGGGSGGGGSGGGGSDVVMTQSPSSVSASVGDRVTITCQASQNIRTYLSWY
QQKPGKAPKLLIYAAANLASGVPSRFSGSGSGTDFTLTISDLEPGDAATYYCQSTYLGTDYVGGAFGGGTKVEIK
>SEQ ID 111 SI-50E1 and SI-50E6 chain B nt
GATATCCAGATGACCCAGTCCCCGAGCTCCCTGTCCGCCTCTGTGGGCGATAGGGTCACCATCACCTGCCGTGCCAG
TCAGGATGTGAATACTGCTGTAGCCTGGTATCAACAGAAACCAGGAAAAGCTCCGAAACTACTGATTTACTCGGCAT
CCTTCCTCTACTCTGGAGTCCCTTCTCGCTTCTCTGGCTCCAGATCTGGGACGGATTTCACTCTGACCATCAGCAGT
CTGCAGCCGGAAGACTTCGCAACTTATTACTGTCAGCAACATTATACTACTCCTCCCACGTTCGGACAGGGTACCAA
GGTGGAGATCAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>SEQ ID 112 SI-50E1 and SI-50E6 chain B aa
DIQMTQSPSSLSASVGDRVTITCRASQDVNTAVAWYQQKPGKAPKLLIYSASFLYSGVPSRFSGSRSGTDFTLTISS
LQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>SEQ ID 113 SI-50E6 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAGGTTCAGCTGGTGGAGTCTGGCGGTGGCCTGGTGCAGCCAGGGGGCTCACTCCGTTTGT
CCTGTGCAGCTTCTGGCTTCAACATTAAAGACACCTATATACACTGGGTGCGTCAGGCCCCGGGTAAGGGCCTGGAA
TGGGTTGCAAGGATTTATCCTACGAATGGTTATACTAGATATGCCGATAGCGTCAAGGGCCGTTTCACTATAAGCGC
AGACACATCCAAAAACACAGCCTACCTGCAGATGAACAGCCTGCGTGCTGAGGACACTGCCGTCTATTATTGTTCTA
GATGGGGAGGGGACGGCTTCTATGCTATGGACTACTGGGGTCAAGGAACCCTGGTCACCGTCTCCTCGGCTAGCACC
AAGGGCCCATCGGTCTTCCCCCTGGCACCCTCCTCCAAGAGCACCTCTGGGGGCACAGCGGCCCTGGGCTGCCTGGT
CAAGGACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAGAGTTGAGCCCAAATCTTGTGACAAAAC
TCACACATGCCCACCGTGCCCAGCACCTGAAGCCGCGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCACGAAGACCCTGAGGTCAAG
TTCAACTGGTACGTGGACGGCGTGGAGGTGCATAATGCCAAGACAAAGCCGCGGGAGGAGCAGTACGCCAGCACGTA
CCGTGTGGTCAGCGTCCTCACCGTCCTGCACCAGGACTGGCTGAATGGCAAGGAGTACAAGTGCGCGGTCTCCAACA
AAGCCCTCCCAGCCCCCATCGAGAAAACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTGGACTCCGACGGCT
CCTTCTTCCTCTATAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTGGCGGTGGAGGGTCCGGCGGTGG
TGGATCCGAGGTGCAGCTGTTGGAGTCTGGGGGAGGCTTGGTACAGCCTGGGGGGTCCCTGAGACTCTCCTGTGCAG
CCTCTGGATTCTCCTTCAGTAGCGGGTACGACATGTGCTGGGTCCGCCAGGCTCCAGGGAAGTGCCTGGAGTGGATC
GCATGCATTGCTGCTGGTAGTGCTGGTATCACTTACGACGCGAACTGGGCGAAAGGCCGGTTCACCATCTCCAGAGA
CAATTCCAAGAACACGCTGTATCTGCAAATGAACAGCCTGAGAGCCGAGGACACGGCCGTATATTACTGTGCGAGAT
CGGCGTTTTCGTTCGACTACGCCATGGACCTCTGGGGCCAGGGAACCCTGGTCACCGTCTCGAGCGGTGGAGGCGGT
TCAGGCGGAGGTGGAAGTGGTGGTGGCGGCTCTGGAGGCGGCGGATCTGACATCCAGATGACCCAGTCTCCTTCCAC
CCTGTCTGCATCTGTAGGAGACAGAGTCACCATCACTTGCCAGGCCAGTCAGAGCATTAGTTCCCACTTAAACTGGT
ATCAGCAGAAACCAGGGAAAGCCCCTAAGCTCCTGATCTATAAGGCATCCACTCTGGCATCTGGGGTCCCATCAAGG
TTCAGCGGCAGTGGATCTGGGACAGAATTTACTCTCACCATCAGCAGCCTGCAGCCTGATGATTTTGCAACTTATTA
CTGCCAACAGGGTTATAGTTGGGGTAATGTTGATAATGTTTTCGGCTGCGGGACCAAGGTGGAGATCAAAGGTGGTG
GCGGCTCTGGAGGAGGAGGGTCCGGACGGTCGCTGGTGGAGTCTGGGGGAGGCTTGGTCCAGCCTGGGGGGTCCCTG
AGACTCTCCTGTACTGCCTCTGGATTCACCATCAGTAGCTACCACATGCAGTGGGTCCGGCAGGCACCTGGGAAGTG
CCTGGAGTACATCGGAACCATTAGTAGTGGTGGTAATGTATACTACGCAAGCTCCGCTAGAGGCAGATTCACCATCT
CCAGACCCTCGTCCAAGAACACGGTGGATCTTCAAATGAACAGCCTGAGAGCCGAGGACACGGCTGTGTATTACTGT
GCGAGAGACTCTGGTTATAGTGATCCTATGTGGGGCCAGGGAACCCTGGTCACCGTCTCTTCAGGCGGTGGCGGTAG
TGGGGGAGGCGGTTCTGGCGGCGGAGGGTCCGGCGGTGGAGGATCAGACGTTGTGATGACCCAGTCTCCATCTTCCG
TGTCTGCATCTGTAGGAGACAGAGTCACCATCACCTGTCAGGCCAGTCAGAACATTAGGACTTACTTATCCTGGTAT
CAGCAGAAACCAGGGAAAGCCCCTAAGCTCCTGATCTATGCTGCAGCCAATCTGGCATCTGGGGTCCCATCAAGGTT
CAGCGGCAGTGGATCTGGGACAGATTTCACTCTCACCATCAGCGACTTGGAACCTGGCGATGCTGCAACTTACTATT
GTCAGTCTACCTATCTTGGTACTGATTATGTTGGCGGTGCTTTCGGCTGTGGGACCAAGGTGGAGATCAAATGA
>SEQ ID 114 SI-50E6 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSEVQLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGGDGFYAMDYWGQGTLVTVSSAST
KGPSVFPLAPSSKSTSGGTAALGCLVKDYFPEPVTVSWNSGALTSGVHTFPAVLQSSGLYSLSSVVTVPSSSLGTQT
YICNVNHKPSNTKVDKRVEPKSCDKTHTCPPCPAPEAAGGPSVFLFPPKPKDTLMISRTPEVTCVVVDVSHEDPEVK
FNWYVDGVEVHNAKTKPREEQYASTYRVVSVLTVLHQDWLNGKEYKCAVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGNVFSCSVM
HEALHNHYTQKSLSLSPGGGGGSGGGGSEVQLLESGGGLVQPGGSLRLSCAASGFSFSSGYDMCWVRQAPGKCLEWI
ACIAAGSAGITYDANWAKGRFTISRDNSKNTLYLQMNSLRAEDTAVYYCARSAFSFDYAMDLWGQGTLVTVSSGGGG
SGGGGSGGGGSGGGGSDIQMTQSPSTLSASVGDRVTITCQASQSISSHLNWYQQKPGKAPKLLIYKASTLASGVPSR
FSGSGSGTEFTLTISSLQPDDFATYYCQQGYSWGNVDNVFGCGTKVEIKGGGGSGGGGSGRSLVESGGGLVQPGGSL
RLSCTASGFTISSYHMQWVRQAPGKCLEYIGTISSGGNVYYASSARGRFTISRPSSKNTVDLQMNSLRAEDTAVYYC
ARDSGYSDPMWGQGTLVTVSSGGGGSGGGGSGGGGSGGGGSDVVMTQSPSSVSASVGDRVTITCQASQNIRTYLSWY
QQKPGKAPKLLIYAAANLASGVPSRFSGSGSGTDFTLTISDLEPGDAATYYCQSTYLGTDYVGGAFGCGTKVEIK
>SEQ ID 115 SI-50E2 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAAGTGCAGCTCGTCGAAAGCGGTGGCGGACTGGTTCAGCCCGGTGGTTCTCTGCGGCTGT
CTTGTGCTGCCTCGGGTTTCACGTTCACTGACTACACAATGGACTGGGTGCGTCAGGCTCCTGGAAAGGGATTGGAG
TGGGTAGCCGACGTTAATCCAAACTCCGGCGGGAGCATCTACAACCAGAGGTTCAAGGGGAGGTTCACTCTGAGCGT
GGATCGCTCCAAGAACACGCTGTACCTCCAGATGAACTCTCTCAGGGCCGAGGACACGGCTGTTTACTATTGCGCGA
GGAACCTGGGTCCTTCCTTCTACTTCGACTACTGGGGACAGGGAACCCTGGTGACCGTCAGCTCCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGCACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGGGGCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGATCT
GGCGGAGGTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATCAAAGGCGGTGGAG
GGTCCGGCGGTGGTGGCTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGCCAGGCTCCAGGGAAGGGGCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACCTGGAGCCTGGCGATGCTGCAACTTACTATTGTC
AGTCTACCTATCTTGGTACTGATTATGTTGGCGGTGCTTTCGGCGGAGGGACCAAGGTGGAGATCAAATGA
>SEQ ID 116 SI-50E2 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSEVQLVESGGGLVQPGGSLRLSCAASGFTFTDYTMDWVRQAPGKGLE
WVADVNPNSGGSIYNQRFKGRFTLSVDRSKNTLYLQMNSLRAEDTAVYYCARNLGPSFYFDYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVEIKGGGGSGGGGSGRSLVESGGGLVQPGGSLR
LSCTASGFTISSYHMQWVRQAPGKGLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGGGTKVEIK
>SEQ ID 117 SI-50E2 and SI-50E7 chain B nt
GACATCCAGATGACACAATCTCCTAGCAGTCTGAGCGCAAGTGTTGGAGATCGTGTCACCATCACATGCAAGGCCAG
CCAGGATGTGAGCATTGGAGTCGCCTGGTATCAGCAGAAACCCGGCAAGGCACCCAAGCTGCTGATCTACTCGGCCA
GTTACAGATACACTGGCGTACCTTCGAGGTTTAGTGGTAGCGGTTCTGGAACCGATTTCACCCTCACCATTAGCTCC
CTCCAACCCGAGGACTTCGCCACCTACTACTGCCAGCAATACTACATCTACCCTTACACGTTCGGCCAAGGCACTAA
GGTCGAGATTAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>SEQ ID 118 SI-50E2 and SI-50E7 chain B aa
DIQMTQSPSSLSASVGDRVTITCKASQDVSIGVAWYQQKPGKAPKLLIYSASYRYTGVPSRFSGSGSGTDFTLTISS
LQPEDFATYYCQQYYIYPYTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>SEQ ID 119 SI-50E7 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAAGTGCAGCTCGTCGAAAGCGGTGGCGGACTGGTTCAGCCCGGTGGTTCTCTGCGGCTGT
CTTGTGCTGCCTCGGGTTTCACGTTCACTGACTACACAATGGACTGGGTGCGTCAGGCTCCTGGAAAGGGATTGGAG
TGGGTAGCCGACGTTAATCCAAACTCCGGCGGGAGCATCTACAACCAGAGGTTCAAGGGGAGGTTCACTCTGAGCGT
GGATCGCTCCAAGAACACGCTGTACCTCCAGATGAACTCTCTCAGGGCCGAGGACACGGCTGTTTACTATTGCGCGA
GGAACCTGGGTCCTTCCTTCTACTTCGACTACTGGGGACAGGGAACCCTGGTGACCGTCAGCTCCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACGCCAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGTGCCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGTTCA
GGCGGAGGTGGAAGTGGTGGTGGCGGCTCTGGAGGCGGCGGATCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCTGCGGGACCAAGGTGGAGATCAAAGGTGGTGGCG
GCTCTGGAGGAGGAGGGTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGGCAGGCACCTGGGAAGTGCCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACTTGGAACCTGGCGATGCTGCAACTTACTATTGTC
AGTCTACCTATCTTGGTACTGATTATGTTGGCGGTGCTTTCGGCTGTGGGACCAAGGTGGAGATCAAATGA
>SEQ ID 120 SI-50E7 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSEVQLVESGGGLVQPGGSLRLSCAASGFTFTDYTMDWVRQAPGKGLE
WVADVNPNSGGSIYNQRFKGRFTLSVDRSKNTLYLQMNSLRAEDTAVYYCARNLGPSFYFDYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKCLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGCGTKVEIKGGGGSGGGGSGRSLVESGGGLVQPGGSLR
LSCTASGFTISSYHMQWVRQAPGKCLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGCGTKVEIK
>SEQ ID 121 SI-51E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTACAACTGCAGCAGTCTGGGCCTGAGCTGGAGAAGCCTGGCGCTTCAGTGAAGATAT
CCTGCAAGGCTTCTGGTTACTCATTCACTGGCTACACCATGAACTGGGTGAAGCAGAGCCATGGAAAGAGCCTTGAG
TGGATTGGACTTATTACTCCTTACAATGGTGCTTCTAGCTACAACCAGAAGTTCAGGGGCAAGGCCACATTAACTGT
AGACAAGTCATCCAGCACAGCCTACATGGACCTCCTCAGTCTGACATCTGAAGACTCTGCAGTCTATTTCTGTGCAA
GGGGGGGTTACGACGGGAGGGGTTTTGACTACTGGGGATCTGGGACCCCGGTCACCGTCTCCTCAGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGCACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGGGGCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGATCT
GGCGGAGGTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATCAAAGGCGGTGGAG
GGTCCGGCGGTGGTGGCTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGCCAGGCTCCAGGGAAGGGGCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACCTGGAGCCTGGCGATGCTGCAACTTACTATTGTC
AGTCTACCTATCTTGGTACTGATTATGTTGGCGGTGCTTTCGGCGGAGGGACCAAGGTGGAGATCAAATGA
>SEQ ID 122 SI-51E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLQQSGPELEKPGASVKISCKASGYSFTGYTMNWVKQSHGKSLE
WIGLITPYNGASSYNQKFRGKATLTVDKSSSTAYMDLLSLTSEDSAVYFCARGGYDGRGFDYWGSGTPVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVEIKGGGGSGGGGSGRSLVESGGGLVQPGGSLR
LSCTASGFTISSYHMQWVRQAPGKGLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGGGTKVEIK
>SEQ ID 123 SI-51E1 and SI-51E4 chain B nt
GACATCGAGCTCACTCAGTCTCCAGCAATCATGTCTGCATCTCCAGGGGAGAAGGTCACCATGACCTGCAGTGCCAG
CTCAAGTGTAAGTTACATGCACTGGTACCAGCAGAAGTCAGGCACCTCCCCCAAAAGATGGATTTATGACACATCCA
AACTGGCTTCTGGAGTCCCAGGTCGCTTCAGTGGCAGTGGGTCTGGAAACTCTTACTCTCTCACAATCAGCAGCGTG
GAGGCTGAAGATGATGCAACTTATTACTGCCAGCAGTGGAGTAAGCACCCTCTCACGTTCGGATCCGGGACCAAGGT
GGAAATCAAACGTACGGTGGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG
>SEQ ID 124 SI-51E1 and SI-51E4 chain B aa
DIELTQSPAIMSASPGEKVTMTCSASSSVSYMHWYQQKSGTSPKRWIYDTSKLASGVPGRFSGSGSGNSYSLTISSV
EAEDDATYYCQQWSKHPLTFGSGTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
>SEQ ID 125 SI-51E4 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTACAACTGCAGCAGTCTGGGCCTGAGCTGGAGAAGCCTGGCGCTTCAGTGAAGATAT
CCTGCAAGGCTTCTGGTTACTCATTCACTGGCTACACCATGAACTGGGTGAAGCAGAGCCATGGAAAGAGCCTTGAG
TGGATTGGACTTATTACTCCTTACAATGGTGCTTCTAGCTACAACCAGAAGTTCAGGGGCAAGGCCACATTAACTGT
AGACAAGTCATCCAGCACAGCCTACATGGACCTCCTCAGTCTGACATCTGAAGACTCTGCAGTCTATTTCTGTGCAA
GGGGGGGTTACGACGGGAGGGGTTTTGACTACTGGGGATCTGGGACCCCGGTCACCGTCTCCTCAGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACGCCAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGTGCCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGTTCA
GGCGGAGGTGGAAGTGGTGGTGGCGGCTCTGGAGGCGGCGGATCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCTGCGGGACCAAGGTGGAGATCAAAGGTGGTGGCG
GCTCTGGAGGAGGAGGGTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGGCAGGCACCTGGGAAGTGCCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACTTGGAACCTGGCGATGCTGCAACTTACTATTGTC
AGTCTACCTATCTTGGTACTGATTATGTTGGCGGTGCTTTCGGCTGTGGGACCAAGGTGGAGATCAAATGA
>SEQ ID 126 SI-51E4 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGETISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLQQSGPELEKPGASVKISCKASGYSFTGYTMNWVKQSHGKSLE
WIGLITPYNGASSYNQKFRGKATLTVDKSSSTAYMDLLSLTSEDSAVYFCARGGYDGRGFDYWGSGTPVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKCLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGCGTKVEIKGGGGSGGGGSGRSLVESGGGLVQPGGSLR
LSCTASGFTISSYHMQWVRQAPGKCLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGCGTKVEIK
>SEQ ID 127 SI-52E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAGGTCCAACTGCTGCAGTCTGGACCTGAGCTGGAGAAGCCTGGCGCTTCAGTGATGATAT
CCTGCAAGGCTTCTGGTTCCTCATTCACTGGCTACAACATGAACTGGGTGAGGCAGAACATTGGAAAGAGCCTTGAA
TGGATTGGAGCTATTGATCCTTACTATGGTGGAACTAGCTACAACCAGAAGTTCAAGGGCAGGGCCACATTGACTGT
AGACAAATCGTCCAGCACAGCCTACATGCACCTCAAGAGCCTGACATCTGAGGACTCTGCAGTCTATTACTGTGTAA
GCGGAATGGAGTACTGGGGTCAAGGAACCTCAGTCACCGTCTCCTCAGCTAGCACCAAGGGCCCATCGGTCTTCCCC
CTGGCACCCTCCTCCAAGAGCACCTCTGGGGGCACAGCGGCCCTGGGCTGCCTGGTCAAGGACTACTTCCCCGAACC
GGTGACGGTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGTGAATCAC
AAGCCCAGCAACACCAAGGTGGACAAGAGAGTTGAGCCCAAATCTTGTGACAAAACTCACACATGCCCACCGTGCCC
AGCACCTGAAGCCGCGGGGGCACCGTCAGTCTTCCTCTTCCCCCCAAAACCCAAGGACACCCTCATGATCTCCCGGA
CCCCTGAGGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGCACGTACCGTGTGGTCAGCGTCCTCAC
CGTCCTGCACCAGGACTGGCTGAATGGCAAGGAGTACAAGTGCGCGGTCTCCAACAAAGCCCTCCCAGCCCCCATCG
AGAAAACCATCTCCAAAGCCAAAGGGCAGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTCCTCTATAGCAAGC
TCACCGTGGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGTGGCGGTGGAGGGTCCGGCGGTGGTGGATCCGAGGTGCAGCTGTT
GGAGTCTGGGGGAGGCTTGGTACAGCCTGGGGGGTCCCTGAGACTCTCCTGTGCAGCCTCTGGATTCTCCTTCAGTA
GCGGGTACGACATGTGCTGGGTCCGCCAGGCTCCAGGGAAGGGGCTGGAGTGGATCGCATGCATTGCTGCTGGTAGT
GCTGGTATCACTTACGACGCGAACTGGGCGAAAGGCCGGTTCACCATCTCCAGAGACAATTCCAAGAACACGCTGTA
TCTGCAAATGAACAGCCTGAGAGCCGAGGACACGGCCGTATATTACTGTGCGAGATCGGCGTTTTCGTTCGACTACG
CCATGGACCTCTGGGGCCAGGGAACCCTGGTCACCGTCTCGAGCGGTGGAGGCGGATCTGGCGGAGGTGGTTCCGGC
GGTGGCGGCTCCGGTGGAGGCGGCTCTGACATCCAGATGACCCAGTCTCCTTCCACCCTGTCTGCATCTGTAGGAGA
CAGAGTCACCATCACTTGCCAGGCCAGTCAGAGCATTAGTTCCCACTTAAACTGGTATCAGCAGAAACCAGGGAAAG
CCCCTAAGCTCCTGATCTATAAGGCATCCACTCTGGCATCTGGGGTCCCATCAAGGTTCAGCGGCAGTGGATCTGGG
ACAGAATTTACTCTCACCATCAGCAGCCTGCAGCCTGATGATTTTGCAACTTATTACTGCCAACAGGGTTATAGTTG
GGGTAATGTTGATAATGTTTTCGGCGGAGGGACCAAGGTGGAGATCAAAGGCGGTGGAGGGTCCGGCGGTGGTGGCT
CCGGACGGTCGCTGGTGGAGTCTGGGGGAGGCTTGGTCCAGCCTGGGGGGTCCCTGAGACTCTCCTGTACTGCCTCT
GGATTCACCATCAGTAGCTACCACATGCAGTGGGTCCGCCAGGCTCCAGGGAAGGGGCTGGAGTACATCGGAACCAT
TAGTAGTGGTGGTAATGTATACTACGCAAGCTCCGCTAGAGGCAGATTCACCATCTCCAGACCCTCGTCCAAGAACA
CGGTGGATCTTCAAATGAACAGCCTGAGAGCCGAGGACACGGCTGTGTATTACTGTGCGAGAGACTCTGGTTATAGT
GATCCTATGTGGGGCCAGGGAACCCTGGTCACCGTCTCTTCAGGCGGTGGCGGTAGTGGGGGAGGCGGTTCTGGCGG
CGGAGGGTCCGGCGGTGGAGGATCAGACGTTGTGATGACCCAGTCTCCATCTTCCGTGTCTGCATCTGTAGGAGACA
GAGTCACCATCACCTGTCAGGCCAGTCAGAACATTAGGACTTACTTATCCTGGTATCAGCAGAAACCAGGGAAAGCC
CCTAAGCTCCTGATCTATGCTGCAGCCAATCTGGCATCTGGGGTCCCATCAAGGTTCAGCGGCAGTGGATCTGGGAC
AGATTTCACTCTCACCATCAGCGACCTGGAGCCTGGCGATGCTGCAACTTACTATTGTCAGTCTACCTATCTTGGTA
CTGATTATGTTGGCGGTGCTTTCGGCGGAGGGACCAAGGTGGAGATCAAATGA
>SEQ ID 128 SI-52E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSEVQLLQSGPELEKPGASVMISCKASGSSFTGYNMNWVRQNIGKSLE
WIGAIDPYYGGTSYNQKFKGRATLTVDKSSSTAYMHLKSLTSEDSAVYYCVSGMEYWGQGTSVTVSSASTKGPSVFP
LAPSSKSTSGGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPEAAGAPSVFLFPPKPKDTLMISRTPEVTCVVVDVSHEDPEVKFNWYVDG
VEVHNAKTKPREEQYNSTYRVVSVLTVLHQDWLNGKEYKCAVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNVFSCSVMHEALHNH
YTQKSLSLSPGGGGGSGGGGSEVQLLESGGGLVQPGGSLRLSCAASGFSFSSGYDMCWVRQAPGKGLEWIACIAAGS
AGITYDANWAKGRFTISRDNSKNTLYLQMNSLRAEDTAVYYCARSAFSFDYAMDLWGQGTLVTVSSGGGGSGGGGSG
GGGSGGGGSDIQMTQSPSTLSASVGDRVTITCQASQSISSHLNWYQQKPGKAPKLLIYKASTLASGVPSRFSGSGSG
TEFTLTISSLQPDDFATYYCQQGYSWGNVDNVFGGGTKVEIKGGGGSGGGGSGRSLVESGGGLVQPGGSLRLSCTAS
GFTISSYHMQWVRQAPGKGLEYIGTISSGGNVYYASSARGRFTISRPSSKNTVDLQMNSLRAEDTAVYYCARDSGYS
DPMWGQGTLVTVSSGGGGSGGGGSGGGGSGGGGSDVVMTQSPSSVSASVGDRVTITCQASQNIRTYLSWYQQKPGKA
PKLLIYAAANLASGVPSRFSGSGSGTDFTLTISDLEPGDAATYYCQSTYLGTDYVGGAFGGGTKVEIK
>SEQ ID 129 SI-52E1 and SI-52E4 chain B nt
GAAATAGTGATGACGCAGTCTCCAGCCACCCTGTCTGTGTCTCCAGGGGAAAGAGCCACCCTCTCCTGCAGATCTAG
TCAGAGTCTTGTACACCGTAATGGAAACACCTATTTACATTGGTACCTGCAGAAGCCAGGCCAGTCTCCAAAGCTCC
TGATTCACAAAGTTTCCAACCGATTTTCTGGGGTCCCAGACAGGTTCAGTGGCAGTGGATCAGGGACAGATTTCACA
CTCAAGATCAGCAGAGTGGAGGCTGAGGATCTGGGAGTTTATTTCTGTTCTCAAAGTACACATGTTCCTCCGCTCAC
GTTCGGTGCTGGGACCAAGCTGGAGCTGAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTGCGAAGTCACCCATCAGG
GCCTGAGCTCGCCCGTCACAAAGAGCTTCAACAGGGGAGAGTGTTAG
>SEQ ID 130 SI-52E1 and SI-52E4 chain B aa
EIVMTQSPATLSVSPGERATLSCRSSQSLVHRNGNTYLHWYLQKPGQSPKLLIHKVSNRFSGVPDRFSGSGSGTDFT
LKISRVEAEDLGVYFCSQSTHVPPLTFGAGTKLELKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
>SEQ ID 131 SI-52E4 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAGGTCCAACTGCTGCAGTCTGGACCTGAGCTGGAGAAGCCTGGCGCTTCAGTGATGATAT
CCTGCAAGGCTTCTGGTTCCTCATTCACTGGCTACAACATGAACTGGGTGAGGCAGAACATTGGAAAGAGCCTTGAA
TGGATTGGAGCTATTGATCCTTACTATGGTGGAACTAGCTACAACCAGAAGTTCAAGGGCAGGGCCACATTGACTGT
AGACAAATCGTCCAGCACAGCCTACATGCACCTCAAGAGCCTGACATCTGAGGACTCTGCAGTCTATTACTGTGTAA
GCGGAATGGAGTACTGGGGTCAAGGAACCTCAGTCACCGTCTCCTCAGCTAGCACCAAGGGCCCATCGGTCTTCCCC
CTGGCACCCTCCTCCAAGAGCACCTCTGGGGGCACAGCGGCCCTGGGCTGCCTGGTCAAGGACTACTTCCCCGAACC
GGTGACGGTGTCGTGGAACTCAGGCGCCCTGACCAGCGGCGTGCACACCTTCCCGGCTGTCCTACAGTCCTCAGGAC
TCTACTCCCTCAGCAGCGTGGTGACCGTGCCCTCCAGCAGCTTGGGCACCCAGACCTACATCTGCAACGTGAATCAC
AAGCCCAGCAACACCAAGGTGGACAAGAGAGTTGAGCCCAAATCTTGTGACAAAACTCACACATGCCCACCGTGCCC
AGCACCTGAAGCCGCGGGGGGACCGTCAGTCTTCCTCTTCCCCCCAAAACCCAAGGACACCCTCATGATCTCCCGGA
CCCCTGAGGTCACATGCGTGGTGGTGGACGTGAGCCACGAAGACCCTGAGGTCAAGTTCAACTGGTACGTGGACGGC
GTGGAGGTGCATAATGCCAAGACAAAGCCGCGGGAGGAGCAGTACGCCAGCACGTACCGTGTGGTCAGCGTCCTCAC
CGTCCTGCACCAGGACTGGCTGAATGGCAAGGAGTACAAGTGCGCGGTCTCCAACAAAGCCCTCCCAGCCCCCATCG
AGAAAACCATCTCCAAAGCCAAAGGGCAGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAGCGACATCGCCGTGGAGTGGGAGAGCAA
TGGGCAGCCGGAGAACAACTACAAGACCACGCCTCCCGTGCTGGACTCCGACGGCTCCTTCTTCCTCTATAGCAAGC
TCACCGTGGACAAGAGCAGGTGGCAGCAGGGGAACGTCTTCTCATGCTCCGTGATGCATGAGGCTCTGCACAACCAC
TACACGCAGAAGAGCCTCTCCCTGTCTCCGGGTGGCGGTGGAGGGTCCGGCGGTGGTGGATCCGAGGTGCAGCTGTT
GGAGTCTGGGGGAGGCTTGGTACAGCCTGGGGGGTCCCTGAGACTCTCCTGTGCAGCCTCTGGATTCTCCTTCAGTA
GCGGGTACGACATGTGCTGGGTCCGCCAGGCTCCAGGGAAGTGCCTGGAGTGGATCGCATGCATTGCTGCTGGTAGT
GCTGGTATCACTTACGACGCGAACTGGGCGAAAGGCCGGTTCACCATCTCCAGAGACAATTCCAAGAACACGCTGTA
TCTGCAAATGAACAGCCTGAGAGCCGAGGACACGGCCGTATATTACTGTGCGAGATCGGCGTTTTCGTTCGACTACG
CCATGGACCTCTGGGGCCAGGGAACCCTGGTCACCGTCTCGAGCGGTGGAGGCGGTTCAGGCGGAGGTGGAAGTGGT
GGTGGCGGCTCTGGAGGCGGCGGATCTGACATCCAGATGACCCAGTCTCCTTCCACCCTGTCTGCATCTGTAGGAGA
CAGAGTCACCATCACTTGCCAGGCCAGTCAGAGCATTAGTTCCCACTTAAACTGGTATCAGCAGAAACCAGGGAAAG
CCCCTAAGCTCCTGATCTATAAGGCATCCACTCTGGCATCTGGGGTCCCATCAAGGTTCAGCGGCAGTGGATCTGGG
ACAGAATTTACTCTCACCATCAGCAGCCTGCAGCCTGATGATTTTGCAACTTATTACTGCCAACAGGGTTATAGTTG
GGGTAATGTTGATAATGTTTTCGGCTGCGGGACCAAGGTGGAGATCAAAGGTGGTGGCGGCTCTGGAGGAGGAGGGT
CCGGACGGTCGCTGGTGGAGTCTGGGGGAGGCTTGGTCCAGCCTGGGGGGTCCCTGAGACTCTCCTGTACTGCCTCT
GGATTCACCATCAGTAGCTACCACATGCAGTGGGTCCGGCAGGCACCTGGGAAGTGCCTGGAGTACATCGGAACCAT
TAGTAGTGGTGGTAATGTATACTACGCAAGCTCCGCTAGAGGCAGATTCACCATCTCCAGACCCTCGTCCAAGAACA
CGGTGGATCTTCAAATGAACAGCCTGAGAGCCGAGGACACGGCTGTGTATTACTGTGCGAGAGACTCTGGTTATAGT
GATCCTATGTGGGGCCAGGGAACCCTGGTCACCGTCTCTTCAGGCGGTGGCGGTAGTGGGGGAGGCGGTTCTGGCGG
CGGAGGGTCCGGCGGTGGAGGATCAGACGTTGTGATGACCCAGTCTCCATCTTCCGTGTCTGCATCTGTAGGAGACA
GAGTCACCATCACCTGTCAGGCCAGTCAGAACATTAGGACTTACTTATCCTGGTATCAGCAGAAACCAGGGAAAGCC
CCTAAGCTCCTGATCTATGCTGCAGCCAATCTGGCATCTGGGGTCCCATCAAGGTTCAGCGGCAGTGGATCTGGGAC
AGATTTCACTCTCACCATCAGCGACTTGGAACCTGGCGATGCTGCAACTTACTATTGTCAGTCTACCTATCTTGGTA
CTGATTATGTTGGCGGTGCTTTCGGCTGTGGGACCAAGGTGGAGATCAAATGA
>SEQ ID 132 SI-52E4 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSEVQLLQSGPELEKPGASVMISCKASGSSFTGYNMNWVRQNIGKSLE
WIGAIDPYYGGTSYNQKFKGRATLTVDKSSSTAYMHLKSLTSEDSAVYYCVSGMEYWGQGTSVTVSSASTKGPSVFP
LAPSSKSTSGGTAALGCLVKDYFPEPVTVSWNSGALTSGVHTFPAVLQSSGLYSLSSVVTVPSSSLGTQTYICNVNH
KPSNTKVDKRVEPKSCDKTHTCPPCPAPEAAGGPSVFLFPPKPKDTLMISRTPEVTCVVVDVSHEDPEVKFNWYVDG
VEVHNAKTKPREEQYASTYRVVSVLTVLHQDWLNGKEYKCAVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNVFSCSVMHEALHNH
YTQKSLSLSPGGGGGSGGGGSEVQLLESGGGLVQPGGSLRLSCAASGFSFSSGYDMCWVRQAPGKCLEWIACIAAGS
AGITYDANWAKGRFTISRDNSKNTLYLQMNSLRAEDTAVYYCARSAFSFDYAMDLWGQGTLVTVSSGGGGSGGGGSG
GGGSGGGGSDIQMTQSPSTLSASVGDRVTITCQASQSISSHLNWYQQKPGKAPKLLIYKASTLASGVPSRFSGSGSG
TEFTLTISSLQPDDFATYYCQQGYSWGNVDNVFGCGTKVEIKGGGGSGGGGSGRSLVESGGGLVQPGGSLRLSCTAS
GFTISSYHMQWVRQAPGKCLEYIGTISSGGNVYYASSARGRFTISRPSSKNTVDLQMNSLRAEDTAVYYCARDSGYS
DPMWGQGTLVTVSSGGGGSGGGGSGGGGSGGGGSDVVMTQSPSSVSASVGDRVTITCQASQNIRTYLSWYQQKPGKA
PKLLIYAAANLASGVPSRFSGSGSGTDFTLTISDLEPGDAATYYCQSTYLGTDYVGGAFGCGTKVEIK
>SEQ ID 133 SI-53E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTCCAACTGCAGCAGCCTGGGGCTGAGCTGGTGAGGCCTGGGGCTTCAGTGAAGCTGT
CCTGCAAGGCTTCTGGCTACACCTTCACCAGCTACTGGATAAACTGGGTGAAGCAGAGGCCTGGACAAGGCCTTGAG
TGGATCGGAAATATTTATCCTTCTGATAGTTATACTAACTACAATCAAAAGTTCAAGGACAAGGCCACATTGACTGT
AGACAAATCCTCCAGCACAGCCTACATGCAGCTCAGCAGCCCGACATCTGAGGACTCTGCGGTCTATTACTGTACAA
GATCGTGGAGGGGTAACTCCTTTGACTACTGGGGCCAAGGCACCACTCTCACAGTCTCCTCAGCTAGCACCAAGGGC
CCATCGGTCTTCCCCCTGGCACCCTCCTCCAAGAGCACCTCTGGGGGCACAGCGGCCCTGGGCTGCCTGGTCAAGGA
CTACTTCCCCGAACCGGTGACGGTGTCGTGGAACTCAGGCGCCCTGACCAGCGGCGTGCACACCTTCCCGGCTGTCC
TACAGTCCTCAGGACTCTACTCCCTCAGCAGCGTGGTGACCGTGCCCTCCAGCAGCTTGGGCACCCAGACCTACATC
TGCAACGTGAATCACAAGCCCAGCAACACCAAGGTGGACAAGAGAGTTGAGCCCAAATCTTGTGACAAAACTCACAC
ATGCCCACCGTGCCCAGCACCTGAAGCCGCGGGGGCACCGTCAGTCTTCCTCTTCCCCCCAAAACCCAAGGACACCC
TCATGATCTCCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAGCACGTACCGTGT
GGTCAGCGTCCTCACCGTCCTGCACCAGGACTGGCTGAATGGCAAGGAGTACAAGTGCGCGGTCTCCAACAAAGCCC
TCCCAGCCCCCATCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCT
TCCTCTATAGCAAGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTGGCGGTGGAGGGTCCGGCGGTGGTGGATC
CGAGGTGCAGCTGTTGGAGTCTGGGGGAGGCTTGGTACAGCCTGGGGGGTCCCTGAGACTCTCCTGTGCAGCCTCTG
GATTCTCCTTCAGTAGCGGGTACGACATGTGCTGGGTCCGCCAGGCTCCAGGGAAGGGGCTGGAGTGGATCGCATGC
ATTGCTGCTGGTAGTGCTGGTATCACTTACGACGCGAACTGGGCGAAAGGCCGGTTCACCATCTCCAGAGACAATTC
CAAGAACACGCTGTATCTGCAAATGAACAGCCTGAGAGCCGAGGACACGGCCGTATATTACTGTGCGAGATCGGCGT
TTTCGTTCGACTACGCCATGGACCTCTGGGGCCAGGGAACCCTGGTCACCGTCTCGAGCGGTGGAGGCGGATCTGGC
GGAGGTGGTTCCGGCGGTGGCGGCTCCGGTGGAGGCGGCTCTGACATCCAGATGACCCAGTCTCCTTCCACCCTGTC
TGCATCTGTAGGAGACAGAGTCACCATCACTTGCCAGGCCAGTCAGAGCATTAGTTCCCACTTAAACTGGTATCAGC
AGAAACCAGGGAAAGCCCCTAAGCTCCTGATCTATAAGGCATCCACTCTGGCATCTGGGGTCCCATCAAGGTTCAGC
GGCAGTGGATCTGGGACAGAATTTACTCTCACCATCAGCAGCCTGCAGCCTGATGATTTTGCAACTTATTACTGCCA
ACAGGGTTATAGTTGGGGTAATGTTGATAATGTTTTCGGCGGAGGGACCAAGGTGGAGATCAAAGGCGGTGGAGGGT
CCGGCGGTGGTGGCTCCGGACGGTCGCTGGTGGAGTCTGGGGGAGGCTTGGTCCAGCCTGGGGGGTCCCTGAGACTC
TCCTGTACTGCCTCTGGATTCACCATCAGTAGCTACCACATGCAGTGGGTCCGCCAGGCTCCAGGGAAGGGGCTGGA
GTACATCGGAACCATTAGTAGTGGTGGTAATGTATACTACGCAAGCTCCGCTAGAGGCAGATTCACCATCTCCAGAC
CCTCGTCCAAGAACACGGTGGATCTTCAAATGAACAGCCTGAGAGCCGAGGACACGGCTGTGTATTACTGTGCGAGA
GACTCTGGTTATAGTGATCCTATGTGGGGCCAGGGAACCCTGGTCACCGTCTCTTCAGGCGGTGGCGGTAGTGGGGG
AGGCGGTTCTGGCGGCGGAGGGTCCGGCGGTGGAGGATCAGACGTTGTGATGACCCAGTCTCCATCTTCCGTGTCTG
CATCTGTAGGAGACAGAGTCACCATCACCTGTCAGGCCAGTCAGAACATTAGGACTTACTTATCCTGGTATCAGCAG
AAACCAGGGAAAGCCCCTAAGCTCCTGATCTATGCTGCAGCCAATCTGGCATCTGGGGTCCCATCAAGGTTCAGCGG
CAGTGGATCTGGGACAGATTTCACTCTCACCATCAGCGACCTGGAGCCTGGCGATGCTGCAACTTACTATTGTCAGT
CTACCTATCTTGGTACTGATTATGTTGGCGGTGCTTTCGGCGGAGGGACCAAGGTGGAGATCAAATGA
>SEQ ID 134 SI-53E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLQQPGAELVRPGASVKLSCKASGYTFTSYWINWVKQRPGQGLE
WIGNIYPSDSYTNYNQKFKDKATLTVDKSSSTAYMQLSSPTSEDSAVYYCTRSWRGNSFDYWGQGTTLTVSSASTKG
PSVFPLAPSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPEAAGAPSVFLFPPKPKDTLMISRTPEVTCVVVDVSHEDPEVKFN
WYVDGVEVHNAKTKPREEQYNSTYRVVSVLTVLHQDWLNGKEYKCAVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQGNVFSCSVMHE
ALHNHYTQKSLSLSPGGGGGSGGGGSEVQLLESGGGLVQPGGSLRLSCAASGFSFSSGYDMCWVRQAPGKGLEWIAC
IAAGSAGITYDANWAKGRFTISRDNSKNTLYLQMNSLRAEDTAVYYCARSAFSFDYAMDLWGQGTLVTVSSGGGGSG
GGGSGGGGSGGGGSDIQMTQSPSTLSASVGDRVTITCQASQSISSHLNWYQQKPGKAPKLLIYKASTLASGVPSRFS
GSGSGTEFTLTISSLQPDDFATYYCQQGYSWGNVDNVFGGGTKVEIKGGGGSGGGGSGRSLVESGGGLVQPGGSLRL
SCTASGFTISSYHMQWVRQAPGKGLEYIGTISSGGNVYYASSARGRFTISRPSSKNTVDLQMNSLRAEDTAVYYCAR
DSGYSDPMWGQGTLVTVSSGGGGSGGGGSGGGGSGGGGSDVVMTQSPSSVSASVGDRVTITCQASQNIRTYLSWYQQ
KPGKAPKLLIYAAANLASGVPSRFSGSGSGTDFTLTISDLEPGDAATYYCQSTYLGTDYVGGAFGGGTKVEIK
>SEQ ID 135 SI-53E1 and SI-53E3 chain B nt
GACATTGTGATGACACAGTCTCCATCCTCCCTGACTGTGACAGCAGGAGAGAAGGTCACTATGAGCTGCAAGTCCAG
TCAGAGTCTGTTAAACAGTGGAAATCAAAAGAACTACTTGACCTGGTACCAGCAGAAACCAGGGCAGCCTCCTAAAC
TGTTGATCTACTGGGCATCCACTAGGGAATCTGGGGTCCCTGATCGCTTCACAGGCAGTGGATCTGGAACAGATTTC
ACTCTCACCATCAGCAGTGTGCAGGCTGAAGACCTGGCAGTTTATTACTGTCAGAATGATTATAGTTATCCATTCAC
GTTCGGCTCGGGGACAAAGTTGGAAATAAAACGTACGGTGGCTGCACCATCTGTCTTCATCTTCCCGCCATCTGATG
AGCAGTTGAAATCTGGAACTGCCTCTGTTGTGTGCCTGCTGAATAACTTCTATCCCAGAGAGGCCAAAGTACAGTGG
AAGGTGGATAACGCCCTCCAATCGGGTAACTCCCAGGAGAGTGTCACAGAGCAGGACAGCAAGGACAGCACCTACAG
CCTCAGCAGCACCCTGACGCTGAGCAAAGCAGACTACGAGAAACACAAAGTCTACGCCTGCGAAGTCACCCATCAGG
GCCTGAGCTCGCCCGTCACAAAGAGCTTCAACAGGGGAGAGTGTTAG
>SEQ ID 136 SI-53E1 and SI-53E3 chain B aa
DIVMTQSPSSLTVTAGEKVTMSCKSSQSLLNSGNQKNYLTWYQQKPGQPPKLLIYWASTRESGVPDRFTGSGSGTDF
TLTISSVQAEDLAVYYCQNDYSYPFTFGSGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
>SEQ ID 137 SI-53E3 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTCCAACTGCAGCAGCCTGGGGCTGAGCTGGTGAGGCCTGGGGCTTCAGTGAAGCTGT
CCTGCAAGGCTTCTGGCTACACCTTCACCAGCTACTGGATAAACTGGGTGAAGCAGAGGCCTGGACAAGGCCTTGAG
TGGATCGGAAATATTTATCCTTCTGATAGTTATACTAACTACAATCAAAAGTTCAAGGACAAGGCCACATTGACTGT
AGACAAATCCTCCAGCACAGCCTACATGCAGCTCAGCAGCCCGACATCTGAGGACTCTGCGGTCTATTACTGTACAA
GATCGTGGAGGGGTAACTCCTTTGACTACTGGGGCCAAGGCACCACTCTCACAGTCTCCTCAGCTAGCACCAAGGGC
CCATCGGTCTTCCCCCTGGCACCCTCCTCCAAGAGCACCTCTGGGGGCACAGCGGCCCTGGGCTGCCTGGTCAAGGA
CTACTTCCCCGAACCGGTGACGGTGTCGTGGAACTCAGGCGCCCTGACCAGCGGCGTGCACACCTTCCCGGCTGTCC
TACAGTCCTCAGGACTCTACTCCCTCAGCAGCGTGGTGACCGTGCCCTCCAGCAGCTTGGGCACCCAGACCTACATC
TGCAACGTGAATCACAAGCCCAGCAACACCAAGGTGGACAAGAGAGTTGAGCCCAAATCTTGTGACAAAACTCACAC
ATGCCCACCGTGCCCAGCACCTGAAGCCGCGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACGCCAGCACGTACCGTGT
GGTCAGCGTCCTCACCGTCCTGCACCAGGACTGGCTGAATGGCAAGGAGTACAAGTGCGCGGTCTCCAACAAAGCCC
TCCCAGCCCCCATCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACTCCGACGGCTCCTTCT
TCCTCTATAGCAAGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTGGCGGTGGAGGGTCCGGCGGTGGTGGATC
CGAGGTGCAGCTGTTGGAGTCTGGGGGAGGCTTGGTACAGCCTGGGGGGTCCCTGAGACTCTCCTGTGCAGCCTCTG
GATTCTCCTTCAGTAGCGGGTACGACATGTGCTGGGTCCGCCAGGCTCCAGGGAAGTGCCTGGAGTGGATCGCATGC
ATTGCTGCTGGTAGTGCTGGTATCACTTACGACGCGAACTGGGCGAAAGGCCGGTTCACCATCTCCAGAGACAATTC
CAAGAACACGCTGTATCTGCAAATGAACAGCCTGAGAGCCGAGGACACGGCCGTATATTACTGTGCGAGATCGGCGT
TTTCGTTCGACTACGCCATGGACCTCTGGGGCCAGGGAACCCTGGTCACCGTCTCGAGCGGTGGAGGCGGTTCAGGC
GGAGGTGGAAGTGGTGGTGGCGGCTCTGGAGGCGGCGGATCTGACATCCAGATGACCCAGTCTCCTTCCACCCTGTC
TGCATCTGTAGGAGACAGAGTCACCATCACTTGCCAGGCCAGTCAGAGCATTAGTTCCCACTTAAACTGGTATCAGC
AGAAACCAGGGAAAGCCCCTAAGCTCCTGATCTATAAGGCATCCACTCTGGCATCTGGGGTCCCATCAAGGTTCAGC
GGCAGTGGATCTGGGACAGAATTTACTCTCACCATCAGCAGCCTGCAGCCTGATGATTTTGCAACTTATTACTGCCA
ACAGGGTTATAGTTGGGGTAATGTTGATAATGTTTTCGGCTGCGGGACCAAGGTGGAGATCAAAGGTGGTGGCGGCT
CTGGAGGAGGAGGGTCCGGACGGTCGCTGGTGGAGTCTGGGGGAGGCTTGGTCCAGCCTGGGGGGTCCCTGAGACTC
TCCTGTACTGCCTCTGGATTCACCATCAGTAGCTACCACATGCAGTGGGTCCGGCAGGCACCTGGGAAGTGCCTGGA
GTACATCGGAACCATTAGTAGTGGTGGTAATGTATACTACGCAAGCTCCGCTAGAGGCAGATTCACCATCTCCAGAC
CCTCGTCCAAGAACACGGTGGATCTTCAAATGAACAGCCTGAGAGCCGAGGACACGGCTGTGTATTACTGTGCGAGA
GACTCTGGTTATAGTGATCCTATGTGGGGCCAGGGAACCCTGGTCACCGTCTCTTCAGGCGGTGGCGGTAGTGGGGG
AGGCGGTTCTGGCGGCGGAGGGTCCGGCGGTGGAGGATCAGACGTTGTGATGACCCAGTCTCCATCTTCCGTGTCTG
CATCTGTAGGAGACAGAGTCACCATCACCTGTCAGGCCAGTCAGAACATTAGGACTTACTTATCCTGGTATCAGCAG
AAACCAGGGAAAGCCCCTAAGCTCCTGATCTATGCTGCAGCCAATCTGGCATCTGGGGTCCCATCAAGGTTCAGCGG
CAGTGGATCTGGGACAGATTTCACTCTCACCATCAGCGACTTGGAACCTGGCGATGCTGCAACTTACTATTGTCAGT
CTACCTATCTTGGTACTGATTATGTTGGCGGTGCTTTCGGCTGTGGGACCAAGGTGGAGATCAAATGA
>SEQ ID 138 SI-53E3 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLQQPGAELVRPGASVKLSCKASGYTFTSYWINWVKQRPGQGLE
WIGNIYPSDSYTNYNQKFKDKATLTVDKSSSTAYMQLSSPTSEDSAVYYCTRSWRGNSFDYWGQGTTLTVSSASTKG
PSVFPLAPSSKSTSGGTAALGCLVKDYFPEPVTVSWNSGALTSGVHTFPAVLQSSGLYSLSSVVTVPSSSLGTQTYI
CNVNHKPSNTKVDKRVEPKSCDKTHTCPPCPAPEAAGGPSVFLFPPKPKDTLMISRTPEVTCVVVDVSHEDPEVKFN
WYVDGVEVHNAKTKPREEQYASTYRVVSVLTVLHQDWLNGKEYKCAVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQGNVFSCSVMHE
ALHNHYTQKSLSLSPGGGGGSGGGGSEVQLLESGGGLVQPGGSLRLSCAASGFSFSSGYDMCWVRQAPGKCLEWIAC
IAAGSAGITYDANWAKGRFTISRDNSKNTLYLQMNSLRAEDTAVYYCARSAFSFDYAMDLWGQGTLVTVSSGGGGSG
GGGSGGGGSGGGGSDIQMTQSPSTLSASVGDRVTITCQASQSISSHLNWYQQKPGKAPKLLIYKASTLASGVPSRFS
GSGSGTEFTLTISSLQPDDFATYYCQQGYSWGNVDNVFGCGTKVEIKGGGGSGGGGSGRSLVESGGGLVQPGGSLRL
SCTASGFTISSYHMQWVRQAPGKCLEYIGTISSGGNVYYASSARGRFTISRPSSKNTVDLQMNSLRAEDTAVYYCAR
DSGYSDPMWGQGTLVTVSSGGGGSGGGGSGGGGSGGGGSDVVMTQSPSSVSASVGDRVTITCQASQNIRTYLSWYQQ
KPGKAPKLLIYAAANLASGVPSRFSGSGSGTDFTLTISDLEPGDAATYYCQSTYLGTDYVGGAFGCGTKVEIK
>SEQ ID 139 SI-54E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGCAGCCCGGCGCCGAGCTGGTGAAGCCCGGCGCCAGCGTGAAGATGA
GCTGCAAGGCCAGCGGCTACACCTTCACCAGCTACAACATGCACTGGGTGAAGCAGACCCCCGGCCGGGGCCTGGAG
TGGATCGGCGCCATCTACCCCGGCAACGGCGACACCAGCTACAACCAGAAGTTCAAGGGCAAGGCCACCCTGACCGC
CGACAAGAGCAGCAGCACCGCCTACATGCAGCTGAGCAGCCTGACCAGCGAGGACAGCGCCGTGTACTACTGCGCCC
GGAGCACCTACTACGGCGGCGACTGGTACTTCAACGTGTGGGGCGCCGGCACCACCGTGACCGTGAGCGCCGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGCACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTG
CAGCCTCTGGATTCTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGTTCACCATCTCCAG
AGACAATTCCAAGAACACGCTGTATCTGCAAATGAACAGCCTGAGAGCCGAGGACACGGCCGTATATTACTGTGCGA
GATCGGCGTTTTCGTTCGACTACGCCATGGACCTCTGGGGCCAGGGAACCCTGGTCACCGTCTCGAGCGGTGGAGGC
GGATCTGGCGGAGGTGGTTCCGGCGGTGGCGGCTCCGGTGGAGGCGGCTCTGACATCCAGATGACCCAGTCTCCTTC
CACCCTGTCTGCATCTGTAGGAGACAGAGTCACCATCACTTGCCAGGCCAGTCAGAGCATTAGTTCCCACTTAAACT
GGTATCAGCAGAAACCAGGGAAAGCCCCTAAGCTCCTGATCTATAAGGCATCCACTCTGGCATCTGGGGTCCCATCA
AGGTTCAGCGGCAGTGGATCTGGGACAGAATTTACTCTCACCATCAGCAGCCTGCAGCCTGATGATTTTGCAACTTA
TTACTGCCAACAGGGTTATAGTTGGGGTAATGTTGATAATGTTTTCGGCGGAGGGACCAAGGTGGAGATCAAAGGCG
GTGGAGGGTCCGGCGGTGGTGGCTCCGGACGGTCGCTGGTGGAGTCTGGGGGAGGCTTGGTCCAGCCTGGGGGGTCC
CTGAGACTCTCCTGTACTGCCTCTGGATTCACCATCAGTAGCTACCACATGCAGTGGGTCCGCCAGGCTCCAGGGAA
GGGGCTGGAGTACATCGGAACCATTAGTAGTGGTGGTAATGTATACTACGCAAGCTCCGCTAGAGGCAGATTCACCA
TCTCCAGACCCTCGTCCAAGAACACGGTGGATCTTCAAATGAACAGCCTGAGAGCCGAGGACACGGCTGTGTATTAC
TGTGCGAGAGACTCTGGTTATAGTGATCCTATGTGGGGCCAGGGAACCCTGGTCACCGTCTCTTCAGGCGGTGGCGG
TAGTGGGGGAGGCGGTTCTGGCGGCGGAGGGTCCGGCGGTGGAGGATCAGACGTTGTGATGACCCAGTCTCCATCTT
CCGTGTCTGCATCTGTAGGAGACAGAGTCACCATCACCTGTCAGGCCAGTCAGAACATTAGGACTTACTTATCCTGG
TATCAGCAGAAACCAGGGAAAGCCCCTAAGCTCCTGATCTATGCTGCAGCCAATCTGGCATCTGGGGTCCCATCAAG
GTTCAGCGGCAGTGGATCTGGGACAGATTTCACTCTCACCATCAGCGACCTGGAGCCTGGCGATGCTGCAACTTACT
ATTGTCAGTCTACCTATCTTGGTACTGATTATGTTGGCGGTGCTTTCGGCGGAGGGACCAAGGTGGAGATCAAATGA
>SEQ ID 140 SI-54E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLQQPGAELVKPGASVKMSCKASGYTFTSYNMHWVKQTPGRGLE
WIGAIYPGNGDTSYNQKFKGKATLTADKSSSTAYMQLSSLTSEDSAVYYCARSTYYGGDWYFNVWGAGTTVTVSAAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVTCVVVDVSHEDPEV
KFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSFDYAMDLWGQGTLVTVSSGGG
GSGGGGSGGGGSGGGGSDIQMTQSPSTLSASVGDRVTITCQASQSISSHLNWYQQKPGKAPKLLIYKASTLASGVPS
RFSGSGSGTEFTLTISSLQPDDFATYYCQQGYSWGNVDNVFGGGTKVEIKGGGGSGGGGSGRSLVESGGGLVQPGGS
LRLSCTASGFTISSYHMQWVRQAPGKGLEYIGTISSGGNVYYASSARGRFTISRPSSKNTVDLQMNSLRAEDTAVYY
CARDSGYSDPMWGQGTLVTVSSGGGGSGGGGSGGGGSGGGGSDVVMTQSPSSVSASVGDRVTITCQASQNIRTYLSW
YQQKPGKAPKLLIYAAANLASGVPSRFSGSGSGTDFTLTISDLEPGDAATYYCQSTYLGTDYVGGAFGGGTKVEIK
>SEQ ID 141 SI-54E1 and SI-54E3 chain B nt
CAGATCGTGCTGAGCCAGAGCCCCGCCATCCTGAGCGCCAGCCCCGGCGAGAAGGTGACCATGACCTGCCGGGCCAG
CAGCAGCGTGAGCTACATCCACTGGTTCCAGCAGAAGCCCGGCAGCAGCCCCAAGCCCTGGATCTACGCCACCAGCA
ACCTGGCCAGCGGCGTGCCCGTGCGGTTCAGCGGCAGCGGCAGCGGCACCAGCTACAGCCTGACCATCAGCCGGGTG
GAGGCCGAGGACGCCGCCACCTACTACTGCCAGCAGTGGACCAGCAACCCCCCCACCTTCGGCGGCGGCACCAAGCT
GGAGATCAAGCGTACGGTGGCTGCACCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAAGGTGGATAACGCCCTCCAA
TCGGGTAACTCCCAGGAGAGTGTCACAGAGCAGGACAGCAAGGACAGCACCTACAGCCTCAGCAGCACCCTGACGCT
GAGCAAAGCAGACTACGAGAAACACAAAGTCTACGCCTGCGAAGTCACCCATCAGGGCCTGAGCTCGCCCGTCACAA
AGAGCTTCAACAGGGGAGAGTGTTAG
>SEQ ID 142 SI-54E1 and SI-54E3 chain B aa
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKLEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC
>SEQ ID 143 SI-54E3 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGCAGCCCGGCGCCGAGCTGGTGAAGCCCGGCGCCAGCGTGAAGATGA
GCTGCAAGGCCAGCGGCTACACCTTCACCAGCTACAACATGCACTGGGTGAAGCAGACCCCCGGCCGGGGCCTGGAG
TGGATCGGCGCCATCTACCCCGGCAACGGCGACACCAGCTACAACCAGAAGTTCAAGGGCAAGGCCACCCTGACCGC
CGACAAGAGCAGCAGCACCGCCTACATGCAGCTGAGCAGCCTGACCAGCGAGGACAGCGCCGTGTACTACTGCGCCC
GGAGCACCTACTACGGCGGCGACTGGTACTTCAACGTGTGGGGCGCCGGCACCACCGTGACCGTGAGCGCCGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACGCCAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTG
CAGCCTCTGGATTCTCCTTCAGTAGCGGGTACGACATGTGCTGGGTCCGCCAGGCTCCAGGGAAGTGCCTGGAGTGG
ATCGCATGCATTGCTGCTGGTAGTGCTGGTATCACTTACGACGCGAACTGGGCGAAAGGCCGGTTCACCATCTCCAG
AGACAATTCCAAGAACACGCTGTATCTGCAAATGAACAGCCTGAGAGCCGAGGACACGGCCGTATATTACTGTGCGA
GATCGGCGTTTTCGTTCGACTACGCCATGGACCTCTGGGGCCAGGGAACCCTGGTCACCGTCTCGAGCGGTGGAGGC
GGTTCAGGCGGAGGTGGAAGTGGTGGTGGCGGCTCTGGAGGCGGCGGATCTGACATCCAGATGACCCAGTCTCCTTC
CACCCTGTCTGCATCTGTAGGAGACAGAGTCACCATCACTTGCCAGGCCAGTCAGAGCATTAGTTCCCACTTAAACT
GGTATCAGCAGAAACCAGGGAAAGCCCCTAAGCTCCTGATCTATAAGGCATCCACTCTGGCATCTGGGGTCCCATCA
AGGTTCAGCGGCAGTGGATCTGGGACAGAATTTACTCTCACCATCAGCAGCCTGCAGCCTGATGATTTTGCAACTTA
TTACTGCCAACAGGGTTATAGTTGGGGTAATGTTGATAATGTTTTCGGCTGCGGGACCAAGGTGGAGATCAAAGGTG
GTGGCGGCTCTGGAGGAGGAGGGTCCGGACGGTCGCTGGTGGAGTCTGGGGGAGGCTTGGTCCAGCCTGGGGGGTCC
CTGAGACTCTCCTGTACTGCCTCTGGATTCACCATCAGTAGCTACCACATGCAGTGGGTCCGGCAGGCACCTGGGAA
GTGCCTGGAGTACATCGGAACCATTAGTAGTGGTGGTAATGTATACTACGCAAGCTCCGCTAGAGGCAGATTCACCA
TCTCCAGACCCTCGTCCAAGAACACGGTGGATCTTCAAATGAACAGCCTGAGAGCCGAGGACACGGCTGTGTATTAC
TGTGCGAGAGACTCTGGTTATAGTGATCCTATGTGGGGCCAGGGAACCCTGGTCACCGTCTCTTCAGGCGGTGGCGG
TAGTGGGGGAGGCGGTTCTGGCGGCGGAGGGTCCGGCGGTGGAGGATCAGACGTTGTGATGACCCAGTCTCCATCTT
CCGTGTCTGCATCTGTAGGAGACAGAGTCACCATCACCTGTCAGGCCAGTCAGAACATTAGGACTTACTTATCCTGG
TATCAGCAGAAACCAGGGAAAGCCCCTAAGCTCCTGATCTATGCTGCAGCCAATCTGGCATCTGGGGTCCCATCAAG
GTTCAGCGGCAGTGGATCTGGGACAGATTTCACTCTCACCATCAGCGACTTGGAACCTGGCGATGCTGCAACTTACT
ATTGTCAGTCTACCTATCTTGGTACTGATTATGTTGGCGGTGCTTTCGGCTGTGGGACCAAGGTGGAGATCAAATGA
>SEQ ID 144 SI-54E3 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLQQPGAELVKPGASVKMSCKASGYTFTSYNMHWVKQTPGRGLE
WIGAIYPGNGDTSYNQKFKGKATLTADKSSSTAYMQLSSLTSEDSAVYYCARSTYYGGDWYFNVWGAGTTVTVSAAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGGPSVFLFPPKPKDTLMISRTPEVTCVVVDVSHEDPEV
KFNWYVDGVEVHNAKTKPREEQYASTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGGGGGSGGGGSEVQLLESGGGLVQPGGSLRLSCAASGFSFSSGYDMCWVRQAPGKCLEW
IACIAAGSAGITYDANWAKGRFTISRDNSKNTLYLQMNSLRAEDTAVYYCARSAFSFDYAMDLWGQGTLVTVSSGGG
GSGGGGSGGGGSGGGGSDIQMTQSPSTLSASVGDRVTITCQASQSISSHLNWYQQKPGKAPKLLIYKASTLASGVPS
RFSGSGSGTEFTLTISSLQPDDFATYYCQQGYSWGNVDNVFGCGTKVEIKGGGGSGGGGSGRSLVESGGGLVQPGGS
LRLSCTASGFTISSYHMQWVRQAPGKCLEYIGTISSGGNVYYASSARGRFTISRPSSKNTVDLQMNSLRAEDTAVYY
CARDSGYSDPMWGQGTLVTVSSGGGGSGGGGSGGGGSGGGGSDVVMTQSPSSVSASVGDRVTITCQASQNIRTYLSW
YQQKPGKAPKLLIYAAANLASGVPSRFSGSGSGTDFTLTISDLEPGDAATYYCQSTYLGTDYVGGAFGCGTKVEIK
>SEQ ID 145 SI-55E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGAAGCAGTCAGGACCTGGCCTAGTGCAGCCCTCACAGAGCCTGTCCATCA
CCTGCACAGTCTCTGGTTTCTCATTAACTAACTATGGTGTACACTGGGTTCGCCAGTCTCCAGGAAAGGGTCTGGAG
TGGCTGGGAGTGATATGGAGTGGTGGAAACACAGACTATAATACACCTTTCACATCCAGACTGAGCATCAACAAGGA
CAATTCCAAGAGCCAAGTTTTCTTTAAAATGAACAGTCTGCAATCTAATGACACAGCCATATATTACTGTGCCAGAG
CCCTCACCTACTATGATTACGAGTTTGCTTACTGGGGCCAAGGGACTCTGGTCACTGTCTCTAGCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGCACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGGGGCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGATCT
GGCGGAGGTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATCAAAGGCGGTGGAG
GGTCCGGCGGTGGTGGCTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGCCAGGCTCCAGGGAAGGGGCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACCTGGAGCCTGGCGATGCTGCAACTTACTATTGTC
AGTCTACCTATCTTGGTACTGATTATGTTGGCGGTGCTTTCGGCGGAGGGACCAAGGTGGAGATCAAATGA
>SEQ ID 146 SI-55E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLKQSGPGLVQPSQSLSITCTVSGFSLTNYGVHWVRQSPGKGLE
WLGVIWSGGNTDYNTPFTSRLSINKDNSKSQVFFKMNSLQSNDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVEIKGGGGSGGGGSGRSLVESGGGLVQPGGSLR
LSCTASGFTISSYHMQWVRQAPGKGLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGGGTKVEIK
>SEQ ID 147 SI-55E1 and SI-55E8 chain B nt
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>SEQ ID 148 SI-55E1 and SI-55E8 chain B aa
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>SEQ ID 149 SI-55E8 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGAAGCAGTCAGGACCTGGCCTAGTGCAGCCCTCACAGAGCCTGTCCATCA
CCTGCACAGTCTCTGGTTTCTCATTAACTAACTATGGTGTACACTGGGTTCGCCAGTCTCCAGGAAAGGGTCTGGAG
TGGCTGGGAGTGATATGGAGTGGTGGAAACACAGACTATAATACACCTTTCACATCCAGACTGAGCATCAACAAGGA
CAATTCCAAGAGCCAAGTTTTCTTTAAAATGAACAGTCTGCAATCTAATGACACAGCCATATATTACTGTGCCAGAG
CCCTCACCTACTATGATTACGAGTTTGCTTACTGGGGCCAAGGGACTCTGGTCACTGTCTCTAGCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACGCCAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGTGCCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGTTCA
GGCGGAGGTGGAAGTGGTGGTGGCGGCTCTGGAGGCGGCGGATCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCTGCGGGACCAAGGTGGAGATCAAAGGTGGTGGCG
GCTCTGGAGGAGGAGGGTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGGCAGGCACCTGGGAAGTGCCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACTTGGAACCTGGCGATGCTGCAACTTACTATTGTC
AGTCTACCTATCTTGGTACTGATTATGTTGGCGGTGCTTTCGGCTGTGGGACCAAGGTGGAGATCAAATGA
>SEQ ID 150 SI-55E8 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLKQSGPGLVQPSQSLSITCTVSGFSLTNYGVHWVRQSPGKGLE
WLGVIWSGGNTDYNTPFTSRLSINKDNSKSQVFFKMNSLQSNDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKCLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGCGTKVEIKGGGGSGGGGSGRSLVESGGGLVQPGGSLR
LSCTASGFTISSYHMQWVRQAPGKCLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGCGTKVEIK
>SEQ ID 151 SI-55E2 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGGAGTCGGGCCCAGGACTGGTGAAGCCTTCGGAGACCCTGTCCCTCA
CCTGCACTGTCTCTGGTGGCTCCGTCAGCAGTGGTGATTACTACTGGACCTGGATCCGGCAGTCCCCAGGGAAGGGA
CTGGAGTGGATTGGACACATCTATTACAGTGGGAACACCAATTATAACCCCTCCCTCAAGAGCCGACTCACCATATC
AATTGACACGTCCAAGACTCAGTTCTCCCTGAAGCTGAGTTCTGTGACCGCTGCGGACACGGCCATTTATTACTGTG
TGCGAGATCGAGTGACTGGTGCTTTTGATATCTGGGGCCAAGGGACAATGGTCACCGTCTCGAGCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGCACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGGGGCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGATCT
GGCGGAGGTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATCAAAGGCGGTGGAG
GGTCCGGCGGTGGTGGCTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGCCAGGCTCCAGGGAAGGGGCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACCTGGAGCCTGGCGATGCTGCAACTTACTATTGTC
AGTCTACCTATCTTGGTACTGATTATGTTGGCGGTGCTTTCGGCGGAGGGACCAAGGTGGAGATCAAATGA
>SEQ ID 152 SI-55E2 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLQESGPGLVKPSETLSLTCTVSGGSVSSGDYYWTWIRQSPGKG
LEWIGHIYYSGNTNYNPSLKSRLTISIDTSKTQFSLKLSSVTAADTAIYYCVRDRVTGAFDIWGQGTMVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVEIKGGGGSGGGGSGRSLVESGGGLVQPGGSLR
LSCTASGFTISSYHMQWVRQAPGKGLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGGGTKVEIK
>SEQ ID 153 SI-55E2 and SI-55E9 chain B nt
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCGGAGGGACCAA
GGTGGAAATTAAACGTACGGTGGCTGCACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTACAGCCTCAGCAGCACCCTGAC
GCTGAGCAAAGCAGACTACGAGAAACACAAAGTCTACGCCTGCGAAGTCACCCATCAGGGCCTGAGCTCGCCCGTCA
CAAAGAGCTTCAACAGGGGAGAGTGTTAG
>SEQ ID 154 SI-55E2 and SI-55E9 chain B aa
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
>SEQ ID 155 SI-55E9 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGGAGTCGGGCCCAGGACTGGTGAAGCCTTCGGAGACCCTGTCCCTCA
CCTGCACTGTCTCTGGTGGCTCCGTCAGCAGTGGTGATTACTACTGGACCTGGATCCGGCAGTCCCCAGGGAAGGGA
CTGGAGTGGATTGGACACATCTATTACAGTGGGAACACCAATTATAACCCCTCCCTCAAGAGCCGACTCACCATATC
AATTGACACGTCCAAGACTCAGTTCTCCCTGAAGCTGAGTTCTGTGACCGCTGCGGACACGGCCATTTATTACTGTG
TGCGAGATCGAGTGACTGGTGCTTTTGATATCTGGGGCCAAGGGACAATGGTCACCGTCTCGAGCGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACGCCAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGTGCCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTCTCGAGCGGTGGAGGCGGTTCA
GGCGGAGGTGGAAGTGGTGGTGGCGGCTCTGGAGGCGGCGGATCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCTGCGGGACCAAGGTGGAGATCAAAGGTGGTGGCG
GCTCTGGAGGAGGAGGGTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGGCAGGCACCTGGGAAGTGCCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACTTGGAACCTGGCGATGCTGCAACTTACTATTGTC
AGTCTACCTATCTTGGTACTGATTATGTTGGCGGTGCTTTCGGCTGTGGGACCAAGGTGGAGATCAAATGA
>SEQ ID 156 SI-55E9 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLQESGPGLVKPSETLSLTCTVSGGSVSSGDYYWTWIRQSPGKG
LEWIGHIYYSGNTNYNPSLKSRLTISIDTSKTQFSLKLSSVTAADTAIYYCVRDRVTGAFDIWGQGTMVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGGPSVFLFPPKPKDTLMISRTPEVTCVVVDVSHEDPEVKF
NWYVDGVEVHNAKTKPREEQYASTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKCLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGCGTKVEIKGGGGSGGGGSGRSLVESGGGLVQPGGSLR
LSCTASGFTISSYHMQWVRQAPGKCLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGCGTKVEIK
>SEQ ID 157 SI-55E3 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGCAGAGCGGCGCCGAGGTGAAGAAGCCCGGCAGCAGCGTGAAGGTGA
GCTGCAAGGCCAGCGGCTACACCTTCACCAACTACTACATCTACTGGGTGCGGCAGGCCCCCGGCCAGGGCCTGGAG
TGGATCGGCGGCATCAACCCCACCAGCGGCGGCAGCAACTTCAACGAGAAGTTCAAGACCCGGGTGACCATCACCGC
CGACGAGAGCAGCACCACCGCCTACATGGAGCTGAGCAGCCTGCGGAGCGAGGACACCGCCTTCTACTTCTGCACCC
GGCAGGGCCTGTGGTTCGACAGCGACGGCCGGGGCTTCGACTTCTGGGGCCAGGGCACCACCGTGACCGTGAGCAGC
GCTAGCACCAAGGGCCCATCGGTCTTCCCCCTGGCACCCTCCTCCAAGAGCACCTCTGGGGGCACAGCGGCCCTGGG
CTGCCTGGTCAAGGACTACTTCCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTGAGCCCAAATCTTG
TGACAAAACTCACACATGCCCACCGTGCCCAGCACCTGAAGCCGCGGGGGCACCGTCAGTCTTCCTCTTCCCCCCAA
AACCCAAGGACACCCTCATGATCTCCCGGACCCCTGAGGTCACATGCGTGGTGGTGGACGTGAGCCACGAAGACCCT
GAGGTCAAGTTCAACTGGTACGTGGACGGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGCTGAATGGCAAGGAGTACAAGTGCGCGG
TCTCCAACAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTGGCGGTGGAGGGTC
CGGCGGTGGTGGATCCGAGGTGCAGCTGTTGGAGTCTGGGGGAGGCTTGGTACAGCCTGGGGGGTCCCTGAGACTCT
CCTGTGCAGCCTCTGGATTCTCCTTCAGTAGCGGGTACGACATGTGCTGGGTCCGCCAGGCTCCAGGGAAGGGGCTG
GAGTGGATCGCATGCATTGCTGCTGGTAGTGCTGGTATCACTTACGACGCGAACTGGGCGAAAGGCCGGTTCACCAT
CTCCAGAGACAATTCCAAGAACACGCTGTATCTGCAAATGAACAGCCTGAGAGCCGAGGACACGGCCGTATATTACT
GTGCGAGATCGGCGTTTTCGTTCGACTACGCCATGGACCTCTGGGGCCAGGGAACCCTGGTCACCGTCTCGAGCGGT
GGAGGCGGATCTGGCGGAGGTGGTTCCGGCGGTGGCGGCTCCGGTGGAGGCGGCTCTGACATCCAGATGACCCAGTC
TCCTTCCACCCTGTCTGCATCTGTAGGAGACAGAGTCACCATCACTTGCCAGGCCAGTCAGAGCATTAGTTCCCACT
TAAACTGGTATCAGCAGAAACCAGGGAAAGCCCCTAAGCTCCTGATCTATAAGGCATCCACTCTGGCATCTGGGGTC
CCATCAAGGTTCAGCGGCAGTGGATCTGGGACAGAATTTACTCTCACCATCAGCAGCCTGCAGCCTGATGATTTTGC
AACTTATTACTGCCAACAGGGTTATAGTTGGGGTAATGTTGATAATGTTTTCGGCGGAGGGACCAAGGTGGAGATCA
AAGGCGGTGGAGGGTCCGGCGGTGGTGGCTCCGGACGGTCGCTGGTGGAGTCTGGGGGAGGCTTGGTCCAGCCTGGG
GGGTCCCTGAGACTCTCCTGTACTGCCTCTGGATTCACCATCAGTAGCTACCACATGCAGTGGGTCCGCCAGGCTCC
AGGGAAGGGGCTGGAGTACATCGGAACCATTAGTAGTGGTGGTAATGTATACTACGCAAGCTCCGCTAGAGGCAGAT
TCACCATCTCCAGACCCTCGTCCAAGAACACGGTGGATCTTCAAATGAACAGCCTGAGAGCCGAGGACACGGCTGTG
TATTACTGTGCGAGAGACTCTGGTTATAGTGATCCTATGTGGGGCCAGGGAACCCTGGTCACCGTCTCTTCAGGCGG
TGGCGGTAGTGGGGGAGGCGGTTCTGGCGGCGGAGGGTCCGGCGGTGGAGGATCAGACGTTGTGATGACCCAGTCTC
CATCTTCCGTGTCTGCATCTGTAGGAGACAGAGTCACCATCACCTGTCAGGCCAGTCAGAACATTAGGACTTACTTA
TCCTGGTATCAGCAGAAACCAGGGAAAGCCCCTAAGCTCCTGATCTATGCTGCAGCCAATCTGGCATCTGGGGTCCC
ATCAAGGTTCAGCGGCAGTGGATCTGGGACAGATTTCACTCTCACCATCAGCGACCTGGAGCCTGGCGATGCTGCAA
CTTACTATTGTCAGTCTACCTATCTTGGTACTGATTATGTTGGCGGTGCTTTCGGCGGAGGGACCAAGGTGGAGATC
AAATGA
>SEQ ID 158 SI-55E3 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQVQLQQSGAEVKKPGSSVKVSCKASGYTFTNYYIYWVRQAPGQGLE
WIGGINPTSGGSNFNEKFKTRVTITADESSTTAYMELSSLRSEDTAFYFCTRQGLWFDSDGRGFDFWGQGTTVTVSS
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPEAAGAPSVFLFPPKPKDTLMISRTPEVTCVVVDVSHEDP
EVKFNWYVDGVEVHNAKTKPREEQYNSTYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQQGNVFSC
SVMHEALHNHYTQKSLSLSPGGGGGSGGGGSEVQLLESGGGLVQPGGSLRLSCAASGFSFSSGYDMCWVRQAPGKGL
EWIACIAAGSAGITYDANWAKGRFTISRDNSKNTLYLQMNSLRAEDTAVYYCARSAFSFDYAMDLWGQGTLVTVSSG
GGGSGGGGSGGGGSGGGGSDIQMTQSPSTLSASVGDRVTITCQASQSISSHLNWYQQKPGKAPKLLIYKASTLASGV
PSRFSGSGSGTEFTLTISSLQPDDFATYYCQQGYSWGNVDNVFGGGTKVEIKGGGGSGGGGSGRSLVESGGGLVQPG
GSLRLSCTASGFTISSYHMQWVRQAPGKGLEYIGTISSGGNVYYASSARGRFTISRPSSKNTVDLQMNSLRAEDTAV
YYCARDSGYSDPMWGQGTLVTVSSGGGGSGGGGSGGGGSGGGGSDVVMTQSPSSVSASVGDRVTITCQASQNIRTYL
SWYQQKPGKAPKLLIYAAANLASGVPSRFSGSGSGTDFTLTISDLEPGDAATYYCQSTYLGTDYVGGAFGGGTKVEI
K
>SEQ ID 159 SI-55E3 and SI-55E10 chain B nt
GATATTCAAATGACTCAATCTCCTTCTTCTCTTTCTGCTTCTGTTGGTGATCGTGTTACTATTACTTGTCGTTCTTC
TCAAAATATTGTTCATTCTAATGGTAATACTTATCTTGATTGGTATCAACAAACTCCTGGTAAAGCTCCTAAACTTC
TTATTTATAAAGTTTCTAATCGTTTTTCTGGTGTTCCTTCTCGTTTTTCTGGTTCTGGTTCTGGTACTGATTTTACT
TTTACTATTTCTTCTCTTCAACCTGAAGATATTGCTACTTATTATTGTTTTCAATATTCTCATGTTCCTTGGACTTT
TGGTCAAGGTACTAAACTTCAAATTACTCGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAG
>SEQ ID 160 SI-55E3 and SI-55E10 chain B aa
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQITRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>SEQ ID 161 SI-55E10 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGGTGCAGCTGCAGCAGAGCGGCGCCGAGGTGAAGAAGCCCGGCAGCAGCGTGAAGGTGA
GCTGCAAGGCCAGCGGCTACACCTTCACCAACTACTACATCTACTGGGTGCGGCAGGCCCCCGGCCAGGGCCTGGAG
TGGATCGGCGGCATCAACCCCACCAGCGGCGGCAGCAACTTCAACGAGAAGTTCAAGACCCGGGTGACCATCACCGC
CGACGAGAGCAGCACCACCGCCTACATGGAGCTGAGCAGCCTGCGGAGCGAGGACACCGCCTTCTACTTCTGCACCC
GGCAGGGCCTGTGGTTCGACAGCGACGGCCGGGGCTTCGACTTCTGGGGCCAGGGCACCACCGTGACCGTGAGCAGC
GCTAGCACCAAGGGCCCATCGGTCTTCCCCCTGGCACCCTCCTCCAAGAGCACCTCTGGGGGCACAGCGGCCCTGGG
CTGCCTGGTCAAGGACTACTTCCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGGACAAGAGAGTTGAGCCCAAATCTTG
TGACAAAACTCACACATGCCCACCGTGCCCAGCACCTGAAGCCGCGGGGGGACCGTCAGTCTTCCTCTTCCCCCCAA
AACCCAAGGACACCCTCATGATCTCCCGGACCCCTGAGGTCACATGCGTGGTGGTGGACGTGAGCCACGAAGACCCT
GAGGTCAAGTTCAACTGGTACGTGGACGGCGTGGAGGTGCATAATGCCAAGACAAAGCCGCGGGAGGAGCAGTACGC
CAGCACGTACCGTGTGGTCAGCGTCCTCACCGTCCTGCACCAGGACTGGCTGAATGGCAAGGAGTACAAGTGCGCGG
TCTCCAACAAAGCCCTCCCAGCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAGCCGGAGAACAACTACAAGACCACGCCTCCCGTGCTGGACT
CCGACGGCTCCTTCTTCCTCTATAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTGGCGGTGGAGGGTC
CGGCGGTGGTGGATCCGAGGTGCAGCTGTTGGAGTCTGGGGGAGGCTTGGTACAGCCTGGGGGGTCCCTGAGACTCT
CCTGTGCAGCCTCTGGATTCTCCTTCAGTAGCGGGTACGACATGTGCTGGGTCCGCCAGGCTCCAGGGAAGTGCCTG
GAGTGGATCGCATGCATTGCTGCTGGTAGTGCTGGTATCACTTACGACGCGAACTGGGCGAAAGGCCGGTTCACCAT
CTCCAGAGACAATTCCAAGAACACGCTGTATCTGCAAATGAACAGCCTGAGAGCCGAGGACACGGCCGTATATTACT
GTGCGAGATCGGCGTTTTCGTTCGACTACGCCATGGACCTCTGGGGCCAGGGAACCCTGGTCACCGTCTCGAGCGGT
GGAGGCGGTTCAGGCGGAGGTGGAAGTGGTGGTGGCGGCTCTGGAGGCGGCGGATCTGACATCCAGATGACCCAGTC
TCCTTCCACCCTGTCTGCATCTGTAGGAGACAGAGTCACCATCACTTGCCAGGCCAGTCAGAGCATTAGTTCCCACT
TAAACTGGTATCAGCAGAAACCAGGGAAAGCCCCTAAGCTCCTGATCTATAAGGCATCCACTCTGGCATCTGGGGTC
CCATCAAGGTTCAGCGGCAGTGGATCTGGGACAGAATTTACTCTCACCATCAGCAGCCTGCAGCCTGATGATTTTGC
AACTTATTACTGCCAACAGGGTTATAGTTGGGGTAATGTTGATAATGTTTTCGGCTGCGGGACCAAGGTGGAGATCA
AAGGTGGTGGCGGCTCTGGAGGAGGAGGGTCCGGACGGTCGCTGGTGGAGTCTGGGGGAGGCTTGGTCCAGCCTGGG
GGGTCCCTGAGACTCTCCTGTACTGCCTCTGGATTCACCATCAGTAGCTACCACATGCAGTGGGTCCGGCAGGCACC
TGGGAAGTGCCTGGAGTACATCGGAACCATTAGTAGTGGTGGTAATGTATACTACGCAAGCTCCGCTAGAGGCAGAT
TCACCATCTCCAGACCCTCGTCCAAGAACACGGTGGATCTTCAAATGAACAGCCTGAGAGCCGAGGACACGGCTGTG
TATTACTGTGCGAGAGACTCTGGTTATAGTGATCCTATGTGGGGCCAGGGAACCCTGGTCACCGTCTCTTCAGGCGG
TGGCGGTAGTGGGGGAGGCGGTTCTGGCGGCGGAGGGTCCGGCGGTGGAGGATCAGACGTTGTGATGACCCAGTCTC
CATCTTCCGTGTCTGCATCTGTAGGAGACAGAGTCACCATCACCTGTCAGGCCAGTCAGAACATTAGGACTTACTTA
TCCTGGTATCAGCAGAAACCAGGGAAAGCCCCTAAGCTCCTGATCTATGCTGCAGCCAATCTGGCATCTGGGGTCCC
ATCAAGGTTCAGCGGCAGTGGATCTGGGACAGATTTCACTCTCACCATCAGCGACTTGGAACCTGGCGATGCTGCAA
CTTACTATTGTCAGTCTACCTATCTTGGTACTGATTATGTTGGCGGTGCTTTCGGCTGTGGGACCAAGGTGGAGATC
AAATGA
>SEQ ID 162 SI-55E10 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQVQLQQSGAEVKKPGSSVKVSCKASGYTFTNYYIYWVRQAPGQGLE
WIGGINPTSGGSNFNEKFKTRVTITADESSTTAYMELSSLRSEDTAFYFCTRQGLWFDSDGRGFDFWGQGTTVTVSS
ASTKGPSVFPLAPSSKSTSGGTAALGCLVKDYFPEPVTVSWNSGALTSGVHTFPAVLQSSGLYSLSSVVTVPSSSLG
TQTYICNVNHKPSNTKVDKRVEPKSCDKTHTCPPCPAPEAAGGPSVFLFPPKPKDTLMISRTPEVTCVVVDVSHEDP
EVKFNWYVDGVEVHNAKTKPREEQYASTYRVVSVLTVLHQDWLNGKEYKCAVSNKALPAPIEKTISKAKGQPREPQV
YTLPPSRDELTKNQVSLTCLVKGFYPSDIAVEWESNGQPENNYKTTPPVLDSDGSFFLYSKLTVDKSRWQQGNVFSC
SVMHEALHNHYTQKSLSLSPGGGGGSGGGGSEVQLLESGGGLVQPGGSLRLSCAASGFSFSSGYDMCWVRQAPGKCL
EWIACIAAGSAGITYDANWAKGRFTISRDNSKNTLYLQMNSLRAEDTAVYYCARSAFSFDYAMDLWGQGTLVTVSSG
GGGSGGGGSGGGGSGGGGSDIQMTQSPSTLSASVGDRVTITCQASQSISSHLNWYQQKPGKAPKLLIYKASTLASGV
PSRFSGSGSGTEFTLTISSLQPDDFATYYCQQGYSWGNVDNVFGCGTKVEIKGGGGSGGGGSGRSLVESGGGLVQPG
GSLRLSCTASGFTISSYHMQWVRQAPGKCLEYIGTISSGGNVYYASSARGRFTISRPSSKNTVDLQMNSLRAEDTAV
YYCARDSGYSDPMWGQGTLVTVSSGGGGSGGGGSGGGGSGGGGSDVVMTQSPSSVSASVGDRVTITCQASQNIRTYL
SWYQQKPGKAPKLLIYAAANLASGVPSRFSGSGSGTDFTLTISDLEPGDAATYYCQSTYLGTDYVGGAFGCGTKVEI
K
>SEQ ID 163 SI-56E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAGGTGCAATTGGTCCAGTCAGGAGCAGAGGTTAAGAAGCCTGGTGCTTCCGTCAAAGTTT
CGTGTAAGGCCAGCGGCTACAGGTTCACAAATTATTGGATTCATTGGGTCAGGCAGGCTCCGGGACAAGGCCTGGAA
TGGATCGGTGGCATTAATCCCGGGAATAACTACGCTACATATAGGAGAAAATTCCAGGGCAGAGTTACGATGACCGC
GGACACCTCCACAAGCACTGTCTACATGGAGCTGTCATCTCTGAGATCCGAGGACACCGCAGTGTACTATTGTACTA
GAGAAGGCTACGGTAATTACGGAGCCTGGTTCGCCTACTGGGGCCAGGGTACCCTAGTCACAGTCTCCTCAGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGCACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACAACAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTG
CAGCCTCTGGATTCTCCTTCAGTAGCGGGTACGACATGTGCTGGGTCCGCCAGGCTCCAGGGAAGGGGCTGGAGTGG
ATCGCATGCATTGCTGCTGGTAGTGCTGGTATCACTTACGACGCGAACTGGGCGAAAGGCCGGTTCACCATCTCCAG
AGACAATTCCAAGAACACGCTGTATCTGCAAATGAACAGCCTGAGAGCCGAGGACACGGCCGTATATTACTGTGCGA
GATCGGCGTTTTCGTTCGACTACGCCATGGACCTCTGGGGCCAGGGAACCCTGGTCACCGTCTCGAGCGGTGGAGGC
GGATCTGGCGGAGGTGGTTCCGGCGGTGGCGGCTCCGGTGGAGGCGGCTCTGACATCCAGATGACCCAGTCTCCTTC
CACCCTGTCTGCATCTGTAGGAGACAGAGTCACCATCACTTGCCAGGCCAGTCAGAGCATTAGTTCCCACTTAAACT
GGTATCAGCAGAAACCAGGGAAAGCCCCTAAGCTCCTGATCTATAAGGCATCCACTCTGGCATCTGGGGTCCCATCA
AGGTTCAGCGGCAGTGGATCTGGGACAGAATTTACTCTCACCATCAGCAGCCTGCAGCCTGATGATTTTGCAACTTA
TTACTGCCAACAGGGTTATAGTTGGGGTAATGTTGATAATGTTTTCGGCGGAGGGACCAAGGTGGAGATCAAAGGCG
GTGGAGGGTCCGGCGGTGGTGGCTCCGGACGGTCGCTGGTGGAGTCTGGGGGAGGCTTGGTCCAGCCTGGGGGGTCC
CTGAGACTCTCCTGTACTGCCTCTGGATTCACCATCAGTAGCTACCACATGCAGTGGGTCCGCCAGGCTCCAGGGAA
GGGGCTGGAGTACATCGGAACCATTAGTAGTGGTGGTAATGTATACTACGCAAGCTCCGCTAGAGGCAGATTCACCA
TCTCCAGACCCTCGTCCAAGAACACGGTGGATCTTCAAATGAACAGCCTGAGAGCCGAGGACACGGCTGTGTATTAC
TGTGCGAGAGACTCTGGTTATAGTGATCCTATGTGGGGCCAGGGAACCCTGGTCACCGTCTCTTCAGGCGGTGGCGG
TAGTGGGGGAGGCGGTTCTGGCGGCGGAGGGTCCGGCGGTGGAGGATCAGACGTTGTGATGACCCAGTCTCCATCTT
CCGTGTCTGCATCTGTAGGAGACAGAGTCACCATCACCTGTCAGGCCAGTCAGAACATTAGGACTTACTTATCCTGG
TATCAGCAGAAACCAGGGAAAGCCCCTAAGCTCCTGATCTATGCTGCAGCCAATCTGGCATCTGGGGTCCCATCAAG
GTTCAGCGGCAGTGGATCTGGGACAGATTTCACTCTCACCATCAGCGACCTGGAGCCTGGCGATGCTGCAACTTACT
ATTGTCAGTCTACCTATCTTGGTACTGATTATGTTGGCGGTGCTTTCGGCGGAGGGACCAAGGTGGAGATCAAATGA
>SEQ ID 164 SI-56E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGFTISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSEVQLVQSGAEVKKPGASVKVSCKASGYRFTNYWIHWVRQAPGQGLE
WIGGINPGNNYATYRRKFQGRVTMTADTSTSTVYMELSSLRSEDTAVYYCTREGYGNYGAWFAYWGQGTLVTVSSAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGAPSVFLFPPKPKDTLMISRTPEVTCVVVDVSHEDPEV
KFNWYVDGVEVHNAKTKPREEQYNSTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGGGGGSGGGGSEVQLLESGGGLVQPGGSLRLSCAASGFSFSSGYDMCWVRQAPGKGLEW
IACIAAGSAGITYDANWAKGRFTISRDNSKNTLYLQMNSLRAEDTAVYYCARSAFSFDYAMDLWGQGTLVTVSSGGG
GSGGGGSGGGGSGGGGSDIQMTQSPSTLSASVGDRVTITCQASQSISSHLNWYQQKPGKAPKLLIYKASTLASGVPS
RFSGSGSGTEFTLTISSLQPDDFATYYCQQGYSWGNVDNVFGGGTKVEIKGGGGSGGGGSGRSLVESGGGLVQPGGS
LRLSCTASGFTISSYHMQWVRQAPGKGLEYIGTISSGGNVYYASSARGRFTISRPSSKNTVDLQMNSLRAEDTAVYY
CARDSGYSDPMWGQGTLVTVSSGGGGSGGGGSGGGGSGGGGSDVVMTQSPSSVSASVGDRVTITCQASQNIRTYLSW
YQQKPGKAPKLLIYAAANLASGVPSRFSGSGSGTDFTLTISDLEPGDAATYYCQSTYLGTDYVGGAFGGGTKVEIK
>SEQ ID 165 SI-56E1 and SI-56E3 chain B nt
GACGTTCAAGTGACCCAGAGCCCATCCAGCCTGAGCGCATCTGTAGGAGACCGGGTCACCATCACTTGTAGATCCAG
TCAGAGTCTTGCAAACAGTTATGGGAACACCTTTTTGTCTTGGTATCTGCACAAACCAGGTAAAGCCCCACAATTGC
TCATCTACGGAATCTCTAACAGATTTAGTGGTGTACCAGACAGGTTCAGCGGTTCCGGAAGTGGTACTGATTTCACC
CTCACGATCTCGTCTCTCCAGCCAGAAGATTTCGCCACTTATTACTGTTTACAAGGTACACATCAGCCGTACACATT
CGGTCAGGGTACTAAAGTAGAAATCAAACGTACGGTGGCTGCACCATCTGTCTTCATCTTCCCGCCATCTGATGAGC
AGTTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGCCT
CAGCAGCACCCTGACGCTGAGCAAAGCAGACTACGAGAAACACAAAGTCTACGCCTGCGAAGTCACCCATCAGGGCC
TGAGCTCGCCCGTCACAAAGAGCTTCAACAGGGGAGAGTGTTAG
>SEQ ID 166 SI-56E1 and SI-56E3 chain B aa
DVQVTQSPSSLSASVGDRVTITCRSSQSLANSYGNTFLSWYLHKPGKAPQLLIYGISNRFSGVPDRFSGSGSGTDFT
LTISSLQPEDFATYYCLQGTHQPYTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
>SEQ ID 167 SI-56E3 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCAGAGGTGCAATTGGTCCAGTCAGGAGCAGAGGTTAAGAAGCCTGGTGCTTCCGTCAAAGTTT
CGTGTAAGGCCAGCGGCTACAGGTTCACAAATTATTGGATTCATTGGGTCAGGCAGGCTCCGGGACAAGGCCTGGAA
TGGATCGGTGGCATTAATCCCGGGAATAACTACGCTACATATAGGAGAAAATTCCAGGGCAGAGTTACGATGACCGC
GGACACCTCCACAAGCACTGTCTACATGGAGCTGTCATCTCTGAGATCCGAGGACACCGCAGTGTACTATTGTACTA
GAGAAGGCTACGGTAATTACGGAGCCTGGTTCGCCTACTGGGGCCAGGGTACCCTAGTCACAGTCTCCTCAGCTAGC
ACCAAGGGCCCATCGGTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAGAGTTGAGCCCAAATCTTGTGACAA
AACTCACACATGCCCACCGTGCCCAGCACCTGAAGCCGCGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGAGGTC
AAGTTCAACTGGTACGTGGACGGCGTGGAGGTGCATAATGCCAAGACAAAGCCGCGGGAGGAGCAGTACGCCAGCAC
GTACCGTGTGGTCAGCGTCCTCACCGTCCTGCACCAGGACTGGCTGAATGGCAAGGAGTACAAGTGCGCGGTCTCCA
ACAAAGCCCTCCCAGCCCCCATCGAGAAAACCATCTCCAAAGCCAAAGGGCAGCCCCGAGAACCACAGGTGTACACC
CTGCCCCCATCCCGGGATGAGCTGACCAAGAACCAGGTCAGCCTGACCTGCCTGGTCAAAGGCTTCTATCCCAGCGA
CATCGCCGTGGAGTGGGAGAGCAATGGGCAGCCGGAGAACAACTACAAGACCACGCCTCCCGTGCTGGACTCCGACG
GCTCCTTCTTCCTCTATAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTGGCGGTGGAGGGTCCGGCGG
TGGTGGATCCGAGGTGCAGCTGTTGGAGTCTGGGGGAGGCTTGGTACAGCCTGGGGGGTCCCTGAGACTCTCCTGTG
CAGCCTCTGGATTCTCCTTCAGTAGCGGGTACGACATGTGCTGGGTCCGCCAGGCTCCAGGGAAGTGCCTGGAGTGG
ATCGCATGCATTGCTGCTGGTAGTGCTGGTATCACTTACGACGCGAACTGGGCGAAAGGCCGGTTCACCATCTCCAG
AGACAATTCCAAGAACACGCTGTATCTGCAAATGAACAGCCTGAGAGCCGAGGACACGGCCGTATATTACTGTGCGA
GATCGGCGTTTTCGTTCGACTACGCCATGGACCTCTGGGGCCAGGGAACCCTGGTCACCGTCTCGAGCGGTGGAGGC
GGTTCAGGCGGAGGTGGAAGTGGTGGTGGCGGCTCTGGAGGCGGCGGATCTGACATCCAGATGACCCAGTCTCCTTC
CACCCTGTCTGCATCTGTAGGAGACAGAGTCACCATCACTTGCCAGGCCAGTCAGAGCATTAGTTCCCACTTAAACT
GGTATCAGCAGAAACCAGGGAAAGCCCCTAAGCTCCTGATCTATAAGGCATCCACTCTGGCATCTGGGGTCCCATCA
AGGTTCAGCGGCAGTGGATCTGGGACAGAATTTACTCTCACCATCAGCAGCCTGCAGCCTGATGATTTTGCAACTTA
TTACTGCCAACAGGGTTATAGTTGGGGTAATGTTGATAATGTTTTCGGCTGCGGGACCAAGGTGGAGATCAAAGGTG
GTGGCGGCTCTGGAGGAGGAGGGTCCGGACGGTCGCTGGTGGAGTCTGGGGGAGGCTTGGTCCAGCCTGGGGGGTCC
CTGAGACTCTCCTGTACTGCCTCTGGATTCACCATCAGTAGCTACCACATGCAGTGGGTCCGGCAGGCACCTGGGAA
GTGCCTGGAGTACATCGGAACCATTAGTAGTGGTGGTAATGTATACTACGCAAGCTCCGCTAGAGGCAGATTCACCA
TCTCCAGACCCTCGTCCAAGAACACGGTGGATCTTCAAATGAACAGCCTGAGAGCCGAGGACACGGCTGTGTATTAC
TGTGCGAGAGACTCTGGTTATAGTGATCCTATGTGGGGCCAGGGAACCCTGGTCACCGTCTCTTCAGGCGGTGGCGG
TAGTGGGGGAGGCGGTTCTGGCGGCGGAGGGTCCGGCGGTGGAGGATCAGACGTTGTGATGACCCAGTCTCCATCTT
CCGTGTCTGCATCTGTAGGAGACAGAGTCACCATCACCTGTCAGGCCAGTCAGAACATTAGGACTTACTTATCCTGG
TATCAGCAGAAACCAGGGAAAGCCCCTAAGCTCCTGATCTATGCTGCAGCCAATCTGGCATCTGGGGTCCCATCAAG
GTTCAGCGGCAGTGGATCTGGGACAGATTTCACTCTCACCATCAGCGACTTGGAACCTGGCGATGCTGCAACTTACT
ATTGTCAGTCTACCTATCTTGGTACTGATTATGTTGGCGGTGCTTTCGGCTGTGGGACCAAGGTGGAGATCAAATGA
>SEQ ID 168 SI-56E3 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSEVQLVQSGAEVKKPGASVKVSCKASGYRFTNYWIHWVRQAPGQGLE
WIGGINPGNNYATYRRKFQGRVTMTADTSTSTVYMELSSLRSEDTAVYYCTREGYGNYGAWFAYWGQGTLVTVSSAS
TKGPSVFPLAPSSKSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPEAAGGPSVFLFPPKPKDTLMISRTPEVTCVVVDVSHEDPEV
KFNWYVDGVEVHNAKTKPREEQYASTYRVVSVLTVLHQDWLNGKEYKCAVSNKALPAPIEKTISKAKGQPREPQVYT
LPPSRDELTKNQVSLTCLVKGFYPSDIAVEWESNGQPENNYKTTPPVLDSDGSFFLYSKLTVDKSRWQQGNVFSCSV
MHEALHNHYTQKSLSLSPGGGGGSGGGGSEVQLLESGGGLVQPGGSLRLSCAASGFSFSSGYDMCWVRQAPGKCLEW
IACIAAGSAGITYDANWAKGRFTISRDNSKNTLYLQMNSLRAEDTAVYYCARSAFSFDYAMDLWGQGTLVTVSSGGG
GSGGGGSGGGGSGGGGSDIQMTQSPSTLSASVGDRVTITCQASQSISSHLNWYQQKPGKAPKLLIYKASTLASGVPS
RFSGSGSGTEFTLTISSLQPDDFATYYCQQGYSWGNVDNVFGCGTKVEIKGGGGSGGGGSGRSLVESGGGLVQPGGS
LRLSCTASGFTISSYHMQWVRQAPGKCLEYIGTISSGGNVYYASSARGRFTISRPSSKNTVDLQMNSLRAEDTAVYY
CARDSGYSDPMWGQGTLVTVSSGGGGSGGGGSGGGGSGGGGSDVVMTQSPSSVSASVGDRVTITCQASQNIRTYLSW
YQQKPGKAPKLLIYAAANLASGVPSRFSGSGSGTDFTLTISDLEPGDAATYYCQSTYLGTDYVGGAFGCGTKVEIK
>SEQ ID 169 SI-57E1 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAAGGCGGTGGCGGTAGTGGGGGAGGCGGTTCTGGCGGCGGAGGGTCCGGCG
GTGGAGGATCAGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGGGGCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGATCCAGCTGCAGCAGTCTGGACCTGAGGTGGTGAAGCCTGGGGCTTCAGTGAAGATAT
CCTGCAAGGCTTCTGGCTACACCTTCACTGACTACTATATAACCTGGGTGAAGCAGAAGCCTGGACAGGGACTTGAG
TGGATTGGATGGATTTATCCTGGAAGCGGTAATACTAAGTACAATGAGAAGTTCAAGGGCAAGGCCACATTGACTGT
AGACACATCCTCCAGCACAGCCTTCATGCAGCTCAGCAGCCTGACATCTGAGGACACTGCTGTCTATTTCTGTGCGA
ACTATGGTAACTACTGGTTTGCTTACTGGGGCCAAGGGACTCAGGTCACTGTCTCTGCAGCTAGCACCAAGGGCCCA
TCGGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTGC
AACGTGAATCACAAGCCCAGCAACACCAAGGTGGACAAGAGAGTTGAGCCCAAATCTTGTGACAAAACTCACACATG
CCCACCGTGCCCAGCACCTGAAGCCGCGGGGGCACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACAACAGCACGTACCGTGTGGT
CAGCGTCCTCACCGTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGTGCTGGACTCCGACGGCTCCTTCTTCC
TCTATAGCAAGCTCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTGGCGGTGGAGGGTCCGGCGGTGGTGGATCCGA
GGTGCAGCTGTTGGAGTCTGGGGGAGGCTTGGTACAGCCTGGGGGGTCCCTGAGACTCTCCTGTGCAGCCTCTGGAT
TCTCCTTCAGTAGCGGGTACGACATGTGCTGGGTCCGCCAGGCTCCAGGGAAGGGGCTGGAGTGGATCGCATGCATT
GCTGCTGGTAGTGCTGGTATCACTTACGACGCGAACTGGGCGAAAGGCCGGTTCACCATCTCCAGAGACAATTCCAA
GAACACGCTGTATCTGCAAATGAACAGCCTGAGAGCCGAGGACACGGCCGTATATTACTGTGCGAGATCGGCGTTTT
CGTTCGACTACGCCATGGACCTCTGGGGCCAGGGAACCCTGGTCACCGTCTCGAGCGGTGGAGGCGGATCTGGCGGA
GGTGGTTCCGGCGGTGGCGGCTCCGGTGGAGGCGGCTCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAACTGGTATCAGCAGA
AACCAGGGAAAGCCCCTAAGCTCCTGATCTATAAGGCATCCACTCTGGCATCTGGGGTCCCATCAAGGTTCAGCGGC
AGTGGATCTGGGACAGAATTTACTCTCACCATCAGCAGCCTGCAGCCTGATGATTTTGCAACTTATTACTGCCAACA
GGGTTATAGTTGGGGTAATGTTGATAATGTTTTCGGCGGAGGGACCAAGGTGGAGATCAAAGGCGGTGGAGGGTCCG
GCGGTGGTGGCTCCGGACGGTCGCTGGTGGAGTCTGGGGGAGGCTTGGTCCAGCCTGGGGGGTCCCTGAGACTCTCC
TGTACTGCCTCTGGATTCACCATCAGTAGCTACCACATGCAGTGGGTCCGCCAGGCTCCAGGGAAGGGGCTGGAGTA
CATCGGAACCATTAGTAGTGGTGGTAATGTATACTACGCAAGCTCCGCTAGAGGCAGATTCACCATCTCCAGACCCT
CGTCCAAGAACACGGTGGATCTTCAAATGAACAGCCTGAGAGCCGAGGACACGGCTGTGTATTACTGTGCGAGAGAC
TCTGGTTATAGTGATCCTATGTGGGGCCAGGGAACCCTGGTCACCGTCTCTTCAGGCGGTGGCGGTAGTGGGGGAGG
CGGTTCTGGCGGCGGAGGGTCCGGCGGTGGAGGATCAGACGTTGTGATGACCCAGTCTCCATCTTCCGTGTCTGCAT
CTGTAGGAGACAGAGTCACCATCACCTGTCAGGCCAGTCAGAACATTAGGACTTACTTATCCTGGTATCAGCAGAAA
CCAGGGAAAGCCCCTAAGCTCCTGATCTATGCTGCAGCCAATCTGGCATCTGGGGTCCCATCAAGGTTCAGCGGCAG
TGGATCTGGGACAGATTTCACTCTCACCATCAGCGACCTGGAGCCTGGCGATGCTGCAACTTACTATTGTCAGTCTA
CCTATCTTGGTACTGATTATGTTGGCGGTGCTTTCGGCGGAGGGACCAAGGTGGAGATCAAATGA
>SEQ ID 170 SI-57E1 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIKGGGGSGGGGSGGGGSGGGGSEVQLVESGGGLVQPGGSLRLSC
AASGETISTNAMSWVRQAPGKGLEWIGVITGRDITYYASWAKGRFTISRDNSKNTLYLQMNSLRAEDTAVYYCARDG
GSSAITSNNIWGQGTLVTVSTGGGGSGGGGSQIQLQQSGPEVVKPGASVKISCKASGYTFTDYYITWVKQKPGQGLE
WIGWIYPGSGNTKYNEKFKGKATLTVDTSSSTAFMQLSSLTSEDTAVYFCANYGNYWFAYWGQGTQVTVSAASTKGP
SVFPLAPSSKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPEAAGAPSVFLFPPKPKDTLMISRTPEVTCVVVDVSHEDPEVKFNW
YVDGVEVHNAKTKPREEQYNSTYRVVSVLTVLHQDWLNGKEYKCAVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQGNVFSCSVMHEA
LHNHYTQKSLSLSPGGGGGSGGGGSEVQLLESGGGLVQPGGSLRLSCAASGFSFSSGYDMCWVRQAPGKGLEWIACI
AAGSAGITYDANWAKGRFTISRDNSKNTLYLQMNSLRAEDTAVYYCARSAFSFDYAMDLWGQGTLVTVSSGGGGSGG
GGSGGGGSGGGGSDIQMTQSPSTLSASVGDRVTITCQASQSISSHLNWYQQKPGKAPKLLIYKASTLASGVPSRFSG
SGSGTEFTLTISSLQPDDFATYYCQQGYSWGNVDNVFGGGTKVEIKGGGGSGGGGSGRSLVESGGGLVQPGGSLRLS
CTASGFTISSYHMQWVRQAPGKGLEYIGTISSGGNVYYASSARGRFTISRPSSKNTVDLQMNSLRAEDTAVYYCARD
SGYSDPMWGQGTLVTVSSGGGGSGGGGSGGGGSGGGGSDVVMTQSPSSVSASVGDRVTITCQASQNIRTYLSWYQQK
PGKAPKLLIYAAANLASGVPSRFSGSGSGTDFTLTISDLEPGDAATYYCQSTYLGTDYVGGAFGGGTKVEIK
>SEQ ID 171 SI-57E1 and SI-57E3 chain B nt
GACATTGTGCTGACCCAATCTCCAGCTTCTTTGGCTGTGTCTCTAGGGCAGAGGGCCACCATCTCCTGCAAGGCCAG
CCAAAGTGTTGATTTTGATGGTGATAGTTATATGAACTGGTACCAACAGAAACCAGGACAGCCACCCAAAGTCCTCA
TCTATGCTGCATCCAATCTAGAATCTGGGATCCCAGCCAGGTTTAGTGGCAGTGGGTCTGGGACAGACTTCACCCTC
AACATCCATCCTGTGGAGGAGGAGGATGCTGCAACCTATTACTGTCAGCAAAGTAATGAGGATCCGTGGACGTTCGG
TGGAGGCACCAAGCTGGAAATCAAACGTACGGTGGCTGCACCATCTGTCTTCATCTTCCCGCCATCTGATGAGCAGT
TGAAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTCAG
CAGCACCCTGACGCTGAGCAAAGCAGACTACGAGAAACACAAAGTCTACGCCTGCGAAGTCACCCATCAGGGCCTGA
GCTCGCCCGTCACAAAGAGCTTCAACAGGGGAGAGTGTTAG
>SEQ ID 172 SI-57E1 and SI-57E3 chain B aa
DIVLTQSPASLAVSLGQRATISCKASQSVDFDGDSYMNWYQQKPGQPPKVLIYAASNLESGIPARFSGSGSGTDFTL
NIHPVEEEDAATYYCQQSNEDPWTFGGGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
>SEQ ID 173 SI-57E3 chain A nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCTGTGGGACCAAGGTGGAGATCAAAGGTGGAGGCGGTTCAGGCGGAGGTGGAAGTGGTGGTGGCGGCTCTGGAG
GCGGCGGATCTGAGGTGCAGCTGGTGGAGTCTGGGGGAGGCTTGGTCCAGCCTGGGGGGTCCCTGAGACTCTCCTGT
GCAGCCTCTGGATTCACCATCAGTACCAATGCAATGAGCTGGGTCCGCCAGGCTCCAGGGAAGTGCCTGGAGTGGAT
CGGAGTCATTACTGGTCGTGATATCACATACTACGCGAGCTGGGCGAAAGGCAGATTCACCATCTCCAGAGACAATT
CCAAGAACACGCTGTATCTTCAAATGAACAGCCTGAGAGCCGAGGACACGGCTGTGTATTACTGTGCGAGAGACGGT
GGTTCTTCTGCTATTACTAGTAACAACATTTGGGGCCAGGGAACCCTGGTCACCGTGTCGACAGGCGGTGGAGGGTC
CGGCGGTGGTGGATCACAGATCCAGCTGCAGCAGTCTGGACCTGAGGTGGTGAAGCCTGGGGCTTCAGTGAAGATAT
CCTGCAAGGCTTCTGGCTACACCTTCACTGACTACTATATAACCTGGGTGAAGCAGAAGCCTGGACAGGGACTTGAG
TGGATTGGATGGATTTATCCTGGAAGCGGTAATACTAAGTACAATGAGAAGTTCAAGGGCAAGGCCACATTGACTGT
AGACACATCCTCCAGCACAGCCTTCATGCAGCTCAGCAGCCTGACATCTGAGGACACTGCTGTCTATTTCTGTGCGA
ACTATGGTAACTACTGGTTTGCTTACTGGGGCCAAGGGACTCAGGTCACTGTCTCTGCAGCTAGCACCAAGGGCCCA
TCGGTCTTCCCCCTGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTCCCGGCTGTCCTAC
AGTCCTCAGGACTCTACTCCCTCAGCAGCGTGGTGACCGTGCCCTCCAGCAGCTTGGGCACCCAGACCTACATCTGC
AACGTGAATCACAAGCCCAGCAACACCAAGGTGGACAAGAGAGTTGAGCCCAAATCTTGTGACAAAACTCACACATG
CCCACCGTGCCCAGCACCTGAAGCCGCGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACGCCAGCACGTACCGTGTGGT
CAGCGTCCTCACCGTCCTGCACCAGGACTGGCTGAATGGCAAGGAGTACAAGTGCGCGGTCTCCAACAAAGCCCTCC
CAGCCCCCATCGAGAAAACCATCTCCAAAGCCAAAGGGCAGCCCCGAGAACCACAGGTGTACACCCTGCCCCCATCC
CGGGATGAGCTGACCAAGAACCAGGTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGTGCTGGACTCCGACGGCTCCTTCTTCC
TCTATAGCAAGCTCACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTGGCGGTGGAGGGTCCGGCGGTGGTGGATCCGA
GGTGCAGCTGTTGGAGTCTGGGGGAGGCTTGGTACAGCCTGGGGGGTCCCTGAGACTCTCCTGTGCAGCCTCTGGAT
TCTCCTTCAGTAGCGGGTACGACATGTGCTGGGTCCGCCAGGCTCCAGGGAAGTGCCTGGAGTGGATCGCATGCATT
GCTGCTGGTAGTGCTGGTATCACTTACGACGCGAACTGGGCGAAAGGCCGGTTCACCATCTCCAGAGACAATTCCAA
GAACACGCTGTATCTGCAAATGAACAGCCTGAGAGCCGAGGACACGGCCGTATATTACTGTGCGAGATCGGCGTTTT
CGTTCGACTACGCCATGGACCTCTGGGGCCAGGGAACCCTGGTCACCGTCTCGAGCGGTGGAGGCGGTTCAGGCGGA
GGTGGAAGTGGTGGTGGCGGCTCTGGAGGCGGCGGATCTGACATCCAGATGACCCAGTCTCCTTCCACCCTGTCTGC
ATCTGTAGGAGACAGAGTCACCATCACTTGCCAGGCCAGTCAGAGCATTAGTTCCCACTTAAACTGGTATCAGCAGA
AACCAGGGAAAGCCCCTAAGCTCCTGATCTATAAGGCATCCACTCTGGCATCTGGGGTCCCATCAAGGTTCAGCGGC
AGTGGATCTGGGACAGAATTTACTCTCACCATCAGCAGCCTGCAGCCTGATGATTTTGCAACTTATTACTGCCAACA
GGGTTATAGTTGGGGTAATGTTGATAATGTTTTCGGCTGCGGGACCAAGGTGGAGATCAAAGGTGGTGGCGGCTCTG
GAGGAGGAGGGTCCGGACGGTCGCTGGTGGAGTCTGGGGGAGGCTTGGTCCAGCCTGGGGGGTCCCTGAGACTCTCC
TGTACTGCCTCTGGATTCACCATCAGTAGCTACCACATGCAGTGGGTCCGGCAGGCACCTGGGAAGTGCCTGGAGTA
CATCGGAACCATTAGTAGTGGTGGTAATGTATACTACGCAAGCTCCGCTAGAGGCAGATTCACCATCTCCAGACCCT
CGTCCAAGAACACGGTGGATCTTCAAATGAACAGCCTGAGAGCCGAGGACACGGCTGTGTATTACTGTGCGAGAGAC
TCTGGTTATAGTGATCCTATGTGGGGCCAGGGAACCCTGGTCACCGTCTCTTCAGGCGGTGGCGGTAGTGGGGGAGG
CGGTTCTGGCGGCGGAGGGTCCGGCGGTGGAGGATCAGACGTTGTGATGACCCAGTCTCCATCTTCCGTGTCTGCAT
CTGTAGGAGACAGAGTCACCATCACCTGTCAGGCCAGTCAGAACATTAGGACTTACTTATCCTGGTATCAGCAGAAA
CCAGGGAAAGCCCCTAAGCTCCTGATCTATGCTGCAGCCAATCTGGCATCTGGGGTCCCATCAAGGTTCAGCGGCAG
TGGATCTGGGACAGATTTCACTCTCACCATCAGCGACTTGGAACCTGGCGATGCTGCAACTTACTATTGTCAGTCTA
CCTATCTTGGTACTGATTATGTTGGCGGTGCTTTCGGCTGTGGGACCAAGGTGGAGATCAAATGA
>SEQ ID 174 SI-57E3 chain A aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGCGTKVEIKGGGGSGGGGSGGGGSGGGGSEVQLVESGGGLVQPGGSLRLSC
AASGFTISTNAMSWVRQAPGKCLEWIGVITGRDITYYASWAKGRFTISRDNSKNTLYLQMNSLRAEDTAVYYCARDG
GSSAITSNNIWGQGTLVTVSTGGGGSGGGGSQIQLQQSGPEVVKPGASVKISCKASGYTFTDYYITWVKQKPGQGLE
WIGWIYPGSGNTKYNEKFKGKATLTVDTSSSTAFMQLSSLTSEDTAVYFCANYGNYWFAYWGQGTQVTVSAASTKGP
SVFPLAPSSKSTSGGTAALGCLVKDYFPEPVTVSWNSGALTSGVHTFPAVLQSSGLYSLSSVVTVPSSSLGTQTYIC
NVNHKPSNTKVDKRVEPKSCDKTHTCPPCPAPEAAGGPSVFLFPPKPKDTLMISRTPEVTCVVVDVSHEDPEVKFNW
YVDGVEVHNAKTKPREEQYASTYRVVSVLTVLHQDWLNGKEYKCAVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQGNVFSCSVMHEA
LHNHYTQKSLSLSPGGGGGSGGGGSEVQLLESGGGLVQPGGSLRLSCAASGFSFSSGYDMCWVRQAPGKCLEWIACI
AAGSAGITYDANWAKGRFTISRDNSKNTLYLQMNSLRAEDTAVYYCARSAFSFDYAMDLWGQGTLVTVSSGGGGSGG
GGSGGGGSGGGGSDIQMTQSPSTLSASVGDRVTITCQASQSISSHLNWYQQKPGKAPKLLIYKASTLASGVPSRFSG
SGSGTEFTLTISSLQPDDFATYYCQQGYSWGNVDNVFGCGTKVEIKGGGGSGGGGSGRSLVESGGGLVQPGGSLRLS
CTASGFTISSYHMQWVRQAPGKCLEYIGTISSGGNVYYASSARGRFTISRPSSKNTVDLQMNSLRAEDTAVYYCARD
SGYSDPMWGQGTLVTVSSGGGGSGGGGSGGGGSGGGGSDVVMTQSPSSVSASVGDRVTITCQASQNIRTYLSWYQQK
PGKAPKLLIYAAANLASGVPSRFSGSGSGTDFTLTISDLEPGDAATYYCQSTYLGTDYVGGAFGCGTKVEIK
>SEQ ID 175 SI-1H1 chain A nt
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCGGTGGGACCAA
GGTGGAAATTAAAGGCGGTGGAGGGTCCGGCGGTGGTGGATCACAGGTGCAGCTGCAGGAGTCGGGCCCAGGACTGG
TGAAGCCTTCGGAGACCCTGTCCCTCACCTGCACTGTCTCTGGTGGCTCCGTCAGCAGTGGTGATTACTACTGGACC
TGGATCCGGCAGTCCCCAGGGAAGGGACTGGAGTGGATTGGACACATCTATTACAGTGGGAACACCAATTATAACCC
CTCCCTCAAGAGCCGACTCACCATATCAATTGACACGTCCAAGACTCAGTTCTCCCTGAAGCTGAGTTCTGTGACCG
CTGCGGACACGGCCATTTATTACTGTGTGCGAGATCGAGTGACTGGTGCTTTTGATATCTGGGGCCAAGGGACAATG
GTCACCGTCTCGAGCGGTGGAGGCGGATCTGGCGGAGGTGGTTCCGGCGGTGGCGGCTCCGGTGGAGGCGGCTCTGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTACACCCTGCCCCCATCCCGGGATGAG
CTGACCAAGAACCAGGTCAGCCTGACCTGCCTGGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGTGGAGGGTCCGGCGGTGGTGGATCCGAGGTGCAGCT
GTTGGAGTCTGGGGGAGGCTTGGTACAGCCTGGGGGGTCCCTGAGACTCTCCTGTGCAGCCTCTGGATTCTCCTTCA
GTAGCGGGTACGACATGTGCTGGGTCCGCCAGGCTCCAGGGAAGGGGCTGGAGTGGATCGCATGCATTGCTGCTGGT
AGTGCTGGTATCACTTACGACGCGAACTGGGCGAAAGGCCGGTTCACCATCTCCAGAGACAATTCCAAGAACACGCT
GTATCTGCAAATGAACAGCCTGAGAGCCGAGGACACGGCCGTATATTACTGTGCGAGATCGGCGTTTTCGTTCGACT
ACGCCATGGACCTCTGGGGCCAGGGAACCCTGGTCACCGTCTCGAGCGGTGGAGGCGGATCTGGCGGAGGTGGTTCC
GGCGGTGGCGGCTCCGGTGGAGGCGGCTCTGACATCCAGATGACCCAGTCTCCTTCCACCCTGTCTGCATCTGTAGG
AGACAGAGTCACCATCACTTGCCAGGCCAGTCAGAGCATTAGTTCCCACTTAAACTGGTATCAGCAGAAACCAGGGA
AAGCCCCTAAGCTCCTGATCTATAAGGCATCCACTCTGGCATCTGGGGTCCCATCAAGGTTCAGCGGCAGTGGATCT
GGGACAGAATTTACTCTCACCATCAGCAGCCTGCAGCCTGATGATTTTGCAACTTATTACTGCCAACAGGGTTATAG
TTGGGGTAATGTTGATAATGTTTTCGGCGGAGGGACCAAGGTGGAGATCAAAGGCGGTGGAGGGTCCGGCGGTGGTG
GCTCCGGACGGTCGCTGGTGGAGTCTGGGGGAGGCTTGGTCCAGCCTGGGGGGTCCCTGAGACTCTCCTGTACTGCC
TCTGGATTCACCATCAGTAGCTACCACATGCAGTGGGTCCGCCAGGCTCCAGGGAAGGGGCTGGAGTACATCGGAAC
CATTAGTAGTGGTGGTAATGTATACTACGCAAGCTCCGCTAGAGGCAGATTCACCATCTCCAGACCCTCGTCCAAGA
ACACGGTGGATCTTCAAATGAACAGCCTGAGAGCCGAGGACACGGCTGTGTATTACTGTGCGAGAGACTCTGGTTAT
AGTGATCCTATGTGGGGCCAGGGAACCCTGGTCACCGTCTCTTCAGGCGGTGGCGGTAGTGGGGGAGGCGGTTCTGG
CGGCGGAGGGTCCGGCGGTGGAGGATCAGACGTTGTGATGACCCAGTCTCCATCTTCCGTGTCTGCATCTGTAGGAG
ACAGAGTCACCATCACCTGTCAGGCCAGTCAGAACATTAGGACTTACTTATCCTGGTATCAGCAGAAACCAGGGAAA
GCCCCTAAGCTCCTGATCTATGCTGCAGCCAATCTGGCATCTGGGGTCCCATCAAGGTTCAGCGGCAGTGGATCTGG
GACAGATTTCACTCTCACCATCAGCGACCTGGAGCCTGGCGATGCTGCAACTTACTATTGTCAGTCTACCTATCTTG
GTACTGATTATGTTGGCGGTGCTTTCGGCGGAGGGACCAAGGTGGAGATCAAATGA
>SEQ ID 176 SI-1H1 chain A aa
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIKGGGGSGGGGSQVQLQESGPGLVKPSETLSLTCTVSGGSVSSGDYYWT
WIRQSPGKGLEWIGHIYYSGNTNYNPSLKSRLTISIDTSKTQFSLKLSSVTAADTAIYYCVRDRVTGAFDIWGQGTM
VTVSSGGGGSGGGGSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNVFSCSVMHEALHN
HYTQKSLSLSPGGGGGSGGGGSEVQLLESGGGLVQPGGSLRLSCAASGFSFSSGYDMCWVRQAPGKGLEWIACIAAG
SAGITYDANWAKGRFTISRDNSKNTLYLQMNSLRAEDTAVYYCARSAFSFDYAMDLWGQGTLVTVSSGGGGSGGGGS
GGGGSGGGGSDIQMTQSPSTLSASVGDRVTITCQASQSISSHLNWYQQKPGKAPKLLIYKASTLASGVPSRFSGSGS
GTEFTLTISSLQPDDFATYYCQQGYSWGNVDNVFGGGTKVEIKGGGGSGGGGSGRSLVESGGGLVQPGGSLRLSCTA
SGFTISSYHMQWVRQAPGKGLEYIGTISSGGNVYYASSARGRFTISRPSSKNTVDLQMNSLRAEDTAVYYCARDSGY
SDPMWGQGTLVTVSSGGGGSGGGGSGGGGSGGGGSDVVMTQSPSSVSASVGDRVTITCQASQNIRTYLSWYQQKPGK
APKLLIYAAANLASGVPSRFSGSGSGTDFTLTISDLEPGDAATYYCQSTYLGTDYVGGAFGGGTKVEIK
>SEQ ID 177 SI-1H1 chain B nt
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTAGGTGGAGGCGGTTCAGGCGGAGGTGGTTCCGGCGGTGGCGGCTCCGGTGGAG
GCGGCTCTCAGGTGCAATTGCAGGAGTCGGGGGGAGGCCTGGTCAAGCCTGGAGGGTCCCTGAGACTCTCCTGTGCA
GCCTCTGGATTCACCTTTAGTAGTTATTGGATGAGCTGGGTCCGCCAGGCTCCAGGGAAGGGGCTGGAGTGGGTGGC
CAACATAAACCGCGATGGAAGTGCGAGTTACTATGTGGACTCTGTGAAGGGCCGATTCACCATCTCCAGAGACGACG
CCAAGAACTCACTGTATCTGCAAATGAACAGCCTGAGAGCTGAGGACACGGCTGTGTATTACTGTGCGAGAGATCGT
GGGGTGGGCTACTTCGATCTCTGGGGCCGTGGCACCCTGGTCACCGTCTCGAGCGGCGGTGGAGGGTCCGGCGGTGG
TGGATCCGACGTCGTGATGACCCAGTCTCCTTCCACCCTGTCTGCATCTGTAGGAGACAGAGTCACCATCAATTGCC
AAGCCAGTGAGAGCATTAGCAGTTGGTTAGCCTGGTATCAGCAGAAACCAGGGAAAGCCCCTAAGCTCCTGATCTAT
GAAGCATCCAAACTGGCATCTGGGGTCCCATCAAGGTTCAGCGGCAGTGGATCTGGGACAGAATTTACTCTCACCAT
CAGCAGCCTGCAGCCTGATGATTTTGCAACTTATTACTGCCAAGGCTATTTTTATTTTATTAGTCGTACTTATGTAA
ATTCTTTCGGCGGAGGGACCAAGGTGGAGATCAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGCAAGGACAGCACCT
ACAGCCTCAGCAGCACCCTGACGCTGAGCAAAGCAGACTACGAGAAACACAAAGTCTACGCCTGCGAAGTCACCCAT
CAGGGCCTGAGCTCGCCCGTCACAAAGAGCTTCAACAGGGGAGAGTGTGGCGGTGGAGGGTCCGGCGGTGGTGGATC
CCAGGTCACATTGAAGGAATCTGGCCCCGGCCTTGTTCAGCCAGGACAGACCCTTAGGCTCACCTGTGCCTTCAGTG
GTTTTTCTCTTAGCACTAGCGGTATGGGGGTCGGCTGGATTCGGCAGCCTCCCGGCAAAGGTCTTGAGTGGTTGGCT
CACATTTGGTGGGACGACGACAAACGGTATAATCCTGCCTTGAAAAGTCGGCTGACCATTAGTAAGGATACCTCAAA
AAATCAAGTGTACTTGCAAATGAATAGCCTTGACGCCGAGGATACGGCTGTATATTATTGCGCGCGGATGGAACTCT
GGTCTTACTACTTTGATTATTGGGGGCAGGGGACTCTCGTCACGGTCTCGAGTGGCGGTGGCGGTAGTGGGGGAGGC
GGTTCTGGCGGCGGAGGGTCCGGCGGTGGAGGATCAGAAAATGTATTGACACAGAGCCCCGCCTCCCTCAGTGCCTC
ACCTGGGGAAAGGGTAACTATCACTTGCTCTGCATCAAGCAGCGTCTCATACATGCATTGGTATCAACAAAAGCCTG
GACAGGCCCCCAAGCTCTGGATATACGATACGAGCAAGCTGGCTTCCGGCGTACCTAGCCGCTTCAGTGGTTCCGGC
TCAGGCAACGATCACACCCTTACGATTTCCAGTATGGAACCCGAAGATTTTGCAACTTATTATTGTTTCCAGGGGAG
CGTGTACCCATTCACTTTCGGGCAGGGGACAAAATTGGAGATAAAGTAG
>SEQ ID 178 SI-1H1 chain B aa
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVLGGGGSGGGGSGGGGSGGGGSQVQLQESGGGLVKPGGSLRLSCA
ASGFTFSSYWMSWVRQAPGKGLEWVANINRDGSASYYVDSVKGRFTISRDDAKNSLYLQMNSLRAEDTAVYYCARDR
GVGYFDLWGRGTLVTVSSGGGGSGGGGSDVVMTQSPSTLSASVGDRVTINCQASESISSWLAWYQQKPGKAPKLLIY
EASKLASGVPSRFSGSGSGTEFTLTISSLQPDDFATYYCQGYFYFISRTYVNSFGGGTKVEIKRTVAAPSVFIFPPS
DEQLKSGTASVVCLLNNFYPREAKVQWKVDNALQSGNSQESVTEQDSKDSTYSLSSTLTLSKADYEKHKVYACEVTH
QGLSSPVTKSFNRGECGGGGSGGGGSQVTLKESGPGLVQPGQTLRLTCAFSGFSLSTSGMGVGWIRQPPGKGLEWLA
HIWWDDDKRYNPALKSRLTISKDTSKNQVYLQMNSLDAEDTAVYYCARMELWSYYFDYWGQGTLVTVSSGGGGSGGG
GSGGGGSGGGGSENVLTQSPASLSASPGERVTITCSASSSVSYMHWYQQKPGQAPKLWIYDTSKLASGVPSRFSGSG
SGNDHTLTISSMEPEDFATYYCFQGSVYPFTFGQGTKLEIK
>SEQ ID 183 SI-49R19 chain A nt
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGTGGTGGGGGTAGTGGCGG
CGGAGGTAGCTTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGT
>SEQ ID 184 SI-49R19 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPG
>SEQ ID 185 SI-49R19 chain B nt
TTCCTAAACTCATTATTCAACCAAGAAGTTCAAATTCCCTTGACCGAAAGTTACTGTGGCCCATGTCCTAAAAACTG
GATATGTTACAAAAATAACTGCTACCAATTTTTTGATGAGAGTAAAAACTGGTATGAGAGCCAGGCTTCTTGTATGT
CTCAAAATGCCAGCCTTCTGAAAGTATACAGCAAAGAGGACCAGGATTTACTTAAACTGGTGAAGTCATATCATTGG
ATGGGACTAGTACACATTCCAACAAATGGATCTTGGCAGTGGGAAGATGGCTCCATTCTCTCACCCAACCTACTAAC
AATAATTGAAATGCAGAAGGGAGACTGTGCACTCTATGCCTCGAGCTTTAAAGGCTATATAGAAAACTGTTCAACTC
CAAATACGTACATCTGCATGCAAAGGACTGTGCGTACGGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGT
>SEQ ID 186 SI-49R19 chain B aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVRTVAAPSVFIFPPSD
EQLKSGTASVVCLLNNFYPREAKVQWKVDNALQSGNSQESVTEQDSKDSTYSLSSTLTLSKADYEKHKVYACEVTHQ
GLSSPVTKSFNRGEC
>SEQ ID 187 SI-49R18 chain A nt
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGTGGTGGGGGTAGTGGCGG
CGGAGGTAGCTTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGT
>SEQ ID 188 SI-49R18 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPG
>SEQ ID 189 SI-49R18 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAATT
GGAGATAAAGGGAAGTACTGGTTCTGGGTCTAAACCCGGTTCCGGCGAAGGTAGTACTAAAGGACAGGTCACATTGA
AGGAATCTGGCCCCGGCCTTGTTCAGCCAGGACAGACCCTTAGGCTCACCTGTGCCTTCAGTGGTTTTTCTCTTAGC
ACTAGCGGTATGGGGGTCGGCTGGATTCGGCAGCCTCCCGGCAAAGGTCTTGAGTGGTTGGCTCACATTTGGTGGGA
CGACGACAAACGGTATAATCCTGCCTTGAAAAGTCGGCTGACCATTAGTAAGGATACCTCAAAAAATCAAGTGTACT
TGCAAATGAATAGCCTTGACGCCGAGGATACGGCTGTATATTATTGCGCGCGGATGGAACTCTGGTCTTACTACTTT
GATTATTGGGGGCAGGGGACTCTCGTCACGGTGTCCTCTGGTGGCGGCGGCTCCGGGGGTGGCGGATCAGGTGGTGG
AGGATCCTTCCTAAACTCATTATTCAACCAAGAAGTTCAAATTCCCTTGACCGAAAGTTACTGTGGCCCATGTCCTA
AAAACTGGATATGTTACAAAAATAACTGCTACCAATTTTTTGATGAGAGTAAAAACTGGTATGAGAGCCAGGCTTCT
TGTATGTCTCAAAATGCCAGCCTTCTGAAAGTATACAGCAAAGAGGACCAGGATTTACTTAAACTGGTGAAGTCATA
TCATTGGATGGGACTAGTACACATTCCAACAAATGGATCTTGGCAGTGGGAAGATGGCTCCATTCTCTCACCCAACC
TACTAACAATAATTGAAATGCAGAAGGGAGACTGTGCACTCTATGCCTCGAGCTTTAAAGGCTATATAGAAAACTGT
TCAACTCCAAATACGTACATCTGCATGCAAAGGACTGTGCGTACGGTGGCTGCACCATCTGTCTTCATCTTCCCGCC
ATCTGATGAGCAGTTGAAATCTGGAACTGCCTCTGTTGTGTGCCTGCTGAATAACTTCTATCCCAGAGAGGCCAAAG
TACAGTGGAAGGTGGATAACGCCCTCCAATCGGGTAACTCCCAGGAGAGTGTCACAGAGCAGGACAGCAAGGACAGC
ACCTACAGCCTCAGCAGCACCCTGACGCTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGTGT
>SEQ ID 190 SI-49R18 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKLEIKGSTGSGSKPGSGEGSTKGQVTLKESGPGLVQPGQTLRLTCAFSGFSLS
TSGMGVGWIRQPPGKGLEWLAHIWWDDDKRYNPALKSRLTISKDTSKNQVYLQMNSLDAEDTAVYYCARMELWSYYF
DYWGQGTLVTVSSGGGGSGGGGSGGGGSFLNSLFNQEVQIPLTESYCGPCPKNWICYKNNCYQFFDESKNWYESQAS
CMSQNASLLKVYSKEDQDLLKLVKSYHWMGLVHIPTNGSWQWEDGSILSPNLLTIIEMQKGDCALYASSFKGYIENG
STPNTYICMQRTVRTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
>SEQ ID 191 SI-49E15 chain A nt
GAGATCGTGATGACACAATCTCCATCTACGCTCTCCGCCTCAGTGGGCGATAGAGTAATTATTACTTGTCAAGCCTC
AGAGAGCATTTCATCATGGCTCGCCTGGTATCAGCAAAAGCCTGGGAAGGCCCCCAAACTTCTCATCTATGAAGCAT
CAAAGCTGGCCTCTGGGGTTCCGTCTCGCTTCTCCGGGTCCGGCAGTGGTGCAGAGTTTACGTTGACTATATCTTCT
TTGCAACCTGACGATTTCGCAACATATTATTGCCAGGGATACTTTTATTTTATTTCCCGAACATATGTTAACTCTTT
TGGGTGCGGGACCAAACTCACTGTGCTGGGGTCTACCGGTAGTGGTTCTAAGCCTGGTTCAGGCGAAGGCAGTACGA
AAGGGGAAGTGCAACTGGTCGAAAGCGGTGGAGGGCTTGTTCAACCTGGAGGAAGCCTCCGCTTGTCCTGCACGGCT
AGCGGCTTTACAATAAGTACGAACGCCATGAGCTGGGTCCGGCAGGCTCCAGGTAAGTGTCTCGAATGGGTGGGGGT
CATAACAGGCAGGGACATTACCTACTACGCCAGTTGGGCCAAGGGTCGATTTACCATTTCTAGAGATACATCCAAGA
ACACGGTGTACCTCCAGATGAATTCTCTTAGGGCGGAAGACACAGCAGTATACTACTGCGCGCGAGATGGCGGGAGC
AGTGCGATCACATCCAACAACATCTGGGGTCAGGGCACTCTTGTCACGGTGTCGACTGGTGGTGGGGGTAGTGGCGG
CGGAGGTAGCTTTCTTAATTCCCTTTTCAACCAAGAGGTTCAGATCCCCTTGACTGAAAGCTATTGCGGCCCTTGTC
CGAAAAACTGGATATGTTACAAGAATAATTGTTACCAATTCTTCGACGAAAGCAAGAACTGGTATGAGAGTCAGGCG
TCTTGTATGAGTCAGAATGCCAGCCTGCTTAAGGTTTATTCAAAAGAAGACCAGGATCTGCTTAAGTTGGTAAAGAG
CTACCACTGGATGGGGCTGGTACATATCCCAACGAATGGGTCATGGCAGTGGGAGGACGGTTCTATTCTGAGTCCAA
ATCTCCTGACGATCATCGAAATGCAGAAAGGGGACTGTGCCCTGTATGCATCATCCTTCAAGGGGTACATCGAGAAC
TGCAGTACCCCAAATACCTACATTTGTATGCAAAGAACGGTTGCTAGCACCAAGGGCCCATCGGTCTTCCCCCTGGC
ACCCTCCTCCAAGAGCACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGACCTACATCTGCAACGTGAATCACAAGCC
CAGCAACACCAAGGTGGACAAGAGAGTTGAGCCCAAATCTTGTGACAAAACTCACACATGCCCACCGTGCCCAGCAC
CTGAAGCCGCGGGGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGTGGACGGCGTGGA
GGTGCATAATGCCAAGACAAAGCCGCGGGAGGAGCAGTACGCCAGCACGTACCGTGTGGTCAGCGTCCTCACCGTCC
TGCACCAGGACTGGCTGAATGGCAAGGAGTACAAGTGCGCGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCTATAGCAAGCTCACC
GTGGACAAGAGCAGGTGGCAGCAGGGGAACGTCTTCTCATGCTCCGTGATGCATGAGGCTCTGCACAACCACTACAC
GCAGAAGAGCCTCTCCCTGTCTCCGGGTGGCGGTGGAGGGTCCGGCGGTGGTGGATCCGAGGTGCAGCTGTTGGAGT
CTGGGGGAGGCTTGGTACAGCCTGGGGGGTCCCTGAGACTCTCCTGTGCAGCCTCTGGATTCTCCTTCAGTAGCGGG
TACGACATGTGCTGGGTCCGCCAGGCTCCAGGGAAGTGCCTGGAGTGGATCGCATGCATTGCTGCTGGTAGTGCTGG
TATCACTTACGACGCGAACTGGGCGAAAGGCCGGTTCACCATCTCCAGAGACAATTCCAAGAACACGCTGTATCTGC
AAATGAACAGCCTGAGAGCCGAGGACACGGCCGTATATTACTGTGCGAGATCGGCGTTTTCGTTCGACTACGCCATG
GACCTCTGGGGCCAGGGAACCCTGGTCACCGTCTCGAGCGGTGGAGGCGGTTCAGGCGGAGGTGGAAGTGGTGGTGG
CGGCTCTGGAGGCGGCGGATCTGACATCCAGATGACCCAGTCTCCTTCCACCCTGTCTGCATCTGTAGGAGACAGAG
TCACCATCACTTGCCAGGCCAGTCAGAGCATTAGTTCCCACTTAAACTGGTATCAGCAGAAACCAGGGAAAGCCCCT
AAGCTCCTGATCTATAAGGCATCCACTCTGGCATCTGGGGTCCCATCAAGGTTCAGCGGCAGTGGATCTGGGACAGA
ATTTACTCTCACCATCAGCAGCCTGCAGCCTGATGATTTTGCAACTTATTACTGCCAACAGGGTTATAGTTGGGGTA
ATGTTGATAATGTTTTCGGCTGCGGGACCAAGGTGGAGATCAAAGGTGGTGGCGGCTCTGGAGGAGGAGGGTCCGGA
CGGTCGCTGGTGGAGTCTGGGGGAGGCTTGGTCCAGCCTGGGGGGTCCCTGAGACTCTCCTGTACTGCCTCTGGATT
CACCATCAGTAGCTACCACATGCAGTGGGTCCGGCAGGCACCTGGGAAGTGCCTGGAGTACATCGGAACCATTAGTA
GTGGTGGTAATGTATACTACGCAAGCTCCGCTAGAGGCAGATTCACCATCTCCAGACCCTCGTCCAAGAACACGGTG
GATCTTCAAATGAACAGCCTGAGAGCCGAGGACACGGCTGTGTATTACTGTGCGAGAGACTCTGGTTATAGTGATCC
TATGTGGGGCCAGGGAACCCTGGTCACCGTCTCTTCAGGCGGTGGCGGTAGTGGGGGAGGCGGTTCTGGCGGCGGAG
GGTCCGGCGGTGGAGGATCAGACGTTGTGATGACCCAGTCTCCATCTTCCGTGTCTGCATCTGTAGGAGACAGAGTC
ACCATCACCTGTCAGGCCAGTCAGAACATTAGGACTTACTTATCCTGGTATCAGCAGAAACCAGGGAAAGCCCCTAA
GCTCCTGATCTATGCTGCAGCCAATCTGGCATCTGGGGTCCCATCAAGGTTCAGCGGCAGTGGATCTGGGACAGATT
TCACTCTCACCATCAGCGACTTGGAACCTGGCGATGCTGCAACTTACTATTGTCAGTCTACCTATCTTGGTACTGAT
TATGTTGGCGGTGCTTTCGGCTGTGGGACCAAGGTGGAGATCAAA
>SEQ ID 192 SI-49E15 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGCGTKLTVLGSTGSGSKPGSGEGSTKGEVQLVESGGGLVQPGGSLRLSCTA
SGFTISTNAMSWVRQAPGKCLEWVGVITGRDITYYASWAKGRFTISRDTSKNTVYLQMNSLRAEDTAVYYCARDGGS
SAITSNNIWGQGTLVTVSTGGGGSGGGGSFLNSLFNQEVQIPLTESYCGPCPKNWICYKNNCYQFFDESKNWYESQA
SCMSQNASLLKVYSKEDQDLLKLVKSYHWMGLVHIPTNGSWQWEDGSILSPNLLTIIEMQKGDCALYASSFKGYIEN
CSTPNTYICMQRTVASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPEAAGGPSVFLFPPKPKDTLMISRTP
EVTCVVVDVSHEDPEVKFNWYVDGVEVHNAKTKPREEQYASTYRVVSVLTVLHQDWLNGKEYKCAVSNKALPAPIEK
TISKAKGQPREPQVYTLPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGGGGGSGGGGSEVQLLESGGGLVQPGGSLRLSCAASGFSFSSG
YDMCWVRQAPGKCLEWIACIAAGSAGITYDANWAKGRFTISRDNSKNTLYLQMNSLRAEDTAVYYCARSAFSFDYAM
DLWGQGTLVTVSSGGGGSGGGGSGGGGSGGGGSDIQMTQSPSTLSASVGDRVTITCQASQSISSHLNWYQQKPGKAP
KLLIYKASTLASGVPSRFSGSGSGTEFTLTISSLQPDDFATYYCQQGYSWGNVDNVFGCGTKVEIKGGGGSGGGGSG
RSLVESGGGLVQPGGSLRLSCTASGFTISSYHMQWVRQAPGKCLEYIGTISSGGNVYYASSARGRFTISRPSSKNTV
DLQMNSLRAEDTAVYYCARDSGYSDPMWGQGTLVTVSSGGGGSGGGGSGGGGSGGGGSDVVMTQSPSSVSASVGDRV
TITCQASQNIRTYLSWYQQKPGKAPKLLIYAAANLASGVPSRFSGSGSGTDFTLTISDLEPGDAATYYCQSTYLGTD
YVGGAFGCGTKVEIK
>SEQ ID 193 SI-49E15 chain B nt
TTCCTAAACTCATTATTCAACCAAGAAGTTCAAATTCCCTTGACCGAAAGTTACTGTGGCCCATGTCCTAAAAACTG
GATATGTTACAAAAATAACTGCTACCAATTTTTTGATGAGAGTAAAAACTGGTATGAGAGCCAGGCTTCTTGTATGT
CTCAAAATGCCAGCCTTCTGAAAGTATACAGCAAAGAGGACCAGGATTTACTTAAACTGGTGAAGTCATATCATTGG
ATGGGACTAGTACACATTCCAACAAATGGATCTTGGCAGTGGGAAGATGGCTCCATTCTCTCACCCAACCTACTAAC
AATAATTGAAATGCAGAAGGGAGACTGTGCACTCTATGCCTCGAGCTTTAAAGGCTATATAGAAAACTGTTCAACTC
CAAATACGTACATCTGCATGCAAAGGACTGTGCGTACGGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGT
>SEQ ID 194 SI-49E15 chain B aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVRTVAAPSVFIFPPSD
EQLKSGTASVVCLLNNFYPREAKVQWKVDNALQSGNSQESVTEQDSKDSTYSLSSTLTLSKADYEKHKVYACEVTHQ
GLSSPVTKSFNRGEC
>SEQ ID 195 SI-49R21 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTGAAGCCGC
GGGGGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAAT
GCCAAGACAAAGCCGCGGGAGGAGCAGTACGCCAGCACGTACCGTGTGGTCAGCGTCCTCACCGTCCTGCACCAGGA
CTGGCTGAATGGCAAGGAGTACAAGTGCGCGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGATGAGCTGACCAAGAACCAGGTC
AGCCTGACCTGCCTGGTCAAAGGCTTCTATCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTATAGCAAGCTCACCGTGGACAAGA
GCAGGTGGCAGCAGGGGAACGTCTTCTCATGCTCCGTGATGCATGAGGCTCTGCACAACCACTACACGCAGAAGAGC
CTCTCCCTGTCTCCGGGTAAA
>SEQ ID 196 SI-49R21 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVASTKGPSVFPLAPSS
KSTSGGTAALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGGPSVFLFPPKPKDTLMISRTPEVTCVVVDVSHEDPEVKFNWYVDGVEVHN
AKTKPREEQYASTYRVVSVLTVLHQDWLNGKEYKCAVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVMHEALHNHYTQKS
LSLSPGK
>SEQ ID 197 SI-49R21 chain B nt
TTCCTAAACTCATTATTCAACCAAGAAGTTCAAATTCCCTTGACCGAAAGTTACTGTGGCCCATGTCCTAAAAACTG
GATATGTTACAAAAATAACTGCTACCAATTTTTTGATGAGAGTAAAAACTGGTATGAGAGCCAGGCTTCTTGTATGT
CTCAAAATGCCAGCCTTCTGAAAGTATACAGCAAAGAGGACCAGGATTTACTTAAACTGGTGAAGTCATATCATTGG
ATGGGACTAGTACACATTCCAACAAATGGATCTTGGCAGTGGGAAGATGGCTCCATTCTCTCACCCAACCTACTAAC
AATAATTGAAATGCAGAAGGGAGACTGTGCACTCTATGCCTCGAGCTTTAAAGGCTATATAGAAAACTGTTCAACTC
CAAATACGTACATCTGCATGCAAAGGACTGTGCGTACGGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGT
>SEQ ID 198 SI-49R21 chain B aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVRTVAAPSVFIFPPSD
EQLKSGTASVVCLLNNFYPREAKVQWKVDNALQSGNSQESVTEQDSKDSTYSLSSTLTLSKADYEKHKVYACEVTHQ
GLSSPVTKSFNRGEC
>SEQ ID 199 SI-49R22 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTGAAGCCGC
GGGGGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAAT
GCCAAGACAAAGCCGCGGGAGGAGCAGTACGCCAGCACGTACCGTGTGGTCAGCGTCCTCACCGTCCTGCACCAGGA
CTGGCTGAATGGCAAGGAGTACAAGTGCGCGGTCTCCAACAAAGCCCTCCCAGCCCCCATCGAGAAAACCATCTCCA
AAGCCAAAGGGCAGCCCCGAGAACCACAGGTGTACACCCTGCCCCCATGTCGGGATGAGCTGACCAAGAACCAGGTC
AGCCTGTGGTGCCTGGTCAAAGGCTTCTATCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCTATAGCAAGCTCACCGTGGACAAGA
GCAGGTGGCAGCAGGGGAACGTCTTCTCATGCTCCGTGATGCATGAGGCTCTGCACAACCACTACACGCAGAAGAGC
CTCTCCCTGTCTCCGGGTAAA
>SEQ ID 200 SI-49R22 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVASTKGPSVFPLAPSS
KSTSGGTAALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGGPSVFLFPPKPKDTLMISRTPEVTCVVVDVSHEDPEVKFNWYVDGVEVHN
AKTKPREEQYASTYRVVSVLTVLHQDWLNGKEYKCAVSNKALPAPIEKTISKAKGQPREPQVYTLPPCRDELTKNQV
SLWCLVKGFYPSDIAVEWESNGQPENNYKTTPPVLDSDGSFFLYSKLTVDKSRWQQGNVFSCSVMHEALHNHYTQKS
LSLSPGK
>SEQ ID 201 SI-49R22 chain B nt
TTCCTAAACTCATTATTCAACCAAGAAGTTCAAATTCCCTTGACCGAAAGTTACTGTGGCCCATGTCCTAAAAACTG
GATATGTTACAAAAATAACTGCTACCAATTTTTTGATGAGAGTAAAAACTGGTATGAGAGCCAGGCTTCTTGTATGT
CTCAAAATGCCAGCCTTCTGAAAGTATACAGCAAAGAGGACCAGGATTTACTTAAACTGGTGAAGTCATATCATTGG
ATGGGACTAGTACACATTCCAACAAATGGATCTTGGCAGTGGGAAGATGGCTCCATTCTCTCACCCAACCTACTAAC
AATAATTGAAATGCAGAAGGGAGACTGTGCACTCTATGCCTCGAGCTTTAAAGGCTATATAGAAAACTGTTCAACTC
CAAATACGTACATCTGCATGCAAAGGACTGTGCGTACGGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGT
>SEQ ID 202 SI-49R22 chain B aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVRTVAAPSVFIFPPSD
EQLKSGTASVVCLLNNFYPREAKVQWKVDNALQSGNSQESVTEQDSKDSTYSLSSTLTLSKADYEKHKVYACEVTHQ
GLSSPVTKSFNRGEC
>SEQ ID 203 SI-49R22 chain C nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGCTAGCACCAAGGGCCCATCGGTCTTCCCCCTGGCACCCTCCTCC
AAGAGCACCTCTGGGGGCACAGCGGCCCTGGGCTGCCTGGTCAAGGACTACTTCCCCGAACCGGTGACGGTGTCGTG
GAACTCAGGCGCCCTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACCCAGACCTACATCTGCAACGTGAATCACAAGCCCAGCAACACC
AAGGTGGACAAGAGAGTTGAGCCCAAATCTTGTGACAAAACTCACACATGCCCACCGTGCCCAGCACCTGAAGCCGC
GGGGGGACCGTCAGTCTTCCTCTTCCCCCCAAAACCCAAGGACACCCTCATGATCTCCCGGACCCCTGAGGTCACAT
GCGTGGTGGTGGACGTGAGCCACGAAGACCCTGAGGTCAAGTTCAACTGGTACGTGGACGGCGTGGAGGTGCATAAT
GCCAAGACAAAGCCGCGGGAGGAGCAGTACGCCAGCACGTACCGTGTGGTCAGCGTCCTCACCGTCCTGCACCAGGA
CTGGCTGAATGGCAAGGAGTACAAGTGCGCGGTCTCCAACAAAGCCCTCCCAGCCCCCATCGAGAAAACCATCTCCA
AAGCCAAAGGGCAGCCCCGAGAACCACAGGTGTGTACCCTGCCCCCATCCCGGGATGAGCTGACCAAGAACCAGGTC
AGCCTGTCCTGCGCCGTCAAAGGCTTCTATCCCAGCGACATCGCCGTGGAGTGGGAGAGCAATGGGCAGCCGGAGAA
CAACTACAAGACCACGCCTCCCGTGCTGGACTCCGACGGCTCCTTCTTCCTCGTGAGCAAGCTCACCGTGGACAAGA
GCAGGTGGCAGCAGGGGAACGTCTTCTCATGCTCCGTGATGCATGAGGCTCTGCACAACCACTACACGCAGAAGAGC
CTCTCCCTGTCTCCGGGTAAA
>SEQ ID 204 SI-49R22 chain C aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVASTKGPSVFPLAPSS
KSTSGGTAALGCLVKDYFPEPVTVSWNSGALTSGVHTFPAVLQSSGLYSLSSVVTVPSSSLGTQTYICNVNHKPSNT
KVDKRVEPKSCDKTHTCPPCPAPEAAGGPSVFLFPPKPKDTLMISRTPEVTCVVVDVSHEDPEVKFNWYVDGVEVHN
AKTKPREEQYASTYRVVSVLTVLHQDWLNGKEYKCAVSNKALPAPIEKTISKAKGQPREPQVCTLPPSRDELTKNQV
SLSCAVKGFYPSDIAVEWESNGQPENNYKTTPPVLDSDGSFFLVSKLTVDKSRWQQGNVFSCSVMHEALHNHYTQKS
LSLSPGK
>SEQ ID 205 SI-49R23 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGCTAGCGAGCCCAAATCTTCTGACAAAACTCACACATGCCCACCG
TGCCCAGCACCTGAAGCCGCGGGGGGACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACGCCAGCACGTACCGTGTGGTCAGCGTC
CTCACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATCCCGGGATG
AGCTGACCAAGAACCAGGTCAGCCTGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTCCGACGGCTCCTTCTTCCTCTATAG
CAAGCTCACCGTGGACAAGAGCAGGTGGCAGCAGGGGAACGTCTTCTCATGCTCCGTGATGCATGAGGCTCTGCACA
ACCACTACACGCAGAAGAGCCTCTCCCTGTCTCCGGGTAAA
>SEQ ID 206 SI-49R23 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVASEPKSSDKTHTCPP
CPAPEAAGGPSVFLFPPKPKDTLMISRTPEVTCVVVDVSHEDPEVKFNWYVDGVEVHNAKTKPREEQYASTYRVVSV
LTVLHQDWLNGKEYKCAVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSLSPGK
>SEQ ID 207 SI-49R24 chain A nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGCTAGCGAGCCCAAATCTTCTGACAAAACTCACACATGCCCACCG
TGCCCAGCACCTGAAGCCGCGGGGGGACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACGCCAGCACGTACCGTGTGGTCAGCGTC
CTCACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTACACCCTGCCCCCATGTCGGGATG
AGCTGACCAAGAACCAGGTCAGCCTGTGGTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTCCGACGGCTCCTTCTTCCTCTATAG
CAAGCTCACCGTGGACAAGAGCAGGTGGCAGCAGGGGAACGTCTTCTCATGCTCCGTGATGCATGAGGCTCTGCACA
ACCACTACACGCAGAAGAGCCTCTCCCTGTCTCCGGGTAAA
>SEQ ID 208 SI-49R24 chain A aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVASEPKSSDKTHTCPP
CPAPEAAGGPSVFLFPPKPKDTLMISRTPEVTCVVVDVSHEDPEVKFNWYVDGVEVHNAKTKPREEQYASTYRVVSV
LTVLHQDWLNGKEYKCAVSNKALPAPIEKTISKAKGQPREPQVYTLPPCRDELTKNQVSLWCLVKGFYPSDIAVEWE
SNGQPENNYKTTPPVLDSDGSFFLYSKLTVDKSRWQQGNVFSCSVMHEALHNHYTQKSLSLSPGK
>SEQ ID 209 SI-49R24 chain B nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGCTAGCGAGCCCAAATCTTCTGACAAAACTCACACATGCCCACCG
TGCCCAGCACCTGAAGCCGCGGGGGGACCGTCAGTCTTCCTCTTCCCCCCAAAACCCAAGGACACCCTCATGATCTC
CCGGACCCCTGAGGTCACATGCGTGGTGGTGGACGTGAGCCACGAAGACCCTGAGGTCAAGTTCAACTGGTACGTGG
ACGGCGTGGAGGTGCATAATGCCAAGACAAAGCCGCGGGAGGAGCAGTACGCCAGCACGTACCGTGTGGTCAGCGTC
CTCACCGTCCTGCACCAGGACTGGCTGAATGGCAAGGAGTACAAGTGCGCGGTCTCCAACAAAGCCCTCCCAGCCCC
CATCGAGAAAACCATCTCCAAAGCCAAAGGGCAGCCCCGAGAACCACAGGTGTGTACCCTGCCCCCATCCCGGGATG
AGCTGACCAAGAACCAGGTCAGCCTGTCCTGCGCCGTCAAAGGCTTCTATCCCAGCGACATCGCCGTGGAGTGGGAG
AGCAATGGGCAGCCGGAGAACAACTACAAGACCACGCCTCCCGTGCTGGACTCCGACGGCTCCTTCTTCCTCGTGAG
CAAGCTCACCGTGGACAAGAGCAGGTGGCAGCAGGGGAACGTCTTCTCATGCTCCGTGATGCATGAGGCTCTGCACA
ACCACTACACGCAGAAGAGCCTCTCCCTGTCTCCGGGTAAA
>SEQ ID 210 SI-49R24 chain B aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVASEPKSSDKTHTCPP
CPAPEAAGGPSVFLFPPKPKDTLMISRTPEVTCVVVDVSHEDPEVKFNWYVDGVEVHNAKTKPREEQYASTYRVVSV
LTVLHQDWLNGKEYKCAVSNKALPAPIEKTISKAKGQPREPQVCTLPPSRDELTKNQVSLSCAVKGFYPSDIAVEWE
SNGQPENNYKTTPPVLDSDGSFFLVSKLTVDKSRWQQGNVFSCSVMHEALHNHYTQKSLSLSPGK
>SEQ ID 217 anti-claudin zolbetuximab VH nt
CAGGTCCAACTGCAGCAGCCTGGGGCTGAGCTGGTGAGGCCTGGGGCTTCAGTGAAGCTGTCCTGCAAGGCTTCTGG
CTACACCTTCACCAGCTACTGGATAAACTGGGTGAAGCAGAGGCCTGGACAAGGCCTTGAGTGGATCGGAAATATTT
ATCCTTCTGATAGTTATACTAACTACAATCAAAAGTTCAAGGACAAGGCCACATTGACTGTAGACAAATCCTCCAGC
ACAGCCTACATGCAGCTCAGCAGCCCGACATCTGAGGACTCTGCGGTCTATTACTGTACAAGATCGTGGAGGGGTAA
CTCCTTTGACTACTGGGGCCAAGGCACCACTCTCACAGTCTCCTCA
>SEQ ID 218 anti-claudin zolbetuximab VH aa
QVQLQQPGAELVRPGASVKLSCKASGYTFTSYWINWVKQRPGQGLEWIGNIYPSDSYTNYNQKFKDKATLTVDKSSS
TAYMQLSSPTSEDSAVYYCTRSWRGNSFDYWGQGTTLTVSS
>SEQ ID 219 anti-claudin zolbetuximab VL nt
GACATTGTGATGACACAGTCTCCATCCTCCCTGACTGTGACAGCAGGAGAGAAGGTCACTATGAGCTGCAAGTCCAG
TCAGAGTCTGTTAAACAGTGGAAATCAAAAGAACTACTTGACCTGGTACCAGCAGAAACCAGGGCAGCCTCCTAAAC
TGTTGATCTACTGGGCATCCACTAGGGAATCTGGGGTCCCTGATCGCTTCACAGGCAGTGGATCTGGAACAGATTTC
ACTCTCACCATCAGCAGTGTGCAGGCTGAAGACCTGGCAGTTTATTACTGTCAGAATGATTATAGTTATCCATTCAC
GTTCGGCTCGGGGACAAAGTTGGAAATAAAA
>SEQ ID 220 anti-claudin zolbetuximab VL aa
DIVMTQSPSSLTVTAGEKVTMSCKSSQSLLNSGNQKNYLTWYQQKPGQPPKLLIYWASTRESGVPDRFTGSGSGTDF
TLTISSVQAEDLAVYYCQNDYSYPFTFGSGTKLEIK
>SEQ ID 221 anti-HER2 trastuzumab VH nt
GAGGTTCAGCTGGTGGAGTCTGGCGGTGGCCTGGTGCAGCCAGGGGGCTCACTCCGTTTGTCCTGTGCAGCTTCTGG
CTTCAACATTAAAGACACCTATATACACTGGGTGCGTCAGGCCCCGGGTAAGGGCCTGGAATGGGTTGCAAGGATTT
ATCCTACGAATGGTTATACTAGATATGCCGATAGCGTCAAGGGCCGTTTCACTATAAGCGCAGACACATCCAAAAAC
ACAGCCTACCTGCAGATGAACAGCCTGCGTGCTGAGGACACTGCCGTCTATTATTGTTCTAGATGGGGAGGGGACGG
CTTCTATGCTATGGACTACTGGGGTCAAGGAACCCTGGTCACCGTCTCCTCG
>SEQ ID 222 anti-HER2 trastuzumab VH aa
EVQLVESGGGLVQPGGSLRLSCAASGFNIKDTYIHWVRQAPGKGLEWVARIYPTNGYTRYADSVKGRFTISADTSKN
TAYLQMNSLRAEDTAVYYCSRWGGDGFYAMDYWGQGTLVTVSS
>SEQ ID 223 anti-HER2 trastuzumab VL nt
GATATCCAGATGACCCAGTCCCCGAGCTCCCTGTCCGCCTCTGTGGGCGATAGGGTCACCATCACCTGCCGTGCCAG
TCAGGATGTGAATACTGCTGTAGCCTGGTATCAACAGAAACCAGGAAAAGCTCCGAAACTACTGATTTACTCGGCAT
CCTTCCTCTACTCTGGAGTCCCTTCTCGCTTCTCTGGCTCCAGATCTGGGACGGATTTCACTCTGACCATCAGCAGT
CTGCAGCCGGAAGACTTCGCAACTTATTACTGTCAGCAACATTATACTACTCCTCCCACGTTCGGACAGGGTACCAA
GGTGGAGATCAAA
>SEQ ID 224 anti-HER2 trastuzumab VL aa
DIQMTQSPSSLSASVGDRVTITCRASQDVNTAVAWYQQKPGKAPKLLIYSASFLYSGVPSRFSGSRSGTDFTLTISS
LQPEDFATYYCQQHYTTPPTFGQGTKVEIK
>SEQ ID 225 anti-HER2 pertuzumab VH nt
GAAGTGCAGCTCGTCGAAAGCGGTGGCGGACTGGTTCAGCCCGGTGGTTCTCTGCGGCTGTCTTGTGCTGCCTCGGG
TTTCACGTTCACTGACTACACAATGGACTGGGTGCGTCAGGCTCCTGGAAAGGGATTGGAGTGGGTAGCCGACGTTA
ATCCAAACTCCGGCGGGAGCATCTACAACCAGAGGTTCAAGGGGAGGTTCACTCTGAGCGTGGATCGCTCCAAGAAC
ACGCTGTACCTCCAGATGAACTCTCTCAGGGCCGAGGACACGGCTGTTTACTATTGCGCGAGGAACCTGGGTCCTTC
CTTCTACTTCGACTACTGGGGACAGGGAACCCTGGTGACCGTCAGCTCC
>SEQ ID 226 anti-HER2 pertuzumab VH aa
EVQLVESGGGLVQPGGSLRLSCAASGFTFTDYTMDWVRQAPGKGLEWVADVNPNSGGSIYNQRFKGRFTLSVDRSKN
TLYLQMNSLRAEDTAVYYCARNLGPSFYFDYWGQGTLVTVSS
>SEQ ID 227 anti-HER2 pertuzumab VL nt
GACATCCAGATGACACAATCTCCTAGCAGTCTGAGCGCAAGTGTTGGAGATCGTGTCACCATCACATGCAAGGCCAG
CCAGGATGTGAGCATTGGAGTCGCCTGGTATCAGCAGAAACCCGGCAAGGCACCCAAGCTGCTGATCTACTCGGCCA
GTTACAGATACACTGGCGTACCTTCGAGGTTTAGTGGTAGCGGTTCTGGAACCGATTTCACCCTCACCATTAGCTCC
CTCCAACCCGAGGACTTCGCCACCTACTACTGCCAGCAATACTACATCTACCCTTACACGTTCGGCCAAGGCACTAA
GGTCGAGATTAAA
>SEQ ID 228 anti-HER2 pertuzumab VL aa
DIQMTQSPSSLSASVGDRVTITCKASQDVSIGVAWYQQKPGKAPKLLIYSASYRYTGVPSRFSGSGSGTDFTLTISS
LQPEDFATYYCQQYYIYPYTFGQGTKVEIK
>SEQ ID 229 anti-mesothelin amatuximab VH nt
CAGGTACAACTGCAGCAGTCTGGGCCTGAGCTGGAGAAGCCTGGCGCTTCAGTGAAGATATCCTGCAAGGCTTCTGG
TTACTCATTCACTGGCTACACCATGAACTGGGTGAAGCAGAGCCATGGAAAGAGCCTTGAGTGGATTGGACTTATTA
CTCCTTACAATGGTGCTTCTAGCTACAACCAGAAGTTCAGGGGCAAGGCCACATTAACTGTAGACAAGTCATCCAGC
ACAGCCTACATGGACCTCCTCAGTCTGACATCTGAAGACTCTGCAGTCTATTTCTGTGCAAGGGGGGGTTACGACGG
GAGGGGTTTTGACTACTGGGGATCTGGGACCCCGGTCACCGTCTCCTCA
>SEQ ID 230 anti-mesothelin amatuximab VH aa
QVQLQQSGPELEKPGASVKISCKASGYSFTGYTMNWVKQSHGKSLEWIGLITPYNGASSYNQKFRGKATLTVDKSSS
TAYMDLLSLTSEDSAVYFCARGGYDGRGFDYWGSGTPVTVSS
>SEQ ID 231 anti-mesothelin amatuximab VL nt
GACATCGAGCTCACTCAGTCTCCAGCAATCATGTCTGCATCTCCAGGGGAGAAGGTCACCATGACCTGCAGTGCCAG
CTCAAGTGTAAGTTACATGCACTGGTACCAGCAGAAGTCAGGCACCTCCCCCAAAAGATGGATTTATGACACATCCA
AACTGGCTTCTGGAGTCCCAGGTCGCTTCAGTGGCAGTGGGTCTGGAAACTCTTACTCTCTCACAATCAGCAGCGTG
GAGGCTGAAGATGATGCAACTTATTACTGCCAGCAGTGGAGTAAGCACCCTCTCACGTTCGGATCCGGGACCAAGGT
GGAAATCAAA
>SEQ ID 232 anti-mesothelin amatuximab VL aa
DIELTQSPAIMSASPGEKVTMTCSASSSVSYMHWYQQKSGTSPKRWIYDTSKLASGVPGRFSGSGSGNSYSLTISSV
EAEDDATYYCQQWSKHPLTFGSGTKVEIK
>SEQ ID 233 anti-GD2 dinutuximab VH nt
GAGGTCCAACTGCTGCAGTCTGGACCTGAGCTGGAGAAGCCTGGCGCTTCAGTGATGATATCCTGCAAGGCTTCTGG
TTCCTCATTCACTGGCTACAACATGAACTGGGTGAGGCAGAACATTGGAAAGAGCCTTGAATGGATTGGAGCTATTG
ATCCTTACTATGGTGGAACTAGCTACAACCAGAAGTTCAAGGGCAGGGCCACATTGACTGTAGACAAATCGTCCAGC
ACAGCCTACATGCACCTCAAGAGCCTGACATCTGAGGACTCTGCAGTCTATTACTGTGTAAGCGGAATGGAGTACTG
GGGTCAAGGAACCTCAGTCACCGTCTCCTCA
>SEQ ID 234 anti-GD2 dinutuximab VH aa
EVQLLQSGPELEKPGASVMISCKASGSSFTGYNMNWVRQNIGKSLEWIGAIDPYYGGTSYNQKFKGRATLTVDKSSS
TAYMHLKSLTSEDSAVYYCVSGMEYWGQGTSVTVSS
>SEQ ID 235 anti-GD2 dinutuximab VL nt
GAAATAGTGATGACGCAGTCTCCAGCCACCCTGTCTGTGTCTCCAGGGGAAAGAGCCACCCTCTCCTGCAGATCTAG
TCAGAGTCTTGTACACCGTAATGGAAACACCTATTTACATTGGTACCTGCAGAAGCCAGGCCAGTCTCCAAAGCTCC
TGATTCACAAAGTTTCCAACCGATTTTCTGGGGTCCCAGACAGGTTCAGTGGCAGTGGATCAGGGACAGATTTCACA
CTCAAGATCAGCAGAGTGGAGGCTGAGGATCTGGGAGTTTATTTCTGTTCTCAAAGTACACATGTTCCTCCGCTCAC
GTTCGGTGCTGGGACCAAGCTGGAGCTGAAA
>SEQ ID 236 anti-GD2 dinutuximab VL aa
EIVMTQSPATLSVSPGERATLSCRSSQSLVHRNGNTYLHWYLQKPGQSPKLLIHKVSNRFSGVPDRFSGSGSGTDFT
LKISRVEAEDLGVYFCSQSTHVPPLTFGAGTKLELK
>SEQ ID 237 anti-CD20 rituximab VH nt
CAGGTGCAGCTGCAGCAGCCCGGCGCCGAGCTGGTGAAGCCCGGCGCCAGCGTGAAGATGAGCTGCAAGGCCAGCGG
CTACACCTTCACCAGCTACAACATGCACTGGGTGAAGCAGACCCCCGGCCGGGGCCTGGAGTGGATCGGCGCCATCT
ACCCCGGCAACGGCGACACCAGCTACAACCAGAAGTTCAAGGGCAAGGCCACCCTGACCGCCGACAAGAGCAGCAGC
ACCGCCTACATGCAGCTGAGCAGCCTGACCAGCGAGGACAGCGCCGTGTACTACTGCGCCCGGAGCACCTACTACGG
CGGCGACTGGTACTTCAACGTGTGGGGCGCCGGCACCACCGTGACCGTGAGCGCC
>SEQ ID 238 anti-CD20 rituximab VH aa
QVQLQQPGAELVKPGASVKMSCKASGYTFTSYNMHWVKQTPGRGLEWIGAIYPGNGDTSYNQKFKGKATLTADKSSS
TAYMQLSSLTSEDSAVYYCARSTYYGGDWYFNVWGAGTTVTVSA
>SEQ ID 239 anti-CD20 rituximab VL nt
CAGATCGTGCTGAGCCAGAGCCCCGCCATCCTGAGCGCCAGCCCCGGCGAGAAGGTGACCATGACCTGCCGGGCCAG
CAGCAGCGTGAGCTACATCCACTGGTTCCAGCAGAAGCCCGGCAGCAGCCCCAAGCCCTGGATCTACGCCACCAGCA
ACCTGGCCAGCGGCGTGCCCGTGCGGTTCAGCGGCAGCGGCAGCGGCACCAGCTACAGCCTGACCATCAGCCGGGTG
GAGGCCGAGGACGCCGCCACCTACTACTGCCAGCAGTGGACCAGCAACCCCCCCACCTTCGGCGGCGGCACCAAGCT
GGAGATCAAG
>SEQ ID 240 anti-CD20 rituximab VL aa
QIVLSQSPAILSASPGEKVTMTCRASSSVSYIHWFQQKPGSSPKPWIYATSNLASGVPVRFSGSGSGTSYSLTISRV
EAEDAATYYCQQWTSNPPTFGGGTKLEIK
>SEQ ID 241 anti-EGFR cetuximab VH nt
CAGGTGCAGCTGAAGCAGTCAGGACCTGGCCTAGTGCAGCCCTCACAGAGCCTGTCCATCACCTGCACAGTCTCTGG
TTTCTCATTAACTAACTATGGTGTACACTGGGTTCGCCAGTCTCCAGGAAAGGGTCTGGAGTGGCTGGGAGTGATAT
GGAGTGGTGGAAACACAGACTATAATACACCTTTCACATCCAGACTGAGCATCAACAAGGACAATTCCAAGAGCCAA
GTTTTCTTTAAAATGAACAGTCTGCAATCTAATGACACAGCCATATATTACTGTGCCAGAGCCCTCACCTACTATGA
TTACGAGTTTGCTTACTGGGGCCAAGGGACTCTGGTCACTGTCTCTAGC
>SEQ ID 242 anti-EGFR cetuximab VH aa
QVQLKQSGPGLVQPSQSLSITCTVSGFSLTNYGVHWVRQSPGKGLEWLGVIWSGGNTDYNTPFTSRLSINKDNSKSQ
VFFKMNSLQSNDTAIYYCARALTYYDYEFAYWGQGTLVTVSS
>SEQ ID 243 anti-EGFR cetuximab VL nt
GACATCTTGCTGACTCAGTCTCCAGTCATCCTGTCTGTGAGTCCAGGAGAAAGAGTCAGTTTCTCCTGCAGGGCCAG
TCAGAGTATTGGCACAAACATACACTGGTATCAGCAAAGAACAAATGGTTCTCCAAGGCTTCTCATAAAGTATGCTT
CTGAGTCTATCTCTGGGATTCCTTCCAGGTTTAGTGGCAGTGGATCAGGGACAGATTTTACTCTTAGCATCAACAGT
GTGGAGTCTGAAGATATTGCAGATTATTACTGTCAACAAAATAATAACTGGCCAACCACGTTCGGTGCTGGGACCAA
GCTGGAGCTGAAA
>SEQ ID 244 anti-EGFR cetuximab VL aa
DILLTQSPVILSVSPGERVSFSCRASQSIGTNIHWYQQRTNGSPRLLIKYASESISGIPSRFSGSGSGTDFTLSINS
VESEDIADYYCQQNNNWPTTFGAGTKLELK
>SEQ ID 245 anti-EGFR panitumumab VH nt
CAGGTGCAGCTGCAGGAGTCGGGCCCAGGACTGGTGAAGCCTTCGGAGACCCTGTCCCTCACCTGCACTGTCTCTGG
TGGCTCCGTCAGCAGTGGTGATTACTACTGGACCTGGATCCGGCAGTCCCCAGGGAAGGGACTGGAGTGGATTGGAC
ACATCTATTACAGTGGGAACACCAATTATAACCCCTCCCTCAAGAGCCGACTCACCATATCAATTGACACGTCCAAG
ACTCAGTTCTCCCTGAAGCTGAGTTCTGTGACCGCTGCGGACACGGCCATTTATTACTGTGTGCGAGATCGAGTGAC
TGGTGCTTTTGATATCTGGGGCCAAGGGACAATGGTCACCGTCTCGAGC
>SEQ ID 246 anti-EGFR panitumumab VH aa
QVQLQESGPGLVKPSETLSLTCTVSGGSVSSGDYYWTWIRQSPGKGLEWIGHIYYSGNTNYNPSLKSRLTISIDTSK
TQFSLKLSSVTAADTAIYYCVRDRVTGAFDIWGQGTMVTVSS
>SEQ ID 247 anti-EGFR panitumumab VL nt
GACATCCAGATGACCCAGTCTCCATCCTCCCTGTCTGCATCTGTAGGAGACAGAGTCACCATCACTTGCCAGGCGAG
TCAGGACATCAGCAACTATTTAAATTGGTATCAGCAGAAACCAGGGAAAGCCCCTAAACTCCTGATCTACGATGCAT
CCAATTTGGAAACAGGGGTCCCATCAAGGTTCAGTGGAAGTGGATCTGGGACAGATTTTACTTTCACCATCAGCAGC
CTGCAGCCTGAAGATATTGCAACATATTTCTGTCAACACTTTGATCATCTCCCGCTCGCTTTCGGCGGAGGGACCAA
GGTGGAAATTAAA
>SEQ ID 248 anti-EGFR panitumumab VL aa
DIQMTQSPSSLSASVGDRVTITCQASQDISNYLNWYQQKPGKAPKLLIYDASNLETGVPSRFSGSGSGTDFTFTISS
LQPEDIATYFCQHFDHLPLAFGGGTKVEIK
>SEQ ID 249 anti-EGFR nimotuzumab VH nt
CAGGTGCAGCTGCAGCAGAGCGGCGCCGAGGTGAAGAAGCCCGGCAGCAGCGTGAAGGTGAGCTGCAAGGCCAGCGG
CTACACCTTCACCAACTACTACATCTACTGGGTGCGGCAGGCCCCCGGCCAGGGCCTGGAGTGGATCGGCGGCATCA
ACCCCACCAGCGGCGGCAGCAACTTCAACGAGAAGTTCAAGACCCGGGTGACCATCACCGCCGACGAGAGCAGCACC
ACCGCCTACATGGAGCTGAGCAGCCTGCGGAGCGAGGACACCGCCTTCTACTTCTGCACCCGGCAGGGCCTGTGGTT
CGACAGCGACGGCCGGGGCTTCGACTTCTGGGGCCAGGGCACCACCGTGACCGTGAGCAGC
>SEQ ID 250 anti-EGFR nimotuzumab VH aa
QVQLQQSGAEVKKPGSSVKVSCKASGYTFTNYYIYWVRQAPGQGLEWIGGINPTSGGSNFNEKFKTRVTITADESST
TAYMELSSLRSEDTAFYFCTRQGLWFDSDGRGFDFWGQGTTVTVSS
>SEQ ID 251 anti-EGFR nimotuzumab VL nt
GATATTCAAATGACTCAATCTCCTTCTTCTCTTTCTGCTTCTGTTGGTGATCGTGTTACTATTACTTGTCGTTCTTC
TCAAAATATTGTTCATTCTAATGGTAATACTTATCTTGATTGGTATCAACAAACTCCTGGTAAAGCTCCTAAACTTC
TTATTTATAAAGTTTCTAATCGTTTTTCTGGTGTTCCTTCTCGTTTTTCTGGTTCTGGTTCTGGTACTGATTTTACT
TTTACTATTTCTTCTCTTCAACCTGAAGATATTGCTACTTATTATTGTTTTCAATATTCTCATGTTCCTTGGACTTT
TGGTCAAGGTACTAAACTTCAAATTACT
>SEQ ID 252 anti-EGFR nimotuzumab VL aa
DIQMTQSPSSLSASVGDRVTITCRSSQNIVHSNGNTYLDWYQQTPGKAPKLLIYKVSNRFSGVPSRFSGSGSGTDFT
FTISSLQPEDIATYYCFQYSHVPWTFGQGTKLQIT
>SEQ ID 253 anti-CD22 inotuzumab VH nt
GAGGTGCAATTGGTCCAGTCAGGAGCAGAGGTTAAGAAGCCTGGTGCTTCCGTCAAAGTTTCGTGTAAGGCCAGCGG
CTACAGGTTCACAAATTATTGGATTCATTGGGTCAGGCAGGCTCCGGGACAAGGCCTGGAATGGATCGGTGGCATTA
ATCCCGGGAATAACTACGCTACATATAGGAGAAAATTCCAGGGCAGAGTTACGATGACCGCGGACACCTCCACAAGC
ACTGTCTACATGGAGCTGTCATCTCTGAGATCCGAGGACACCGCAGTGTACTATTGTACTAGAGAAGGCTACGGTAA
TTACGGAGCCTGGTTCGCCTACTGGGGCCAGGGTACCCTAGTCACAGTCTCCTCA
>SEQ ID 254 anti-CD22 inotuzumab VH aa
EVQLVQSGAEVKKPGASVKVSCKASGYRFTNYWIHWVRQAPGQGLEWIGGINPGNNYATYRRKFQGRVTMTADTSTS
TVYMELSSLRSEDTAVYYCTREGYGNYGAWFAYWGQGTLVTVSS
>SEQ ID 255 anti-CD22 inotuzumab VL nt
GACGTTCAAGTGACCCAGAGCCCATCCAGCCTGAGCGCATCTGTAGGAGACCGGGTCACCATCACTTGTAGATCCAG
TCAGAGTCTTGCAAACAGTTATGGGAACACCTTTTTGTCTTGGTATCTGCACAAACCAGGTAAAGCCCCACAATTGC
TCATCTACGGAATCTCTAACAGATTTAGTGGTGTACCAGACAGGTTCAGCGGTTCCGGAAGTGGTACTGATTTCACC
CTCACGATCTCGTCTCTCCAGCCAGAAGATTTCGCCACTTATTACTGTTTACAAGGTACACATCAGCCGTACACATT
CGGTCAGGGTACTAAAGTAGAAATCAAA
>SEQ ID 256 anti-CD22 inotuzumab VL aa
DVQVTQSPSSLSASVGDRVTITCRSSQSLANSYGNTFLSWYLHKPGKAPQLLIYGISNRFSGVPDRFSGSGSGTDFT
LTISSLQPEDFATYYCLQGTHQPYTFGQGTKVEIK
>SEQ ID 257 anti-CD30 brentuximab VH nt
CAGATCCAGCTGCAGCAGTCTGGACCTGAGGTGGTGAAGCCTGGGGCTTCAGTGAAGATATCCTGCAAGGCTTCTGG
CTACACCTTCACTGACTACTATATAACCTGGGTGAAGCAGAAGCCTGGACAGGGACTTGAGTGGATTGGATGGATTT
ATCCTGGAAGCGGTAATACTAAGTACAATGAGAAGTTCAAGGGCAAGGCCACATTGACTGTAGACACATCCTCCAGC
ACAGCCTTCATGCAGCTCAGCAGCCTGACATCTGAGGACACTGCTGTCTATTTCTGTGCGAACTATGGTAACTACTG
GTTTGCTTACTGGGGCCAAGGGACTCAGGTCACTGTCTCTGCA
>SEQ ID 258 anti-CD30 brentuximab VH aa
QIQLQQSGPEVVKPGASVKISCKASGYTETDYYITWVKQKPGQGLEWIGWIYPGSGNTKYNEKFKGKATLTVDTSSS
TAFMQLSSLTSEDTAVYFCANYGNYWFAYWGQGTQVTVSA
>SEQ ID 259 anti-CD30 brentuximab VL nt
GACATTGTGCTGACCCAATCTCCAGCTTCTTTGGCTGTGTCTCTAGGGCAGAGGGCCACCATCTCCTGCAAGGCCAG
CCAAAGTGTTGATTTTGATGGTGATAGTTATATGAACTGGTACCAACAGAAACCAGGACAGCCACCCAAAGTCCTCA
TCTATGCTGCATCCAATCTAGAATCTGGGATCCCAGCCAGGTTTAGTGGCAGTGGGTCTGGGACAGACTTCACCCTC
AACATCCATCCTGTGGAGGAGGAGGATGCTGCAACCTATTACTGTCAGCAAAGTAATGAGGATCCGTGGACGTTCGG
TGGAGGCACCAAGCTGGAAATCAAA
>SEQ ID 260 anti-CD30 brentuximab VL aa
DIVLTQSPASLAVSLGQRATISCKASQSVDFDGDSYMNWYQQKPGQPPKVLIYAASNLESGIPARFSGSGSGTDFTL
NIHPVEEEDAATYYCQQSNEDPWTFGGGTKLEIK
>SEQ ID 261 anti-HER3 MM-111 VH nt
CAGGTGCAATTGCAGGAGTCGGGGGGAGGCCTGGTCAAGCCTGGAGGGTCCCTGAGACTCTCCTGTGCAGCCTCTGG
ATTCACCTTTAGTAGTTATTGGATGAGCTGGGTCCGCCAGGCTCCAGGGAAGGGGCTGGAGTGGGTGGCCAACATAA
ACCGCGATGGAAGTGCGAGTTACTATGTGGACTCTGTGAAGGGCCGATTCACCATCTCCAGAGACGACGCCAAGAAC
TCACTGTATCTGCAAATGAACAGCCTGAGAGCTGAGGACACGGCTGTGTATTACTGTGCGAGAGATCGTGGGGTGGG
CTACTTCGATCTCTGGGGCCGTGGCACCCTGGTCACCGTCTCGAGC
>SEQ ID 262 anti-HER3 MM-111 VH aa
QVQLQESGGGLVKPGGSLRLSCAASGFTFSSYWMSWVRQAPGKGLEWVANINRDGSASYYVDSVKGRFTISRDDAKN
SLYLQMNSLRAEDTAVYYCARDRGVGYFDLWGRGTLVTVSS
>SEQ ID 263 anti-HER3 MM-111 VL nt
CAGTCTGCCCTGACTCAGCCTGCCTCCGTGTCTGGGTCTCCTGGACAGTCGATCACCATCTCCTGCACTGGAACCAG
CAGTGACGTTGGTGGTTATAACTTTGTCTCCTGGTACCAACAACACCCAGGCAAAGCCCCCAAACTCATGATCTATG
ATGTCAGTGATCGGCCCTCAGGGGTGTCTGATCGCTTCTCCGGCTCCAAGTCTGGCAACACGGCCTCCCTGATCATC
TCTGGCCTCCAGGCTGACGACGAGGCTGATTATTACTGCAGCTCATATGGGAGCAGCAGCACTCATGTGATTTTCGG
CGGAGGGACCAAGGTGACCGTCCTA
>SEQ ID 264 anti-HER3 MM-111 VL aa
QSALTQPASVSGSPGQSITISCTGTSSDVGGYNFVSWYQQHPGKAPKLMIYDVSDRPSGVSDRFSGSKSGNTASLII
SGLQADDEADYYCSSYGSSSTHVIFGGGTKVTVL
>SEQ ID 265 anti-EGFRvIII ABT-806 VH nt
GATGTGCAGCTTCAGGAGTCGGGACCTAGCCTGGTGAAACCTTCTCAGTCTCTGTCCCTCACCTGCACTGTCACTGG
CTACTCAATCACCAGTGATTTTGCCTGGAACTGGATTCGGCAGTTTCCAGGAAACAAGCTGGAGTGGATGGGCTACA
TAAGTTATAGTGGTAACACTAGGTACAACCCATCTCTCAAAAGTCGAATCTCTATCACTCGCGACACATCCAAGAAC
CAATTCTTCCTGCAGTTGAACTCTGTGACTATTGAGGACACAGCCACATATTACTGTGTAACGGCGGGACGCGGGTT
TCCTTATTGGGGCCAAGGGACTCTGGTCACTGTCTCTGCA
>SEQ ID 266 anti-EGFRvIII ABT-806 VH aa
DVQLQESGPSLVKPSQSLSLTCTVTGYSITSDFAWNWIRQFPGNKLEWMGYISYSGNTRYNPSLKSRISITRDTSKN
QFFLQLNSVTIEDTATYYCVTAGRGFPYWGQGTLVTVSA
>SEQ ID 267 anti-EGFRvIII ABT-806 VL nt
GACATCCTGATGACCCAATCTCCATCCTCCATGTCTGTATCTCTGGGAGACACAGTCAGCATCACTTGCCATTCAAG
TCAGGACATTAACAGTAATATAGGGTGGTTGCAGCAGAGACCAGGGAAATCATTTAAGGGCCTGATCTATCATGGAA
CCAACTTGGACGATGAAGTTCCATCAAGGTTCAGTGGCAGTGGATCTGGAGCCGATTATTCTCTCACCATCAGCAGC
CTGGAATCTGAAGATTTTGCAGACTATTACTGTGTACAGTATGCTCAGTTTCCGTGGACGTTCGGTGGAGGCACCAA
GCTGGAAATCAAA
>SEQ ID 268 anti-EGFRvIII ABT-806 VL aa
DILMTQSPSSMSVSLGDTVSITCHSSQDINSNIGWLQQRPGKSFKGLIYHGTNLDDEVPSRFSGSGSGADYSLTISS
LESEDFADYYCVQYAQFPWTFGGGTKLEIK
>SEQ ID 269 anti-CD19 21D4 VH nt
GAGGTGCAGCTGGTGCAGTCTGGAGCAGAGGTGAAGAAACCAGGAGAGTCTCTGAAGATCTCCTGTAAGGGTTCTGG
ATACAGCTTTAGCAGTTCATGGATCGGCTGGGTGCGCCAGGCACCTGGGAAAGGCCTGGAATGGATGGGGATCATCT
ATCCTGATGACTCTGATACCAGATACAGTCCATCCTTCCAAGGCCAGGTCACCATCTCAGCCGACAAGTCCATCAGG
ACTGCCTACCTGCAGTGGAGTAGCCTGAAGGCCTCGGACACCGCTATGTATTACTGTGCGAGACATGTTACTATGAT
TTGGGGAGTTATTATTGACTTCTGGGGCCAGGGAACCCTGGTCACCGTCTCCTCA
>SEQ ID 270 anti-CD19 21D4 VH aa
EVQLVQSGAEVKKPGESLKISCKGSGYSFSSSWIGWVRQAPGKGLEWMGIIYPDDSDTRYSPSFQGQVTISADKSIR
TAYLQWSSLKASDTAMYYCARHVTMIWGVIIDFWGQGTLVTVSS
>SEQ ID 271 anti-CD19 21D4 VL nt
GCCATCCAGTTGACCCAGTCTCCATCCTCCCTGTCTGCATCTGTAGGAGACAGAGTCACCATCACTTGCCGGGCAAG
TCAGGGCATTAGCAGTGCTTTAGCCTGGTATCAGCAGAAACCAGGGAAAGCTCCTAAGCTCCTGATCTATGATGCCT
CCAGTTTGGAAAGTGGGGTCCCATCAAGGTTCAGCGGCAGTGGATCTGGGACAGATTTCACTCTCACCATCAGCAGC
CTGCAGCCTGAAGATTTTGCAACTTATTACTGTCAACAGTTTAATAGTTACCCATTCACTTTCGGCCCTGGGACCAA
AGTGGATATCAAA
>SEQ ID 272 anti-CD19 21D4 VL aa
AIQLTQSPSSLSASVGDRVTITCRASQGISSALAWYQQKPGKAPKLLIYDASSLESGVPSRFSGSGSGTDFTLTISS
LQPEDFATYYCQQFNSYPFTFGPGTKVDIK
>SEQ ID 273 anti-CD33 gemtuzumab VH nt
GAGGTCCAATTGGTACAATCCGGCGCTGAGGTTAAGAAACCGGGTTCCTCAGTCAAAGTTAGTTGTAAGGCATCAGG
TTACACGATCACTGATAGTAACATACATTGGGTGAGACAGGCTCCTGGGCAGTCCCTTGAGTGGATTGGATACATTT
ATCCATATAATGGCGGCACGGATTACAACCAGAAATTTAAGAATAGAGCGACACTGACCGTTGACAACCCCACCAAT
ACCGCGTATATGGAATTGTCAAGCCTGAGGTCTGAGGACACGGCCTTCTACTATTGCGTAAACGGTAACCCGTGGCT
GGCCTACTGGGGGCAGGGGACACTCGTAACCGTCTCTTCT
>SEQ ID 274 anti-CD33 gemtuzumab VH aa
EVQLVQSGAEVKKPGSSVKVSCKASGYTITDSNIHWVRQAPGQSLEWIGYIYPYNGGTDYNQKFKNRATLTVDNPTN
TAYMELSSLRSEDTAFYYCVNGNPWLAYWGQGTLVTVSS
>SEQ ID 275 anti-CD33 gemtuzumab VL nt
GATATACAACTTACGCAGTCTCCCTCCACTCTGTCAGCGTCCGTAGGGGACAGAGTAACGATAACATGCAGGGCCTC
AGAAAGTCTTGACAACTACGGCATAAGGTTCCTCACATGGTTTCAACAGAAGCCGGGTAAAGCACCAAAACTCCTGA
TGTATGCTGCGTCTAACCAGGGGTCCGGTGTTCCTAGTCGGTTTTCAGGCAGCGGTAGCGGTACCGAGTTCACTCTC
ACAATTTCCAGTCTTCAGCCTGACGATTTCGCAACGTATTATTGCCAACAAACTAAGGAAGTGCCTTGGAGCTTTGG
GCAAGGCACCAAGGTCGAGGTGAAG
>SEQ ID 276 anti-CD33 gemtuzumab VL aa
DIQLTQSPSTLSASVGDRVTITCRASESLDNYGIRFLTWFQQKPGKAPKLLMYAASNQGSGVPSRFSGSGSGTEFTL
TISSLQPDDFATYYCQQTKEVPWSFGQGTKVEVK
>SEQ ID 277 anti-CD3 284A10 VH nt
GAGGTGCAGCTGGTGGAGTCTGGGGGAGGCTTGGTCCAGCCTGGGGGGTCCCTGAGACTCTCCTGTGCAGCCTCTGG
ATTCACCATCAGTACCAATGCAATGAGCTGGGTCCGCCAGGCTCCAGGGAAGGGGCTGGAGTGGATCGGAGTCATTA
CTGGTCGTGATATCACATACTACGCGAGCTGGGCGAAAGGCAGATTCACCATCTCCAGAGACAATTCCAAGAACACG
CTGTATCTTCAAATGAACAGCCTGAGAGCCGAGGACACGGCTGTGTATTACTGTGCGAGAGACGGTGGTTCTTCTGC
TATTACTAGTAACAACATTTGGGGCCAGGGAACCCTGGTCACCGTGTCGACA
>SEQ ID 278 anti-CD3 284A10 VH aa
EVQLVESGGGLVQPGGSLRLSCAASGFTISTNAMSWVRQAPGKGLEWIGVITGRDITYYASWAKGRFTISRDNSKNT
LYLQMNSLRAEDTAVYYCARDGGSSAITSNNIWGQGTLVTVST
>SEQ ID 279 anti-CD3 284A10 VL nt
GACGTCGTGATGACCCAGTCTCCTTCCACCCTGTCTGCATCTGTAGGAGACAGAGTCACCATCAATTGCCAAGCCAG
TGAGAGCATTAGCAGTTGGTTAGCCTGGTATCAGCAGAAACCAGGGAAAGCCCCTAAGCTCCTGATCTATGAAGCAT
CCAAACTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAAGGCTATTTTTATTTTATTAGTCGTACTTATGTAAATTCTTT
CGGCGGAGGGACCAAGGTGGAGATCAAA
>SEQ ID 280 anti-CD3 284A10 VL aa
DVVMTQSPSTLSASVGDRVTINCQASESISSWLAWYQQKPGKAPKLLIYEASKLASGVPSRFSGSGSGTEFTLTISS
LQPDDFATYYCQGYFYFISRTYVNSFGGGTKVEIK
>SEQ ID 281 anti-PD-L1 PL221G5 VH nt
GAGGTGCAGCTGTTGGAGTCTGGGGGAGGCTTGGTACAGCCTGGGGGGTCCCTGAGACTCTCCTGTGCAGCCTCTGG
ATTCTCCTTCAGTAGCGGGTACGACATGTGCTGGGTCCGCCAGGCTCCAGGGAAGGGGCTGGAGTGGATCGCATGCA
TTGCTGCTGGTAGTGCTGGTATCACTTACGACGCGAACTGGGCGAAAGGCCGGTTCACCATCTCCAGAGACAATTCC
AAGAACACGCTGTATCTGCAAATGAACAGCCTGAGAGCCGAGGACACGGCCGTATATTACTGTGCGAGATCGGCGTT
TTCGTTCGACTACGCCATGGACCTCTGGGGCCAGGGAACCCTGGTCACCGTCTCGAGC
>SEQ ID 282 anti-PD-L1 PL221G5 VH aa
EVQLLESGGGLVQPGGSLRLSCAASGFSFSSGYDMCWVRQAPGKGLEWIACIAAGSAGITYDANWAKGRFTISRDNS
KNTLYLQMNSLRAEDTAVYYCARSAFSFDYAMDLWGQGTLVTVSS
>SEQ ID 283 anti-PD-L1 PL221G5 VL nt
GACATCCAGATGACCCAGTCTCCTTCCACCCTGTCTGCATCTGTAGGAGACAGAGTCACCATCACTTGCCAGGCCAG
TCAGAGCATTAGTTCCCACTTAAACTGGTATCAGCAGAAACCAGGGAAAGCCCCTAAGCTCCTGATCTATAAGGCAT
CCACTCTGGCATCTGGGGTCCCATCAAGGTTCAGCGGCAGTGGATCTGGGACAGAATTTACTCTCACCATCAGCAGC
CTGCAGCCTGATGATTTTGCAACTTATTACTGCCAACAGGGTTATAGTTGGGGTAATGTTGATAATGTTTTCGGCGG
AGGGACCAAGGTGGAGATCAAA
>SEQ ID 284 anti-PD-L1 PL221G5 VL aa
DIQMTQSPSTLSASVGDRVTITCQASQSISSHLNWYQQKPGKAPKLLIYKASTLASGVPSRFSGSGSGTEFTLTISS
LQPDDFATYYCQQGYSWGNVDNVFGGGTKVEIK
>SEQ ID 285 anti-4-1BB 466F6 VH nt
CGGTCGCTGGTGGAGTCTGGGGGAGGCTTGGTCCAGCCTGGGGGGTCCCTGAGACTCTCCTGTACTGCCTCTGGATT
CACCATCAGTAGCTACCACATGCAGTGGGTCCGCCAGGCTCCAGGGAAGGGGCTGGAGTACATCGGAACCATTAGTA
GTGGTGGTAATGTATACTACGCAAGCTCCGCTAGAGGCAGATTCACCATCTCCAGACCCTCGTCCAAGAACACGGTG
GATCTTCAAATGAACAGCCTGAGAGCCGAGGACACGGCTGTGTATTACTGTGCGAGAGACTCTGGTTATAGTGATCC
TATGTGGGGCCAGGGAACCCTGGTCACCGTCTCTTCA
>SEQ ID 286 anti-4-1BB 466F6 VH aa
RSLVESGGGLVQPGGSLRLSCTASGFTISSYHMQWVRQAPGKGLEYIGTISSGGNVYYASSARGRFTISRPSSKNTV
DLQMNSLRAEDTAVYYCARDSGYSDPMWGQGTLVTVSS
>SEQ ID 287 anti-4-1BB 466F6 VL nt
GACGTTGTGATGACCCAGTCTCCATCTTCCGTGTCTGCATCTGTAGGAGACAGAGTCACCATCACCTGTCAGGCCAG
TCAGAACATTAGGACTTACTTATCCTGGTATCAGCAGAAACCAGGGAAAGCCCCTAAGCTCCTGATCTATGCTGCAG
CCAATCTGGCATCTGGGGTCCCATCAAGGTTCAGCGGCAGTGGATCTGGGACAGATTTCACTCTCACCATCAGCGAC
CTGGAGCCTGGCGATGCTGCAACTTACTATTGTCAGTCTACCTATCTTGGTACTGATTATGTTGGCGGTGCTTTCGG
CGGAGGGACCAAGGTGGAGATCAAA
>SEQ ID 288 anti-4-1BB 466F6 VL aa
DWMTQSPSSVSASVGDRVTITCQASQNIRTYLSWYQQKPGKAPKLLIYAAANLASGVPSRFSGSGSGTDFTLTISD
LEPGDAATYYCQSTYLGTDYVGGAFGGGTKVEIK
>SEQ ID 289 anti-CD19 SI-BU12 VH nt
CAGGTCACATTGAAGGAATCTGGCCCCGGCCTTGTTCAGCCAGGACAGACCCTTAGCCTCACCTGTGCCTTCAGTGG
TTTTTCTCTTAGCACTAGCGGTATGGGGGTCGGCTGGATTCGGCAGCCTCCCGGCAAAGGTCTTGAGTGGTTGGCTC
ACATTTGGTGGGACGACGACAAACGGTATAATCCTGCCTTGAAAAGTCGGCTGACCATTAGTAAGGATACCTCAAAA
AATCAAGTGTACTTGCAAATGAATAGCCTTGACGCCGAGGATACGGCTGTATATTATTGCGCTCGGATGGAACTCTG
GTCTTACTACTTTGATTATTGGGGGCAGGGGACTCTCGTCACGGTCTCGAGT
>SEQ ID 290 anti-CD19 SI-BU12 VH aa
QVTLKESGPGLVQPGQTLSLTCAFSGFSLSTSGMGVGWIRQPPGKGLEWLAHIWWDDDKRYNPALKSRLTISKDTSK
NQVYLQMNSLDAEDTAVYYCARMELWSYYFDYWGQGTLVTVSS
>SEQ ID 291 anti-CD19 SI-BU12 VL nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTA
>SEQ ID 292 anti-CD19 SI-BU12 VL aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVL
>SEQ ID 293 NKG2D nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTT
>SEQ ID 294 NKG2D aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTV
>SEQ ID 295 4-1BB ligand nt
CGAGAGGGCCCCGAGCTGTCTCCTGATGACCCAGCAGGCCTCTTGGACTTGCGGCAGGGTATGTTCGCTCAACTTGT
GGCTCAGAATGTTCTGCTCATTGATGGACCACTCTCTTGGTATAGTGACCCCGGTCTGGCCGGGGTGAGTCTGACCG
GCGGGCTCTCTTATAAAGAGGATACTAAGGAACTGGTCGTAGCAAAAGCGGGCGTTTATTACGTTTTTTTTCAGCTG
GAGCTCAGGCGCGTGGTGGCCGGCGAGGGCAGTGGCTCTGTGTCCCTGGCCCTGCACTTACAGCCCTTGAGAAGCGC
TGCAGGTGCTGCCGCACTGGCTTTAACTGTTGACCTCCCTCCGGCCTCTTCTGAAGCTAGAAACAGCGCTTTCGGCT
TCCAAGGGCGCCTGCTGCACCTGAGCGCAGGCCAGCGCTTAGGTGTGCACCTTCATACAGAGGCCAGGGCCCGACAC
GCTTGGCAGCTCACACAGGGTGCCACGGTTCTCGGACTTTTCCGCGTTACTCCCGAGATCCCCGCTGGCCTCGGAAG
TACTGGTTCTGGGTCTAAACCCGGTTCCGGCGAAGGTAGTACTAAAGGACGAGAAGGGCCAGAGTTAAGTCCAGATG
ACCCTGCTGGGCTTTTGGACCTGCGGCAGGGCATGTTCGCTCAACTGGTGGCTCAGAACGTGCTGCTGATCGATGGC
CCCCTGAGTTGGTACAGCGATCCCGGGCTGGCAGGCGTGTCACTTACAGGGGGCCTCTCTTACAAGGAAGACACCAA
GGAGTTAGTGGTCGCTAAGGCTGGCGTGTATTACGTGTTCTTCCAACTGGAGCTGAGAAGGGTTGTGGCAGGAGAGG
GTAGCGGCAGCGTGTCTTTAGCCCTTCACTTGCAGCCCCTGAGGTCTGCTGCAGGTGCAGCCGCTCTCGCGCTCACC
GTGGATCTCCCCCCAGCCTCATCTGAAGCTAGGAACAGTGCATTTGGCTTTCAGGGACGCTTGCTGCACCTCTCCGC
TGGACAGAGGCTGGGCGTGCACCTTCACACAGAGGCCCGTGCCAGGCATGCATGGCAGCTCACTCAGGGGGCAACAG
TGCTGGGTCTCTTCCGCGTGACTCCTGAAATACCAGCTGGACTTGGCGGTGGAGGCAGCGGCGGAGGAGGATCTCGT
GAGGGGCCAGAACTGTCCCCCGATGACCCAGCCGGACTGCTCGATCTCAGACAGGGCATGTTCGCTCAGCTTGTAGC
CCAAAATGTCCTCCTGATTGACGGCCCTTTGAGCTGGTATAGTGATCCCGGCTTGGCCGGGGTATCTCTGACCGGAG
GCCTCTCCTACAAGGAAGACACCAAAGAGCTGGTGGTGGCAAAAGCGGGGGTGTATTATGTGTTCTTTCAGCTCGAG
CTGCGGAGAGTTGTGGCCGGGGAAGGGTCTGGGAGCGTATCTCTTGCACTTCACCTGCAGCCCCTGCGCAGCGCCGC
TGGAGCCGCCGCCCTTGCTCTTACTGTGGATCTGCCTCCTGCTTCCTCAGAAGCACGCAACAGCGCCTTCGGCTTTC
AAGGACGTCTCCTGCACTTGTCCGCAGGACAGAGGTTGGGCGTCCATTTACACACTGAGGCACGGGCACGGCACGCT
TGGCAGCTTACCCAGGGAGCCACCGTGCTGGGACTCTTTAGAGTGACACCCGAGATCCCCGCTGGCTTG
>SEQ ID 296 4-1BB ligand aa
REGPELSPDDPAGLLDLRQGMFAQLVAQNVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSAFGFQGRLLHLSAGQRLGVHLHTEARARH
AWQLTQGATVLGLFRVTPEIPAGLGSTGSGSKPGSGEGSTKGREGPELSPDDPAGLLDLRQGMFAQLVAQNVLLIDG
PLSWYSDPGLAGVSLTGGLSYKEDTKELVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGATVLGLFRVTPEIPAGLGGGGSGGGGSR
EGPELSPDDPAGLLDLRQGMFAQLVAQNVLLIDGPLSWYSDPGLAGVSLTGGLSYKEDTKELVVAKAGVYYVFFQLE
LRRVVAGEGSGSVSLALHLQPLRSAAGAAALALTVDLPPASSEARNSAFGFQGRLLHLSAGQRLGVHLHTEARARHA
WQLTQGATVLGLFRVTPEIPAGL
>SEQ ID 301 SI-55P9 chain A nt
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGCACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTACACCCTGCCCCCATCCCGGGATGAG
CTGACCAAGAACCAGGTCAGCCTGACCTGCCTGGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGTGGAGGGTCCGGCGGTGGTGGATCCGAGGTGCAGCT
GTTGGAGTCTGGGGGAGGCTTGGTACAGCCTGGGGGGTCCCTGAGACTCTCCTGTGCAGCCTCTGGATTCTCCTTCA
GTAGCGGGTACGACATGTGCTGGGTCCGCCAGGCTCCAGGGAAGGGGCTGGAGTGGATCGCATGCATTGCTGCTGGT
AGTGCTGGTATCACTTACGACGCGAACTGGGCGAAAGGCCGGTTCACCATCTCCAGAGACAATTCCAAGAACACGCT
GTATCTGCAAATGAACAGCCTGAGAGCCGAGGACACGGCCGTATATTACTGTGCGAGATCGGCGTTTTCGTTCGACT
ACGCCATGGACCTCTGGGGCCAGGGAACCCTGGTCACCGTGTCGAGCGGTGGAGGCGGATCTGGCGGAGGTGGTTCC
GGCGGTGGCGGCTCCGGTGGAGGCGGCTCTGACATCCAGATGACCCAGTCTCCTTCCACCCTGTCTGCATCTGTAGG
AGACAGAGTCACCATCACTTGCCAGGCCAGTCAGAGCATTAGTTCCCACTTAAACTGGTATCAGCAGAAACCAGGGA
AAGCCCCTAAGCTCCTGATCTATAAGGCATCCACTCTGGCATCTGGGGTCCCATCAAGGTTCAGCGGCAGTGGATCT
GGGACAGAATTTACTCTCACCATCAGCAGCCTGCAGCCTGATGATTTTGCAACTTATTACTGCCAACAGGGTTATAG
TTGGGGTAATGTTGATAATGTTTTCGGCGGAGGGACCAAGGTGGAGATCAAAGGCGGTGGAGGGTCCGGCGGTGGTG
GCTCCGGACGGTCGCTGGTGGAGTCTGGGGGAGGCTTGGTCCAGCCTGGGGGGTCCCTGAGACTCTCCTGTACTGCC
TCTGGATTCACCATCAGTAGCTACCACATGCAGTGGGTCCGCCAGGCTCCAGGGAAGGGGCTGGAGTACATCGGAAC
CATTAGTAGTGGTGGTAATGTATACTACGCAAGCTCCGCTAGAGGCAGATTCACCATCTCCAGACCCTCGTCCAAGA
ACACGGTGGATCTTCAAATGAACAGCCTGAGAGCCGAGGACACGGCTGTGTATTACTGTGCGAGAGACTCTGGTTAT
AGTGATCCTATGTGGGGCCAGGGAACCCTGGTCACCGTCTCTTCAGGCGGTGGCGGTAGTGGGGGAGGCGGTTCTGG
CGGCGGAGGGTCCGGCGGTGGAGGATCAGACGTTGTGATGACCCAGTCTCCATCTTCCGTGTCTGCATCTGTAGGAG
ACAGAGTCACCATCACCTGTCAGGCCAGTCAGAACATTAGGACTTACTTATCCTGGTATCAGCAGAAACCAGGGAAA
GCCCCTAAGCTCCTGATCTATGCTGCAGCCAATCTGGCATCTGGGGTCCCATCAAGGTTCAGCGGCAGTGGATCTGG
GACAGATTTCACTCTCACCATCAGCGACCTGGAGCCTGGCGATGCTGCAACTTACTATTGTCAGTCTACCTATCTTG
GTACTGATTATGTTGGCGGTGCTTTCGGCGGAGGGACCAAGGTGGAGATCAAATGA
>SEQ ID 302 SI-55P9 chain A aa
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGAPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNVFSCSVMHEALHN
HYTQKSLSLSPGGGGGSGGGGSEVQLLESGGGLVQPGGSLRLSCAASGFSFSSGYDMCWVRQAPGKGLEWIACIAAG
SAGITYDANWAKGRFTISRDNSKNTLYLQMNSLRAEDTAVYYCARSAFSFDYAMDLWGQGTLVTVSSGGGGSGGGGS
GGGGSGGGGSDIQMTQSPSTLSASVGDRVTITCQASQSISSHLNWYQQKPGKAPKLLIYKASTLASGVPSRFSGSGS
GTEFTLTISSLQPDDFATYYCQQGYSWGNVDNVFGGGTKVEIKGGGGSGGGGSGRSLVESGGGLVQPGGSLRLSCTA
SGFTISSYHMQWVRQAPGKGLEYIGTISSGGNVYYASSARGRFTISRPSSKNTVDLQMNSLRAEDTAVYYCARDSGY
SDPMWGQGTLVTVSSGGGGSGGGGSGGGGSGGGGSDVVMTQSPSSVSASVGDRVTITCQASQNIRTYLSWYQQKPGK
APKLLIYAAANLASGVPSRFSGSGSGTDFTLTISDLEPGDAATYYCQSTYLGTDYVGGAFGGGTKVEIK
>SEQ ID 303 SI-55P9 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACGGTACTGGGTGGAGGCGGTTCAGGCGGAGGTGGTTCCGGCGGTGGCGGCTCCGGTGGAGGCGGCTCTCAGGTCA
CATTGAAGGAATCTGGCCCCGGCCTTGTTCAGCCAGGACAGACCCTTAGGCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGCGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTCACTCTCACCATCAGCAGCCT
GCAGCCTGATGATTTTGCAACTTATTACTGCCAAGGCTATTTTTATTTTATTAGTCGTACTTATGTAAATTCTTTCG
GCGGAGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTTGA
>SEQ ID 304 SI-55P9 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLRLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGEC
>SEQ ID 305 SI-55P10 chain A nt
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGCGGTGGAGGGTCCGGCGGTGGTGGATCAGA
GGTGCAGCTGGTGGAGTCTGGGGGAGGCTTGGTCCAGCCTGGGGGGTCCCTGAGACTCTCCTGTGCAGCCTCTGGAT
TCACCATCAGTACCAATGCAATGAGCTGGGTCCGCCAGGCTCCAGGGAAGGGGCTGGAGTGGATCGGAGTCATTACT
GGTCGTGATATCACATACTACGCGAGCTGGGCGAAAGGCAGATTCACCATCTCCAGAGACAATTCCAAGAACACGCT
GTATCTTCAAATGAACAGCCTGAGAGCCGAGGACACGGCTGTGTATTACTGTGCGAGAGACGGTGGTTCTTCTGCTA
TTACTAGTAACAACATTTGGGGCCAGGGAACCCTGGTCACCGTGTCCTCAGCTAGCACCAAGGGCCCATCGGTCTTC
CCCCTGGCACCCTCCTCCAAGAGCACCTCTGGGGGCACAGCGGCCCTGGGCTGCCTGGTCAAGGACTACTTCCCCGA
ACCGGTGACGGTGTCGTGGAACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAGACCTACATCTGCAACGTGAAT
CACAAGCCCAGCAACACCAAGGTGGACAAGAGAGTTGAGCCCAAATCTTGTGACAAAACTCACACATGCCCACCGTG
CCCAGCACCTGAAGCCGCGGGGGCACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCGCGGTCTCCAACAAAGCCCTCCCAGCCCCCA
TCGAGAAAACCATCTCCAAAGCCAAAGGGCAGCCCCGAGAACCACAGGTGTACACCCTGCCCCCATCCCGGGATGAG
CTGACCAAGAACCAGGTCAGCCTGACCTGCCTGGTCAAAGGCTTCTATCCCAGCGACATCGCCGTGGAGTGGGAGAG
CAATGGGCAGCCGGAGAACAACTACAAGACCACGCCTCCCGTGCTGGACTCCGACGGCTCCTTCTTCCTCTATAGCA
AGCTCACCGTGGACAAGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTGGCGGTGGAGGGTCCGGCGGTGGTGGATCCGAGGTGCAGCT
GTTGGAGTCTGGGGGAGGCTTGGTACAGCCTGGGGGGTCCCTGAGACTCTCCTGTGCAGCCTCTGGATTCTCCTTCA
GTAGCGGGTACGACATGTGCTGGGTCCGCCAGGCTCCAGGGAAGGGGCTGGAGTGGATCGCATGCATTGCTGCTGGT
AGTGCTGGTATCACTTACGACGCGAACTGGGCGAAAGGCCGGTTCACCATCTCCAGAGACAATTCCAAGAACACGCT
GTATCTGCAAATGAACAGCCTGAGAGCCGAGGACACGGCCGTATATTACTGTGCGAGATCGGCGTTTTCGTTCGACT
ACGCCATGGACCTCTGGGGCCAGGGAACCCTGGTCACCGTGTCGAGCGGTGGAGGCGGATCTGGCGGAGGTGGTTCC
GGCGGTGGCGGCTCCGGTGGAGGCGGCTCTGACATCCAGATGACCCAGTCTCCTTCCACCCTGTCTGCATCTGTAGG
AGACAGAGTCACCATCACTTGCCAGGCCAGTCAGAGCATTAGTTCCCACTTAAACTGGTATCAGCAGAAACCAGGGA
AAGCCCCTAAGCTCCTGATCTATAAGGCATCCACTCTGGCATCTGGGGTCCCATCAAGGTTCAGCGGCAGTGGATCT
GGGACAGAATTTACTCTCACCATCAGCAGCCTGCAGCCTGATGATTTTGCAACTTATTACTGCCAACAGGGTTATAG
TTGGGGTAATGTTGATAATGTTTTCGGCGGAGGGACCAAGGTGGAGATCAAAGGCGGTGGAGGGTCCGGCGGTGGTG
GCTCCGGACGAGAGGGCCCCGAGCTGTCTCCTGATGACCCAGCAGGCCTCTTGGACTTGCGGCAGGGTATGTTCGCT
CAACTTGTGGCTCAGAATGTTCTGCTCATTGATGGACCACTCTCTTGGTATAGTGACCCCGGTCTGGCCGGGGTGAG
TCTGACCGGCGGGCTCTCTTATAAAGAGGATACTAAGGAACTGGTCGTAGCAAAAGCGGGCGTTTATTACGTTTTTT
TTCAGCTGGAGCTCAGGCGCGTGGTGGCCGGCGAGGGCAGTGGCTCTGTGTCCCTGGCCCTGCACTTACAGCCCTTG
AGAAGCGCTGCAGGTGCTGCCGCACTGGCTTTAACTGTTGACCTCCCTCCGGCCTCTTCTGAAGCTAGAAACAGCGC
TTTCGGCTTCCAAGGGCGCCTGCTGCACCTGAGCGCAGGCCAGCGCTTAGGTGTGCACCTTCATACAGAGGCCAGGG
CCCGACACGCTTGGCAGCTCACACAGGGTGCCACGGTTCTCGGACTTTTCCGCGTTACTCCCGAGATCCCCGCTGGC
CTCGGAAGTACTGGTTCTGGGTCTAAACCCGGTTCCGGCGAAGGTAGTACTAAAGGACGAGAAGGGCCAGAGTTAAG
TCCAGATGACCCTGCTGGGCTTTTGGACCTGCGGCAGGGCATGTTCGCTCAACTGGTGGCTCAGAACGTGCTGCTGA
TCGATGGCCCCCTGAGTTGGTACAGCGATCCCGGGCTGGCAGGCGTGTCACTTACAGGGGGCCTCTCTTACAAGGAA
GACACCAAGGAGTTAGTGGTCGCTAAGGCTGGCGTGTATTACGTGTTCTTCCAACTGGAGCTGAGAAGGGTTGTGGC
AGGAGAGGGTAGCGGCAGCGTGTCTTTAGCCCTTCACTTGCAGCCCCTGAGGTCTGCTGCAGGTGCAGCCGCTCTCG
CGCTCACCGTGGATCTCCCCCCAGCCTCATCTGAAGCTAGGAACAGTGCATTTGGCTTTCAGGGACGCTTGCTGCAC
CTCTCCGCTGGACAGAGGCTGGGCGTGCACCTTCACACAGAGGCCCGTGCCAGGCATGCATGGCAGCTCACTCAGGG
GGCAACAGTGCTGGGTCTCTTCCGCGTGACTCCTGAAATACCAGCTGGACTTGGCGGTGGAGGCAGCGGCGGAGGAG
GATCTCGTGAGGGGCCAGAACTGTCCCCCGATGACCCAGCCGGACTGCTCGATCTCAGACAGGGCATGTTCGCTCAG
CTTGTAGCCCAAAATGTCCTCCTGATTGACGGCCCTTTGAGCTGGTATAGTGATCCCGGCTTGGCCGGGGTATCTCT
GACCGGAGGCCTCTCCTACAAGGAAGACACCAAAGAGCTGGTGGTGGCAAAAGCGGGGGTGTATTATGTGTTCTTTC
AGCTCGAGCTGCGGAGAGTTGTGGCCGGGGAAGGGTCTGGGAGCGTATCTCTTGCACTTCACCTGCAGCCCCTGCGC
AGCGCCGCTGGAGCCGCCGCCCTTGCTCTTACTGTGGATCTGCCTCCTGCTTCCTCAGAAGCACGCAACAGCGCCTT
CGGCTTTCAAGGACGTCTCCTGCACTTGTCCGCAGGACAGAGGTTGGGCGTCCATTTACACACTGAGGCACGGGCAC
GGCACGCTTGGCAGCTTACCCAGGGAGCCACCGTGCTGGGACTCTTTAGAGTGACACCCGAGATCCCCGCTGGCTTG
TGA
>SEQ ID 306 SI-55P10 chain A aa
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSEVQLVESGGGLVQPGGSLRLSCAASGFTISTNAMSWVRQAPGKGLEWIGVIT
GRDITYYASWAKGRFTISRDNSKNTLYLQMNSLRAEDTAVYYCARDGGSSAITSNNIWGQGTLVTVSSASTKGPSVF
PLAPSSKSTSGGTAALGCLVKDYFPEPVTVSWNSGALTSGVHTFPAVLQSSGLYSLSSVVTVPSSSLGTQTYICNVN
HKPSNTKVDKRVEPKSCDKTHTCPPCPAPEAAGAPSVFLFPPKPKDTLMISRTPEVTCVVVDVSHEDPEVKFNWYVD
GVEVHNAKTKPREEQYNSTYRVVSVLTVLHQDWLNGKEYKCAVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNVFSCSVMHEALHN
HYTQKSLSLSPGGGGGSGGGGSEVQLLESGGGLVQPGGSLRLSCAASGFSFSSGYDMCWVRQAPGKGLEWIACIAAG
SAGITYDANWAKGRFTISRDNSKNTLYLQMNSLRAEDTAVYYCARSAFSFDYAMDLWGQGTLVTVSSGGGGSGGGGS
GGGGSGGGGSDIQMTQSPSTLSASVGDRVTITCQASQSISSHLNWYQQKPGKAPKLLIYKASTLASGVPSRFSGSGS
GTEFTLTISSLQPDDFATYYCQQGYSWGNVDNVFGGGTKVEIKGGGGSGGGGSGREGPELSPDDPAGLLDLRQGMFA
QLVAQNVLLIDGPLSWYSDPGLAGVSLTGGLSYKEDTKELVVAKAGVYYVFFQLELRRVVAGEGSGSVSLALHLQPL
RSAAGAAALALTVDLPPASSEARNSAFGFQGRLLHLSAGQRLGVHLHTEARARHAWQLTQGATVLGLFRVTPEIPAG
LGSTGSGSKPGSGEGSTKGREGPELSPDDPAGLLDLRQGMFAQLVAQNVLLIDGPLSWYSDPGLAGVSLTGGLSYKE
DTKELVVAKAGVYYVFFQLELRRVVAGEGSGSVSLALHLQPLRSAAGAAALALTVDLPPASSEARNSAFGFQGRLLH
LSAGQRLGVHLHTEARARHAWQLTQGATVLGLFRVTPEIPAGLGGGGSGGGGSREGPELSPDDPAGLLDLRQGMFAQ
LVAQNVLLIDGPLSWYSDPGLAGVSLTGGLSYKEDTKELVVAKAGVYYVFFQLELRRVVAGEGSGSVSLALHLQPLR
SAAGAAALALTVDLPPASSEARNSAFGFQGRLLHLSAGQRLGVHLHTEARARHAWQLTQGATVLGLFRVTPEIPAGL
>SEQ ID 307 SI-55P10 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACGGTACTGGGTGGAGGCGGTTCAGGCGGAGGTGGTTCCGGCGGTGGCGGCTCCGGTGGAGGCGGCTCTCAGGTCA
CATTGAAGGAATCTGGCCCCGGCCTTGTTCAGCCAGGACAGACCCTTAGGCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCGCGGATGGAACTCTGGTCTTAC
TACTTTGATTATTGGGGGCAGGGGACTCTCGTCACGGTCTCGAGCGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTCACTCTCACCATCAGCAGCCT
GCAGCCTGATGATTTTGCAACTTATTACTGCCAAGGCTATTTTTATTTTATTAGTCGTACTTATGTAAATTCTTTCG
GCGGAGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTTGA
>SEQ ID 308 SI-55P10 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLRLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGEC
>SEQ ID 309 NKG2D dimer nt
TTTCTTAATTCCCTTTTCAACCAAGAGGTTCAGATCCCCTTGACTGAAAGCTATTGCGGCCCTTGTCCGAAAAACTG
GATATGTTACAAGAATAATTGTTACCAATTCTTCGACGAAAGCAAGAACTGGTATGAGAGTCAGGCGTCTTGTATGA
GTCAGAATGCCAGCCTGCTTAAGGTTTATTCAAAAGAAGACCAGGATCTGCTTAAGTTGGTAAAGAGCTACCACTGG
ATGGGGCTGGTACATATCCCAACGAATGGGTCATGGCAGTGGGAGGACGGTTCTATTCTGAGTCCAAATCTCCTGAC
GATCATCGAAATGCAGAAAGGGGACTGTGCCCTGTATGCATCATCCTTCAAGGGGTACATCGAGAACTGCAGTACCC
CAAATACCTACATTTGTATGCAAAGAACGGTTGGAGGCGGTGGCTCAGGCGGAGGCGGCTCAGGAGGTGGCGGTTCA
GGAGGCGGCGGATCTTTCCTAAACTCATTATTCAACCAAGAAGTTCAAATTCCCTTGACCGAAAGTTACTGTGGCCC
ATGTCCTAAAAACTGGATATGTTACAAAAATAACTGCTACCAATTTTTTGATGAGAGTAAAAACTGGTATGAGAGCC
AGGCTTCTTGTATGTCTCAAAATGCCAGCCTTCTGAAAGTATACAGCAAAGAGGACCAGGATTTACTTAAACTGGTG
AAGTCATATCATTGGATGGGACTAGTACACATTCCAACAAATGGATCTTGGCAGTGGGAAGATGGCTCCATTCTCTC
ACCCAACCTACTAACAATAATTGAAATGCAGAAGGGAGACTGTGCACTCTATGCCTCGAGCTTTAAAGGCTATATAG
AAAACTGTTCAACTCCAAATACGTACATCTGCATGCAAAGGACTGTG
>SEQ ID 310 NKG2D dimer aa
FLNSLFNQEVQIPLTESYCGPCPKNWICYKNNCYQFFDESKNWYESQASCMSQNASLLKVYSKEDQDLLKLVKSYHW
MGLVHIPTNGSWQWEDGSILSPNLLTIIEMQKGDCALYASSFKGYIENCSTPNTYICMQRTVGGGGSGGGGSGGGGS
GGGGSFLNSLFNQEVQIPLTESYCGPCPKNWICYKNNCYQFFDESKNWYESQASCMSQNASLLKVYSKEDQDLLKLV
KSYHWMGLVHIPTNGSWQWEDGSILSPNLLTIIEMQKGDCALYASSFKGYIENCSTPNTYICMQRTV
>SEQ ID 311 anti-FITC 4-4-20 VH nt
GAGGTGAAGCTGGATGAGACTGGAGGAGGCTTGGTGCAACCTGGGAGGCCCATGAAACTCTCCTGTGTTGCCTCTGG
ATTCACTTTTAGTGACTACTGGATGAACTGGGTCCGCCAGTCTCCAGAGAAAGGACTGGAGTGGGTAGCACAAATTA
GAAACAAACCTTATAATTATGAAACATATTATTCAGATTCTGTGAAAGGCAGATTCACCATCTCAAGAGATGATTCC
AAAAGTAGTGTCTACCTGCAAATGAACAACTTAAGAGTTGAAGACATGGGTATCTATTACTGTACGGGTTCTTACTA
TGGTATGGACTACTGGGGTCAAGGAACCTCAGTCACCGTCTCCTCA
>SEQ ID 312 anti-FITC 4-4-20 VH aa
EVKLDETGGGLVQPGRPMKLSCVASGFTFSDYWMNWVRQSPEKGLEWVAQIRNKPYNYETYYSDSVKGRFTISRDDS
KSSVYLQMNNLRVEDMGIYYCTGSYYGMDYWGQGTSVTVSS
>SEQ ID 313 anti-FITC 4-4-20 VL nt
GATGTCGTGATGACCCAAACTCCACTCTCCCTGCCTGTCAGTCTTGGAGATCAAGCCTCCATCTCTTGCAGATCTAG
TCAGAGCCTTGTACACAGTAATGGAAACACCTATTTACGTTGGTACCTGCAGAAGCCAGGCCAGTCTCCAAAGGTCC
TGATCTACAAAGTTTCCAACCGATTTTCTGGGGTCCCAGACAGGTTCAGTGGCAGTGGATCAGGGACAGATTTCACA
CTCAAGATCAGCAGAGTGGAGGCTGAGGATCTGGGAGTTTATTTCTGCTCTCAAAGTACACATGTTCCGTGGACGTT
CGGTGGAGGCACCAAGCTGGAAATCAAA
>SEQ ID 314 anti-FITC 4-4-20 VL aa
DVVMTQTPLSLPVSLGDQASISCRSSQSLVHSNGNTYLRWYLQKPGQSPKVLIYKVSNRFSGVPDRFSGSGSGTDFT
LKISRVEAEDLGVYFCSQSTHVPWTFGGGTKLEIK
>SEQ ID 315 anti-CD3 284A10 FR 1 VH nt
GAGGTGCAGCTGGTGGAGAGCGGCGGCGGCCTGGTGCAGCCCGGCGGCAGCCTGAGGCTGAGCTGCACCGCCAGCGG
CTTCACCATCAGCACCAACGCCATGAGCTGGGTGAGGCAGGCCCCCGGCAAGGGCCTGGAGTGGGTGGGCGTGATCA
CCGGCAGGGACATCACCTACTACGCCAGCTGGGCCAAGGGCAGGTTCACCATCAGCAGGGACACCAGCAAGAACACC
GTGTACCTGCAGATGAACAGCCTGAGGGCCGAGGACACCGCCGTGTACTACTGCGCCAGGGACGGCGGCAGCAGCGC
CATCACCAGCAACAACATCTGGGGCCAGGGCACCCTGGTGACCGTGAGCAGC
>SEQ ID 316 anti-CD3 284A10 FR 1 VH aa
EVQLVESGGGLVQPGGSLRLSCTASGFTISTNAMSWVRQAPGKGLEWVGVITGRDITYYASWAKGRFTISRDTSKNT
VYLQMNSLRAEDTAVYYCARDGGSSAITSNNIWGQGTLVTVSS
>SEQ ID 317 anti-CD3 284A10 FR 1 VL nt
GAGATCGTGATGACCCAGAGCCCCAGCACCCTGAGCGCCAGCGTGGGCGACAGGGTGATCATCACCTGCCAGGCCAG
CGAGAGCATCAGCAGCTGGCTGGCCTGGTACCAGCAGAAGCCCGGCAAGGCCCCCAAGCTGCTGATCTACGAGGCCA
GCAAGCTGGCCAGCGGCGTGCCCAGCAGGTTCAGCGGCAGCGGCAGCGGCGCCGAGTTCACCCTGACCATCAGCAGC
CTGCAGCCCGACGACTTCGCCACCTACTACTGCCAGGGCTACTTCTACTTCATCAGCAGGACCTACGTGAACAGCTT
CGGCCAGGGCACCAAGCTGACCGTGCTG
>SEQ ID 318 anti-CD3 284A10 FR 1 VL aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGQGTKLTVL
>SEQ ID 319 anti-EGFR H7 VH nt
CAAGTACAGTTGCAGCAATCCGGTCCCGGTCTCGTCAAACCGAGTGAGACGCTTAGTATAACGTGTACTGTTTCAGG
CTTTAGCCTTACGAACTATGGAGTTCACTGGATTCGGCAGGCACCCGGCAAAGGTTTGGAATGGCTGGGTGTTATTT
GGTCAGGTGGAAATACAGACTATAACACCCCCTTTACAAGTCGGTTCACAATTACGAAAGATAATTCCAAAAATCAA
GTTTATTTCAAGTTGAGATCCGTCCGCGCGGACGACACTGCGATCTACTATTGTGCGAGGGCACTGACCTACTACGA
TTACGAATTTGCGTATTGGGGGCAAGGGACTCTTGTAACAGTCTCGAGC
>SEQ ID 320 anti-EGFR H7 VH aa
QVQLQQSGPGLVKPSETLSITCTVSGFSLTNYGVHWIRQAPGKGLEWLGVIWSGGNTDYNTPFTSRFTITKDNSKNQ
VYFKLRSVRADDTAIYYCARALTYYDYEFAYWGQGTLVTVSS
>SEQ ID 321 anti-EGFR H7 VL nt
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTA
>SEQ ID 322 anti-EGFR H7 VL aa
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVL
>SEQ ID 323 SI-77H4 chain A nt
GAAATCGTTATGACGCAGAGTCCCTCCACGCTCTCCGCTAGTGTCGGGGATCGCGTCATTATCACATGCCAGGCCTC
CGAGTCAATCAGCAGCTGGCTTGCATGGTATCAACAGAAGCCGGGAAAAGCTCCTAAATTGCTGATCTATGAAGCGT
CAAAATTGGCGTCTGGTGTCCCATCTAGGTTCTCCGGCTCTGGGTCTGGTGCGGAATTTACTTTGACAATCTCCAGT
CTTCAACCAGACGATTTCGCTACCTACTACTGCCAAGGGTATTTCTATTTTATAAGCCGGACATATGTAAACTCCTT
CGGCCAAGGAACAAAGTTGACTGTTCTTGGTGGCGGAGGCAGTGGTGGCGGGGGCAGCGGAGGTGGTGGTTCAGGGG
GTGGTGGGAGCGAAGTCCAATTGGTAGAAAGTGGCGGTGGTCTGGTGCAACCTGGTGGATCTCTTCGCCTCTCATGC
GCCGCTAGTGGCTTTACTATTTCAACTAATGCGATGAGCTGGGTTCGCCAGGCCCCCGGCAAAGGACTTGAGTGGGT
CGGCGTCATCACCGGCAGGGACATTACATACTATGCGAGTTGGGCAAAGGGCAGGTTCACGATTAGCCGCGATACTT
CAAAGAATACCGTTTACCTTCAAATGAATAGCTTGAGGGCGGAAGACACAGCTGTGTATTACTGCGCGAGGGATGGA
GGTAGTTCCGCCATAACTTCCAACAACATATGGGGACAAGGCACGCTGGTTACTGTCTCGAGTGGCGGTGGAGGGTC
CGGCGGTGGTGGATCACAAGTACAGTTGCAGCAATCCGGTCCCGGTCTCGTCAAACCGAGTGAGACGCTTAGTATAA
CGTGTACTGTTTCAGGCTTTAGCCTTACGAACTATGGAGTTCACTGGATTCGGCAGGCACCCGGCAAATGTTTGGAA
TGGCTGGGTGTTATTTGGTCAGGTGGAAATACAGACTATAACACCCCCTTTACAAGTCGGTTCACAATTACGAAAGA
TAATTCCAAAAATCAAGTTTATTTCAAGTTGAGATCCGTCCGCGCGGACGACACTGCGATCTACTATTGTGCGAGGG
CACTGACCTACTACGATTACGAATTTGCGTATTGGGGGCAAGGGACTCTTGTAACAGTCTCCAGTGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGCACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGGGGCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTGTCGAGCGGTGGAGGCGGATCT
GGCGGAGGTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATCAAAGGCGGTGGAG
GGTCCGGCGGTGGTGGCTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGCCAGGCTCCAGGGAAGGGGCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACCTGGAGCCTGGCGATGCTGCAACTTACTATTGTC
AGTCTACCTATCTTGGTACTGATTATGTTGGCGGTGCTTTCGGCGGAGGGACCAAGGTGGAGATCAAATGA
>SEQ ID 324 SI-77H4 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGQGTKLTVLGGGGSGGGGSGGGGSGGGGSEVQLVESGGGLVQPGGSLRLSC
AASGFTISTNAMSWVRQAPGKGLEWVGVITGRDITYYASWAKGRFTISRDTSKNTVYLQMNSLRAEDTAVYYCARDG
GSSAITSNNIWGQGTLVTVSSGGGGSGGGGSQVQLQQSGPGLVKPSETLSITCTVSGFSLTNYGVHWIRQAPGKCLE
WLGVIWSGGNTDYNTPFTSRFTITKDNSKNQVYFKLRSVRADDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVEIKGGGGSGGGGSGRSLVESGGGLVQPGGSLR
LSCTASGFTISSYHMQWVRQAPGKGLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGGGTKVEIK
>SEQ ID 325 SI-77H4 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCTCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
AATCGTCCTTACACAATCTCCTAGCACACTGAGTGTGAGCCCCGGCGAACGCGCGACTTTCTCTTGCAGGGCAAGTC
AATCCATAGGGACTAATATACATTGGTATCAACAAAAGCCAGGTAAACCACCCAGGCTTTTGATTAAGTATGCAAGT
GAGTCTATTTCCGGTATCCCTGACCGCTTCTCTGGATCAGGCAGTGGCACAGAGTTCACACTCACCATATCTAGTGT
GCAATCAGAGGACTTCGCCGTGTATTACTGCCAACAGAATAATAACTGGCCGACTACCTTCGGATGCGGTACAAAGC
TGACCGTTTTACGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCTCGCCCGTCACA
AAGAGCTTCAACAGGGGAGAGTGTGGCGGTGGCGGTAGCGGTGGCGGCGGAAGTGGTGGCGGAGGATCCCAGTCTGC
CCTGACTCAGCCTGCCTCCGTGTCTGGGTCTCCTGGACAGTCGATCACCATCTCCTGCACTGGAACCAGCAGTGACG
TTGGTGGTTATAACTTTGTCTCCTGGTACCAACAACACCCAGGCAAAGCCCCCAAACTCATGATCTATGATGTCAGT
GATCGGCCCTCAGGGGTGTCTGATCGCTTCTCCGGCTCCAAGTCTGGCAACACGGCCTCCCTGATCATCTCTGGCCT
CCAGGCTGACGACGAGGCTGATTATTACTGCAGCTCATATGGGAGCAGCAGCACTCATGTGATTTTCGGCGGAGGGA
CCAAGGTGACCGTCCTAGGTGGAGGCGGTTCAGGCGGAGGTGGTTCCGGCGGTGGCGGCTCCGGTGGAGGCGGCTCT
CAGGTGCAATTGCAGGAGTCGGGGGGAGGCCTGGTCAAGCCTGGAGGGTCCCTGAGTCTCTCCTGTGCAGCCTCTGG
ATTCACCTTTAGTAGTTATTGGATGAGCTGGGTCCGCCAGGCTCCAGGGAAGGGGCTGGAGTGGGTGGCCAACATAA
ACCGCGATGGAAGTGCGAGTTACTATGTGGACTCTGTGAAGGGCCGATTCACCATCTCCAGAGACGACGCCAAGAAC
TCACTGTATCTGCAAATGAACAGCCTGAGAGCTGAGGACACGGCTGTGTATTACTGTGCGAGAGATCGTGGGGTGGG
CTACTTCGATCTCTGGGGCCGTGGCACCCTGGTCACCGTCTCTAGCTGA
>SEQ ID 326 SI-77H4 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSEIVLTQSPSTLSVSPGERATFSCRASQSIGTNIHWYQQKPGKPPRLLIKYAS
ESISGIPDRFSGSGSGTEFTLTISSVQSEDFAVYYCQQNNNWPTTFGCGTKLTVLRTVAAPSVFIFPPSDEQLKSGT
ASVVCLLNNFYPREAKVQWKVDNALQSGNSQESVTEQDSKDSTYSLSSTLTLSKADYEKHKVYACEVTHQGLSSPVT
KSFNRGECGGGGSGGGGSGGGGSQSALTQPASVSGSPGQSITISCTGTSSDVGGYNFVSWYQQHPGKAPKLMIYDVS
DRPSGVSDRFSGSKSGNTASLIISGLQADDEADYYCSSYGSSSTHVIFGGGTKVTVLGGGGSGGGGSGGGGSGGGGS
QVQLQESGGGLVKPGGSLSLSCAASGFTFSSYWMSWVRQAPGKGLEWVANINRDGSASYYVDSVKGRFTISRDDAKN
SLYLQMNSLRAEDTAVYYCARDRGVGYFDLWGRGTLVTVSS
>SEQ ID 327 SI-77H5 chain A nt
GAAATCGTTATGACGCAGAGTCCCTCCACGCTCTCCGCTAGTGTCGGGGATCGCGTCATTATCACATGCCAGGCCTC
CGAGTCAATCAGCAGCTGGCTTGCATGGTATCAACAGAAGCCGGGAAAAGCTCCTAAATTGCTGATCTATGAAGCGT
CAAAATTGGCGTCTGGTGTCCCATCTAGGTTCTCCGGCTCTGGGTCTGGTGCGGAATTTACTTTGACAATCTCCAGT
CTTCAACCAGACGATTTCGCTACCTACTACTGCCAAGGGTATTTCTATTTTATAAGCCGGACATATGTAAACTCCTT
CGGCCAAGGAACAAAGTTGACTGTTCTTGGTGGCGGAGGCAGTGGTGGCGGGGGCAGCGGAGGTGGTGGTTCAGGGG
GTGGTGGGAGCGAAGTCCAATTGGTAGAAAGTGGCGGTGGTCTGGTGCAACCTGGTGGATCTCTTCGCCTCTCATGC
GCCGCTAGTGGCTTTACTATTTCAACTAATGCGATGAGCTGGGTTCGCCAGGCCCCCGGCAAAGGACTTGAGTGGGT
CGGCGTCATCACCGGCAGGGACATTACATACTATGCGAGTTGGGCAAAGGGCAGGTTCACGATTAGCCGCGATACTT
CAAAGAATACCGTTTACCTTCAAATGAATAGCTTGAGGGCGGAAGACACAGCTGTGTATTACTGCGCGAGGGATGGA
GGTAGTTCCGCCATAACTTCCAACAACATATGGGGACAAGGCACGCTGGTTACTGTCTCGAGTGGCGGTGGAGGGTC
CGGCGGTGGTGGATCACAGGTGCAGCTGAAGCAGTCAGGACCTGGCCTAGTGCAGCCCTCACAGAGCCTGTCCATCA
CCTGCACAGTCTCTGGTTTCTCATTAACTAACTATGGTGTACACTGGGTTCGCCAGTCTCCAGGAAAGTGTCTGGAG
TGGCTGGGAGTGATATGGAGTGGTGGAAACACAGACTATAATACACCTTTCACATCCAGACTGAGCATCAACAAGGA
CAATTCCAAGAGCCAAGTTTTCTTTAAAATGAACAGTCTGCAATCTAATGACACAGCCATATATTACTGTGCCAGAG
CCCTCACCTACTATGATTACGAGTTTGCTTACTGGGGCCAAGGGACTCTGGTCACTGTCTCGAGTGCTAGCACCAAG
GGCCCATCGGTCTTCCCCCTGGCACCCTCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAGAGTTGAGCCCAAATCTTGTGACAAAACTCA
CACATGCCCACCGTGCCCAGCACCTGAAGCCGCGGGGGCACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGCAGTACAACAGCACGTACCG
TGTGGTCAGCGTCCTCACCGTCCTGCACCAGGACTGGCTGAATGGCAAGGAGTACAAGTGCGCGGTCTCCAACAAAG
CCCTCCCAGCCCCCATCGAGAAAACCATCTCCAAAGCCAAAGGGCAGCCCCGAGAACCACAGGTGTACACCCTGCCC
CCATCCCGGGATGAGCTGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGACTCCGACGGCTCCT
TCTTCCTCTATAGCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTGGCGGTGGAGGGTCCGGCGGTGGTGG
ATCCGAGGTGCAGCTGTTGGAGTCTGGGGGAGGCTTGGTACAGCCTGGGGGGTCCCTGAGACTCTCCTGTGCAGCCT
CTGGATTCTCCTTCAGTAGCGGGTACGACATGTGCTGGGTCCGCCAGGCTCCAGGGAAGGGGCTGGAGTGGATCGCA
TGCATTGCTGCTGGTAGTGCTGGTATCACTTACGACGCGAACTGGGCGAAAGGCCGGTTCACCATCTCCAGAGACAA
TTCCAAGAACACGCTGTATCTGCAAATGAACAGCCTGAGAGCCGAGGACACGGCCGTATATTACTGTGCGAGATCGG
CGTTTTCGTTCGACTACGCCATGGACCTCTGGGGCCAGGGAACCCTGGTCACCGTGTCGAGCGGTGGAGGCGGATCT
GGCGGAGGTGGTTCCGGCGGTGGCGGCTCCGGTGGAGGCGGCTCTGACATCCAGATGACCCAGTCTCCTTCCACCCT
GTCTGCATCTGTAGGAGACAGAGTCACCATCACTTGCCAGGCCAGTCAGAGCATTAGTTCCCACTTAAACTGGTATC
AGCAGAAACCAGGGAAAGCCCCTAAGCTCCTGATCTATAAGGCATCCACTCTGGCATCTGGGGTCCCATCAAGGTTC
AGCGGCAGTGGATCTGGGACAGAATTTACTCTCACCATCAGCAGCCTGCAGCCTGATGATTTTGCAACTTATTACTG
CCAACAGGGTTATAGTTGGGGTAATGTTGATAATGTTTTCGGCGGAGGGACCAAGGTGGAGATCAAAGGCGGTGGAG
GGTCCGGCGGTGGTGGCTCCGGACGGTCGCTGGTGGAGTCTGGGGGAGGCTTGGTCCAGCCTGGGGGGTCCCTGAGA
CTCTCCTGTACTGCCTCTGGATTCACCATCAGTAGCTACCACATGCAGTGGGTCCGCCAGGCTCCAGGGAAGGGGCT
GGAGTACATCGGAACCATTAGTAGTGGTGGTAATGTATACTACGCAAGCTCCGCTAGAGGCAGATTCACCATCTCCA
GACCCTCGTCCAAGAACACGGTGGATCTTCAAATGAACAGCCTGAGAGCCGAGGACACGGCTGTGTATTACTGTGCG
AGAGACTCTGGTTATAGTGATCCTATGTGGGGCCAGGGAACCCTGGTCACCGTCTCTTCAGGCGGTGGCGGTAGTGG
GGGAGGCGGTTCTGGCGGCGGAGGGTCCGGCGGTGGAGGATCAGACGTTGTGATGACCCAGTCTCCATCTTCCGTGT
CTGCATCTGTAGGAGACAGAGTCACCATCACCTGTCAGGCCAGTCAGAACATTAGGACTTACTTATCCTGGTATCAG
CAGAAACCAGGGAAAGCCCCTAAGCTCCTGATCTATGCTGCAGCCAATCTGGCATCTGGGGTCCCATCAAGGTTCAG
CGGCAGTGGATCTGGGACAGATTTCACTCTCACCATCAGCGACCTGGAGCCTGGCGATGCTGCAACTTACTATTGTC
AGTCTACCTATCTTGGTACTGATTATGTTGGCGGTGCTTTCGGCGGAGGGACCAAGGTGGAGATCAAATGA
>SEQ ID 328 SI-77H5 chain A aa
EIVMTQSPSTLSASVGDRVIITCQASESISSWLAWYQQKPGKAPKLLIYEASKLASGVPSRFSGSGSGAEFTLTISS
LQPDDFATYYCQGYFYFISRTYVNSFGQGTKLTVLGGGGSGGGGSGGGGSGGGGSEVQLVESGGGLVQPGGSLRLSC
AASGFTISTNAMSWVRQAPGKGLEWVGVITGRDITYYASWAKGRFTISRDTSKNTVYLQMNSLRAEDTAVYYCARDG
GSSAITSNNIWGQGTLVTVSSGGGGSGGGGSQVQLKQSGPGLVQPSQSLSITCTVSGFSLTNYGVHWVRQSPGKCLE
WLGVIWSGGNTDYNTPFTSRLSINKDNSKSQVFFKMNSLQSNDTAIYYCARALTYYDYEFAYWGQGTLVTVSSASTK
GPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKRVEPKSCDKTHTCPPCPAPEAAGAPSVFLFPPKPKDTLMISRTPEVTCVVVDVSHEDPEVKF
NWYVDGVEVHNAKTKPREEQYNSTYRVVSVLTVLHQDWLNGKEYKCAVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMH
EALHNHYTQKSLSLSPGGGGGSGGGGSEVQLLESGGGLVQPGGSLRLSCAASGFSFSSGYDMCWVRQAPGKGLEWIA
CIAAGSAGITYDANWAKGRFTISRDNSKNTLYLQMNSLRAEDTAVYYCARSAFSFDYAMDLWGQGTLVTVSSGGGGS
GGGGSGGGGSGGGGSDIQMTQSPSTLSASVGDRVTITCQASQSISSHLNWYQQKPGKAPKLLIYKASTLASGVPSRF
SGSGSGTEFTLTISSLQPDDFATYYCQQGYSWGNVDNVFGGGTKVEIKGGGGSGGGGSGRSLVESGGGLVQPGGSLR
LSCTASGFTISSYHMQWVRQAPGKGLEYIGTISSGGNVYYASSARGRFTISRPSSKNTVDLQMNSLRAEDTAVYYCA
RDSGYSDPMWGQGTLVTVSSGGGGSGGGGSGGGGSGGGGSDVVMTQSPSSVSASVGDRVTITCQASQNIRTYLSWYQ
QKPGKAPKLLIYAAANLASGVPSRFSGSGSGTDFTLTISDLEPGDAATYYCQSTYLGTDYVGGAFGGGTKVEIK
>SEQ ID 329 SI-77H5 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCTCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
CATCTTGCTGACTCAGTCTCCAGTCATCCTGTCTGTGAGTCCAGGAGAAAGAGTCAGTTTCTCCTGCAGGGCCAGTC
AGAGTATTGGCACAAACATACACTGGTATCAGCAAAGAACAAATGGTTCTCCAAGGCTTCTCATAAAGTATGCTTCT
GAGTCTATCTCTGGGATTCCTTCCAGGTTTAGTGGCAGTGGATCAGGGACAGATTTTACTCTTAGCATCAACAGTGT
GGAGTCTGAAGATATTGCAGATTATTACTGTCAACAAAATAATAACTGGCCAACCACGTTCGGTTGTGGGACCAAGC
TGGAGCTGAAACGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCCCTCCA
ATCGGGTAACTCCCAGGAGAGTGTCACAGAGCAGGACAGCAAGGACAGCACCTACAGCCTCAGCAGCACCCTGACGC
TGAGCAAAGCAGACTACGAGAAACACAAAGTCTACGCCTGCGAAGTCACCCATCAGGGCCTGAGCTCGCCCGTCACA
AAGAGCTTCAACAGGGGAGAGTGTGGCGGTGGCGGTAGCGGTGGCGGCGGAAGTGGTGGCGGAGGATCCCAGTCTGC
CCTGACTCAGCCTGCCTCCGTGTCTGGGTCTCCTGGACAGTCGATCACCATCTCCTGCACTGGAACCAGCAGTGACG
TTGGTGGTTATAACTTTGTCTCCTGGTACCAACAACACCCAGGCAAAGCCCCCAAACTCATGATCTATGATGTCAGT
GATCGGCCCTCAGGGGTGTCTGATCGCTTCTCCGGCTCCAAGTCTGGCAACACGGCCTCCCTGATCATCTCTGGCCT
CCAGGCTGACGACGAGGCTGATTATTACTGCAGCTCATATGGGAGCAGCAGCACTCATGTGATTTTCGGCGGAGGGA
CCAAGGTGACCGTCCTAGGTGGAGGCGGTTCAGGCGGAGGTGGTTCCGGCGGTGGCGGCTCCGGTGGAGGCGGCTCT
CAGGTGCAATTGCAGGAGTCGGGGGGAGGCCTGGTCAAGCCTGGAGGGTCCCTGAGTCTCTCCTGTGCAGCCTCTGG
ATTCACCTTTAGTAGTTATTGGATGAGCTGGGTCCGCCAGGCTCCAGGGAAGGGGCTGGAGTGGGTGGCCAACATAA
ACCGCGATGGAAGTGCGAGTTACTATGTGGACTCTGTGAAGGGCCGATTCACCATCTCCAGAGACGACGCCAAGAAC
TCACTGTATCTGCAAATGAACAGCCTGAGAGCTGAGGACACGGCTGTGTATTACTGTGCGAGAGATCGTGGGGTGGG
CTACTTCGATCTCTGGGGCCGTGGCACCCTGGTCACCGTCTCTAGCTGA
>SEQ ID 330 SI-77H5 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDILLTQSPVILSVSPGERVSFSCRASQSIGTNIHWYQQRTNGSPRLLIKYAS
ESISGIPSRFSGSGSGTDFTLSINSVESEDIADYYCQQNNNWPTTFGCGTKLELKRTVAAPSVFIFPPSDEQLKSGT
ASVVCLLNNFYPREAKVQWKVDNALQSGNSQESVTEQDSKDSTYSLSSTLTLSKADYEKHKVYACEVTHQGLSSPVT
KSFNRGECGGGGSGGGGSGGGGSQSALTQPASVSGSPGQSITISCTGTSSDVGGYNFVSWYQQHPGKAPKLMIYDVS
DRPSGVSDRFSGSKSGNTASLIISGLQADDEADYYCSSYGSSSTHVIFGGGTKVTVLGGGGSGGGGSGGGGSGGGGS
QVQLQESGGGLVKPGGSLSLSCAASGFTFSSYWMSWVRQAPGKGLEWVANINRDGSASYYVDSVKGRFTISRDDAKN
SLYLQMNSLRAEDTAVYYCARDRGVGYFDLWGRGTLVTVSS
>SEQ ID 331 SI-55H12 chain A nt
GAAATCGTCCTTACACAATCTCCTAGCACACTGAGTGTGAGCCCCGGCGAACGCGCGACTTTCTCTTGCAGGGCAAG
TCAATCCATAGGGACTAATATACATTGGTATCAACAAAAGCCAGGTAAACCACCCAGGCTTTTGATTAAGTATGCAA
GTGAGTCTATTTCCGGTATCCCTGACCGCTTCTCTGGATCAGGCAGTGGCACAGAGTTCACACTCACCATATCTAGT
GTGCAATCAGAGGACTTCGCCGTGTATTACTGCCAACAGAATAATAACTGGCCGACTACCTTCGGACCCGGTACAAA
GCTGACCGTTTTAGGCGGTGGCGGTAGTGGGGGAGGCGGTTCTGGCGGCGGAGGGTCCGGCGGTGGAGGATCACAAG
TACAGTTGCAGCAATCCGGTCCCGGTCTCGTCAAACCGAGTGAGACGCTTAGTATAACGTGTACTGTTTCAGGCTTT
AGCCTTACGAACTATGGAGTTCACTGGATTCGGCAGGCACCCGGCAAAGGTTTGGAATGGCTGGGTGTTATTTGGTC
AGGTGGAAATACAGACTATAACACCCCCTTTACAAGTCGGTTCACAATTACGAAAGATAATTCCAAAAATCAAGTTT
ATTTCAAGTTGAGATCCGTCCGCGCGGACGACACTGCGATCTACTATTGTGCGAGGGCACTGACCTACTACGATTAC
GAATTTGCGTATTGGGGGCAAGGGACTCTTGTAACAGTCTCGAGCGGTGGAGGCGGATCTGGCGGAGGTGGTTCCGG
CGGTGGCGGCTCCGGTGGAGGCGGCTCTGAGGTGCAGCTGGTGGAGTCTGGGGGAGGCTTGGTCCAGCCTGGGGGGT
CCCTGAGACTCTCCTGTGCAGCCTCTGGATTCACCATCAGTACCAATGCAATGAGCTGGGTCCGCGAGGCTCCAGGG
AAGGGCCTGGAGTGGATCGGAGTCATTACTGGTCGTGATATCACATACTACGCGAGCTGGGCGAAAGGCAGATTCAC
CATCTCCAGAGACAATTCCAAGAACACGCTGTATCTTCAAATGAACAGCCTGAGAGCCGAGGACACGGCTGTGTATT
ACTGTGCGAGAGACGGTGGTTCTTCTGCTATTACTAGTAACAACATTTGGGGCCAGGGAACCCTGGTCACCGTGTCC
TCAGCTAGCACCAAGGGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCCTGACCAGCGGCGTGC
ACACCTTCCCGGCTGTCCTACAGTCCTCAGGACTCTACTCCCTCAGCAGCGTGGTGACCGTGCCCTCCAGCAGCTTG
GGCACCCAGACCTACATCTGCAACGTGAATCACAAGCCCAGCAACACCAAGGTGGACAAGAGAGTTGAGCCCAAATC
TTGTGACAAAACTCACACATGCCCACCGTGCCCAGCACCTGAAGCCGCGGGGGCACCGTCAGTCTTCCTCTTCCCCC
CAAAACCCAAGGACACCCTCATGATCTCCCGGACCCCTGAGGTCACATGCGTGGTGGTGGACGTGAGCCACGAAGAC
CCTGAGGTCAAGTTCAACTGGTACGTGGACGGCGTGGAGGTGCATAATGCCAAGACAAAGCCGCGGGAGGAGCAGTA
CAACAGCACGTACCGTGTGGTCAGCGTCCTCACCGTCCTGCACCAGGACTGGCTGAATGGCAAGGAGTACAAGTGCG
CGGTCTCCAACAAAGCCCTCCCAGCCCCCATCGAGAAAACCATCTCCAAAGCCAAAGGGCAGCCCCGAGAACCACAG
GTGTACACCCTGCCCCCATCCCGGGATGAGCTGACCAAGAACCAGGTCAGCCTGACCTGCCTGGTCAAAGGCTTCTA
TCCCAGCGACATCGCCGTGGAGTGGGAGAGCAATGGGCAGCCGGAGAACAACTACAAGACCACGCCTCCCGTGCTGG
ACTCCGACGGCTCCTTCTTCCTCTATAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGGTGGCGGTGGAGG
GTCCGGCGGTGGTGGATCCGAGGTGCAGCTGTTGGAGTCTGGGGGAGGCTTGGTACAGCCTGGGGGGTCCCTGAGAC
TCTCCTGTGCAGCCTCTGGATTCTCCTTCAGTAGCGGGTACGACATGTGCTGGGTCCGCCAGGCTCCAGGGAAGGGG
CTGGAGTGGATCGCATGCATTGCTGCTGGTAGTGCTGGTATCACTTACGACGCGAACTGGGCGAAAGGCCGGTTCAC
CATCTCCAGAGACAATTCCAAGAACACGCTGTATCTGCAAATGAACAGCCTGAGAGCCGAGGACACGGCCGTATATT
ACTGTGCGAGATCGGCGTTTTCGTTCGACTACGCCATGGACCTCTGGGGCCAGGGAACCCTGGTCACCGTGTCGAGC
GGTGGAGGCGGATCTGGCGGAGGTGGTTCCGGCGGTGGCGGCTCCGGTGGAGGCGGCTCTGACATCCAGATGACCCA
GTCTCCTTCCACCCTGTCTGCATCTGTAGGAGACAGAGTCACCATCACTTGCCAGGCCAGTCAGAGCATTAGTTCCC
ACTTAAACTGGTATCAGCAGAAACCAGGGAAAGCCCCTAAGCTCCTGATCTATAAGGCATCCACTCTGGCATCTGGG
GTCCCATCAAGGTTCAGCGGCAGTGGATCTGGGACAGAATTTACTCTCACCATCAGCAGCCTGCAGCCTGATGATTT
TGCAACTTATTACTGCCAACAGGGTTATAGTTGGGGTAATGTTGATAATGTTTTCGGCGGAGGGACCAAGGTGGAGA
TCAAAGGCGGTGGAGGGTCCGGCGGTGGTGGCTCCGGACGGTCGCTGGTGGAGTCTGGGGGAGGCTTGGTCCAGCCT
GGGGGGTCCCTGAGACTCTCCTGTACTGCCTCTGGATTCACCATCAGTAGCTACCACATGCAGTGGGTCCGCCAGGC
TCCAGGGAAGGGGCTGGAGTACATCGGAACCATTAGTAGTGGTGGTAATGTATACTACGCAAGCTCCGCTAGAGGCA
GATTCACCATCTCCAGACCCTCGTCCAAGAACACGGTGGATCTTCAAATGAACAGCCTGAGAGCCGAGGACACGGCT
GTGTATTACTGTGCGAGAGACTCTGGTTATAGTGATCCTATGTGGGGCCAGGGAACCCTGGTCACCGTCTCTTCAGG
CGGTGGCGGTAGTGGGGGAGGCGGTTCTGGCGGCGGAGGGTCCGGCGGTGGAGGATCAGACGTTGTGATGACCCAGT
CTCCATCTTCCGTGTCTGCATCTGTAGGAGACAGAGTCACCATCACCTGTCAGGCCAGTCAGAACATTAGGACTTAC
TTATCCTGGTATCAGCAGAAACCAGGGAAAGCCCCTAAGCTCCTGATCTATGCTGCAGCCAATCTGGCATCTGGGGT
CCCATCAAGGTTCAGCGGCAGTGGATCTGGGACAGATTTCACTCTCACCATCAGCGACCTGGAGCCTGGCGATGCTG
CAACTTACTATTGTCAGTCTACCTATCTTGGTACTGATTATGTTGGCGGTGCTTTCGGCGGAGGGACCAAGGTGGAG
ATCAAATGA
>SEQ ID 332 SI-55H12 chain A aa
EIVLTQSPSTLSVSPGERATFSCRASQSIGTNIHWYQQKPGKPPRLLIKYASESISGIPDRFSGSGSGTEFTLTISS
VQSEDFAVYYCQQNNNWPTTFGPGTKLTVLGGGGSGGGGSGGGGSGGGGSQVQLQQSGPGLVKPSETLSITCTVSGF
SLTNYGVHWIRQAPGKGLEWLGVIWSGGNTDYNTPFTSRFTITKDNSKNQVYFKLRSVRADDTAIYYCARALTYYDY
EFAYWGQGTLVTVSSGGGGSGGGGSGGGGSGGGGSEVQLVESGGGLVQPGGSLRLSCAASGFTISTNAMSWVREAPG
KGLEWIGVITGRDITYYASWAKGRFTISRDNSKNTLYLQMNSLRAEDTAVYYCARDGGSSAITSNNIWGQGTLVTVS
SASTKGPSVFPLAPSSKSTSGGTAALGCLVKDYFPEPVTVSWNSGALTSGVHTFPAVLQSSGLYSLSSVVTVPSSSL
GTQTYICNVNHKPSNTKVDKRVEPKSCDKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYKCAVSNKALPAPIEKTISKAKGQPREPQ
VYTLPPSRDELTKNQVSLTCLVKGFYPSDIAVEWESNGQPENNYKTTPPVLDSDGSFFLYSKLTVDKSRWQQGNVFS
CSVMHEALHNHYTQKSLSLSPGGGGGSGGGGSEVQLLESGGGLVQPGGSLRLSCAASGFSFSSGYDMCWVRQAPGKG
LEWIACIAAGSAGITYDANWAKGRFTISRDNSKNTLYLQMNSLRAEDTAVYYCARSAFSFDYAMDLWGQGTLVTVSS
GGGGSGGGGSGGGGSGGGGSDIQMTQSPSTLSASVGDRVTITCQASQSISSHLNWYQQKPGKAPKLLIYKASTLASG
VPSRFSGSGSGTEFTLTISSLQPDDFATYYCQQGYSWGNVDNVFGGGTKVEIKGGGGSGGGGSGRSLVESGGGLVQP
GGSLRLSCTASGFTISSYHMQWVRQAPGKGLEYIGTISSGGNVYYASSARGRFTISRPSSKNTVDLQMNSLRAEDTA
VYYCARDSGYSDPMWGQGTLVTVSSGGGGSGGGGSGGGGSGGGGSDVVMTQSPSSVSASVGDRVTITCQASQNIRTY
LSWYQQKPGKAPKLLIYAAANLASGVPSRFSGSGSGTDFTLTISDLEPGDAATYYCQSTYLGTDYVGGAFGGGTKVE
IK
>SEQ ID 333 SI-55H12 chain B nt
GAAAATGTATTGACACAGAGCCCCGCCTCCCTCAGTGCCTCACCTGGGGAAAGGGTAACTATCACTTGCTCTGCATC
AAGCAGCGTCTCATACATGCATTGGTATCAACAAAAGCCTGGACAGGCCCCCAAGCTCTGGATATACGATACGAGCA
AGCTGGCTTCCGGCGTACCTAGCCGCTTCAGTGGTTCCGGCTCAGGCAACGATCACACCCTTACGATTTCCAGTATG
GAACCCGAAGATTTTGCAACTTATTATTGTTTCCAGGGGAGCGTGTACCCATTCACTTTCGGGCAGGGGACAAAAGT
GACCGTCCTAGGCGGTGGCGGTAGTGGGGGAGGCGGTTCTGGCGGCGGAGGGTCCGGCGGTGGAGGATCACAGGTCA
CATTGAAGGAATCTGGCCCCGGCCTTGTTCAGCCAGGACAGACCCTTAGCCTCACCTGTGCCTTCAGTGGTTTTTCT
CTTAGCACTAGCGGTATGGGGGTCGGCTGGATTCGGCAGCCTCCCGGCAAAGGTCTTGAGTGGTTGGCTCACATTTG
GTGGGACGACGACAAACGGTATAATCCTGCCTTGAAAAGTCGGCTGACCATTAGTAAGGATACCTCAAAAAATCAAG
TGTACTTGCAAATGAATAGCCTTGACGCCGAGGATACGGCTGTATATTATTGCGCTCGGATGGAACTCTGGTCTTAC
TACTTTGATTATTGGGGGCAGGGGACTCTCGTCACGGTCTCGAGTGGCGGTGGAGGGTCCGGCGGTGGTGGATCAGA
CGTCGTGATGACCCAGTCTCCTTCCACCCTGTCTGCATCTGTAGGAGACAGAGTCACCATCAATTGCCAAGCCAGTG
AGAGCATTAGCAGTTGGTTAGCCTGGTATCAGCAGAAACCAGGGAAAGCCCCTAAGCTCCTGATCTATGAAGCATCC
AAACTGGCATCTGGGGTCCCATCAAGGTTCAGCGGCAGTGGATCTGGGACAGAATTTACTCTCACCATCAGCAGCCT
GCAGCCTGATGATTTTGCAACTTATTACTGCCAAGGCTATTTTTATTTTATTAGTCGTACTTATGTAAATTCTTTCG
GCGGCGGGACCAAGGTGGAGATCAAACGTACGGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAGGT
GGATAACGCCCTCCAATCGGGTAACTCCCAGGAGAGTGTCACAGAGCAGGACAGCAAGGACAGCACCTACAGCCTCA
GCAGCACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGCGGTGGCGGTAGCGGTGGCGGCGGAAGTGGTGGCGG
AGGATCCCAGTCTGCCCTGACTCAGCCTGCCTCCGTGTCTGGGTCTCCTGGACAGTCGATCACCATCTCCTGCACTG
GAACCAGCAGTGACGTTGGTGGTTATAACTTTGTCTCCTGGTACCAACAACACCCAGGCAAAGCCCCCAAACTCATG
ATCTATGATGTCAGTGATCGGCCCTCAGGGGTGTCTGATCGCTTCTCCGGCTCCAAGTCTGGCAACACGGCCTCCCT
GATCATCTCTGGCCTCCAGGCTGACGACGAGGCTGATTATTACTGCAGCTCATATGGGAGCAGCAGCACTCATGTGA
TTTTCGGCGGAGGGACCAAGGTGACCGTCCTAGGTGGAGGCGGTTCAGGCGGAGGTGGTTCCGGCGGTGGCGGCTCC
GGTGGAGGCGGCTCTCAGGTGCAATTGCAGGAGTCGGGGGGAGGCCTGGTCAAGCCTGGAGGGTCCCTGAGTCTCTC
CTGTGCAGCCTCTGGATTCACCTTTAGTAGTTATTGGATGAGCTGGGTCCGCCAGGCTCCAGGGAAGGGGCTGGAGT
GGGTGGCCAACATAAACCGCGATGGAAGTGCGAGTTACTATGTGGACTCTGTGAAGGGCCGATTCACCATCTCCAGA
GACGACGCCAAGAACTCACTGTATCTGCAAATGAACAGCCTGAGAGCTGAGGACACGGCTGTGTATTACTGTGCGAG
AGATCGTGGGGTGGGCTACTTCGATCTCTGGGGCCGTGGCACCCTGGTCACCGTCTCTAGCTGA
>SEQ ID 334 SI-55H12 chain B aa
ENVLTQSPASLSASPGERVTITCSASSSVSYMHWYQQKPGQAPKLWIYDTSKLASGVPSRFSGSGSGNDHTLTISSM
EPEDFATYYCFQGSVYPFTFGQGTKVTVLGGGGSGGGGSGGGGSGGGGSQVTLKESGPGLVQPGQTLSLTCAFSGFS
LSTSGMGVGWIRQPPGKGLEWLAHIWWDDDKRYNPALKSRLTISKDTSKNQVYLQMNSLDAEDTAVYYCARMELWSY
YFDYWGQGTLVTVSSGGGGSGGGGSDVVMTQSPSTLSASVGDRVTINCQASESISSWLAWYQQKPGKAPKLLIYEAS
KLASGVPSRFSGSGSGTEFTLTISSLQPDDFATYYCQGYFYFISRTYVNSFGGGTKVEIKRTVAAPSVFIFPPSDEQ
LKSGTASVVCLLNNFYPREAKVQWKVDNALQSGNSQESVTEQDSKDSTYSLSSTLTLSKADYEKHKVYACEVTHQGL
SSPVTKSFNRGECGGGGSGGGGSGGGGSQSALTQPASVSGSPGQSITISCTGTSSDVGGYNFVSWYQQHPGKAPKLM
IYDVSDRPSGVSDRFSGSKSGNTASLIISGLQADDEADYYCSSYGSSSTHVIFGGGTKVTVLGGGGSGGGGSGGGGS
GGGGSQVQLQESGGGLVKPGGSLSLSCAASGFTFSSYWMSWVRQAPGKGLEWVANINRDGSASYYVDSVKGRFTISR
DDAKNSLYLQMNSLRAEDTAVYYCARDRGVGYFDLWGRGTLVTVSS

What is claimed is:

1. A multi-specific antibody-like protein having a N-terminal and a C-terminal, comprising in tandem from the N-terminal to the C-terminal,
a first binding domain (D1) at the N-terminal,
a Fab region as a second binding domain (D2) comprising a light chain,
a Fc region,
a third binding domain (D3) having a binding affinity to PD-L1, and
a fourth binding domain (D4) having a binding affinity to 4-1BB at the C-terminal,
wherein the light chain comprises a fifth binding domain (D5) covalently attached to the C-terminal and a sixth binding domain (D6) covalently attached to the N-terminal, and
wherein D1, D2, D5, and D6 each independently has a binding affinity to a tumor associated antigen (TAA) or CD3.

2. The multi-specific antibody-like protein of claim 1, wherein D1 or D2 has a binding affinity to CD3.

3. The multi-specific antibody-like protein of claim 1, comprising an amino acid sequence having sequence identity to SEQ ID NO. 176, 178, 106, 108, 332, 334, 324, 326, 328, or 330.

4. The multi-specific antibody-like protein of claim 1, wherein the D1, D3, D4, D5, and D6 is independently a scFv domain, a receptor, or a ligand.

5. The multi-specific antibody monomer of claim 1, wherein the D1 has a binding specificity against CD3, D2 has a binding specificity against EGFR, EGFRvIII, CD20, mesothelin, Claudin18.2, HER2, CD33or a combination thereof, D3 has a binding specificity against PD-L1, D4 has a binding specificity against 4-1BB, and D5 and D6 each independently has a binding specificity against a tumor associated antigen.

5. The multi-specific antibody monomer of claim 1, wherein D1 has a binding specificity against CD3, D2 has a binding specificity against a tumor associated antigen, D3 has a binding specificity against PD-L1, D4 has a binding specificity against 4-1BB, and D5 and D6 each independently has a binding specificity against NKG2D ligands, HER3, CD19 or a combination thereof.

7. The multi-specific antibody-like protein of claim 1, wherein the D1 has a binding specificity against EGFR, D2 has a binding specificity against CD3, D3 has a binding specificity against PD-L1, D4 has a binding specificity against 4-1BB, and D5 has a binding specificity against CD19, and D6 has a binding specificity against HER3.

8. The multi-specific antibody-like protein of claim 1, wherein the D1 has a binding specificity against EGFR, D2 has a binding specificity against CD3, D3 has a binding specificity against PD-L1, D4 has a binding specificity against 4-1BB, and D5 has a binding specificity against HER3, and D6 has a binding specificity against CD19.

9. The multi-specific antibody-like protein of claim 1, wherein the D1 has a binding specificity against CD3, D2 has a binding specificity against EGFR, D3 has a binding specificity against PD-L1, D4 has a binding specificity against 4-1BB, and D5 has a binding specificity against HER3, and D6 has a binding specificity against CD19.

10. A multi-specific antibody-like protein having a N-terminal and a C-terminal, comprising in tandem from the N-terminal to the C-terminal,
a first binding domain (D1) at the N-terminal,
a Fab region as a second binding domain (D2) comprising a light chain, wherein the light chain optionally comprises a fifth binding domain (D5) covalently attached to the C-terminal or a sixth binding domain (D6) covalently attached to the N-terminal,
a Fc region,
a third binding domain (D3), and
a fourth binding domain (D4) at the C-terminal,
wherein multi-specific antibody-like protein comprise an amino acid sequence having sequence identity to SEQ ID NO. 110, 112, 116, 118, 122, 124, 128, 130, 134, 136, 140, 142, 146, 148, 152, 154, 158, 160, 164, 166, 170, 172, 112,114, 118, 120, 124, 126, 130, 132, 136, 138, 142, 144, 148, 150, 154, 156, 160, 162, 166, 168, 172, 174, 34, 36, 38, 40, 42, 44, 46, 48, 50, 52, 54, 56, 302, 304, 306, or 308.

11. The multi-specific antibody-like protein of claim 10, wherein D2, D5, and D6 each independently has a binding affinity to a tumor associate antigen (TAA).

12. The multi-specific antibody-like protein of claim 10, wherein the D1 has a binding specificity against CD3, D2 has a binding specificity against a tumor-associated antigen, D3 has a binding specificity against PD-L1, and D4 has a binding specificity against 4-1BB.

13. The multi-specific antibody-like protein of claim 10, wherein the D1 has a binding specificity against CD3, D2 has a binding specificity against an antigen selected from a group consisting of EGFR, HER2, CD19, CD20, CD22, CD30, CD22, mesothelin, GD2, and Claudin 18.2, D3 has a binding specificity against PD-L1, and D4 has a binding specificity against 4-1BB.

14. The multi-specific antibody-like protein of claim 10, wherein the D1 has a binding specificity against CD3, D2 and D5 independently each has a binding specificity against a tumor-associated antigen, D3 has a binding specificity against PD-L1, and D4 has a binding specificity against 4-1BB.

15. The multi-specific antibody-like protein of claim 10, wherein the D1 has a binding specificity against CD3, D2 has a binding specificity against a tumor-associated antigen, D3 has a binding specificity against PD-L1, D4 has a binding specificity against 4-1BB, and D5 has a binding specificity against HER3.

16. The multi-specific antibody-like protein of claim 10, wherein the D1 has a binding specificity against CD3, D2 has a binding specificity against EGFR or EGFRvIII, D3 has a binding specificity against PD-L1, D4 has a binding specificity against 4-1BB, and D5 has a binding specificity against HER3.

17. The multi-specific antibody-like protein of claim 10, wherein the D1 has a binding specificity against CD3, D2 has a binding specificity against CD20, D3 has a binding specificity against PD-L1, D4 has a binding specificity against 4-1BB, and D5 has a binding specificity against CD19.

18. The multi-specific antibody-like protein of claim 10, wherein the D1 and D6 independently have a binding specificity against a tumor-associated antigen, D2 has a binding specificity against CD3, D3 has a binding specificity against PD-L1, and D4 has a binding specificity against 4-1BB.

19. The multi-specific antibody-like protein of claim 10, wherein the D1 has a binding specificity against EGFR, D2 has a binding specificity against CD3, D3 has a binding specificity against PD-L1, D4 has a binding specificity against 4-1BB, and D6 has a binding specificity against CD19.

20. A multi-specific antibody-like protein having a N-terminal and a C-terminal, comprising in tandem from the N-terminal to the C-terminal,
optionally a first binding domain (D1) at the N-terminal,
a second binding domain (D2) comprising a light chain, wherein the light chain optionally comprises a fifth binding domain (D5) covalently attached to the C-terminal, a sixth binding domain (D6) covalently attached to the N-terminal, or both,
a Fc region,
optionally a third binding domain (D3), and
optionally a fourth binding domain (D4) at the C-terminal,
wherein at least one of D1, D2, D3, D4, D5, and D6 is a NKG2D, and
wherein D1, D2, D3, D4, D5, and D6 each independently has a binding affinity to specificity against a T cell activating receptor, an immune cell receptor, an immune checkpoint molecule, a co-stimulation factor, a receptor of a leukocyte, a tumor antigen, a tumor associated antigen (TAA), a receptor of a tissue cell, a receptor of a cancer cell, or a combination thereof.

21. The multi-specific antibody-like protein of claim 20, wherein the D2 comprises a dimer connected to CL and CH1, wherein the dimer is NKG2D.

22. The multi-specific antibody-like protein of claim 20, comprising an amino acid sequence having sequence identity to SEQ ID NO. 196 or 198.

23. The multi-specific antibody-like protein of claim 20, wherein the binding domain for a T cell activating receptor is adjacent to the binding domain for a tumor associated antigen (TAA).

24. The multi-specific antibody-like protein of claim 20, wherein the D1, D2, D3, D4, D5 and D6 each independently has a binding specificity against an antigen selected from EGFR, HER2, HER3, EGFRvIII, ROR1, CD3, CD28, CEA, LMP1, LMP2A, Mesothelin, PSMA, EpCAM, glypican-3, gpA33, GD2, TROP2, NKG2D, NKG2D ligand, BCMA, CD19, CD20, CD33, CD123, CD22, CD30, PD-L1, PD1, OX40, 4-1BB, GITR, TIGIT, TIM-3, LAG-3, CTLA4, CD40, CD40L, VISTA, ICOS, BTLA, LIGHT, HVEM, CSF1R, CD73, CD39, CLDN18.2, DLL3, HLA-G, FcRH5, GPRC5D, LIV-1, MUC1, CD138, CD70, CD16, uPAR, Siglec-15, CD47, CD38, NKp46, PD-L2, CD160, LOX-1, SIRPα, CD27, and wherein the Fc domain comprises a human IgG Fc domain.

25. The multi-specific antibody-like protein of claim 20, wherein D1, D2, D3, and D4 each independently has a binding specificity against NKG2D ligands, CD3, PD-L1, 4-1BB or a combination thereof.

26. The multi-specific antibody-like protein of claim 20, comprising an amino acid sequence having sequence identity to SEQ ID NO. 2, 4, 6, 8, 10, 12, 14, 16, 18, 20, 22, 24, 26, 28, 58, 60, 62, 64, 66, 68, 70, 72, 74, 76, 184, 186, 188, 190, 192, 194, 196, 198, 200, 202, 204, 206, 208, 210, 78, 80, 82, 84, 86, 88, 30 or 32.

27. An isolated nucleic acid sequence, encoding an amino acid sequence of the multi-specific antibody-like protein of claim 1.

28. An expression vector, comprising the isolated nucleic acid sequence of claim 27.

29. A host cell comprising the isolated nucleic acid sequence of claim 28, wherein the host cell is a prokaryotic cell or a eukaryotic cell.

30. An immuno-conjugate comprising a cytotoxic agent or an imaging agent linked to the multi-specific antibody of claim 1 through a linker, wherein the linker comprises an ester bond, an ether bond, an amid bond, a disulphide bond, an imide bond, a sulfone bond, a phosphate bond, a phosphorus ester bond, a peptide bond, a hydrophobic poly(ethylene glycol) linker, or a combination thereof.

31. A pharmaceutical composition, comprising a pharmaceutically acceptable carrier and one of the multi-specific antibodies of claim 1.

32. A method of treating a human subject with a cancer, an autoimmune disease, or an infectious disease comprising administering to the subject an effective amount of the multi-specific antibody-like protein of claim 1.

33. (canceled)
